(19) United States
(12) Patent Application Publication
(10) Pub. No.: US 2018/0244643 A1
(43) Pub. Date: Aug. 30, 2018

(54) HIGHLY SENSITIVE DETECTION OF BIOMOLECULES USING PROXIMITY INDUCED BIOORTHOGONAL REACTIONS

(71) Applicant: The Regents of the University of California, Oakland, CA (US)

(72) Inventors: Neal K. Devaraj, La Jolla, CA (US); Haoxing Wu, San Diego, CA (US)

(21) Appl. No.: 15/540,428

(22) PCT Filed: Dec. 30, 2015
(86) PCT No.: PCT/US2015/068165

§ 371 Date: Jun. 28, 2017

Related U.S. Application Data
(60) Provisional application No. 62/097,934, filed on Dec. 30, 2014.

(51) Int. Cl. C07D 311/90; C07D 209/12; C07D 403/14; C07D 405/12; C07D 405/10; C07D 491/16; C12Q 1/6804; C12Q 1/6827; G01N 33/58; G01N 21/64

(52) U.S. Cl. CPC C07D 311/90; C12Q 2565/107; C07D 403/14; C07D 405/12; C07D 405/10; C07D 491/16; C12Q 1/6804; C12Q 1/6827; G01N 33/582; G01N 21/64; C12Q 2525/197; C12Q 2523/109; C12Q 2525/205; C12Q 2527/125; C12Q 2563/131; C12Q 2565/101; C07D 209/12

(57) ABSTRACT

There provided, inter alia, reagents and methods for modulation fluorescence of a tetrazine containing compound and/or a dienophile-containing compound. There is provided a method of detecting binding of a first affmity ligand and a second affinity ligand, the method includes the follow steps. Contacting a tetrazine-containing compound with a dienophile-containing compound, the tetrazine-containing compound comprising a first affinity ligand covalently attached to a tetrazine moiety and the dienophile-containing comprising a second affmity ligand covalently attached to a dienophile moiety.

### CROSS-REFERENCES TO RELATED APPLICATIONS

This application claims the benefit of U.S. Provisional Application No. 62/097,934, filed Dec. 30, 2014, the content of which is incorporated hereby by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant No. K01EB010078 awarded by the National Institutes of Health. The Government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 48537-546001WO_ST25.TXT, created Dec. 28, 2015, 10,920 bytes, machine format IBM-PC, MS-Windows operating system, is hereby incorporated herein by reference in its entirety and for all purposes.

### BACKGROUND OF THE INVENTION

Fluorogenic bioorthogonal ligations offer a promising route towards the fast and robust fluorescent detection of specific DNA or RNA sequences. There is tremendous interest in the use of fluorogenic reactions for detecting and imaging nucleic acids, especially specific DNA and RNA sequences. Applications include time-resolved imaging of transcription, detection of disease-related single nucleotide polymorphisms, and tracking RNA fragments such as microRNAs. Despite advances in the use of molecular beacons, aptamers and antisense agents, the rapid detection and imaging of oligonucleotides in live cells and physiologically relevant media remains challenging. Current methods, although powerful, suffer from numerous drawbacks. For example, previous ligation reactions have been hampered by slow kinetics and autohydrolysis, often relying on nucleophilic/electrophilic reactions, which allow cellular or solvent nucleophiles to compete for reactivity.

Tetrazine bioorthogonal cycloadditions benefit from rapid tunable reaction rates and high stability against hydrolysis in buffer and serum. Furthermore, tetrazines act as both a fluorescent quencher and a reactive group, minimizing the complexity of fluorogenic ligation probe design. Based on these properties, there is disclosed an oligonucleotide-templated fluorogenic tetrazine ligation approach for the rapid fluorescent detection of specific DNA and RNA sequences.

There are provided reagents and method which enable the homogenous no-wash detection of biomolecules (i.e., proteins, DNA, RNA, etc.) using affinity ligands (e.g., aptamers, antibodies, oligonucleotides, small molecules) that bind in close proximity in the presence of target biomolecules, eliciting a strong fluorescent response with high signal to background ratios. The target biomolecules can be detected at femtomolar concentrations. The methods have no requirement for enzymes or the use of enzymatic amplification. Signal is achieved and amplification is performed in the absence of enzymes. Applications of the reagents and methods disclosed herein include inter alfa point of care diagnostics, detection of pathogens, clinical diagnostic tests, imaging biomarkers, immunohistochemistry, molecular imaging/image guided surgery, and telomere and telomerase assays.

### BRIEF SUMMARY OF THE INVENTION

There is provided a method of detecting binding of a first affinity ligand and a second affinity ligand, the method includes the follow steps.

Contacting a tetrazine-containing compound with a dienophile-containing compound, the tetrazine-containing compound comprising a first affinity ligand covalently attached to a tetrazine moiety and the dienophile-containing comprising a second affinity ligand covalently attached to a dienophile moiety.

(ii) Allowing the first affinity ligand to bind to the second affinity ligand or allowing the first affinity ligand and the second affinity ligand to bind to a third affinity ligand.

(iii) Allowing the tetrazine moiety to react with the dienophile moiety to form a pyridazine moiety and a detectable compound (e.g., the product of step (ii) formed from binding of a first affinity ligand, a second affinity ligand, and a third affinity ligand). In embodiments, the pyridazine moiety is within the detectable compound (e.g. the pyridazine moiety forms part of a detectable compound). In embodiemtns, the pyridazine moiety forms part of a compound distinct form the detectable compound.

### BRIEF DESCRIPTION OF THE DRAWINGS

FIG. 1 provides a schematic for the characterization of d27-Tz.

FIG. 2 provides a schematic for the characterization of d27′-ABN.

FIG. 3 provides a schematic for the characterization of d21′-Tz.

FIG. 4 provides a schematic for the characterization of d21′-ABN.

FIG. 5 provides a schematic for the characterization of d21′-Cyp.

FIG. 6 provides a schematic for the characterization of mir21′-Tz.

FIG. 7 provides a schematic for the characterization of mir21′-ABN.

FIG. 8. Reaction kinetics measurement of 1 μM d27′-Tz with d27′-ABN and d27 in Tris-HCl buffer (pH═7.4) at 25° C.

FIG. 9 Fluorescence emission spectra of 1 μM 13BpTz2 with 13BpNd1 before and 1.5 hour after the adding of 27T, in Tris-HCl pH═7.4 buffer at 25 ° C.

FIG. 10. Comparison of fluorescence emission intensity between DrD reaction (d21′-Tz+d21′-ABN), and ligation reaction (d21′-Tz+d21′-Cyp) at different template concentration. (A) Normalized fluorescence intensity of tetrazine transfer reaction at 0.1 eq of template d21 after 7 h. (B) Normalized fluorescence intensity of ligation reaction at 0.1 eq of template d21 after 7 h. (C) Normalized fluorescence intensity of the tetrazine transfer reaction at 0.01 eq of template d21 after 7 h. (D) Normalized fluorescence intensity of ligation reaction at 0.1 eq of template d21 after 7 h. (E) Normalized fluorescence intensity of tetrazine transfer reaction at 0 eq of template d21 after 7 h.

FIG. 11 The normalized fluorescence emission signal of d21′-Tz and d21′-ABN incubated with perfect match template (d21), mismatch templates (d21a and d21b), and no template after 37 ° C. for 1.5 hour.

FIG. 12 The normalized fluorescence emission signal of d21′-Tz and d21′-ABN with 0.01 eq template (d21) in different additive reaction solution after 7 hours incubation.

FIG. 13. Oligonucleotide probe stability in DMEM. mir21′-Tz and mir21-ABN were incubated for different periods of time (shown under each column) in DMEM (Dulbecco's Modified Eagle Medium) at room temperature, then 1 eq of mir21 was added. Fluorescence intensity was measured after 1.5 hour incubation at 37 ° C. RNA concentrations were kept at 1 μM.

FIG. 14. Normalized fluorescence intensity of oligonucleotide probes mir21′-Tz and mir21′-ABN upon reaction with different cell lysates. Column D: 10 eq of unmodified oligonucleotide probe added as a competitive inhibitor.

FIG. 15. Normalized fluorescence intensity of oligonucleotide probe 10 BpMeTz2, 11 BpMeNd2 reacted with 0.01 eq of match or mismatch template after 7 hours incubation. Sequence legend: RT: SEQ ID NO:27; RM1: SEQ ID NO:28; RM2: SEQ ID NO:29.

FIG. 16 provides a reaction schemefor ESI-TOFMS characterization of d27′-Tz with d27′-ABN.

FIG. 17 provides a characterization scheme for the reaction products of d21′-Tz with d21′-ABN.

FIG. 18 provides a characterization scheme for the reaction products of mir21′-Tz with mir21′-ABN.

FIG. 19 provides an exemplary scheme depicting an inverse Diels-Alder reaction between 7-azabenzonorbornadiene derivatives as novel strained dienophiles with tetrazines to release dinitrogen and form a dihydropyridazine coupling adducts. The product dihydropyridazine does not remain a stable conjugate but instead spontaneously undergoes a retro-Diels-Alder reaction to aromatize and fragment. Along with loss of dinitrogen, the net result of this process is effectively a functional group transfer between the dienophile and the tetrazine.

FIG. 20 illustrates designed compounds Tz-NHS and ABN-NHS for oligonucleotide modification, which are obtained through straightforward NHS coupling chemistry.

FIG. 21 illustrates antisense probes that were designed such that the 5′ tetrazine and 3′ dienophile would be brought into close proximity when hybridized to a complementary template oligonucleotide strand.

FIG. 22 provides an exemplary signal amplification cycle.

FIGS. 23A-23B. FIG. 23A: Normalized fluorescence intensity of DNA-templated Tetrazine-Azanorbornadiene transfer reaction after 7 hours and the turn over numbers. FIG. 23B: Normalized fluorescence intensity of RNA-templated Tetrazine-Azanorbornadiene transfer reaction after 7 hours and the turn over numbers. The fluorescence intensities were reported as average of values from three experiments. Turnover numbers are on the top of each column.

FIGS. 24A-24D. FIG. 24A: SKBR³ cells that have been incubated with mir21′-Tz and mir21′-ABN for two hours. FIG. 24B: MCF-7 cells incubated with the probes for two hours. FIG. 24C: HeLa cells incubated with the probes for two hours. FIG. 24D: Mean fluorescence of 20 cells after two-hour incubation. Cells where randomly selected from bright field images and their boundaries were traced to get the mean fluorescence.

FIG. 25 depicts fluorescence intensity upon reaction of avidin using methods disclosed herein.

FIG. 26 depicts picomolar determination of target DNA by methods disclosed herein.

FIG. 27A illustrates various fluorescent moieties having a phenol or phenoxide group.

FIG. 27B illustrates phenyl vinyl ethers as novel dienophiles that can undergo tetrazine-mediated transfer (TMT) reactions.

FIG. 28A illustrates the reaction between VE-1 and dipyridyl tetrazine Tz-0 proceeded with high-efficiency, resulting two products Cy-1 and Pz-0.

FIG. 28B illustrates the change in fluorescence when Cy-1 was dissolved into PBS buffer: a 70-fold of fluorescence increase was observed compare to caged precursor VE-1

FIGS. 29A-29D. FIG. 29A illustrates the structures of vinyl ethers VE-2, VE-3, and VE-4 and tetrazine-containing compound Tz-1. FIG. 29B provides sequences of d31-mis (SEQ ID NO:35), d31 (SEQ ID NO:34), and r31 (SEQ ID NO:36). FIG. 29C shows outcomes of DNA templated bio-orthogonal reactions. FIG. 29D shows outcomes of RNA templated bio-orthogonal reactions.

FIG. 30 illustrates a kinetic experiment that was conducted under pseudo-first order conditions using a phenyl vinyl ether (VE-0, 250 mM) that was reacted with 3,6-di-(2-pyridyl)-s-tetrazine (Tz-0, 5 mM) in a 1 mL solution of DMSO/H₂O═9:1.

FIG. 31 provides an exemplary general schematic for oligonucleotide probe modification.

FIG. 32 provides an exemplary scheme for characterization of d31-Tz, which was characterized by HPLC and ESI-TOF-MS.

FIG. 33 provides an exemplary scheme for characterization of d31-VE2, which was characterized by HPLC and ESI-TOF-MS.

FIG. 34 provides an exemplary scheme for characterization of d31-VE3, which was characterized by HPLC and ESI-TOF-MS.

FIG. 35 provides an exemplary scheme for characterization of d31-VE4, which was characterized by HPLC and ESI-TOF-MS.

FIG. 36 provides an exemplary scheme for characterization of mRNA-Tz1, which was characterized by HPLC and ESI-TOF-MS.

FIG. 37 provides an exemplary scheme for characterization of mRNA-VE4, which was characterized by HPLC and ESI-TOF-MS.

FIG. 38 illustrates the increase of the fluorescence intensity when a solution of d31′-Tz and d31′-VE1 at 1 μM was reacted with 1 μM of d31 in a 100 mM Tris-HCl (pH═7.4) buffer containing 200 mM MgCl₂.

### DETAILED DESCRIPTION OF THE INVENTION

II. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a non-cyclic straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom (e.g. selected from the group consisting of O, N, P, S, Se and Si, and wherein the nitrogen, selenium, and sulfur atoms may optionally be oxidized, and the nitrogen heteroatom may optionally be quaternized). The heteroatom(s) O, N, P, S, Se, and Si may be placed at any interior position of the heteroalkyl group or at the position at which the alkyl group is attached to the remainder of the molecule. Examples include, but are not limited

to: —CH₂-CH₂—O—CH₃, —CH₂-CH₂—NH—CH₃, —CH₂-CH₂—N(CH₃)—CH₃, —CH₂—S—CH₂-CH₃, —CH₂-CH₂, —SO)—CH₃, —CH₂-CH₂—SO)₂-CH₃, —CH═CH—O—CH₃, —Si(CH₃)₃, —CH₂-CH═N—OCH₃, —CH═CH —N(CH₃)—CH₃, —O—CH₃, —O—CH₂-CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂-CH₂—S—CH₂-CH₂-— and —CH₂—S—CH₂-CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SeR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively.

Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms (e.g. selected from N, O, and S, wherein the nitrogen and sulfur atoms are optionally oxidized, and the nitrogen atom(s) are optionally quaternized). Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

The term “alkylsulfonyl,” as used herein, means a moiety having the formula —SO₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —SO)R′, —SO)₂R′, —SO)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —SO)R′, —SO)₂R′, —SO)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one or more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A—(CH₂),—B—, wherein A and B are independently —CRR′—, —O—, —NR—, —S—, —(O)—, —(O)₂—, —SO)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —SO)—, —SO)₂—, or —SO)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)-or, as (D)-or (L)- for amino acids, and individual isomers are encompassed within the scope of the present-invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R_{13A}, R^{13B}, R^{13C}, R^{13D} etc., wherein each of R_{13A}, R^{13B}, R^{13C}, R^{13D} etc. is defined within the scope of the definition of R¹³ and optionally differently.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “tetrazine” or “tetrazine moiety” refers in the customary sense to a six-membered ring containing four nitrogen atoms. Absent express indication otherwise, the term tetrazine as used herein refers to the isomer of tetrazine with formula 1,2,4,5-tetrazine. The term “symmetric” in the context of substitution of a chemical moiety, e.g., substitution of tetrazine, refers in the customary sense to disubstitution with the same substituent, e.g., 3,6-dimethyl-1,2,4,5-tetrazine. Conversely, the term “asymmetric” in this context refers to disubstitution with different substituents.

A “dienophile” or “dienophile moiety” as used herein refers in the customary sense to a substituted alkene capable or reacting with a tetrazine or tetrazine moiety to form a pyridazine or pyridazine moiety.

A “nitrile” refers to a organic compound having a —CN group.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

“NucR^{11C}, acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

“Synthetic mRNA” as used herein refers to any mRNA derived through non-natural means such as standard oligonucleotide synthesis techniques or cloning techniques. Such mRNA may also include non-proteinogenic derivatives of naturally occurring nucleotides. Additionally, “synthetic mRNA” herein also includes mRNA that has been expressed through recombinant techniques or exogenously, using any expression vehicle, including but not limited to prokaryotic cells, eukaryotic cell lines, and viral methods. “Synthetic mRNA” includes such mRNA that has been purified or otherwise obtained from an expression vehicle or system.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The sensitivity of the hybridization assays may be enhanced through use of a nucleic acid amplification system that multiplies the target nucleic acid being detected. Amplification can also be used for direct detection techniques. Examples of such systems include the polymerase chain reaction (PCR) system and the ligase chain reaction (LCR) system. Other methods include the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including TAQMAN® and molecular beacon probes can be used to monitor amplification reaction products in real time.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The word “protein” denotes an amino acid polymer or a set of two or more interacting or bound amino acid polymers.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally non-adherent or have been treated not to adhere to surfaces, for example by trypsinization. In some embodiments the cell is a cancer cell line such as SKBR³ or LS174T.

A “biomolecule” as used herein refers any molecule produced in a living cell or any synthetically derived molecule that mimics or is an analogue of a molecule produced in a living cell. Biomolecules herein include nucleotides, polynucleotides (e.g. RNA, DNA), amino acids, peptides, polypeptides, proteins, polysaccharides, lipids. glycans, and small molecules (e.g. vitamins, primary and secondary metabolites, hormones, neurotransmitters). Amino acids may include moieties other than those found in the naturally occurring 20 amino acids (e.g. selenocysteine, pyrrolysine, carnitine, ornithine, GABA, and taurine). Amino acids may also include non-proteinogenic functional groups (e.g. CF₃, N₃, F, NO₂). Likewise, polypeptides and proteins may contain such amino acids. “Polysaccharides” include mono-, di-, and oligo- saccharides including O- and N- glycosyl- linkages. Polysaccharides may include functional group moieties not commonly found in a cellular environment (e.g. cyclopropene, halogens, and nitriles). Lipids include amphipathic-, phospho-, and glycol- lipids and sterols such as cholesterol. An “amphipathic lipid” refers to a lipid having hydrophilic and hydrophobic characteristics. A “phospholipid” refers to a lipid bound to a phosphate group and carries a charge. Exemplary phospholipids include phosphatidic acid, phosphatidylethanolamine, phosphatidylcholine, phosphatidylserine, and phosphatidylinositol. A “glycolipid” refers to a lipid bound to a poly- or oligo-saccharide. Exemplary glycolipids include galactolipids, sulfolipids, glycosphingolipids, and glycosylphosphatidylinositol. Lipids may include substituents not commonly found in the cellular environment (e.g. cyclopropene, halogens, and nitriles). A “small molecule” as used herein refers to any small molecule produced naturally in a biological environment and may contain unnatural moieties or linkages not typically found in a cell but tolerated during processing within a cell (e.g. cyclopropene, halogens, nitriles).

A “detectable moiety” as used herein refers to a moiety that can be covalently or noncovalently attached to a compound or biomolecule that can be detected for instance, using techniques known in the art. The detection moiety may provide for imaging of the attached compound or biomolecule. The detection moiety may indicate the contacting between two compounds. Exemplary detectable moieties are fluorescent moieties, antibodies, reactive dyes, radio-labeled moieties, magnetic contrast agents, and quantum dots. In other embodiments, a detectable moiety may be detected using colorimetric detection methods. In still other embodiments, a detectable moiety produces a separate chemical species (e.g., singlet oxygen) that may be detected using techniques known in the art. Exemplary fluorescent moieties include fluorescein, BODIPY®, and cyanine dyes. Exemplary radionuclides include Fluorine-18, Gallium-68, and Copper-64. Exemplary magnetic contrast agents include gadolinium, iron oxide and iron platinum, and manganese.

A “fluorophore” as used herein refers to a moiety that emits light upon excitiation.

A “fluorophore precursor” as used herein refers to a moiety from which a fluorophore is produced. In embodiments, a fluorophore is produced from a precursor moiety by a chemical reaction (e.g., a cycloaddition reaction between a tetrazine-containing compound and a dienophile-containing compound as described herein).

A “fluorescent moiety” as used herein refers to a fluorophore and/or a fluorophore precursor.

A “water soluble moiety” as used herein refers to any moiety that enhances the water solubility of the compound or molecule to which it is bound. A water soluble moiety may alter the partitioning coefficient of a compound or molecule to which it is bound thereby making the molecule more or less hydrophilic.

### III. Methods and Compounds

In an aspect, the invention provides a method of detecting binding of a first affinity ligand and a second affinity ligand, the method including the following steps.
Contacting a tetrazine-containing compound with a dienophile-containing compound, said tetrazine-containing compound comprising a first affinity ligand covalently attached to a tetrazine moiety and said dienophile-containing comprising a second affinity ligand covalently attached to a dienophile moiety.
(ii) Allowing said first affinity ligand to bind to the second affinity ligand or allowing the first affinity ligand and the second affinity ligand to bind to a third affinity ligand.
(iii) Allowing the tetrazine moiety to react with the dienophile moiety to form a pyridazine moiety within a detectable compound (e.g., the product of step (ii) formed from binding of a first affinity ligand, a second affinity ligand, and a third affinity ligand).

In embodiments, the tetrazine-containing compound has a detectable moiety.

In embodiments, the dienophile-containing compound has a detectable moiety.

In embodiments, the tetrazine-containing compound has a chromogenic moiety that is rendered detectable (e.g., by measuring absorbance) upon formation of said pyridazine moiety.

In embodiments, the dienophile-containing compound has a chromogenic moiety that is rendered detectable (e.g., by measuring absorbance) upon formation of said pyridazine moiety.

In embodiments, the chromogenic moiety is 3,3′,5,5′ tetramethylbenzidine (MB); 3,3′,4,4′ diaminobenzidine (DAB); 4-chloro-1-naphthol (4CN); 2,2′-azino-di [3-ethylbenzthiazoline] sulfonate (ABTS); or o-phenylenediamine (OPD).

In embodiments, the tetrazine-containing compound has a moiety that is rendered detectable by the production of singlet oxygen upon formation of said pyridazine moiety.

In embodiments, the dienophile-containing compound has a moiety that is rendered detectable by the production of singlet oxygen upon formation of said pyridazine moiety.

In embodiments, the singlet oxygen is detected indirectly (e.g., using spectrophotometric, fluorescent or chemiluminescent probes).

In embodiments, the tetrazine-containing compound has a fluorescent moiety that is rendered detectable upon formation of said pyridazine moiety.

In embodiments, the dienophile-containing compound has a fluorescent moiety that is rendered detectable upon formation of said pyridazine moiety.

In embodiments, the fluorescent moiety is a non-protein fluorescent moiety.

In embodiments, the fluorescent moiety is an acridine moiety.

In embodiments, the fluorescent moiety is an Alexa Fluor® moiety.

In embodiments, the fluorescent moiety is an anthracene (e.g., an anthraquinone) moiety.

In embodiments, the fluorescent moiety is an arylmethine moiety.

In embodiments, the fluorescent moiety is a BODIPY® moiety.

In embodiments, the fluorescent moiety is a coumarin moiety.

In embodiments, the fluorescent moiety is a cyanine moiety. In embodiments, the fluorescent moiety comprises cyanine, indocarboycanine, merocyanine, oxacarbocyanine, or thiacarbocyanine.

In embodiments, the fluorescent moiety is a dansyl moiety.

In embodiments, the fluorescent moiety is an oxadiazole moiety.

In embodiments, the fluorescent moiety is an oxazine moiety.

In embodiments, the fluorescent moiety is a pyrene moiety.

In embodiments, the fluorescent moiety is a squaraine moiety. In embodiments, the fluorescent moiety comprises cyanine, indocarboycanine, merocyanine, oxacarbocyanine, or thiacarbocyanine.

In embodiments, the fluorescent moiety is a tetrapyrrole moiety.

In embodiments, the fluorescent moiety is a xanthene moiety. In embodiments, the fluorescent moiety comprises eosin, fluorescein, Oregon green, rhodamine, or Texas red.

In embodiments, the fluorescent moiety is quenched upon formation of said pyridazine moiety.

In embodiments, the fluorescent moiety is activated upon formation of said pyridazine moiety.

In embodiments, said tetrazine-containing compound has a structure according to formula (I),

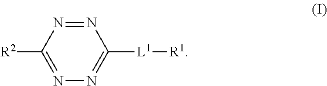

L¹ is independently a bond, —NR^{A}-, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ is independently a binding-detecting group comprising a detectable moiety (e.g., a fluorescent moiety).

L^{R1} is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Each R^{A} and R^{B} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently hydrogen or substituted or unsubstituted alkyl.

In embodiments, R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently -L^{R1}-X¹.

In embodiments, L¹ is independently substituted or unsubstituted alkenylene.

In embodiments, R¹ is a xanthene, cyanine, or a boron-dipyrromethene (BODIPY) group.

In embodiments, R¹ is a fluorescein or a rhodamine group.

In embodiments, the fluorescent moiety is a tricyclic structure having an aryl (e.g., a phenyl) substituent. In embodiments, the compound of formula (I) has the following structure,

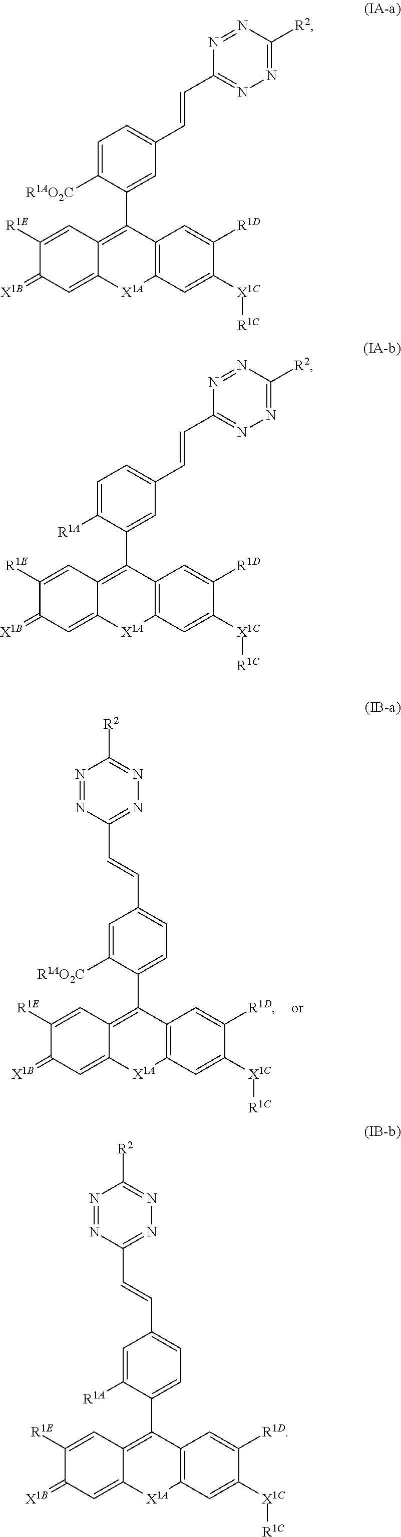

X^{1A} is independently O, SiMe₂, GeMe₂, SnMe₂, or TeO.
X^{1B} is independently O or NR^{1B}R^{1B}′.
X^{1C} is independently O or NR^{1C}′.
R^{1A} is independently hydrogen or substituted or unsubstituted alkyl.
Each of R^{1B}, R^{1B′}, R^{1C}, and R^{1C}′ is independently hydrogen or substituted or unsubstituted alkyl.
X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Each of R^{1D} and R^{1E} is independently hydrogen, halogen, —SO₃H, or -L^{R1}-X¹, wherein one and only one of R^{1D} and R^{1E} is -L^{R1}-X¹.

In embodiments, the compound of formula (I) has the following structure,

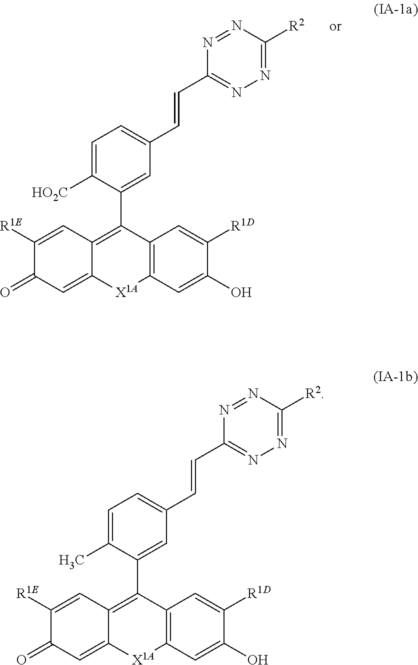

X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound of formula (I) has the following structure,

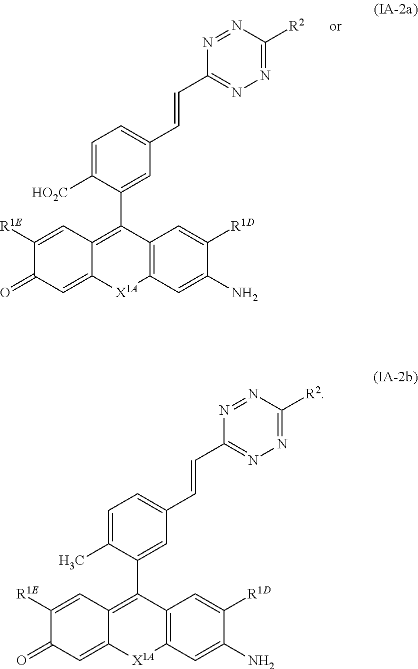

X′ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound of formula (I) has the following structure,

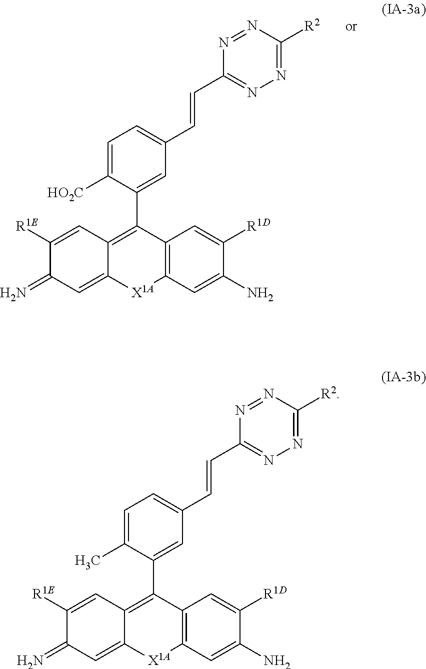

X′ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound of formula (I) has the following structure,

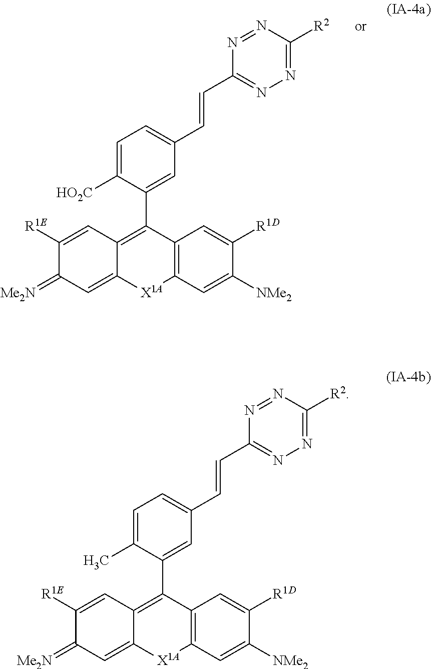

X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound of formula (I) has the following structure,

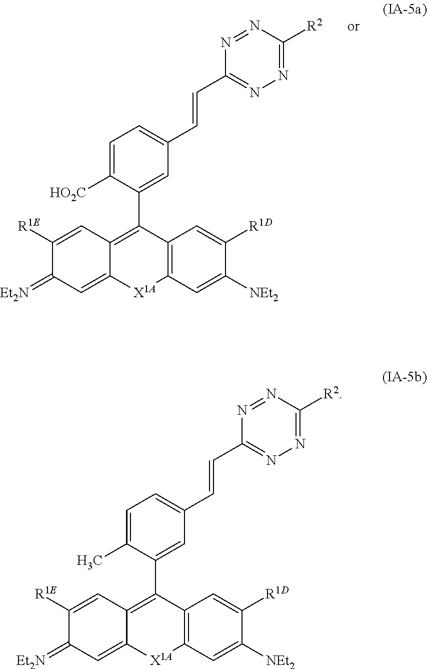

X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, NHNH₂, —ONH₂, —OCH₃, NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1D} and R^{1E} are independently hydrogen, F, Br, I, SO₃H, or -L^{R1}—X¹.

In embodiments, the compound of formula (I) has the following structure,

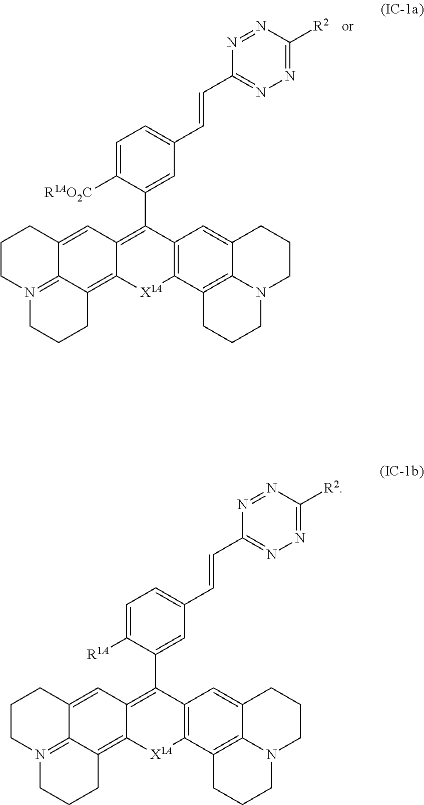

X^{1A} is independently O, SiMe₂, GeMe₂, SnMe₂, or TeO.
R² is -L^{R1}-X¹.
R^{1A} is independently hydrogen or substituted or unsubstituted alkyl.
L^{R1} is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
X¹ is said first affinity ligand.

In embodiments, X^{1A} is O, and R^{1A} is hydrogen.

In embodiments, the compound of formula (I) has the following structure,

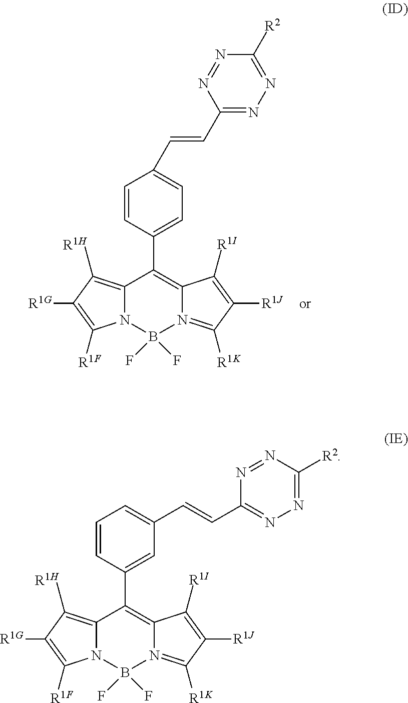

Each R^{1F}, R^{1G}, R^{1I}, R^{1J}, and R^{1K} is independently hydrogen, halogen, substituted or unsubstituted alkyl, substituted or unsubstituted aryl, substituted or unsubstituted heteroaryl, or -L^{R1}-X^{l}.
X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, NHNH₂, ONH₂, —OCH₃, NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
One and only one of RIF, _{R}ic-_{, R}ill_{, R}″_{, R}u_{, and R}IK _{is 4-,}R¹₋ₓl_{.}

In embodiments, the compound of formula (I) has the following structure,

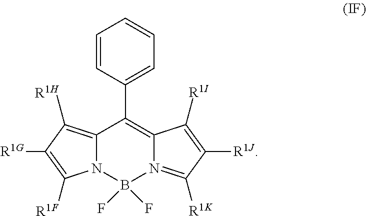

Each R^{1F}, R^{1G}, and R^{1H} is independently hydrogen, substituted or unsubstituted alkyl, substituted or unsubstituted aryl, substituted or unsubstituted heteroaryl, -L^{R1}-X^{l}, or

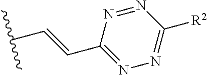

Each R^{1I}, R^{1J}, and R^{1K} is independently hydrogen, substituted or unsubstituted alkyl, substituted or unsubstituted aryl, substituted or unsubstituted heteroaryl, or -L^{R1}-X¹.
X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
One and only one of R^{1F}, R^{1G}, R^{1H}, R^{1I}, R^{1J}, and R^{1K} is -L^{R1}-X^{l}.
One and only one of R^{1F}, R^{1G}, and R^{1H} is

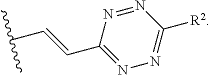

In embodiments, the compound of formula (I) has the following structure,

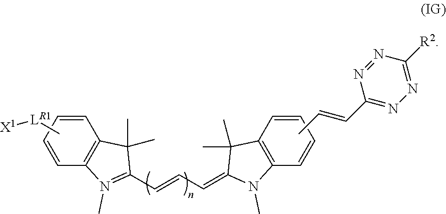

X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
n is 1, 2, or 3.

In embodiments, the compound of formula (I) has the following structure,

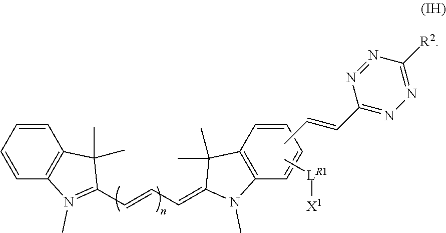

X¹ is said first affinity ligand.

R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

n is 1, 2, or 3.

In embodiments, the compound of formula (I) has the following structure,

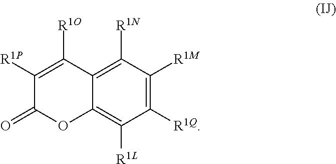

Each R^{1L}, R^{1M}, R^{1N}, R^{1O}, and R^{1P} is independently hydrogen, -L^{R1}-X¹, or

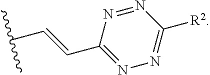

R^{1Q} is independently OR^{1Q′}, or NR^{1Q′}R^{1Q″}.
Each R^{1Q′} and R^{1Q″} is independently hydrogen or substituted or unsubstituted alkyl.
X¹ is said first affinity ligand.
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
One and only one of R^{1L}, R^{1M}, R^{1N}, R^{1O}, and R^{1P} is -L^{R1}-X¹.
One and only one of R^{1L}, R^{1M}, R^{1N}, R^{1O}, and R^{1P} is

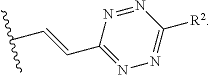

In embodiments, L^{R1} is independently a bond, substituted or unsubstituted alkylene, substituted or unsubstituted alkenylene, substituted or unsubstituted alkynylene, or substituted or unsubstituted heteroalkylene.

In embodiments, L^{R1} is independently a polyethylene glycol (PEG) linker, an amide linker, or a thioether linker.

In embodiments, said dienophile-containing compound has the following structure,

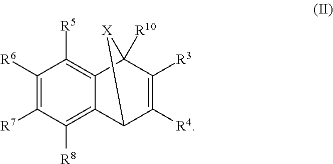

X is independently O, S, NR^{XA}, or CR^{XB}R^{XC}.
Each of R³ and R⁴ is independently hydrogen, substituted or unsubstituted alkyl, substituted or unsubstituted heteroalkyl, or -L²-R⁹.
R⁵ is independently hydrogen, halogen, —N₃, —NO₂, —CF₃, —CCl₃, —CBr₃, —Cl₃, —CN, —OR^{5A}, —NR^{5A}R^{5B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²—R⁹.
R⁶ is independently hydrogen, halogen, —N₃, —NO₂, —CF₃, —CCl₃, —CBr₃, —Cl₃, —CN, —OR^{6A}, —NR^{6A}R^{6B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²—R⁹.
R⁷ is independently hydrogen, halogen, —N₃, —NO₂, —CF₃, —CCl₃, —CBr₃, —Cl₃, —CN, —OR^{7A}, —NR^{7A}R^{7B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²-R⁹.
R⁸ is independently hydrogen, halogen, —N₃, —NO₂, —CF₃, —CCl₃, —CBr₃, —Cl₃, —CN, —OR^{8A}, —NR^{8A}, R^{8B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²-R⁹.
L² is independently a bond, —NR^{1.2}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
R⁹ is independently said second affinity ligand.
R¹⁰ is independently hydrogen, —OR^{10A}, —NR^{10A}R^{10B}, or substituted or unsubstituted alkyl.
Each R^{5A}, R_{5B}, R^{6A}, R^{6B}, R^{7A}, R^{7B}, R^{8A}, R^{8B}, R_{10A}, and R^{10B}, and is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Each R^{XA}, R^{XB}, and R^{XC} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²R⁹.
R^{1.2} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
One and only one of R³, R⁴, R⁵, R⁶, R⁷, R⁸, R^{XA}, R^{XB}, and R^{XC} is -L²-R⁹.

In embodiments, R³, R⁴, and R¹⁰ are hydrogen.

In embodiments, R⁵, R⁶, R⁷ and R⁸ are each hydrogen.

In embodiments, one of R⁵, R⁶, R⁷, and R⁸ is -L²-R⁹, and the others are hydrogen.

In embodiments, X is NR^{XA}.

In embodiments, R^{xA} is -L²-R⁹.

In embodiments, L² is independently a polyethylene glycol (PEG) linker, an amide linker, or a thioether linker.

In embodiments, said dienophile-containing compound has a detectable moiety (e.g., a fluorescent moiety) and said dienophile moiety is a vinyl ether functional group.

In embodiments, said dienophile-containing compound has a fluorescent moiety and said dienophile moiety is a vinyl ether functional group.

In embodiments, said fluorescent moiety is a xanthene, a coumarin, or a cyanine group.

In embodiments, said xanthene group is a fluorescein or a rhodamine group.

In embodiments, said dienophile-containing compound has a structure according to formula (III),

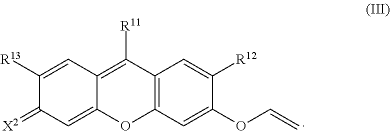

X² is O or NR^{X2}R^{X2}′.
R¹¹ is hydrogen, substituted or unsubsituted alkyl, substituted or unsubstituted heteroalkyl, or -L³-R¹⁴.
R¹² is independently hydrogen, halogen, —N₃, —NO₂, —CF₃, —CCl₃, —CBr₃, —Cl₃, —CN, —OR¹²A, —NR^{12A}R^{12B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
R¹³ is independently hydrogen, halogen, —N₃, —NO₂, —CF₃, —CCl₃, —CBr₃, —Cl₃, —CN, —OR^{13A}, —NR^{13A}R^{13B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
R¹⁴ is hydrogen or -L⁴-R¹⁵.
L³ is independently a bond, substituted or unsubstituted cycloalkylene, substituted or unsubstituted heterocycloalkylene, substituted or unsubstituted arylene, or substituted or unsubstituted heteroarylene.
L⁴ is independently a bond, —NR^{L4}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
R¹⁵ is independently said second affinity ligand.
Each R^{12A},R^{12B}, R^{13A}, R^{13B}, R^{L4}, R^{X2}, andR^{X2′}is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
   wherein one and only one of R¹², R¹³, and R¹⁴ is -L⁴-R¹⁵.

In embodiments, R¹¹ is hydrogen, substituted or unsubsituted alkyl, or substituted or unsubstituted heteroalkyl.

In embodiments, L⁴ is independently a polyethylene glycol (PEG) linker, an amide linker, or a thioether linker.

In embodiments, R¹³ is -L⁴-R¹⁵.

In embodiments, R¹² and R¹³ are independently hydrogen, F, Br, I, or SO₃H.

In embodiments, the compound of formula (III) has the following structure,

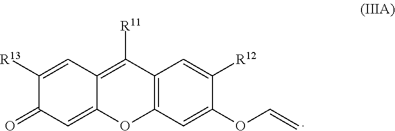

In embodiments, the compound of formula (III) has the following structure,

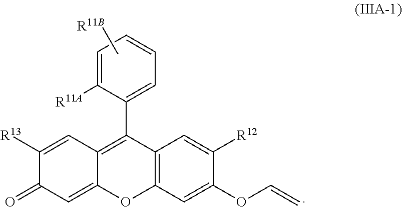

R^{11A} is independently hydrogen, substituted or unsubstituted alkyl, —CO₂R^{11C}, or -L⁴, -R¹⁵.
R^{11B} is independently hydrogen, halogen, —N₃, —NO₂, —CF₃, —CCl₃, —CBr₃, —Cl₃, —CN, —OR^{11D}R^{11E}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
Each R^{11C}, R^{11D}, and R^{11E} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound of formula (III) has the following structure,

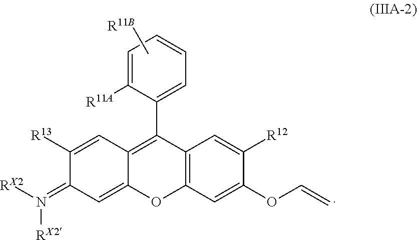

R^{11A} is independently hydrogen, substituted or unsubstituted alkyl, —CO₂R^{11C}, or -L⁴-R¹⁵.
R^{11B} is independently hydrogen, halogen, —N₃, —NO₂, —CF₃, —CCl₃, —CBr₃, —Cl₃, —CN, —OR^{11D}, —NR_{11D}R^{11E}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
Each R^{11C}, R^{11D}, and R^{11E} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Each R^{X2} andR^{X2}′ is independently hydrogen or unsubstituted alkyl.

In embodiments, R¹² and R¹³ are independently hydrogen, F, Br, I, or SO₃H.

In embodiments, said dienophile-containing compound has a structure according to formula (IV),

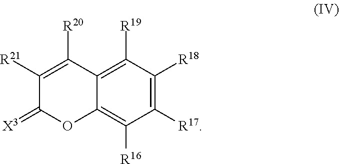

X³ is O or NR^{X3}R^{X3}′;
R¹⁶ is independently hydrogen, halogen, —N₃, —NO₂, —CF₃, —CCl₃, —CBr₃, —Cl₃, —CN, —OR¹⁶A, —NR^{16A}R^{16B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵.
R¹⁷ is independently hydrogen, halogen, —N₃, —NO₂, —CF₃, —CCl₃, —CBr₃, —Cl₃, —CN, —OR^{17A}, —NR^{17A}R^{17B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵.
R ¹⁸ is independently hydrogen, halogen, —N₃, —NO₂, —CF₃, —CCl₃, —CBr₃, —Cl₃, —CN, —OR¹⁸A, —NR^{18B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵.
R¹⁹ is independently hydrogen, halogen, —N₃, —NO₂, —CF₃, —CCl₃, —CBr₃, —Cl₃, —CN, —OR^{19A}, —NR^{19A}R^{19B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵.
R²⁰ is independently hydrogen, halogen, —N₃, —NO₂, —CF₃, —CCl₃, —CBr₃, —Cl₃, —CN, —OR^{20A}, —NR^{20A}R^{20B} , —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
R²¹ is independently hydrogen, halogen, —N₃, —NO₂, —CF₃, —CCl₃, —CBr₃, —Cl₃, —CN, —OR^{21A}, —NR^{21A}R^{21B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
Each R^{16A}, R^{16B}, R^{17A}, R^{17B}, R^{18A}, R^{18B}, R^{19A}, R^{19B}, R^{20A}, R^{20B}, R^{21A}, and R^{21B} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Each R^{X3} and R^{X3}′ is independently hydrogen or substituted or unsubstituted alkyl.
L⁴ is independently a bond, —NR¹⁴—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
R¹⁵ is independently said second affinity ligand.
One and only one of R¹⁶, R¹⁷, R¹⁸, and R¹⁹ is —OCH═CH₂.
One and only R¹⁶, R¹⁷, R₁₈, R¹⁹, R²⁰, and R²¹ is -L⁴-R¹⁵.

In embodiments, L⁴ is independently a polyethylene glycol (PEG) linker, an amide linker, or a thioether linker.

In embodiments, said dienophile-containing compound has a structure according to formula (V),

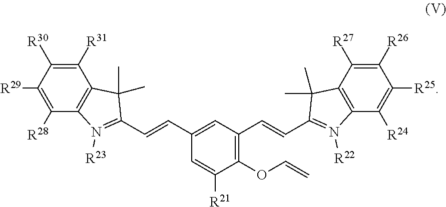

R²¹ is independently hydrogen, OR^{21A}, NR^{21A}R^{21B}, substituted or unsubstituted alkyl, or -L⁴-R¹⁵.
R²² and R²³ are independently hydrogen, substituted or unsubstituted alkyl, or -L⁴-R¹⁵.
Each R²⁴, R²⁵, R²⁶, R²⁷, R²⁸, R²⁹, R³⁰, and R³¹ is independently hydrogen, halogen, —N₃, —NO₂, —CF₃, —CCl₃, —CBr₃, —Cl₃, —CN, —OH, —NH₂, —NMc₂, —NEt₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
Each R^{21A} and R^{21B} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
L⁴ is independently a bond, —NR^{L4}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
R¹⁵ is independently said second affinity ligand.
One and only one of R²¹, R²², R²³, R²⁴, R²⁵, R²⁶, R²⁷, R²⁸, R²⁹, R^{30,} and R³¹ is -L⁴-R¹⁵.

In embodiments, L⁴ is independently a polyethylene glycol (PEG) linker, an amide linker, or a thioether linker.

In embodiments, R²² and R²³ are independently substituted or unsubstituted alkyl.

In embodiments, R²² is independently methyl, —(CH₂)ₙ₂₂SO₃H, or —(CH₂),ₙ₂₂CO₂H.

In embodiments, R²³ is independently methyl, —(CH₂)ₙ₂₃SO₃H, or —(CH₂),ₙ₂₃CO₂H.

In embodiments, n22 and n23 are independently an integer from 1 to 5.

In embodiments, each R²⁴, R²⁵, R²⁶, R²⁷, R²⁸, R²⁹, R³⁰, and R³¹ is independently hydrogen or —SO₃H.

In embodiments, R²¹ is OR^{21A}, NR^{21A}R^{21B}, —(CH₂)ₙ₂₁NR^{21C}R^{21D}, —(CH₂)ₙ₂₁COR^{21C}, or —(CH₂)ₙ₂₃CO₂R^{21C}, wherein each R^{21C} and R^{21D} is independently hydrogen or substituted or unsubstituted alkyl, and n21 is an integer from 1 to 5.

In embodiments, said dienophile-containing compound has a structure according to formula (VI-a) or (VI-b),

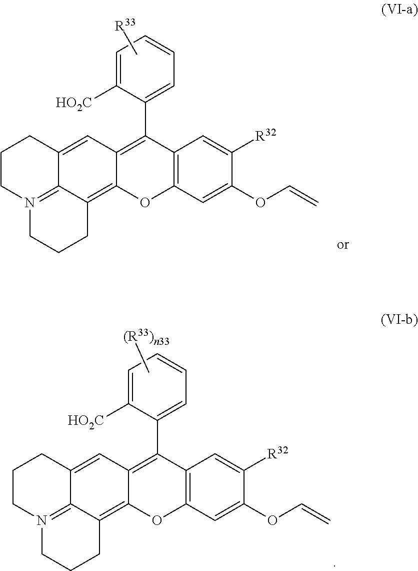

Each R³² and R³³ is independently hydrogen, halogen, —N₃, —NO₂, —CF₃, —CCl₃, —CBr₃, —Cl₃, —CN, —OH, —NH₂, —NMe₂, —NEt₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵.
L⁴ is independently a bond, —NR^{L4}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
R¹⁵ is independently said second affinity ligand.
n33 is independently 0, 1, or 2.
One and only one or R³² and R³³ is -L⁴-R^{15.}

In embodiments, L⁴ is independently a polyethylene glycol (PEG) linker, an amide linker, or a thioether linker.

In embodiments, said tetrazine-containing compound has a structure according to the following formula (VII),

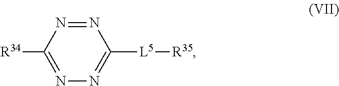

R³⁴ is independently hydrogen, halogen, —N₃, —NO₂, —CF₃, —CCl₃, —CBr₃, —Cl₃, —CN, —OR^{34A}, —NR^{34A}R^{34B}, —COOH, —CONH₂, —NO₂, —SH, —SO₂Cl, —SO₃H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L⁵ is independently a bond, —NR^{L5}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
R³⁵ is independently said first affinity ligand; and each R^{34A}, R^{34B}, and R^{L5} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, L⁵ is independently a polyethylene glycol (PEG) linker, an amide linker, or a thioether linker.

In embodiments, the tetrazine-containing compound comprises a first affinity ligand that is a biomolecule or nanomaterial.

In embodiments, the biomolecule is a nucleic acid (e.g. RNA or DNA), peptide, small molecules, protein (e.g., an antibody), lipid, or sugar.

In embodiments, the dienophile-containing compound comprises a second affinity ligand that is a biomolecule or nanomaterial.

In embodiments, the biomolecule is a nucleic acid (e.g. RNA or DNA), peptide, small molecules, protein (e.g., an antibody), lipid, or sugar.

In embodiments, the method further comprises the detection of a biomolecule.

In embodiments, said biomolecule is a nucleic acid (e.g., DNA, RNA, or PNA), protein (e.g., an antibody such as mAb, scFv, Fab, or Fab2), lipid, or sugar.

In embodiments, said biomolecule is DNA or RNA.

In embodiments, said protein of diagnostic utility is Hemoglobin Al c, Glucagon, Leptin, Haptoglobin, histidine rich protein II, pLDH, C reactive protein, or Epo.

In an aspect, the invention features a method of synthesizing a compound having a structure according to formula (I′),

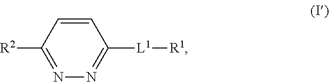

wherein each L^{l}, R¹, and R² is independently as defined herein, comprising contacting a compound having a structure according to formula (I),

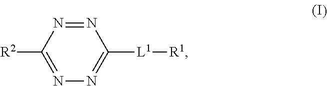

wherein each L^{l}, R¹, and R² is independently as defined herein, with a compound having a structure according to formula (II),

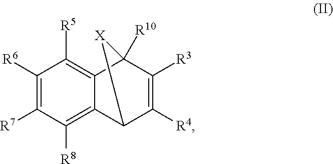

wherein each R³, R⁴, R⁵, R⁶, R⁷, R⁸, R¹⁰, and X is independently as defined herein.

In embodiments, the compound of formula (I′) has a structure according to the following formula,

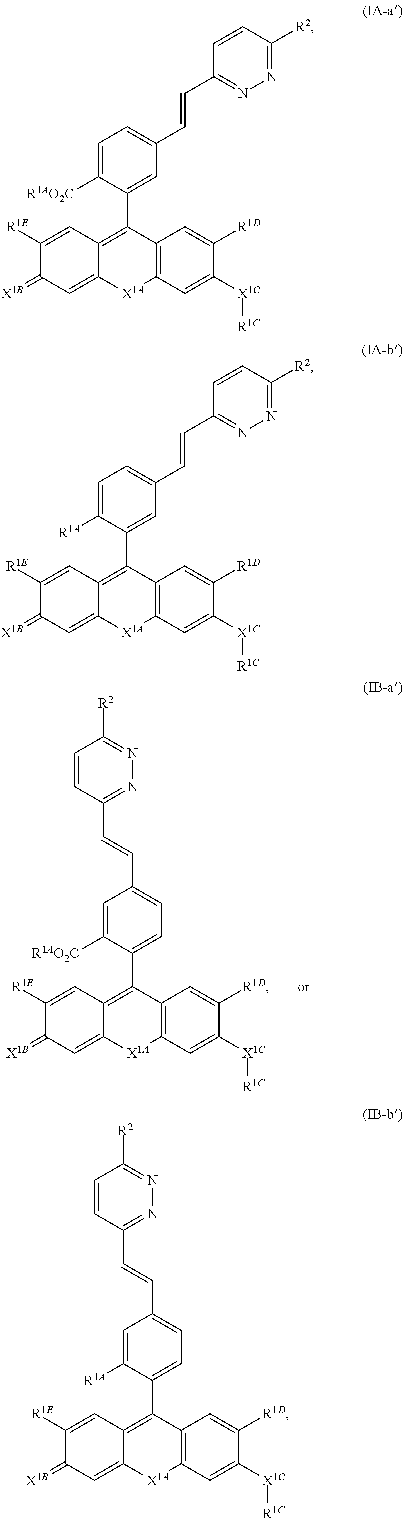

wherein each of R^{1C}, X_{1C}, R^{1D}, X^{1A}, X^{1B}, R^{1E}, R^{1A}, and R² is independently as defined herein.

In embodiments, the compound of formula (I′) has a structure according to the following formula,

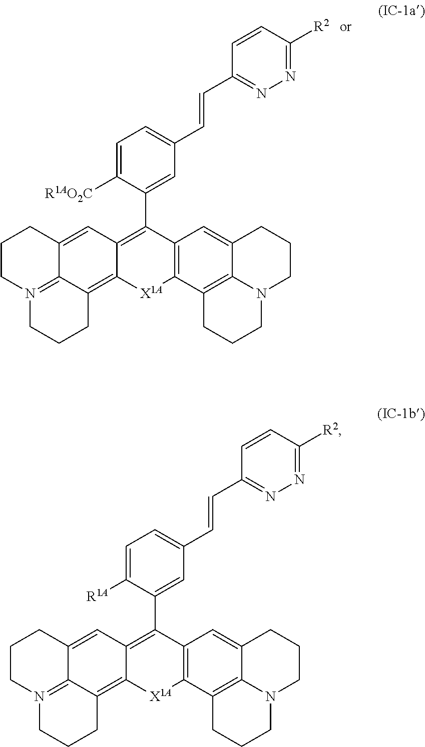

wherein each of R^{1A} and R² is independently as defined herein.

In embodiments, the compound of formula (I′) has a structure according to the following formula,

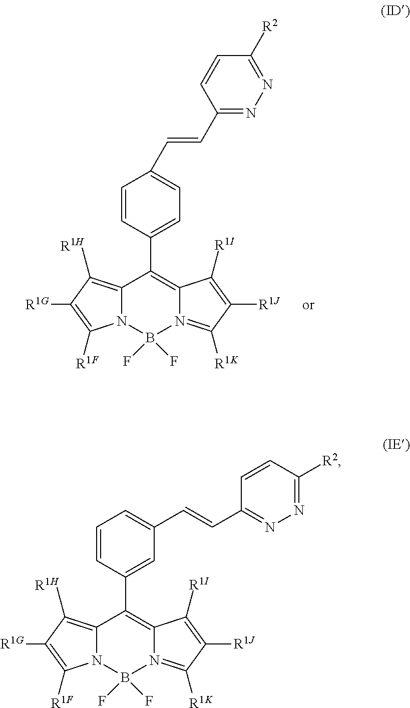

wherein each R², R^{1F}, R_{1G}, R^{1H}, R^{1I}, R^{1J}, and R^{1K} is as independently defined herein.

In embodiments, the compound of formula (I′) has a structure according to the following formula,

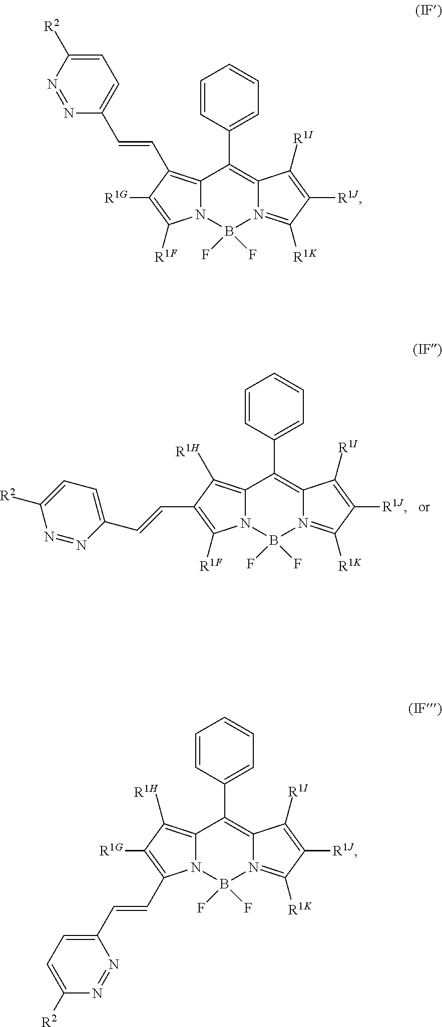

wherein each R², R^{1G}, R^{1H}, R^{1I}, R^{1J}, and R^{1K} is as independently defined herein.

In embodiments, the compound of formula (I′) has a structure according to the following formula,

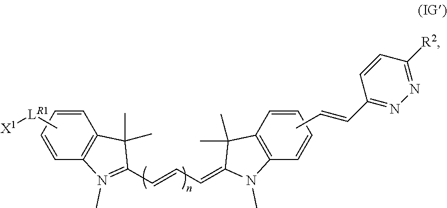

(1G′), wherein each R², X¹, L^{R1}, and n is independently as defined herein.

In embodiments, the compound of formula (I′) has a structure according to the following formula,

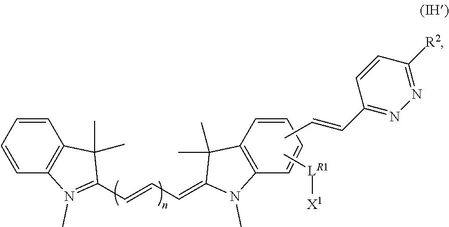

wherein each R², X¹, L^{R1}, and n is independently as defined herein.

In embodiments, the compound of formula (I′) has a structure according to the following formula,

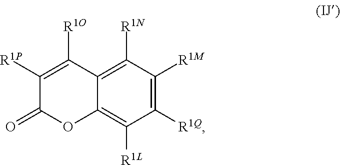

wherein each R^{1L}, R^{1Q}, R^{1M}, R^{1N}, R^{1O}, and R^{1P} is independently as defined herein, and one and only one of R^{1L}, R^{1M}, R^{1N}, R^{1O}, and R^{1P} is

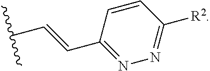

In embodiments, said contacting is in a cell.

In embodiments, the method further comprises detecting the compound of formula (I′).

In an aspect the invention features a method of synthesizing a compound having a structure according to formula (III′),

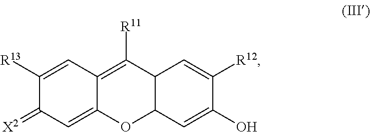

or a salt thereof, wherein each of X², R¹¹, R¹², and R¹³, is independently as defined herein, comprising contacting a compound having a structure according to formula (III),

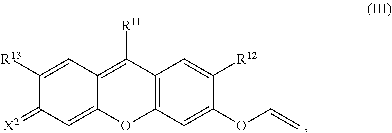

wherein each of X², R¹¹, R¹², and R¹³ is independently as defined herein,
with a compound having a structure according to formula (VII),

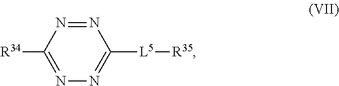

wherein each of L⁵, R³⁴, and R³⁵ is independently as defined herein.

In embodiments, the compound of formula (III′) has the following structure,

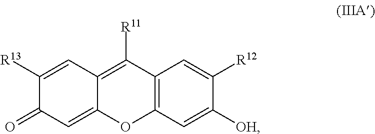

or a salt thereof.

In embodiments, the compound of formula (III′) has the following structure,

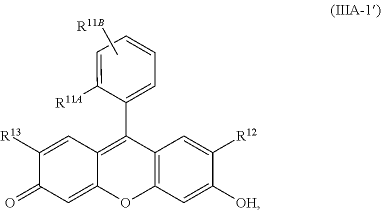

or a salt thereof.

In embodiments, the compound of formula (III′) has the following structure,

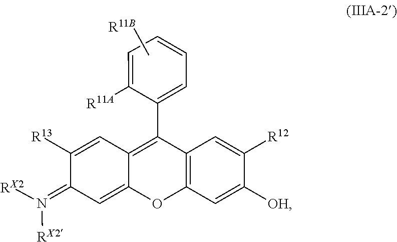

or a salt thereof

In embodiments, said contacting is in a cell.

In embodiments, the method further comprises detecting the compound of formula (III′).

In an aspect, the invention features a method of synthesizing a compound having a structure according to formula (IV′),

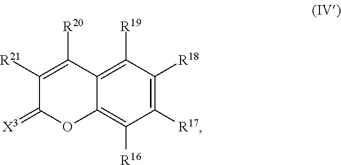

or a salt thereof, wherein each of R¹⁶, R¹⁷, R¹⁸, R¹⁹, R²⁰, R²¹, and X³ is independently as defined herein, and one and only one of R¹⁶, R¹⁷, R¹⁸, and R¹⁹ is —OH; comprising contacting a compound having a structure according to formula (III),

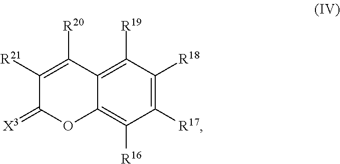

wherein each of R¹⁶, R¹⁷, R¹⁸, R¹⁹, R²⁰, R²¹, and X³ is independently as defined herein, and one and only one of R¹⁶, R¹⁷, R¹⁸, and R¹⁹ is —OCH═CH₂;
with a comnound having a structure according to formula (VII),

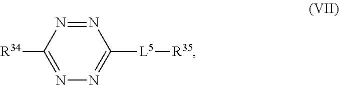

wherein each of L⁵, R³⁴, and R³⁵ is independently as defined herein.

In embodiments, said contacting is in a cell

In embodiments, the method further comprises detecting the compound of formula (IV′).

In an aspect, the invention features a method of synthesizing a compound having a structure according to formula (V′),

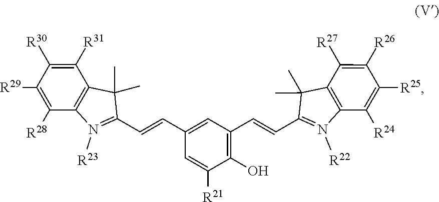

or a salt thereof, wherein each of R²¹, R²², R²³, R²⁴, R²⁵, R²⁶, R²⁷, R²⁸, R²⁹, R³⁰, and R³¹ is independently as defined herein, comprising contacting a compound having a structure according to formula (V),

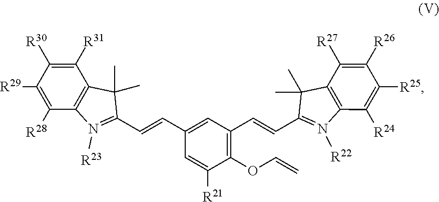

wherein each of R²¹, R²², R²³_{, R}²⁴, R²⁵, R²⁶, R²⁷, R²⁸, R²⁹, R³⁰, and R³¹ is independently as defined herein,
with a compound having a structure according to formula (VII),

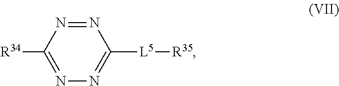

wherein each of L⁵, R³⁴, and R³⁵ is independently as defined herein.

In embodiments, said contacting is in a cell.

In embodiments, the method further comprises detecting the compound of formula (V′).

In an aspect, the invention features a method of synthesizing a compound having a structure according to formula (VI-a′),

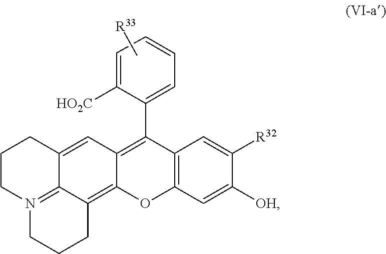

or a salt thereof, wherein each of R³² and R³³ is independently as defined herein,
comprising contacting a compound having a structure according to formula (VI-a),

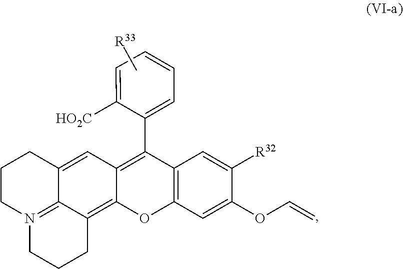

wherein each of R³² and R³³ is independently as defined herein, with a compound having a structure according to formula (VII),

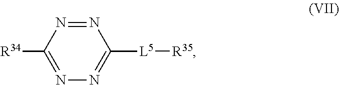

wherein each of L⁵, R³⁴, and R³⁵ is independently as defined herein.

In an aspect, the invention features a method of synthesizing a compound having a structure according to formula (VI-b′),

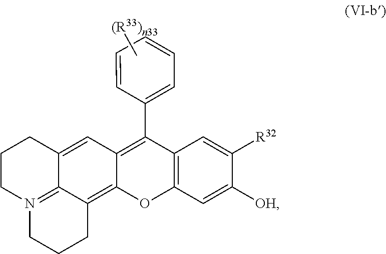

or a salt thereof, wherein each of R³², R³³, and n33 is independently as defined herein,
comprising contacting a compound having a structure according to formula (VI-b),

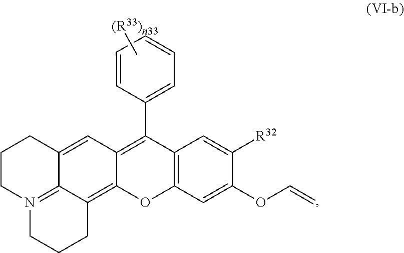

wherein each of R³², R³³, and n33 is independently as defined herein, with a compound having a structure according to formula (VII),

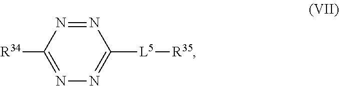

wherein each of L⁵, R³⁴, and R³⁵ is independently as defined herein.

In embodiments, said contacting is in a cell

In embodiments, the method further comprises detecting the compound of formula (VI′).

In an aspect, the invention features a compound having a structure according to formula (I).

In embodiments, the compound has a structure according to formula (IA-a) or (IA-b),

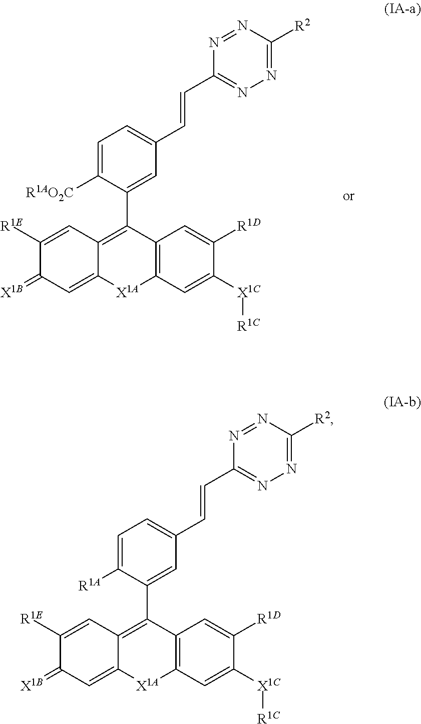

wherein X^{1A}, X^{1B}, X^{1C}, R^{1A}, R^{1C}, R^{1D}, R^{1E}, and R² are independently as described herein.

In embodiments, the compound has a structure according to formula (IB-a) or (IB-b),

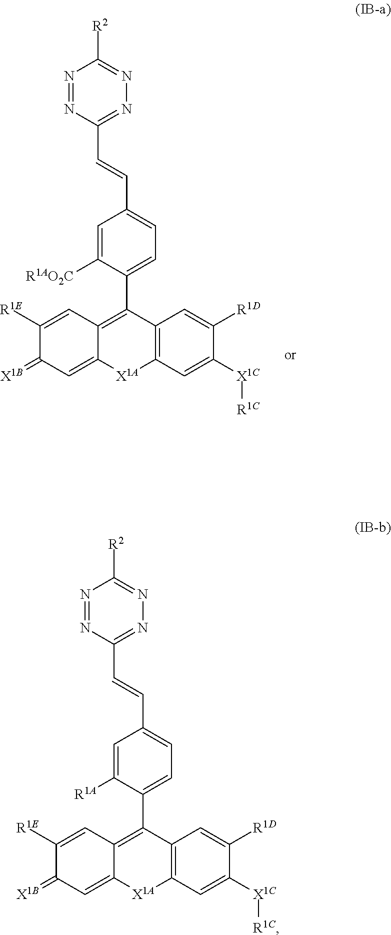

wherein X^{1A}, X^{1B}, X^{1C}, R^{1A}, R_{1C}, R^{1D}, R^{1E}, and R² are independently as described herein.

In embodiments, the compound has a structure according to formula (IA-1a) or (IA-1b),

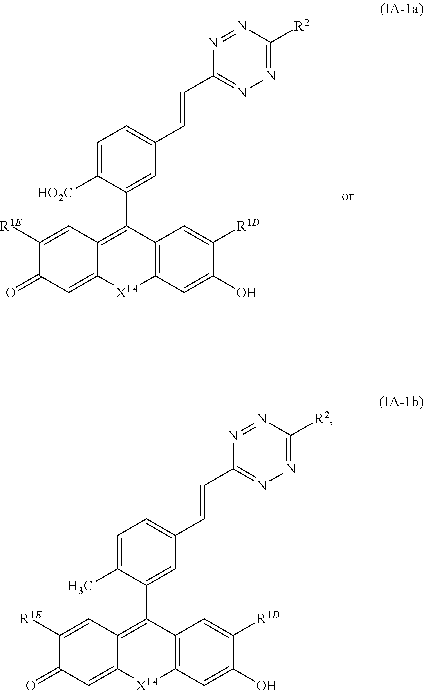

or a salt thereof, wherein X^{1A}, R^{1D}, R^{1E}, and R² are independently as described herein.

In embodiments, the compound has a structure according to formula (IA-2a) or (IA-2b),

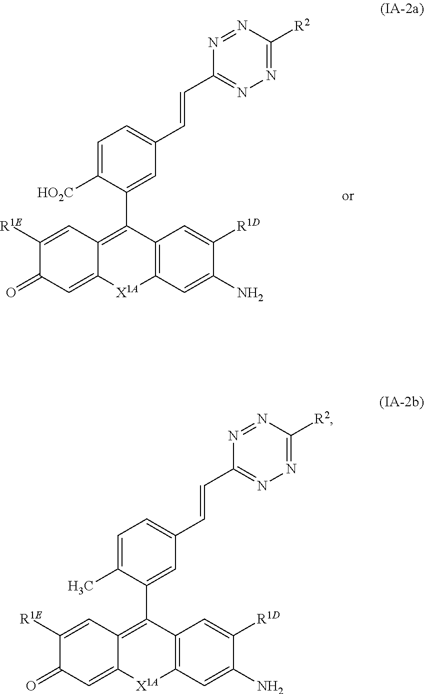

or a salt thereof, wherein X^{1A}, R^{1D}, R^{1E}, and R² are independently as described herein.

In embodiments, the compound has a structure according to formula (IA-3a) or (IA-3b),

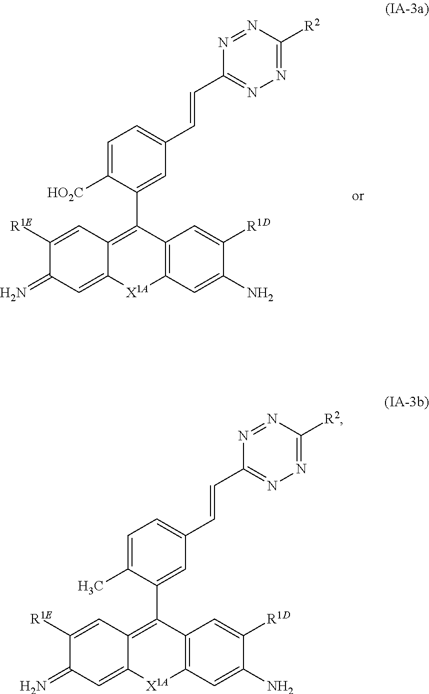

or a salt thereof, wherein X^{1A}, R^{1D}, R^{1E}, and R² are independently as described herein.

In embodiments, the compound has a structure according to formula (IA-4a) or (IA-4b),

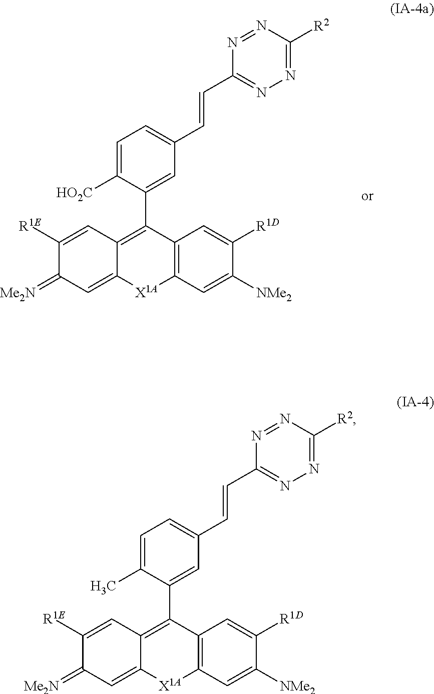

or a salt thereof, wherein X^{1A}, R^{1D}, R^{1E}, and R² are independently as described herein.

In embodiments, the compound has a structure according to formula (IA-5a) or (IA-5b),

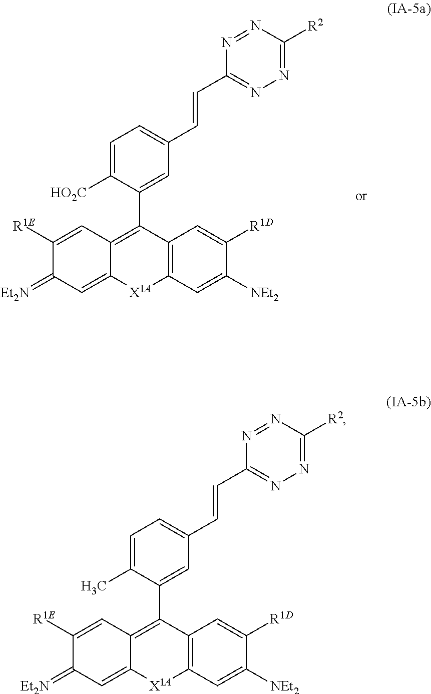

or a salt thereof, wherein X^{1A}, R^{1D}, R^{1E}, and R² are independently as described herein.

In embodiments, the compound has a structure according to formula (IC-1a) or (IC-1b),

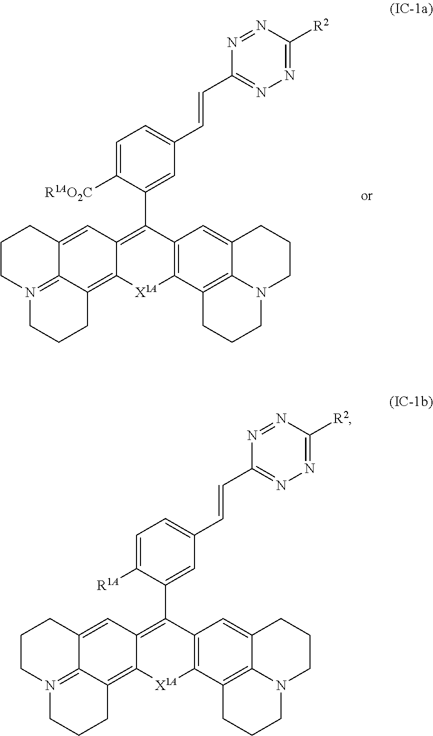

wherein X^{1A}, R^{1A}, and R² are independently as described herein.

In embodiments, the compound has a structure according to formula (ID),

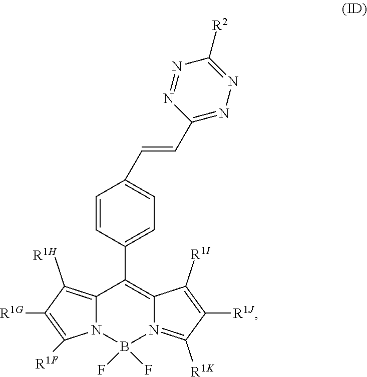

or a salt thereof, wherein R^{1F}, R^{1G}, R^{1H}, R^{1I}, R^{1J}, R^{1K}, and R² are independently as described herein.

In embodiments, the compound has a structure according to formula (IE),

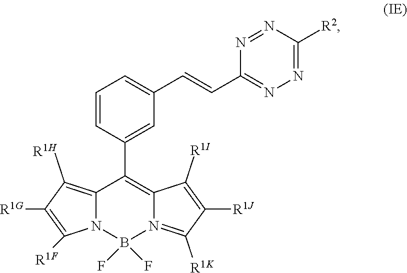

or a salt thereof, wherein R^{1F}, R^{1G}, R^{1H}, R^{1I}, R^{1J}, R^{1K} and R² are independently as described herein.

In embodiments, the compound has a structure according to formula (IF),

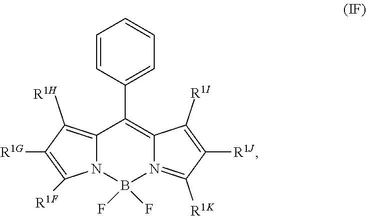

or a salt thereof, wherein R^{1F}, R^{1G}, R^{1H}, R^{1I}, R^{1J} and R^{1K} are independently as described herein.

In embodiments, the compound has a structure according to formula (IG),

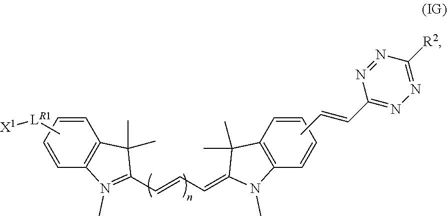

or a salt thereof, wherein n, R², X^{l}, and L^{R1} are independently as described herein.

In embodiments, the compound has a structure according to formula (IH),

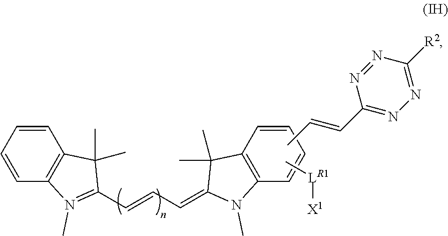

or a salt thereof, wherein n, R², X^{l}, and L^{R1} are independently as described herein.

In embodiments, the compound has a structure according to formula (IJ),

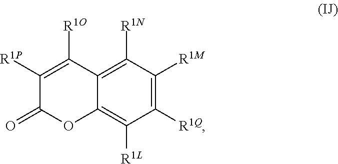

wherein R^{1L}, R^{1M}, R^{1N}, R^{1O}, R^{1P}, and R^{1Q} are independently as described herein.

In an aspect the invention features a compound having a structure according to formula (II),

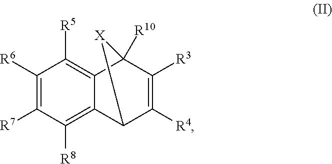

wherein R³, R⁴, R⁵, R⁶, R⁷, R⁸, and X are independently as described herein.

In an aspect the invention features a compound having a structure according to formula (III),

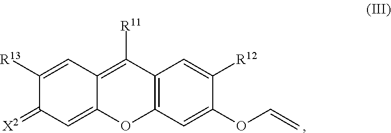

wherein X², R¹¹, R¹² , and R¹³, are independently as described herein.

In embodiments, the compound has a structure according to formula (IIIA),

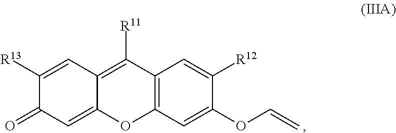

wherein R¹¹, R¹², and R¹³ are independently as described herein.

In embodiments, the compound has a structure according to formula (IIIA-1),

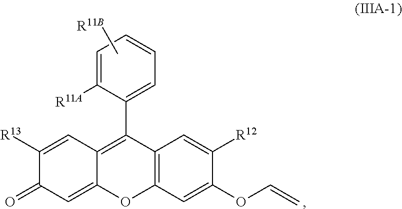

wherein R^{11A}, R^{11B}, R¹², and R¹³ are independently as described herein.

In embodiments, the compound has a structure according to formula (IIIA-2),

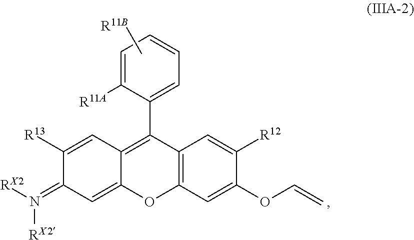

wherein R^{11A}, R^{11B}, R¹², R¹³, R^{X2}, and R^{X2} are independently as described herein.

In an aspect the invention features a compound having a structure according to formula (IV),

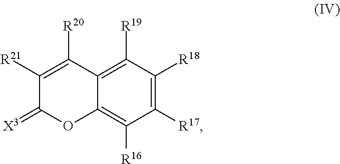

wherein X³, R¹⁶, R¹⁷, R¹⁸, R¹⁹, R²⁰, and R²¹ are independently as described herein.

In an aspect the invention features a compound having a structure according to formula (V),

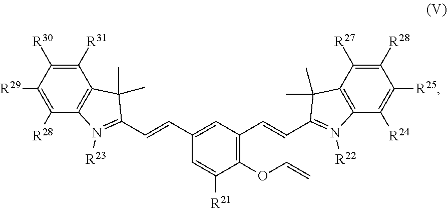

wherein R²¹, R²², R²³, R²⁴, R²⁵, R²⁶, R²⁷, R²⁸, R²⁹, R³⁰, and R³¹ are independently as described herein.

In an aspect the invention features a compound having a structure according to formula (VI-a) or formula (VI-b),

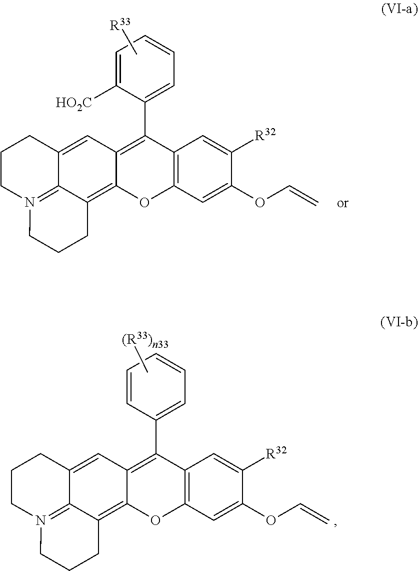

wherein R³², R³³, and n33 are independently as described herein.

In an aspect the invention features a compound having a structure according to formula (VII),

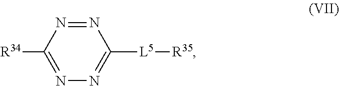

wherein L⁵, R³⁴, and R³⁵ are independently as described herein.

In an aspect the invention features a compound having a structure according to formula (I′),

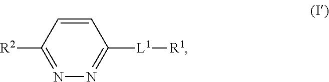

wherein L¹, R¹, and R² are independently as described herein.

In embodiments, the compound has a structure according to formula (IA-a′) or (IA-b′),

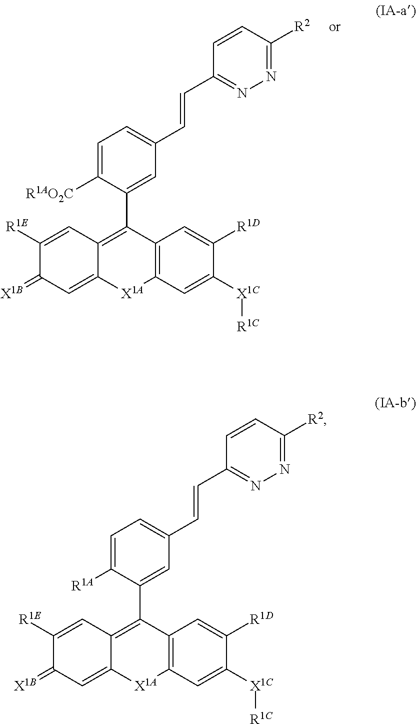

wherein X^{1A}, X^{1B}, X^{1C}, R^{1A}, R^{1C}, R^{1D}, R^{1E}, and R² are independently as described herein.

In embodiments, the compound has a structure according to formula (IB-a′) or (IB-b′),

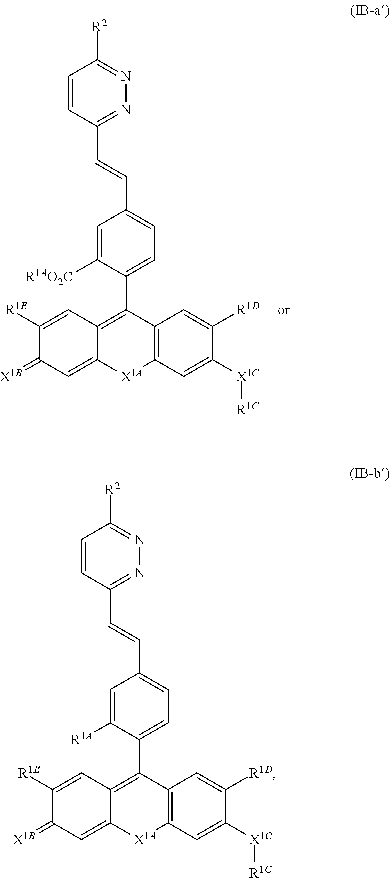

wherein X^{1A}, X^{1B}, X^{1C}, X^{1A}, R^{1C}, R^{1D}, R^{1E}, and R² are independently as described herein.

In embodiments, the compound has a structure according to formula (IC-1a′) or (IC-1b′),

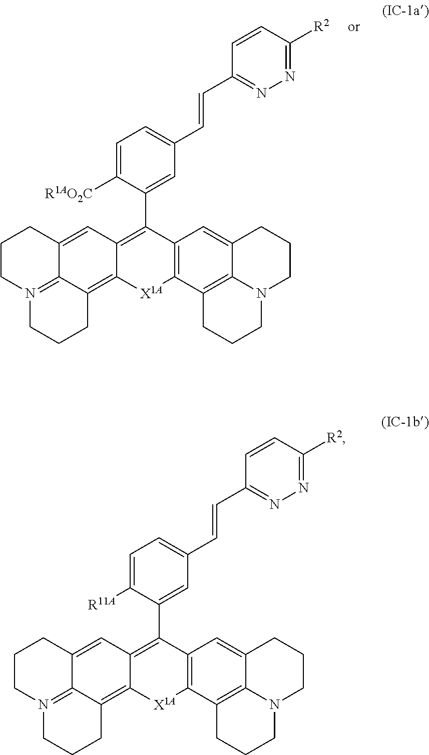

wherein X^{1A}, X^{1B}, X^{1C}, R^{1A}, R^{1C}, R^{1D}, R^{1E}, and R² are independently as described herein.

In embodiments, the compound has a structure according to formula (ID′),

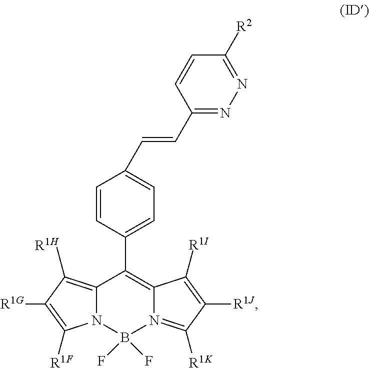

wherein R^{1F}, R^{1G}, R^{1J}, R^{1I}, R^{1J}, R^{1K} , and R² are independently as described herein.

In embodiments, the compound has a structure according to formula (IE′),

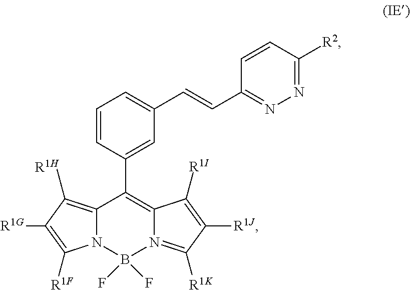

wherein R_{1F}, R^{1G}, R^{1J}, R^{1I}, R^{1J} , R^{1K}, and R² are independently as described herein.

In embodiments, the compound has a structure according to one of the following formulas,

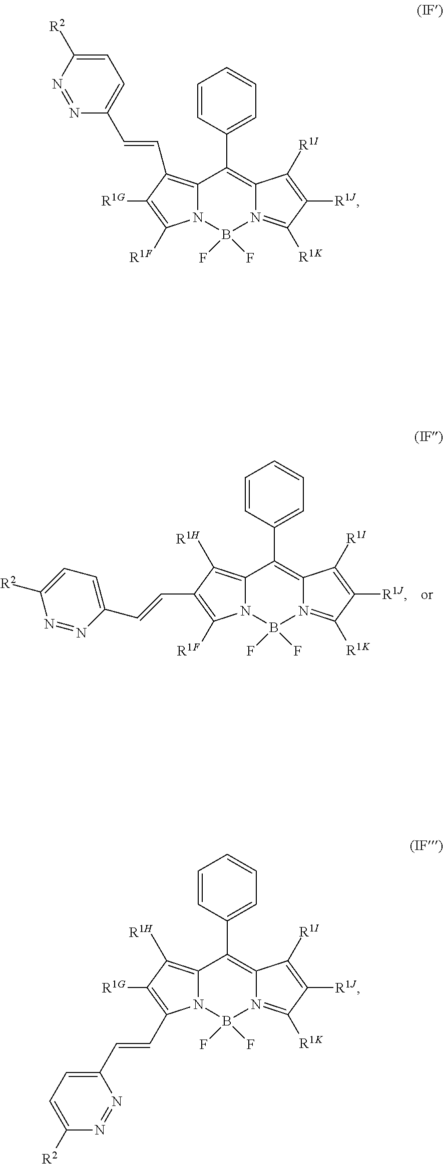

wherein R^{1F}, R^{1G}, R^{1J}, R^{1I}, R^{1J}, R^{1K} , and R² are independently as described herein.

In embodiments, the compound has a structure according to formula (IG′),

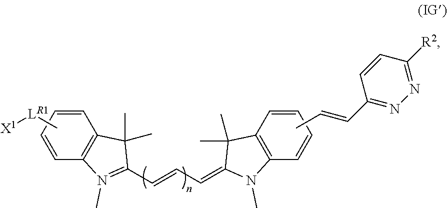

wherein n, R², X¹, and L^{R1} are independently as described herein.

In embodiments, the compound has a structure according to formula (IH′),

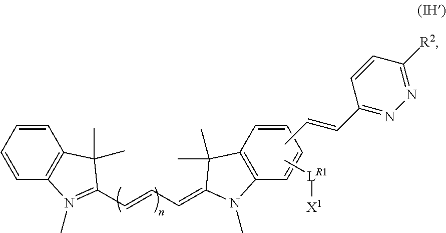

wherein n, R², X¹, and L^{R1} are independently as described herein.

In embodiments, the compound has a structure according to formula (IJ′),

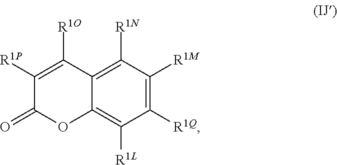

wherein R^{1L}, R^{1M}, R^{1N}_{, R}^{1O}, R^{1P}, and R^{1Q} are independently as described herein.

In an aspect, the invention features a compound having a structure according to formula (III′),

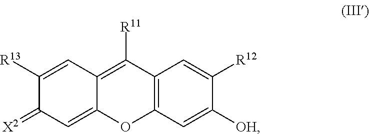

wherein X², R¹¹, R¹², and R¹³ areindependently as described herein.

In embodiments, compound has a structure according to formula (IIIA′),

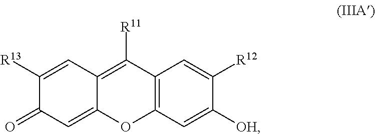

wherein R¹¹, R¹², and R¹³ are independently as described herein.

In embodiments, the compound has a structure according to formula (IIIA-1′),

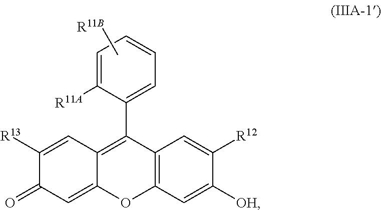

wherein R¹¹A, R^{11B}, R¹², and R¹³ are independently as described herein.

In embodiments, the compound has a structure according to formula (IIIA-2′),

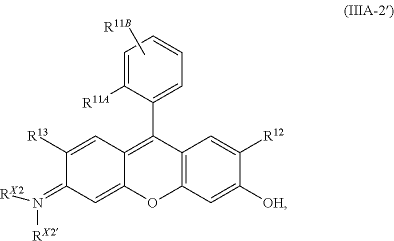

wherein R^{11A}, R^{11B}, R¹², R^{X2}, and R^{X2}′ are independently as described herein.

In an aspect, the invention features a compound having a structure according to formula (IV′),

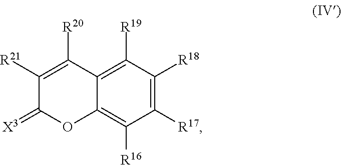

wherein R¹⁶, R¹⁷ , R¹⁸ , R¹⁹, R²⁰, R²¹, and X³ are independently as described herein.

In an aspect, the invention features a compound having a structure according to formula (V′),

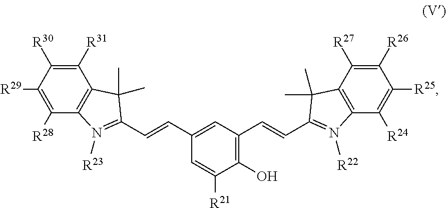

wherein R²¹, R²², R²³, R²⁴, R²⁵, R²⁶, R²⁷, R²⁸, R²⁹, R³⁰, and R³¹ are independently as described herein.

In an aspect, the invention features a compound having a structure according to formula (VI-a′) or (VI-b′),

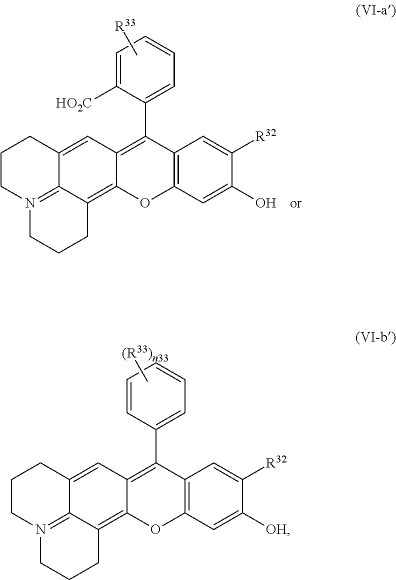

wherein R³², R³³, and n33 are independently as described herein.

In one aspect, there is provided a method of modulating fluorescence of a tetrazine containing compound and/or a dienophile-containing compound, wherein each tetrazine containing compound and dienophile-containing compound is attached to a separate affinity ligand, wherein the fluorescence modulation results from close proximity of the tetrazine containing compound and the dienophile-containing compound, and wherein the close proximity results from affinity ligand interactions with a biomolecule. In embodiments, fluorescence is increased. In embodiments, fluorescence is quenched.

In embodiments, the tetrazine is bonded to a fluorescent moiety, thereby quenching the fluorescent moiety. Exemplary fluorescent moieties include coumarin, xanthene, cyanine, or related dyes.

In embodiments, the dienophile is a cyclopropene, alkene, norbornadiene, azonorbornadiene, oxonorbornadiene, trans-cyclooctene, norbornene, or a vinyl ether.

In embodiments, fluorescence modulation is determined using FRET (fluorescence resonance energy transfer). In embodiments, both the tetrazine and dienophile are associated with quenched fluorescent moieties (e.g., quenched fluorescent moieties such as quenched fluorophores).

In embodiments, one or more of the affinity ligands is independently a probe. In embodiments, the probe is an oligonucleotide, peptide, small molecules, antibody, protein, lipid, sugar, or nanomaterial.

In embodiments, the probe targets a biomolecule. In embodiments, the target is nucleic acids(DNA/RNA), protein, antibody, lipid, or sugar.

In embodiments, the target is microRNA (e.g., mir-21), telomeres, genomic loci, non-coding RNA, mRNA, disease associated antibodies, or proteins of diagnostic utility (e.g., Hemoglobin Alc, Glucagon, Leptin, Haptoglobin, histidine rich protein II, pLDH, C reactive protein, Epo).

In embodiments, L¹ is independently a bond. In embodiments, L¹ is independently —NR^{A}—. In embodiments, L¹ is independently O. In embodiments, L¹ is independently S. In embodiments, L¹ is independently substituted or unsubstituted alkylene. In embodiments, L¹ is independently substituted or unsubstituted alkenylene. In embodiments, L¹ is independently substituted or unsubstituted alkynylene. In embodiments, L¹ is independently substituted or unsubstituted heteroalkylene. In embodiments, L¹ is independently substituted or unsubstituted cycloalkylene. In embodiments, L¹ is independently substituted or unsubstituted heterocycloalkylene. In embodiments, L¹ is independently substituted or unsubstituted arylene. In embodiments, L¹ is independently substituted or unsubstituted heteroarylene. In embodiments, L¹ is independently substituted alkylene. In embodiments, L¹ is independently substituted alkenylene. In embodiments, L¹ is independently substituted alkynylene. In embodiments, L¹ is independently substituted heteroalkylene. In embodiments, L¹ is independently substituted cycloalkylene. In embodiments, L¹ is independently substituted heterocycloalkylene. In embodiments, L¹ is independently substituted arylene. In embodiments, L¹ is independently substituted heteroarylene. In embodiments, L¹ is independently unsubstituted alkylene. In embodiments, L¹ is independently unsubstituted alkenylene. In embodiments, L¹ is independently unsubstituted alkynylene. In embodiments, L¹ is independently unsubstituted heteroalkylene. In embodiments, L¹ is independently unsubstituted cycloalkylene. In embodiments, L¹ is independently unsubstituted heterocycloalkylene. In embodiments, L¹ is independently unsubstituted arylene. In embodiments, L¹ is independently unsubstituted heteroarylene. In embodiments, L¹ is independently substituted or unsubstituted C₁-C₆ alkylene. In embodiments, L¹ is independently substituted or unsubstituted C₂-C₆ alkenylene. In embodiments, L¹ is independently substituted or unsubstituted C₂-C₆ alkynylene. In embodiments, L¹ is independently substituted or unsubstituted 2 to 6 membered heteroalkylene. In embodiments, L¹ is independently substituted or unsubstituted C₃-C₈ cycloalkylene. In embodiments, L¹ is independently substituted or unsubstituted 3 to 8 membered heterocycloalkylene. In embodiments, L¹ is independently substituted or unsubstituted C₆ arylene. In embodiments, L¹ is independently substituted or unsubstituted 5 to 6 membered heteroarylene. In embodiments, L¹ is independently substituted C₁-C₆ alkylene. In embodiments, L¹ is independently substituted C₂-C₆ alkenylene. In embodiments, L¹ is independently substituted C₂-C₆ alkynylene. In embodiments, L¹ is independently substituted 2 to 6 membered heteroalkylene. In embodiments, L¹ is independently substituted C₃-C₈ cycloalkylene. In embodiments,

L¹ is independently substituted 3 to 8 membered heterocycloalkylene. In embodiments, L¹ is independently substituted C₆ arylene. In embodiments, L¹ is independently substituted 5 to 6 membered heteroarylene. In embodiments, L¹ is independently unsubstituted C₁-C₆ alkylene. In embodiments, L¹ is independently unsubstituted C₂-C₆ alkenylene. In embodiments, L¹ is independently unsubstituted C₂-C₆ alkynylene. In embodiments, L¹ is independently unsubstituted 2 to 6 membered heteroalkylene. In embodiments, L¹ is independently unsubstituted C₃-C₈ cycloalkylene. In embodiments, L¹ is independently unsubstituted 3 to 8 membered heterocycloalkylene. In embodiments, L¹ is independently unsubstituted C₆ arylene. In embodiments, L¹ is independently unsubstituted 5 to 6 membered heteroarylene.

In embodiments, L^{R1} is independently a bond. In embodiments, L^{R1} is independently —NR^{B}—. In embodiments, L^{R1} is independently O. In embodiments, L^{R1} is independently S. In embodiments, L^{R1} is independently substituted or unsubstituted alkylene. In embodiments, L^{R1} is independently substituted or unsubstituted alkenylene. In embodiments, L^{R1} is independently substituted or unsubstituted alkynylene. In embodiments, L^{R1} is independently substituted or unsubstituted heteroalkylene. In embodiments, L^{R1} is independently substituted or unsubstituted cycloalkylene. In embodiments, L^{R1} is independently substituted or unsubstituted heterocycloalkylene. In embodiments, L^{R1} is independently substituted or unsubstituted arylene. In embodiments, L^{R1} is independently substituted or unsubstituted heteroarylene. In embodiments, L^{R1} is independently substituted alkylene. In embodiments, L^{R1} is independently substituted alkenylene. In embodiments, L^{R1} is independently substituted alkynylene. In embodiments, L^{R1} is independently substituted heteroalkylene. In embodiments, L^{R1} is independently substituted cycloalkylene. In embodiments, L^{R1} is independently substituted heterocycloalkylene. In embodiments, L^{R1} is independently substituted arylene. In embodiments, L^{R1} is independently substituted heteroarylene. In embodiments, L^{R1} is independently unsubstituted alkylene. In embodiments, L^{R1} is independently unsubstituted alkenylene. In embodiments, L^{R1} is independently unsubstituted alkynylene. In embodiments, L^{R1} is independently unsubstituted heteroalkylene. In embodiments, L^{R1} is independently unsubstituted cycloalkylene. In embodiments, L^{R1} is independently unsubstituted heterocycloalkylene. In embodiments, L^{R1} is independently unsubstituted arylene. In embodiments, L^{R1} is independently unsubstituted heteroarylene. In embodiments, L^{R1} is independently substituted or unsubstituted C₁-C₆ alkylene. In embodiments, L^{R1} is independently substituted or unsubstituted C₂-C₆ alkenylene. In embodiments, L^{R1} is independently substituted or unsubstituted C₂-C₆ alkynylene. In embodiments, L^{R1} is independently substituted or unsubstituted 2 to 6 membered heteroalkylene. In embodiments, L^{R1} is independently substituted or unsubstituted C₃-C₈ cycloalkylene. In embodiments, L^{R1} is independently substituted or unsubstituted 3 to 8 membered heterocycloalkylene. In embodiments, L^{R1} is independently substituted or unsubstituted C₆ arylene. In embodiments, L^{R1} is independently substituted or unsubstituted 5 to 6 membered heteroarylene. In embodiments, L^{R1} is independently substituted C₁-C₆ alkylene. In embodiments, L^{R1} is independently substituted C₂-C₆ alkenylene. In embodiments, L^{R1} is independently substituted C₂-C₆ alkynylene. In embodiments, L^{R1} is independently substituted 2 to 6 membered heteroalkylene. In embodiments, L^{R1} is independently substituted C₃-C₈ cycloalkylene. In embodiments, L^{R1} is independently substituted 3 to 8 membered heterocycloalkylene. In embodiments, L^{R1} is independently substituted C₆ arylene. In embodiments, L^{R1} is independently substituted 5 to 6 membered heteroarylene. In embodiments, L^{R1} is independently unsubstituted C₁-C₆ alkylene. In embodiments, L^{R1} is independently unsubstituted C₂-C₆ alkenylene. In embodiments, L^{R1} is independently unsubstituted C₂-C₆ alkynylene. In embodiments, L^{R1} is independently unsubstituted 2 to 6 membered heteroalkylene. In embodiments, L^{R1} is independently unsubstituted C₃-C₈ cycloalkylene. In embodiments, L^{R1} is independently unsubstituted 3 to 8 membered heterocycloalkylene. In embodiments, L^{R1} is independently unsubstituted C₆ arylene. In embodiments, L^{R1} is independently unsubstituted 5 to 6 membered heteroarylene.

In embodiments, R² is independently hydrogen. In embodiments, R² is independently halogen. In embodiments, R² is independently —N₃. In embodiments, R² is independently —NO₂. In embodiments, R² is independently —CF₃. In embodiments, R² is independently —CCl₃. In embodiments, R² is independently —CBr₃. In embodiments, R² is independently —Cl₃. In embodiments, R² is independently —CN. In embodiments, R² is independently —OH. In embodiments, R² is independently —NH₂. In embodiments, R² is independently —COOH. In embodiments, R² is independently —CONH₂. In embodiments, R² is independently —NO₂. In embodiments, R² is independently —SH. In embodiments, R² is independently —SO₂Cl. In embodiments, R² is independently —SO₃H. In embodiments, R² is independently —SO₄H. In embodiments, R² is independently —SO₂NH₂. In embodiments, R² is independently —NHNH₂. In embodiments, R² is independently —ONH₂. In embodiments, R² is independently —OCH₃. In embodiments, R² is independently NHCNHNH₂. In embodiments, R² is independently substituted or unsubstituted alkyl. In embodiments, R² is independently substituted or unsubstituted heteroalkyl. In embodiments, R² is independently substituted or unsubstituted cycloalkyl. In embodiments, R² is independently substituted or unsubstituted heterocycloalkyl. In embodiments, R² is independently substituted or unsubstituted aryl. In embodiments, R² is independently substituted or unsubstituted heteroaryl. In embodiments, R² is independently substituted alkyl. In embodiments, R² is independently substituted heteroalkyl. In embodiments, R² is independently substituted cycloalkyl. In embodiments, R² is independently substituted heterocycloalkyl. In embodiments, R² is independently substituted aryl. In embodiments, R² is independently substituted heteroaryl. In embodiments, R² is independently unsubstituted alkyl. In embodiments, R² is independently unsubstituted heteroalkyl. In embodiments, R² is independently unsubstituted cycloalkyl. In embodiments, R² is independently unsubstituted heterocycloalkyl. In embodiments, R² is independently unsubstituted aryl. In embodiments, R² is independently unsubstituted heteroaryl. In embodiments, R² is independently substituted or unsubstituted C₁-C₆ alkyl. In embodiments, R² is independently substituted or unsubstituted 2 to 6 membered heteroalkyl. In embodiments, R² is independently substituted or unsubstituted C₃-C₈ cycloalkyl. In embodiments, R² is independently substituted or unsubstituted 3 to 8 membered heterocycloalkyl. In embodiments, R² is independently substituted or unsubstituted C₆ aryl. In embodiments, R² is independently substituted or unsubstituted 5 to 6 membered heteroaryl. In embodiments, R² is independently substituted C₁-C₆ alkyl. In embodiments, R² is independently substituted 2 to 6 membered heteroalkyl. In embodiments, R² is independently substituted C₃-C₈ cycloalkyl. In embodiments, R² is independently substituted 3 to 8 membered heterocycloalkyl. In embodiments, R² is independently substituted C₆ aryl. In embodiments, R² is independently substituted 5 to 6 membered heteroaryl. In embodiments, R² is independently unsubstituted C₁-C₆ alkyl. In embodiments, R² is independently unsubstituted 2 to 6 membered heteroalkyl. In embodiments, R² is independently unsubstituted C₃-C₈ cycloalkyl. In embodiments, R² is independently unsubstituted 3 to 8 membered heterocycloalkyl. In embodiments, R² is independently unsubstituted C₆ aryl. In embodiments, R² is independently unsubstituted 5 to 6 membered heteroaryl.

In embodiments, each of R^{A} and R^{B} is independently hydrogen. In embodiments, each of R^{A} and R^{B} is independently substituted or unsubstituted alkyl. In embodiments, each of R^{A} and R^{B} is independently substituted or unsubstituted heteroalkyl. In embodiments, each of R^{A} and R^{B} is independently substituted or unsubstituted cycloalkyl. In embodiments, each of R^{A} and R^{B} is independently substituted or unsubstituted heterocycloalkyl. In embodiments, each of R^{A} and R^{B} is independently substituted or unsubstituted aryl. In embodiments, each of R^{A} and R^{B} is independently substituted or unsubstituted heteroaryl. In embodiments, each of R^{A} and R^{B} is independently substituted alkyl. In embodiments, each of R^{A} and R^{B} is independently substituted heteroalkyl. In embodiments, each of R^{A} and R^{B} is independently substituted cycloalkyl. In embodiments, each of R^{A} and R^{B} is independently substituted heterocycloalkyl. In embodiments, each of R^{A} and R^{B} is independently substituted aryl. In embodiments, each of R^{A} and R^{B} is independently substituted heteroaryl. In embodiments, each of R^{A} and R^{B} is independently unsubstituted alkyl. In embodiments, each of R^{A} and R^{B} is independently unsubstituted heteroalkyl. In embodiments, each of R^{A} and R^{B} is independently unsubstituted cycloalkyl. In embodiments, each of R^{A} and R^{B} is independently unsubstituted heterocycloalkyl. In embodiments, each of R^{A} and R^{B} is independently unsubstituted aryl. In embodiments, each of R^{A} and R^{B} is independently unsubstituted heteroaryl. In embodiments, each of R^{A} and R^{B} is independently substituted or unsubstituted C₁-C₆ alkyl. In embodiments, each of R^{A} and R^{B} is independently substituted or unsubstituted 2 to 6 membered heteroalkyl. In embodiments, each of R^{A} and R^{B} is independently substituted or unsubstituted C₃-C₈ cycloalkyl. In embodiments, each of R^{A} and R^{B} is independently substituted or unsubstituted 3 to 8 membered heterocycloalkyl. In embodiments, each of R^{A} and R^{B} is independently substituted or unsubstituted C₆ aryl. In embodiments, each of R^{A} and R^{B} is independently substituted or unsubstituted 5 to 6 membered heteroaryl. In embodiments, each of R^{A} and

R^{B} is independently substituted C₁-C₆ alkyl. In embodiments, each of R^{A} and R^{B} is independently substituted 2 to 6 membered heteroalkyl. In embodiments, each of R^{A} and R^{B} is independently substituted C₃-C₈ cycloalkyl. In embodiments, each of R^{A} and R^{B} is independently substituted 3 to 8 membered heterocycloalkyl. In embodiments, each of R^{A} and R^{B} is independently substituted C₆ aryl. In embodiments, each of R^{A} and R^{B} is independently substituted 5 to 6 membered heteroaryl. In embodiments, each of R^{A} and R^{B} is independently unsubstituted C₁-C₆ alkyl. In embodiments, each of R^{A} and R^{B} is independently unsubstituted 2 to 6 membered heteroalkyl. In embodiments, each of R^{A} and R^{B} is independently unsubstituted C₃-C₈ cycloalkyl. In embodiments, each of R^{A} and R^{B} is independently unsubstituted 3 to 8 membered heterocycloalkyl. In embodiments, each of R^{A} and R^{B} is independently unsubstituted C₆ aryl. In embodiments, each of R^{A} and R^{B} is independently unsubstituted 5 to 6 membered heteroaryl.

In embodiments, X^{1A} is independently O. In embodiments, X^{1A} is independently SiMe₂. In embodiments, X^{1A} is independently GeMe₂. In embodiments, X^{1A} is independently SnMe₂. In embodiments, X^{1A} is independently TeO.

In embodiments, X^{1B} is independently O. In embodiments, X^{1B} is independently NR^{1B}R^{1B′}.

In embodiments, X^{1C} is independently O. In embodiments, X^{1C} is independently NR^{1C′}.

In embodiments, each of R^{1A, R}^{1B}, R^{1B′}, R^{1C} , and R^{1C′} is independently hydrogen. In embodiments, each of R_{1A}, R^{1B}, R^{1B′}, R^{1C′} and is independently substituted or unsubstituted alkyl. In embodiments, R^{1A} is independently substituted alkyl. In embodiments, each of R^{1A}, R^{1B}, R^{1B′}, R^{1C}, and R^{1C′} is independently unsubstituted alkyl. In embodiments, each of R^{1A}, R^{1B}, R^{1B′}, R^{1C}, and R^{1C′} is independently substituted or unsubstituted C₁-C₆ alkyl. In embodiments, each of R^{1A}, R^{1B}, R^{1B′}, R^{1C} and R^{1C} is independently substituted C₁-C₆ alkyl. In embodiments, each of R^{1A}, R^{1B}, R^{1B′}, R^{1C} and R^{1C′} is independently unsubstituted C₁-C₆ alkyl.

In embodiments, each of R^{1D} and R^{1E} is independently hydrogen. In embodiments, each of R^{1D} and R^{1E} is independently halogen. In embodiments, each of R^{1D} and R^{1E} is independently —SO₃H. In embodiments, each of R^{1D} and R^{1E} is independently -L^{R1}-X¹.

In embodiments, each of R^{1F}, R^{1G}R^{1H}, R^{1I}, R^{1J}, and R^{1K} is independently hydrogen. In embodiments, each of R^{1F}, R^{1G}, R^{1H}, R^{1I}, R^{1J}, and R^{1K} is independently halogen. In embodiments, each of R^{1F}, R^{1G}, R^{1H}, R^{1I}, R^{1J}, and R^{1K} is independently substituted or unsubstituted alkyl. In embodiments, each of R^{1F}, R^{1G}, R^{1H}, R^{1I}, R^{1J}, and R^{1K} is independently substituted or unsubstituted aryl. In embodiments, each of R^{1F}, R^{1G},R^{1H}, R^{1I}, R^{1J}, and R^{1K} is independently substituted or unsubstituted heteroaryl. In embodiments, R^{1F} is independently -L^{R1}-X^{1.} In embodiments, each of R^{1F}, R^{1G}, R^{1H}, R^{1I}, R^{1J}, and R^{1K} is independently substituted alkyl. In embodiments, each of R^{1F}, R^{1G}, R^{1H}, R^{1I}, R^{1J}, and R^{1K} is independently substituted aryl. In embodiments, each of R^{1F}, R^{1G}, R^{1H}, R^{1I}, R^{1J} and R^{1K} is independently substituted heteroaryl. In embodiments, each of R^{1F}, R^{1G}, R^{1H}, R^{1I}, R^{1J}, and R^{1K} is independently unsubstituted alkyl. In embodiments, each of R^{1F}, R^{1G}, R^{1H}, R^{1I}, R^{1J} and R^{1K} is independently unsubstituted aryl. In embodiments, each of R^{1F}, R^{1G}, R^{1H}, R^{1I}, R^{1J}, and R^{1K} is independently unsubstituted heteroaryl. In embodiments, each of R^{1F}, R^{1G}, R^{1H}, R^{1I}, R^{1J}, and R^{1K} is independently substituted or unsubstituted C₁-C₆ alkyl. In embodiments, each of R^{1F}, R^{1G}, R^{1H}, R^{1I}, R^{1J}, and R^{1K} is independently substituted or unsubstituted C₆ aryl. In embodiments, each of R^{1F}, R^{1G}, R^{1H}, R^{1I}, R^{1J}, and R^{1K} is independently substituted or unsubstituted 5 to 6 membered heteroaryl. In embodiments, each of R^{1F}, R^{1G}, R^{1H}, R^{1I}, R^{1J}, and R^{1K} is independently substituted C₁-C₆ alkyl. In embodiments, each of R^{1F}, R^{1G}, R^{1H}, R^{1I}, R^{1J}, and R^{1K} is independently substituted C₆ aryl. In embodiments, each of R^{1F}, R^{1G}, R^{1H}, R^{1I}, R^{1J}, and R^{1K} is independently substituted 5 to 6 membered heteroaryl. In embodiments, each of R^{1F}, R^{1G} , R^{1H}, R^{1I}, R^{1J}, and R^{1K} is independently unsubstituted C₁-C₆ alkyl. In embodiments, each of R^{1F}, R^{1G}, R^{1H}, R^{1I}, R^{1J}, and R^{1K} is independently unsubstituted C₆ aryl. In embodiments, each of R^{1F}, R^{1G}, R^{1H}, R^{1I}, R^{1J}, and R^{1K} is independently unsubstituted 5 to 6 membered heteroaryl.

In embodiments, n is 1. In embodiments, n is 2. In embodiments, n is 3.

In embodiments, each of R^{1L}, R^{1M}, R^{1N}, R^{1O}, and R^{1P} is independently hydrogen. In embodiments, each of R^{1L}, R^{1M}, R^{1N}, R^{1O} and R^{1P} is independently -L^{R1}-X¹. In embodiments, each of R^{1L}, R^{1M}, R^{1N}, R^{1O}, and R^{1P} is independently

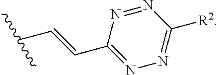

In embodiments, R^{1Q} is independently OR^{1Q′}. In embodiments, R^{1Q} is independently NR^{1Q′}, R^{1Q″}.

In embodiments, each of R^{1Q′} and R^{1Q″} is independently hydrogen. In embodiments, each of R^{1Q′} and R^{1Q″} is independently substituted or unsubstituted alkyl. In embodiments, each of R^{1Q′} and R^{1Q″} is independently substituted alkyl. In embodiments, each of R^{1Q′} and R^{1Q″} is independently unsubstituted alkyl. In embodiments, each of R^{1Q′} and R^{1Q″} is independently substituted or unsubstituted C₁-C₆ alkyl. In embodiments, each of R^{1Q′} and R^{1Q″} is independently substituted C₁-C₆ alkyl. In embodiments, each of R^{1Q′} and R^{1Q″} is independently unsubstituted C₁-C₆ alkyl.

In embodiments, X is independently O. In embodiments, X is independently S. In embodiments, X is independently NR^{XA}. In embodiments, X is independently CR^{XB}R^{XC}.

In embodiments, each of R³ and R⁴ is independently hydrogen. In embodiments, each of R³ and R⁴ is independently substituted or unsubstituted alkyl. In embodiments, each of R³ and R⁴ is independently substituted or unsubstituted heteroalkyl. In embodiments, each of R³ and R⁴ is independently -L²-R⁹. In embodiments, each of R³ and R⁴ is independently substituted alkyl. In embodiments, each of R³ and R⁴ is independently substituted heteroalkyl. In embodiments, each of R³ and R⁴ is independently unsubstituted alkyl. In embodiments, each of R³ and R⁴ is independently unsubstituted heteroalkyl. In embodiments, each of R³ and R⁴ is independently substituted or unsubstituted C₁-C₆ alkyl. In embodiments, each of R³ and R⁴ is independently substituted or unsubstituted 2 to 6 membered heteroalkyl. In embodiments, each of R³ and R⁴ is independently substituted C₁-C₆ alkyl. In embodiments, each of R³ and R⁴ is independently substituted 2 to 6 membered heteroalkyl. In embodiments, each of R³ and R⁴ is independently unsubstituted C₁-C₆ alkyl. In embodiments, each of R³ and R⁴ is independently unsubstituted 2 to 6 membered heteroalkyl.

In embodiments, R⁵ is independently hydrogen. In embodiments, R⁵ is independently halogen. In embodiments, R⁵ is independently —N₃. In embodiments, R⁵ is independently —NO₂. In embodiments, R⁵ is independently —CF₃. In embodiments, R⁵ is independently —CCl₃. In embodiments, R⁵ is independently —CBr₃. In embodiments, R⁵ is independently —Cl₃. In embodiments, R⁵ is independently —CN. In embodiments, R⁵ is independently —OR^{5A}. In embodiments, R⁵ is independently —NR^{5A}R^{5B}. In embodiments, R⁵ is independently —COOH. In embodiments, R⁵ is independently —CONH₂. In embodiments, R⁵ is independently —NO₂. In embodiments, R⁵ is independently —SH. In embodiments, R⁵ is independently —SO₂Cl. In embodiments, R⁵ is independently —SO₃H. In embodiments, R⁵ is independently —SO₄H. In embodiments, R⁵ is independently —SO₂NH₂. In embodiments, R⁵ is independently —NHNH₂. In embodiments, R⁵ is independently —ONH₂. In embodiments, R⁵ is independently —OCH₃. In embodiments, R⁵ is independently —NHCNHNH₂. In embodiments, R⁵ is independently substituted or unsubstituted alkyl. In embodiments, R⁵ is independently substituted or unsubstituted heteroalkyl. In embodiments, R⁵ is independently substituted or unsubstituted cycloalkyl. In embodiments, R⁵ is independently substituted or unsubstituted heterocycloalkyl. In embodiments, R⁵ is independently substituted or unsubstituted aryl. In embodiments, R⁵ is independently substituted or unsubstituted heteroaryl. In embodiments, R⁵ is independently -L²-R⁹. In embodiments, R⁵ is independently substituted alkyl. In embodiments, R⁵ is independently substituted heteroalkyl. In embodiments, R⁵ is independently substituted cycloalkyl. In embodiments, R⁵ is independently substituted heterocycloalkyl. In embodiments, R⁵ is independently substituted aryl. In embodiments, R⁵ is independently substituted heteroaryl. In embodiments, R⁵ is independently unsubstituted alkyl. In embodiments, R⁵ is independently unsubstituted heteroalkyl. In embodiments, R⁵ is independently unsubstituted cycloalkyl. In embodiments, R⁵ is independently unsubstituted heterocycloalkyl. In embodiments, R⁵ is independently unsubstituted aryl. In embodiments, R⁵ is independently unsubstituted heteroaryl. In embodiments, R⁵ is independently substituted or unsubstituted C₁-C₆ alkyl. In embodiments, R⁵ is independently substituted or unsubstituted 2 to 6 membered heteroalkyl. In embodiments, R⁵ is independently substituted or unsubstituted C₃-C₈ cycloalkyl. In embodiments, R⁵ is independently substituted or unsubstituted 3 to 8 membered heterocycloalkyl. In embodiments, R⁵ is independently substituted or unsubstituted C₆ aryl. In embodiments, R⁵ is independently substituted or unsubstituted 5 to 6 membered heteroaryl. In embodiments, R⁵ is independently substituted C₁-C₆ alkyl. In embodiments, R⁵ is independently substituted 2 to 6 membered heteroalkyl. In embodiments, R⁵ is independently substituted C₃-C₈ cycloalkyl. In embodiments, R⁵ is independently substituted 3 to 8 membered heterocycloalkyl. In embodiments, R⁵ is independently substituted C₆ aryl. In embodiments, R⁵ is independently substituted 5 to 6 membered heteroaryl. In embodiments, R⁵ is independently unsubstituted C₁-C₆ alkyl. In embodiments, R⁵ is independently unsubstituted 2 to 6 membered heteroalkyl. In embodiments, R⁵ is independently unsubstituted C₃-C₈ cycloalkyl. In embodiments, R⁵ is independently unsubstituted 3 to 8 membered heterocycloalkyl. In embodiments, R⁵ is independently unsubstituted C₆ aryl. In embodiments, R⁵ is independently unsubstituted 5 to 6 membered heteroaryl.

In embodiments, R⁶ is independently hydrogen. In embodiments, R⁶ is independently halogen. In embodiments, R⁶ is independently —N₃. In embodiments, R⁶ is independently —NO₂. In embodiments, R⁶ is independently —CF₃. In embodiments, R⁶ is independently —CCl₃. In embodiments, R⁶ is independently —CBr₃. In embodiments, R⁶ is independently —Cl₃. In embodiments, R⁶ is independently —CN. In embodiments, R⁶ is independently —OR^{6A}. In embodiments, R⁶ is independently —NR^{6A}R^{6B}. In embodiments, R⁶ is independently —COOH. In embodiments, R⁶ is independently —CONH₂. In embodiments, R⁶ is independently —NO₂. In embodiments, R⁶ is independently —SH. In embodiments, R⁶ is independently —SO₂Cl. In embodiments, R⁶ is independently —SO₃H. In embodiments, R⁶ is independently —SO₄H. In embodiments, R⁶ is independently —SO₂NH₂. In embodiments, R⁶ is independently —NHNH₂. In embodiments, R⁶ is independently —ONH₂. In embodiments, R⁶ is independently —OCH₃. In embodiments, R⁶ is independently —NHCNHNH₂. In embodiments, R⁶ is independently substituted or unsubstituted alkyl. In embodiments, R⁶ is independently substituted or unsubstituted heteroalkyl. In embodiments, R⁶ is independently substituted or unsubstituted cycloalkyl. In embodiments, R⁶ is independently substituted or unsubstituted heterocycloalkyl. In embodiments, R⁶ is independently substituted or unsubstituted aryl. In embodiments, R⁶ is independently substituted or unsubstituted heteroaryl. In embodiments, R⁶ is independently -L²-R⁹. In embodiments, R⁶ is independently substituted alkyl. In embodiments, R⁶ is independently substituted heteroalkyl. In embodiments, R⁶ is independently substituted cycloalkyl. In embodiments, R⁶ is independently substituted heterocycloalkyl. In embodiments, R⁶ is independently substituted aryl. In embodiments, R⁶ is independently substituted heteroaryl. In embodiments, R⁶ is independently unsubstituted alkyl. In embodiments, R⁶ is independently unsubstituted heteroalkyl. In embodiments, R⁶ is independently unsubstituted cycloalkyl. In embodiments, R⁶ is independently unsubstituted heterocycloalkyl. In embodiments, R⁶ is independently unsubstituted aryl. In embodiments, R⁶ is independently unsubstituted heteroaryl. In embodiments, R⁶ is independently substituted or unsubstituted C₁-C₆ alkyl. In embodiments, R⁶ is independently substituted or unsubstituted 2 to 6 membered heteroalkyl. In embodiments, R⁶ is independently substituted or unsubstituted C₃-C₈ cycloalkyl. In embodiments, R⁶ is independently substituted or unsubstituted 3 to 8 membered heterocycloalkyl. In embodiments, R⁶ is independently substituted or unsubstituted C₆ aryl. In embodiments, R⁶ is independently substituted or unsubstituted 5 to 6 membered heteroaryl. In embodiments, R⁶ is independently substituted C₁-C₆ alkyl.

In embodiments, R⁶ is independently substituted 2 to 6 membered heteroalkyl. In embodiments, R⁶ is independently substituted C₃-C₈ cycloalkyl. In embodiments, R⁶ is independently substituted 3 to 8 membered heterocycloalkyl. In embodiments, R⁶ is independently substituted C₆ aryl. In embodiments, R⁶ is independently substituted 5 to 6 membered heteroaryl. In embodiments, R⁶ is independently unsubstituted C₁-C₆ alkyl. In embodiments, R⁶ is independently unsubstituted 2 to 6 membered heteroalkyl. In embodiments, R⁶ is independently unsubstituted C₃-C₈ cycloalkyl. In embodiments, R⁶ is independently unsubstituted 3 to 8 membered heterocycloalkyl. In embodiments, R⁶ is independently unsubstituted C₆ aryl. In embodiments, R⁶ is independently unsubstituted 5 to 6 membered heteroaryl.

In embodiments, R⁷ is independently hydrogen. In embodiments, R⁷ is independently halogen. In embodiments, R⁷ is independently —N₃. In embodiments, R⁷ is independently —NO₂. In embodiments, R⁷ is independently —CF₃. In embodiments, R⁷ is independently —CCl₃. In embodiments, R⁷ is independently —CBr₃. In embodiments, R⁷ is independently —Cl₃. In embodiments, R⁷ is independently —CN. In embodiments, R⁷ is independently OR^{7A}. In embodiments, R⁷ is independently—NR^{7A}R^{7B}. In embodiments, R⁷ is independently COOH. In embodiments, R⁷ is independently —CONH₂. In embodiments, R⁷ is independently —NO₂. In embodiments, R⁷ is independently —SH. In embodiments, R⁷ is independently —SO₂Cl. In embodiments, R⁷ is independently —SO₃H. In embodiments, R⁷ is independently —SO₄H. In embodiments, R⁷ is independently —SO₂NH₂. In embodiments, R⁷ is independently —NHNH₂. In embodiments, R⁷ is independently —ONH₂. In embodiments, R⁷ is independently —OCH₃. In embodiments, R⁷ is independently —NHCNHNH₂. In embodiments, R⁷ is independently substituted or unsubstituted alkyl. In embodiments, R⁷ is independently substituted or unsubstituted heteroalkyl. In embodiments, R⁷ is independently substituted or unsubstituted cycloalkyl. In embodiments, R⁷ is independently substituted or unsubstituted heterocycloalkyl. In embodiments, R⁷ is independently substituted or unsubstituted aryl. In embodiments, R⁷ is independently substituted or unsubstituted heteroaryl. In embodiments, R⁷ is independently -L²—R⁹. In embodiments, R⁷ is independently substituted alkyl. In embodiments, R⁷ is independently substituted heteroalkyl. In embodiments, R⁷ is independently substituted cycloalkyl. In embodiments, R⁷ is independently substituted heterocycloalkyl. In embodiments, R⁷ is independently substituted aryl. In embodiments, R⁷ is independently substituted heteroaryl. In embodiments, R⁷ is independently unsubstituted alkyl. In embodiments, R⁷ is independently unsubstituted heteroalkyl. In embodiments, R⁷ is independently unsubstituted cycloalkyl. In embodiments, R⁷ is independently unsubstituted heterocycloalkyl. In embodiments, R⁷ is independently unsubstituted aryl. In embodiments, R⁷ is independently unsubstituted heteroaryl. In embodiments, R⁷ is independently substituted or unsubstituted C₁-C₆ alkyl. In embodiments, R⁷ is independently substituted or unsubstituted 2 to 6 membered heteroalkyl. In embodiments, R⁷ is independently substituted or unsubstituted C₃-C₈ cycloalkyl. In embodiments, R⁷ is independently substituted or unsubstituted 3 to 8 membered heterocycloalkyl. In embodiments, R⁷ is independently substituted or unsubstituted C₆ aryl. In embodiments, R⁷ is independently substituted or unsubstituted 5 to 6 membered heteroaryl. In embodiments, R⁷ is independently substituted C₁-C₆ alkyl. In embodiments, R⁷ is independently substituted 2 to 6 membered heteroalkyl. In embodiments, R⁷ is independently substituted C₃-C₈ cycloalkyl. In embodiments, R⁷ is independently substituted 3 to 8 membered heterocycloalkyl. In embodiments, R⁷ is independently substituted C₆ aryl. In embodiments, R⁷ is independently substituted 5 to 6 membered heteroaryl. In embodiments, R⁷ is independently unsubstituted C₁-C₆ alkyl. In embodiments, R⁷ is independently unsubstituted 2 to 6 membered heteroalkyl. In embodiments, R⁷ is independently unsubstituted C₃-C₈ cycloalkyl. In embodiments, R⁷ is independently unsubstituted 3 to 8 membered heterocycloalkyl. In embodiments, R⁷ is independently unsubstituted C₆ aryl. In embodiments, R⁷ is independently unsubstituted 5 to 6 membered heteroaryl.

In embodiments, R⁸ is independently hydrogen. In embodiments, R⁸ is independently halogen. In embodiments, R⁸ is independently —N₃. In embodiments, R⁸ is independently —NO₂. In embodiments, R⁸ is independently —CF₃. In embodiments, R⁸ is independently —CCl₃. In embodiments, R⁸ is independently —CBr₃. In embodiments, R⁸ is independently —Cl₃. In embodiments, R⁸ is independently —CN. In embodiments, R⁸ is independently —OR^{8A}. In embodiments, R⁸ is independently —NR^{8A}R^{8B}. In embodiments, R⁸ is independently —COOH. In embodiments, R⁸ is independently —CONH₂. In embodiments, R⁸ is independently —NO₂. In embodiments, R⁸ is independently —SH. In embodiments, R⁸ is independently —SO₂Cl. In embodiments, R⁸ is independently —SO₃H. In embodiments, R⁸ is independently —SO₄H. In embodiments, R⁸ is independently —SO₂NH₂. In embodiments, R⁸ is independently —NHNH₂. In embodiments, R⁸ is independently —ONH₂. In embodiments, R⁸ is independently —OCH₃. In embodiments, R⁸ is independently —NHCNHNH₂. In embodiments, R⁸ is independently substituted or unsubstituted alkyl. In embodiments, R⁸ is independently substituted or unsubstituted heteroalkyl. In embodiments, R⁸ is independently substituted or unsubstituted cycloalkyl. In embodiments, R⁸ is independently substituted or unsubstituted heterocycloalkyl. In embodiments, R⁸ is independently substituted or unsubstituted aryl. In embodiments, R⁸ is independently substituted or unsubstituted heteroaryl. In embodiments, R⁸ is independently L²—R⁹. In embodiments, R⁸ is independently substituted alkyl. In embodiments, R⁸ is independently substituted heteroalkyl. In embodiments, R⁸ is independently substituted cycloalkyl. In embodiments, R⁸ is independently substituted heterocycloalkyl. In embodiments, R⁸ is independently substituted aryl. In embodiments, R⁸ is independently substituted heteroaryl. In embodiments, R⁸ is independently unsubstituted alkyl. In embodiments, R⁸ is independently unsubstituted heteroalkyl. In embodiments, R⁸ is independently unsubstituted cycloalkyl. In embodiments, R⁸ is independently unsubstituted heterocycloalkyl. In embodiments, R⁸ is independently unsubstituted aryl. In embodiments, R⁸ is independently unsubstituted heteroaryl. In embodiments, R⁸ is independently substituted or unsubstituted C₁-C₆ alkyl. In embodiments, R⁸ is independently substituted or unsubstituted 2 to 6 membered heteroalkyl. In embodiments, R⁸ is independently substituted or unsubstituted C₃-C₈ cycloalkyl. In embodiments, R⁸ is independently substituted or unsubstituted 3 to 8 membered heterocycloalkyl. In embodiments, R⁸ is independently substituted or unsubstituted C₆ aryl. In embodiments, R⁸ is independently substituted or unsubstituted 5 to 6 membered heteroaryl. In embodiments, R⁸ is independently substituted C₁-C₆ alkyl. In embodiments, R⁸ is independently substituted 2 to 6 membered heteroalkyl. In embodiments, R⁸ is independently substituted C₃-C₈ cycloalkyl. In embodiments, R⁸ is independently substituted 3 to 8 membered heterocycloalkyl. In embodiments, R⁸ is independently substituted C₆ aryl. In embodiments, R⁸ is independently substituted 5 to 6 membered heteroaryl. In embodiments, R⁸ is independently unsubstituted C₁-C₆ alkyl. In embodiments, R⁸ is independently unsubstituted 2 to 6 membered heteroalkyl. In embodiments, R⁸ is independently unsubstituted C₃-C₈ cycloalkyl. In embodiments, R⁸ is independently unsubstituted 3 to 8 membered heterocycloalkyl. In embodiments, R⁸ is independently unsubstituted C₆ aryl. In embodiments, R⁸ is independently unsubstituted 5 to 6 membered heteroaryl.

In embodiments, L² is independently a bond. In embodiments, L² is independently —NR’ ²—. In embodiments, L² is independently substituted or unsubstituted alkylene. In embodiments, L² is independently substituted or unsubstituted heteroalkylene. In embodiments, L² is independently substituted or unsubstituted cycloalkylene. In embodiments, L² is independently substituted or unsubstituted heterocycloalkylene. In embodiments, L² is independently substituted or unsubstituted arylene. In embodiments, L² is independently substituted or unsubstituted heteroarylene. In embodiments, L² is independently substituted alkylene. In embodiments, L² is independently substituted heteroalkylene. In embodiments, L² is independently substituted cycloalkylene. In embodiments, L² is independently substituted heterocycloalkylene. In embodiments, L² is independently substituted arylene. In embodiments, L² is independently substituted heteroarylene. In embodiments, L² is independently unsubstituted alkylene. In embodiments, L² is independently unsubstituted heteroalkylene. In embodiments, L² is independently unsubstituted cycloalkylene. In embodiments, L² is independently unsubstituted heterocycloalkylene. In embodiments, L² is independently unsubstituted arylene. In embodiments, L² is independently unsubstituted heteroarylene. In embodiments, L² is independently substituted or unsubstituted C₁-C₆ alkylene. In embodiments, L² is independently substituted or unsubstituted 2 to 6 membered heteroalkylene. In embodiments, L² is independently substituted or unsubstituted C₃-C₈ cycloalkylene. In embodiments, L² is independently substituted or unsubstituted 3 to 8 membered heterocycloalkylene. In embodiments, L² is independently substituted or unsubstituted C₆ arylene. In embodiments, L² is independently substituted or unsubstituted 5 to 6 membered heteroarylene. In embodiments, L² is independently substituted C₁-C₆ alkylene. In embodiments, L² is independently substituted 2 to 6 membered heteroalkylene. In embodiments, L² is independently substituted C₃-C₈ cycloalkylene. In embodiments, L² is independently substituted 3 to 8 membered heterocycloalkylene. In embodiments, L² is independently substituted C₆ arylene. In embodiments, L² is independently substituted 5 to 6 membered heteroarylene. In embodiments, L² is independently unsubstituted C₁-C₆ alkylene. In embodiments, L² is independently unsubstituted 2 to 6 membered heteroalkylene. In embodiments, L² is independently unsubstituted C₃-C₈ cycloalkylene. In embodiments, L² is independently unsubstituted 3 to 8 membered heterocycloalkylene. In embodiments, L² is independently unsubstituted C₆ arylene. In embodiments, L² is independently unsubstituted 5 to 6 membered heteroarylene.

In embodiments, R¹⁰ is independently hydrogen. In embodiments, R¹⁰ is independently —OR^{10A} . In embodiments, R¹⁰ is independently —NR^{10A}R^{10B.} In embodiments, R¹⁰ is independently substituted or unsubstituted alkyl. In embodiments, R¹⁰ is independently substituted alkyl. In embodiments, R¹⁰ is independently unsubstituted alkyl. In embodiments, R¹⁰ is independently substituted or unsubstituted C₁-C₆ alkyl. In embodiments, R¹⁰ is independently substituted C₁-C₆ alkyl. In embodiments, R¹⁰ is independently unsubstituted C₁-C₆ alkyl.

In embodiments, each of R^{5A}, R^{5B}, R^{6A}, R^{6B}, R^{7A}, R^{7B}, R^{8A}, R^{8B}, R^{10A}, and R^{10B} is independently hydrogen. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted alkyl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted heteroalkyl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted cycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted heterocycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted aryl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted heteroaryl. In embodiments, each of R^{5A}, R^{5B}, R^{6A},R^{6B}, R^{7A}, R^{7B}, R^{8A}, R^{8B}, R^{10A}, and R^{10B} is independently substituted alkyl. In embodiments, each of R^{5A}, R^{5B}, R^{6A}, R^{6B}, R^{7A}, R^{7B}, R^{8A}, R^{8B}, R^{10A}, and R^{10B} is independently substituted heteroalkyl. In embodiments, each of R^{5A}, R^{5B}, R^{6A}, R^{6B}, R^{7A}, R^{7B}, R^{8A}, R^{8B}, R^{10A}, and R^{10B} is independently substituted cycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted heterocycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted aryl. In embodiments, each of R^{5A}, R^{5B}, R^{6A}, R^{6B}, R^{7A}, R^{7B}, R^{8A}, R^{8B}, R^{10A}, and R^{10B} is independently substituted heteroaryl. In embodiments, each of R^{5A}, R^{5B}, R^{6A}, R^{6B}, R^{7A}, R^{7B}, R^{8A}, R^{8B}, R^{10A}, and R^{10B} is independently unsubstituted alkyl. In embodiments, each of R^{5A}, R^{5B}, R^{6A}, R^{6B}, R^{7A}, R^{7B}, R^{8A}, R^{8B}, R^{10A}, and R^{10B} is independently unsubstituted aryl. In embodiments, each of R^{5A}, R^{5B}, R^{6A}, R^{6B}, R^{7A}, R^{7B}, R^{8A}, R^{8B}, R^{10A}, and R^{10B} is independently unsubstituted aryl. In embodiments, each of R^{5A}, R^{5B}, R^{6A}, R^{6B}, R^{7A}, R^{7B}, R^{8A}, R^{8B}, R^{10A}, and R^{10B} is independently unsubstituted heterocycloalkyl. In embodiments, each of R^{5A}, R^{6A}, R^{6B}, R^{7A}, R^{7B}, R^{8A}, R^{8B}, R^{10A}, and R^{10B} is independently unsubstituted aryl. In embodiments, each of R^{5A}, R^{5B}, R^{6A}, R⁶,R^{7A}, R^{7B}, R^{8A}, R^{8B}, R^{10A}, and R^{10B} is independently unsubstituted heteroaryl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted C₁-C₆alkyl. In embodiments, each of R^{5A}, R^{5B}, R^{6A}, R^{6B}, R^{7A}, R^{7B}, R^{8A}, R^{8B}, R^{10A}, and R^{10B}, is independently substituted or unsubstituted 2 to 6 membered heteroalkyl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted C₃-C₈ cycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted 3 to 8 membered heterocycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted C₆ aryl. In embodiments, each of R^{5A}, R^{5B}, R^{6A}, R^{6B}, R^{7A}, R^{7B}, R^{8A}, R^{8B}, R^{10A}, and R^{10B} is independently substituted or unsubstituted 5 to 6 membered heteroaryl. In embodiments, R^{5A} is independently substituted C₁-C₆ alkyl. In embodiments, each of R^{5A}, R^{5B}, R^{6A}, R^{6B}, R^{7A}, R^{7B}, R^{8A}, R^{8B}, R^{10A}, and R^{10B} is independently substituted 2 to 6 membered heteroalkyl. In embodiments, each of R^{5A}, R^{5B}, R^{6A}, R^{6B}, R^{7A}, R^{7B}, R^{8A}, R^{8B}, R^{10A}, and R^{10B} is independently substituted C₃-C₈ cycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted 3 to 8 membered heterocycloalkyl. In embodiments, each of R^{5A}, R^{5B}, R^{6A}, R^{6B}, R^{7A}, R^{7B}, R^{8A}, R^{8B}, R^{10A}, and R^{10B} is independently substituted C₆ aryl. In embodiments, each of R^{5A}, R^{5B}, R^{6A}, R^{6B}, R^{7A}, R^{7B}, R^{8A}, R^{8B}, R^{10A}, and R^{10B} is independently substituted 5 to 6 membered heteroaryl. In R^{5A}, R^{5B}, R^{6A}, R^{6B}, R^{7A}, R^{7B}, R^{8A}, R^{8B}, R^{10A}, and R^{10B} embodiments, each of R^{5A}, R^{5B}, R^{6A}, R^{6B}, R^{7A}, R^{7B}, R^{8A}, R^{8B}, R^{10A}, and R^{10B} is independently unsubstituted C₁-C₆ alkyl. In embodiments, each of R^{5A}, R^{5B}, R^{6A}, R^{6B}, R^{7A}, R^{7B}, R^{8A}, R^{8B}, R^{10A}, and R^{10B} is independently unsubstituted 2 to 6 membered heteroalkyl. In embodiments, each of R^{5A}, R^{5B}, R^{6A}, R^{6B}, R^{7A}, R^{7B}, R^{8A}, R^{8B}, R^{10A}, and R^{10B} is independently unsubstituted C₃-C₈ cycloalkyl. In embodiments, each of R^{5A}, R^{5B}, R^{6A}, R^{6B}, R^{7A}, R^{7B}, R^{8A}, R^{8B}, R^{10A}, and R^{10B} is independently unsubstituted 3 to 8 membered heterocycloalkyl. In embodiments, each of R^{5A}, R^{5B}, R^{6A}, R^{6B}, R^{7A}, R^{7B}, R^{8A}, R^{8B}, R^{10A}, and R^{10B} is independently unsubstituted C₆ aryl. In embodiments, each of R^{5A}, R^{5B}, R^{6A}, R^{6B}, R^{7A}, R^{7B}, R^{8A}, R^{8B}, R^{10A}, and R^{10B} is independently unsubstituted 5 to 6 membered heteroaryl.

In embodiments, each of R^{XA}, R^{XB}, and R^{XC} is independently hydrogen. In embodiments, each of R^{XA}, R^{XB}, and R^{XC} is independently substituted or unsubstituted alkyl. In embodiments, each of R^{XA}, R^{XB}, and R^{XC} is independently substituted or unsubstituted heteroalkyl. In embodiments, each of R^{XA}, R^{XB}, and R^{XC} is independently substituted or unsubstituted cycloalkyl. In embodiments, each of R^{XA}, R^{XB}, and R^{XC} is independently substituted or unsubstituted heterocycloalkyl. In embodiments, each of R^{XA}, R^{XB}, and R^{XC} is independently substituted or unsubstituted aryl. In embodiments, each of R^{XA}, R^{XB}, and R^{XC} is independently substituted or unsubstituted heteroaryl. In embodiments, each of R^{XA}, R^{XB}, and R^{XC} is independently -L²-R⁹. In embodiments, each of R^{XA}, R^{XB}, and R^{XC} is independently substituted alkyl. In embodiments, each of R^{XA}, R^{XB}, and R^{XC} is independently substituted heteroalkyl. In embodiments, each of R^{XA}, R^{XB}, and R^{XC} is independently substituted cycloalkyl. In embodiments, each of R^{XA}, R^{XB}, and R^{XC} is independently substituted heterocycloalkyl. In embodiments, each of R^{XA}, R^{XB}, and R^{XC} is independently substituted aryl. In embodiments, each of R^{XA}, R^{XB}, and R^{XC} is independently substituted heteroaryl. In embodiments, each of R^{XA}, R^{XB}, and R^{XC} is independently unsubstituted alkyl. In embodiments, each of R^{XA}, R^{XB}, and R^{XC} is independently unsubstituted heteroalkyl. In embodiments, each of R^{XA}, R^{XB}, and R^{XC} is independently unsubstituted cycloalkyl. In embodiments, each of R^{XA}, R^{XB}, and R^{XC} is independently unsubstituted heterocycloalkyl. In embodiments, each of R^{XA}, R^{XB}, and R^{XC} is independently unsubstituted aryl. In embodiments, each of R^{XA}, R^{XB},and R^{XC} is independently unsubstituted heteroaryl. In embodiments, each of R^{XA}, R^{XB}, and R^{XC} is independently substituted or unsubstituted C₁-C₆ alkyl. In embodiments, each of R^{XA}, R^{XB},and R^{XC} is independently substituted or unsubstituted 2 to 6 membered heteroalkyl. In embodiments, each of R^{XA}, R^{XB}, and R^{XC} is independently substituted or unsubstituted C₃-C₈ cycloalkyl. In embodiments, each of R^{XA}, R^{XB},and R^{XC} is independently substituted or unsubstituted 3 to 8 membered heterocycloalkyl. In embodiments, each of R^{XA}, R^{XB}, and R^{XC} is independently substituted or unsubstituted C₆ aryl. In embodiments, each of R^{XA}, R^{XB}, and R^{XC} is independently substituted or unsubstituted 5 to 6 membered heteroaryl. In embodiments, each of R^{XA}, R^{XB}, and R^{XC} is independently substituted C₁-C₆ alkyl. In embodiments, each of R^{XA}, R^{XB}, and R^{XC} is independently substituted 2 to 6 membered heteroalkyl. In embodiments, each of R^{XA}, R^{XB}, and R^{XC} is independently substituted C₃-C₈ cycloalkyl. In embodiments, each of R^{XA}, R^{XB}, and R^{XC} is independently substituted 3 to 8 membered heterocycloalkyl. In embodiments, each of R^{XA}, R^{XB}, and R^{XC} is independently substituted C₆ aryl. In embodiments, each of R^{XA}, R^{XB},and R^{XC} is independently substituted 5 to 6 membered heteroaryl. In embodiments, each of R^{XA}, R^{XB}, and R^{XC} is independently unsubstituted C₁-C₆ alkyl. In embodiments, each of R^{XA}, R^{XB},and R^{XC} is independently unsubstituted 2 to 6 membered heteroalkyl. In embodiments, each of R^{XA}, R^{XB}, and R^{XC} is independently unsubstituted C₃-C₈ cycloalkyl. In embodiments, each of R^{XA}, R^{XB}, and R^{XC} is independently unsubstituted 3 to 8 membered heterocycloalkyl. In embodiments, each of R^{XA}, R^{XB}, and R^{XC} is independently unsubstituted C₆ aryl. In embodiments, each of R^{XA}, R^{XB},and R^{XC} is independently unsubstituted 5 to 6 membered heteroaryl.

In embodiments, R^{L2} is independently hydrogen. In embodiments, R^{L2} is independently substituted or unsubstituted alkyl. In embodiments, R^{L2} is independently substituted or unsubstituted heteroalkyl. In embodiments, R^{L2} is independently substituted or unsubstituted cycloalkyl. In embodiments, R^{L2} is independently substituted or unsubstituted heterocycloalkyl. In embodiments, R^{L2} is independently substituted or unsubstituted aryl. In embodiments, R^{L2} is independently substituted or unsubstituted heteroaryl. In embodiments, R^{L2} is independently substituted alkyl. In embodiments, R^{L2} is independently substituted heteroalkyl. In embodiments, R^{L2} is independently substituted cycloalkyl. In embodiments, R^{L2} is independently substituted heterocycloalkyl. In embodiments, R^{L2} is independently substituted aryl. In embodiments, R^{L2} is independently substituted heteroaryl. In embodiments, R^{L2} is independently unsubstituted alkyl. In embodiments, R^{L2} is independently unsubstituted heteroalkyl. In embodiments, R^{L2} is independently unsubstituted cycloalkyl. In embodiments, R^{L2} is independently unsubstituted heterocycloalkyl. In embodiments, R^{L2} is independently unsubstituted aryl. In embodiments, R^{L2} is independently unsubstituted heteroaryl. In embodiments, R^{L2} is independently substituted or unsubstituted C₁-C₆ alkyl. In embodiments, R^{L2} is independently substituted or unsubstituted 2 to 6 membered heteroalkyl. In embodiments, R^{L2} is independently substituted or unsubstituted C₃-C₈ cycloalkyl. In embodiments, R^{L2} is independently substituted or unsubstituted 3 to 8 membered heterocycloalkyl. In embodiments, R^{L2} is independently substituted or unsubstituted C₆ aryl. In embodiments, R^{L2} is independently substituted or unsubstituted 5 to 6 membered heteroaryl. In embodiments, R^{L2} is independently substituted C₁-C₆ alkyl. In embodiments, R^{L2} is independently substituted 2 to 6 membered heteroalkyl. In embodiments, R^{L2} is independently substituted C₃-C₈ cycloalkyl. In embodiments, R^{L2} is independently substituted 3 to 8 membered heterocycloalkyl. In embodiments, R^{L2} is independently substituted C₆ aryl. In embodiments, R^{L2} is independently substituted 5 to 6 membered heteroaryl. In embodiments, R^{L2} is independently unsubstituted C₁-C₆ alkyl. In embodiments, R^{L2} is independently unsubstituted 2 to 6 membered heteroalkyl. In embodiments, R^{L2} is independently unsubstituted C₃-C₈ cycloalkyl. In embodiments, R^{L2} is independently unsubstituted 3 to 8 membered heterocycloalkyl. In embodiments, R^{L2} is independently unsubstituted C₆ aryl. In embodiments, R^{L2} is independently unsubstituted 5 to 6 membered heteroaryl.

In embodiments, X² is O. In embodiments, X² is NR^{X2}R^{X2′}.

In embodiments, R¹¹ is hydrogen. In embodiments, R¹¹ is substituted or unsubsituted alkyl. In embodiments, R¹¹ is substituted or unsubstituted heteroalkyl. In embodiments, R¹¹ is -L³-R^{14.} In embodiments, R¹¹ is independently substituted alkyl. In embodiments, R¹¹ is independently substituted heteroalkyl. In embodiments, R¹¹ is independently unsubstituted alkyl. In embodiments, R¹¹ is independently unsubstituted heteroalkyl. In embodiments, R¹¹ is independently substituted or unsubstituted C₁-C₆ alkyl. In embodiments, R¹¹ is independently substituted or unsubstituted 2 to 6 membered heteroalkyl. In embodiments, R¹¹ is independently substituted C₁-C₆ alkyl. In embodiments, R¹¹ is independently substituted 2 to 6 membered heteroalkyl. In embodiments, R¹¹ is independently unsubstituted C₁-C₆ alkyl. In embodiments, R¹¹ is independently unsubstituted 2 to 6 membered heteroalkyl.

In embodiments, R¹² is independently hydrogen. In embodiments, R¹² is independently halogen. In embodiments, R¹² is independently —N₃. In embodiments, R¹² is independently —NO₂. In embodiments, R¹² is independently —CF₃. In embodiments, R¹² is independently —CCl₃. In embodiments, R¹² is independently —CBr₃. In embodiments, R¹² is independently —Cl₃. In embodiments, R¹² is independently —CN. In embodiments, R¹² is independently —OR^{12A.} In embodiments, R¹² is independently —NR^{12A}R^{12B.} In embodiments, R¹² is independently —COOH. In embodiments, R¹² is independently —CONH₂. In embodiments, R¹² is independently —NO₂. In embodiments, R¹² is independently —SH. In embodiments, R¹² is independently —SO₂Cl. In embodiments, R¹² is independently —SO₃H. In embodiments, R¹² is independently —SO₄H. In embodiments, R¹² is independently —SO₂NH₂. In embodiments, R¹² is independently —NHNH₂. In embodiments, R¹² is independently —ONH₂. In embodiments, R¹² is independently —OCH₃. In embodiments, R¹² is independently —NHCNHNH₂. In embodiments, R¹² is independently substituted or unsubstituted alkyl. In embodiments, R¹² is independently substituted or unsubstituted heteroalkyl. In embodiments, R¹² is independently substituted or unsubstituted cycloalkyl. In embodiments, R¹² is independently substituted or unsubstituted heterocycloalkyl. In embodiments, R¹² is independently substituted or unsubstituted aryl. In embodiments, R¹² is independently substituted or unsubstituted heteroaryl. In embodiments, R¹² is independently -L⁴-R¹⁵. In embodiments, R¹² is independently substituted alkyl. In embodiments, R¹² is independently substituted heteroalkyl. In embodiments, R¹² is independently substituted cycloalkyl. In embodiments, R¹² is independently substituted heterocycloalkyl. In embodiments, R¹² is independently substituted aryl. In embodiments, R¹² is independently substituted heteroaryl. In embodiments, R¹² is independently unsubstituted alkyl. In embodiments, R¹² is independently unsubstituted heteroalkyl. In embodiments, R¹² is independently unsubstituted cycloalkyl. In embodiments, R¹² is independently unsubstituted heterocycloalkyl. In embodiments, R¹² is independently unsubstituted aryl. In embodiments, R¹² is independently unsubstituted heteroaryl. In embodiments, R¹² is independently substituted or unsubstituted C₁-C₆ alkyl. In embodiments, R¹² is independently substituted or unsubstituted 2 to 6 membered heteroalkyl. In embodiments, R¹² is independently substituted or unsubstituted C₃-C₈ cycloalkyl. In embodiments, R¹² is independently substituted or unsubstituted 3 to 8 membered heterocycloalkyl. In embodiments, R¹² is independently substituted or unsubstituted C₆ aryl. In embodiments, R¹² is independently substituted or unsubstituted 5 to 6 membered heteroaryl. In embodiments, R¹² is independently substituted C₁-C₆ alkyl. In embodiments, R¹² is independently substituted 2 to 6 membered heteroalkyl. In embodiments, R¹² is independently substituted C₃-C₈ cycloalkyl. In embodiments, R¹² is independently substituted 3 to 8 membered heterocycloalkyl. In embodiments, R¹² is independently substituted C₆ aryl. In embodiments, R¹² is independently substituted 5 to 6 membered heteroaryl. In embodiments, R¹² is independently unsubstituted C₁-C₆ alkyl. In embodiments, R¹² is independently unsubstituted 2 to 6 membered heteroalkyl. In embodiments, R¹² is independently unsubstituted C₃-C₈ cycloalkyl. In embodiments, R¹² is independently unsubstituted 3 to 8 membered heterocycloalkyl. In embodiments, R¹² is independently unsubstituted C₆ aryl. In embodiments, R¹² is independently unsubstituted 5 to 6 membered heteroaryl.

In embodiments, R¹³ is independently hydrogen. In embodiments, R¹³ is independently halogen. In embodiments, R¹³ is independently —N₃. In embodiments, R¹³ is independently —NO₂. In embodiments, R¹³ is independently —CF₃. In embodiments, R¹³ is independently —CCl₃. In embodiments, R¹³ is independently —CBr₃. In embodiments, R¹³ is independently —Cl₃. In embodiments, R¹³ is independently —CN. In embodiments, R¹³ is independently —OR^{13A}. In embodiments, R¹³ is independently —NR^{13A}R^{13B.} In embodiments, R¹³ is independently —COOH. In embodiments, R¹³ is independently —CONH₂. In embodiments, R¹³ is independently —NO₂. In embodiments, R¹³ is independently —SH. In embodiments, R¹³ is independently —SO₂Cl. In embodiments, R¹³ is independently —SO₃H. In embodiments, R¹³ is independently —SO₄H. In embodiments, R¹³ is independently —SO₂NH₂. In embodiments, R¹² is independently —NHNH₂. In embodiments, R¹³ is independently —ONH₂. In embodiments, R¹³ is independently —OCH₃. In embodiments, R¹³ is independently —NHCNHNH₂. In embodiments, R¹³ is independently substituted or unsubstituted alkyl. In embodiments, R¹³ is independently substituted or unsubstituted heteroalkyl. In embodiments, R¹³ is independently substituted or unsubstituted cycloalkyl. In embodiments, R¹³ is independently substituted or unsubstituted heterocycloalkyl. In embodiments, R¹³ is independently substituted or unsubstituted aryl. In embodiments, R¹³ is independently substituted or unsubstituted heteroaryl. In embodiments, R¹³ is independently -L⁴-R¹⁵. In embodiments, R¹³ is independently substituted alkyl. In embodiments, R¹³ is independently substituted heteroalkyl. In embodiments, R¹³ is independently substituted cycloalkyl. In embodiments, R¹³ is independently substituted heterocycloalkyl. In embodiments, R¹³ is independently substituted aryl. In embodiments, R¹³ is independently substituted heteroaryl. In embodiments, R¹³ is independently unsubstituted alkyl. In embodiments, R¹³ is independently unsubstituted heteroalkyl. In embodiments, R¹³ is independently unsubstituted cycloalkyl. In embodiments, R¹³ is independently unsubstituted heterocycloalkyl. In embodiments, R¹³ is independently unsubstituted aryl. In embodiments, R¹³ is independently unsubstituted heteroaryl. In embodiments, R¹³ is independently substituted or unsubstituted C₁-C₆ alkyl. In embodiments, R¹³ is independently substituted or unsubstituted 2 to 6 membered heteroalkyl. In embodiments, R¹³ is independently substituted or unsubstituted C₃-C₈ cycloalkyl. In embodiments, R¹³ is independently substituted or unsubstituted 3 to 8 membered heterocycloalkyl. In embodiments, R¹³ is independently substituted or unsubstituted C₆ aryl. In embodiments, R¹³ is independently substituted or unsubstituted 5 to 6 membered heteroaryl. In embodiments, R¹³ is independently substituted C₁-C₆ alkyl. In embodiments, R¹³ is independently substituted 2 to 6 membered heteroalkyl. In embodiments, R¹³ is independently substituted C₃-C₈ cycloalkyl. In embodiments, R¹³ is independently substituted 3 to 8 membered heterocycloalkyl. In embodiments, R¹³ is independently substituted C₆ aryl. In embodiments, R¹³ is independently substituted 5 to 6 membered heteroaryl. In embodiments, R¹³ is independently unsubstituted C₁-C₆ alkyl. In embodiments, R¹³ is independently unsubstituted 2 to 6 membered heteroalkyl. In embodiments, R¹³ is independently unsubstituted C₃-C₈ cycloalkyl. In embodiments, R¹³ is independently unsubstituted 3 to 8 membered heterocycloalkyl. In embodiments, R¹³ is independently unsubstituted C₆ aryl. In embodiments, R¹³ is independently unsubstituted 5 to 6 membered heteroaryl.

In embodiments, R¹⁴ is hydrogen. In embodiments, R¹⁴ is -L⁴ -R¹⁵.

In embodiments, L³ is independently a bond. In embodiments, In embodiments, L³ is independently substituted or unsubstituted cycloalkylene. In embodiments, L³ is independently substituted or unsubstituted heterocycloalkylene.In embodiments, L³ is independently substituted or unsubstituted arylene. In embodiments, L³ is independently substituted or unsubstituted heteroarylene. In embodiments, L³ is independently substituted cycloalkylene. In embodiments, L³ is independently substituted heterocycloalkylene. In embodiments, L³ is independently substituted arylene. In embodiments, L³ is independently substituted heteroarylene. In embodiments, L³ is independently unsubstituted cycloalkylene. In embodiments, L³ is independently unsubstituted heterocycloalkylene. In embodiments, L³ is independently unsubstituted arylene. In embodiments, L³ is independently unsubstituted heteroarylene. In embodiments, L³ is independently substituted or unsubstituted C₃-C₈ cycloalkylene. In embodiments, L³ is independently substituted or unsubstituted 3 to 8 membered heterocycloalkylene. In embodiments, L³ is independently substituted or unsubstituted C₆ arylene. In embodiments, L³ is independently substituted or unsubstituted 5 to 6 membered heteroarylene. In embodiments, L³ is independently substituted C₃-C₈ cycloalkylene. In embodiments, L³ is independently substituted 3 to 8 membered heterocycloalkylene. In embodiments, L³ is independently substituted C₆ arylene. In embodiments, L³ is independently substituted 5 to 6 membered heteroarylene. In embodiments, L³ is independently unsubstituted C₃-C₈ cycloalkylene. In embodiments, L³ is independently unsubstituted 3 to 8 membered heterocycloalkylene. In embodiments, L³ is independently unsubstituted C₆ arylene. In embodiments, L³ is independently unsubstituted 5 to 6 membered heteroarylene.

In embodiments, L⁴ is independently a bond. In embodiments, L⁴ is independently —NR^{L4}—. In embodiments, L⁴ is independently substituted or unsubstituted alkylene. In embodiments, L⁴ is independently substituted or unsubstituted heteroalkylene. In embodiments, L⁴ is independently substituted or unsubstituted cycloalkylene. In embodiments, L⁴ is independently substituted or unsubstituted heterocycloalkylene. In embodiments, L⁴ is independently substituted or unsubstituted arylene. In embodiments, L⁴ is independently substituted or unsubstituted heteroarylene. In embodiments, L⁴ is independently substituted alkylene. In embodiments, L⁴ is independently substituted heteroalkylene. In embodiments, L⁴ is independently substituted cycloalkylene. In embodiments, L⁴ is independently substituted heterocycloalkylene. In embodiments, L⁴ is independently substituted arylene. In embodiments, L⁴ is independently substituted heteroarylene. In embodiments, L⁴ is independently unsubstituted alkylene. In embodiments, L⁴ is independently unsubstituted heteroalkylene. In embodiments, L⁴ is independently unsubstituted cycloalkylene. In embodiments, L⁴ is independently unsubstituted heterocycloalkylene. In embodiments, L⁴ is independently unsubstituted arylene. In embodiments, L⁴ is independently unsubstituted heteroarylene. In embodiments, L⁴ is independently substituted or unsubstituted C₁-C₆ alkylene. In embodiments, L⁴ is independently substituted or unsubstituted 2 to 6 membered heteroalkylene. In embodiments, L⁴ is independently substituted or unsubstituted C₃-C₈ cycloalkylene. In embodiments, L⁴ is independently substituted or unsubstituted 3 to 8 membered heterocycloalkylene. In embodiments, L⁴ is independently substituted or unsubstituted C₆ arylene. In embodiments, L⁴ is independently substituted or unsubstituted 5 to 6 membered heteroarylene. In embodiments, L⁴ is independently substituted C₁-C₆ alkylene. In embodiments, L⁴ is independently substituted 2 to 6 membered heteroalkylene. In embodiments, L⁴ is independently substituted C₃-C₈ cycloalkylene. In embodiments, L⁴ is independently substituted 3 to 8 membered heterocycloalkylene. In embodiments, L⁴ is independently substituted C₆ arylene. In embodiments, L⁴ is independently substituted 5 to 6 membered heteroarylene. In embodiments, L⁴ is independently unsubstituted C₁-C₆ alkylene. In embodiments, L⁴ is independently unsubstituted 2 to 6 membered heteroalkylene. In embodiments, L⁴ is independently unsubstituted C₃-C₈ cycloalkylene. In embodiments, L⁴ is independently unsubstituted 3 to 8 membered heterocycloalkylene. In embodiments, L⁴ is independently unsubstituted C₆ arylene. In embodiments, L⁴ is independently unsubstituted 5 to 6 membered heteroarylene.

In embodiments, each of R^{12A}, R^{12B}, R^{13A}, R^{13B}, R^{L4}, R^{X2}and R^{X2} is independently hydrogen. In embodiments, each of R^{12A}, R^{12B}, R^{13A}, R^{13B}, R^{L4}, R^{X2}, and R^{X2′} is independently substituted or unsubstituted alkyl. In embodiments, each of R^{12A}, R^{12B}, R^{13A}, R^{13B}, R^{L4}, R^{X2}, and R^{X2′} is independently substituted or unsubstituted heteroalkyl. In embodiments, each of R^{12A}, R^{12B}, R^{13A}, R^{13B}, R^{L4}, R^{X2}, and R^{X2′} is independently substituted or unsubstituted cycloalkyl. In embodiments, each of R^{12A}, R^{12B}, R^{13A}, R^{13B}, R^{L4}, R^{X2}, and R^{X2′} is independently substituted or unsubstituted heterocycloalkyl. In embodiments, each of R^{12A}, R^{12B}, R^{13A}, R^{13B}, R^{L4}, R^{X2},and R^{X2′} is independently substituted or unsubstituted aryl. In embodiments, each of R^{12A}, R^{12B}, R^{13A}, R^{13B}, R^{L4}, R^{X2}, and R^{X2′} is independently substituted or unsubstituted heteroaryl. In embodiments, each of R^{12A}, R^{12B}, R^{13A}, R^{13B}, R^{L4}, R^{X2}, and R^{X2′} is independently substituted alkyl. In embodiments, each of R^{12A}, R^{12B}, R^{13A}, R^{13B}, R^{L4}, R^{X2}, and R^{X2′} is independently substituted heteroalkyl. In embodiments, each of R^{12A}, R^{12B}, R^{13A}, R^{13B}, R^{L4}, R^{X2}, and R^{X2′} is independently substituted cycloalkyl. In embodiments, each of R^{12A}, R^{12B}, R^{13A}, R^{13B}, R^{L4}, R^{X2}, and R^{X2′} is independently substituted heterocycloalkyl. In embodiments, each of R^{12A}, R^{12B}, R^{13A}, R^{13B}, R^{L4}, R^{X2}, and R^{X2′} is independently substituted aryl. In embodiments, R^{12A} is independently substituted heteroaryl. In embodiments, each of R^{12A}, R^{12B}, R^{13A}, R^{13B}, R^{L4}, R^{X2}, and R^{X2′} is independently unsubstituted alkyl. In embodiments, each of R^{12A}, R^{12B}, R^{13A}, R^{13B}, R^{L4}, R^{X2}, and R^{X2′} is independently unsubstituted heteroalkyl. In embodiments, each of R^{12A}, R^{12B}, R^{13A}, R^{13B}, R^{L4}, and R^{X2′} is independently unsubstituted cycloalkyl. In embodiments, each of R^{12A}, R^{12B}, R^{13A}, R^{13B}, R^{L4}, R^{X2}, and R^{X2′} is independently unsubstituted heterocycloalkyl. In embodiments, each of R^{12A}, R^{12B}, R^{13A}, R^{13B}, R^{L4}, R^{X2}, and R^{X2′} is independently unsubstituted aryl. In embodiments, each of R^{12A}, R^{12B}, R^{13A}, R^{13B}, R^{L4}, R^{X2}, and R^{X2′} is independently unsubstituted heteroaryl. In embodiments, each of R^{12A}, R^{12B}, R^{13A}, R^{13B}, R^{L4}, R^{X2}, and R^{X2′} is independently substituted or unsubstituted C₁-C₆ alkyl. In embodiments, each of R^{12A}, R^{12B}, R^{13A}, R^{13B}, R^{L4}, R^{X2}, and R^{X2′} is independently substituted or unsubstituted 2 to 6 membered heteroalkyl. In embodiments, each of R^{12A}, R^{12B}, R^{13A}, R^{13B}, R^{L4}, R^{X2}, and R^{X2′} is independently substituted or unsubstituted C₃-C₈ cycloalkyl. In embodiments, each of R^{12A}, R^{12B}, R^{13A}, R^{13B}, R^{L4}, R^{X2}, and R^{X2′} is independently substituted or unsubstituted 3 to 8 membered heterocycloalkyl. In embodiments, each of R^{12A}, R^{12B}, R^{13A}, R^{13B}, R^{L4}, R^{X2}, and R^{X2′} is independently substituted or unsubstituted C₆ aryl. In embodiments, each of R^{12A}, R^{12B}, R^{13A}, R^{13B}, R^{L4}, R^{X2}, and R^{X2′} is independently substituted or unsubstituted 5 to 6 membered heteroaryl. In embodiments, each of R^{12A}, R^{12B}, R^{13A}, R^{13B}, R^{L4}, R^{X2}, and R^{X2′} is independently substituted C₁-C₆ alkyl. In embodiments, each of R^{12A}, R^{12B}, R^{13A}, R^{13B}, R^{L4}, R^{X2}, and R^{X2′} is independently substituted 2 to 6 membered heteroalkyl. In embodiments, each of R^{12A}, R^{12B}, R^{13A}, R^{13B}, R^{L4}, R^{X2 , and R}^{X2′} is independently substituted C₃-C₈ cycloalkyl. In embodiments, each of R^{12A}, R^{12B}, R^{13A}, R^{13B}, R^{L4}, R^{X2}, and R^{X2′} is independently substituted 3 to 8 membered heterocycloalkyl. In embodiments, each of R^{12A}, R^{12B}, R^{13A}, R^{13B}, R^{L4}, R^{X2}, and R^{X2′} is independently substituted C₆ aryl. In embodiments, each of R^{12A}, R^{12B}, R^{13A}, R^{13B}, R^{L4}, R^{X2}, and R^{X2′} is independently substituted 5 to 6 membered heteroaryl. In embodiments, each of R^{12A, R}^{12B}, R^{13A}, R^{13B}, R^{L4}, R^{X2}, and R^{X2′} is independently unsubstituted C₁-C₆ alkyl. In embodiments, each of R^{12A}, R^{12B}, R^{13A}, R^{13B}, R^{L4}, R^{X2}, and R^{X2′} is independently unsubstituted 2 to 6 membered heteroalkyl. In embodiments, each of R^{12A}, R^{12B}, R^{13A}, R^{13B}, R^{L4}, R^{X2}, and R^{X2′} is independently unsubstituted C₃-C₈ cycloalkyl. In embodiments, each of R^{12A}, R^{12B}, R^{13A}, R^{13B}, R^{L4}, R^{X2}, and R^{X2′} is independently unsubstituted 3 to 8 membered heterocycloalkyl. In embodiments, each of R^{12A}, R^{12B}, R^{13A}, R^{13B}, R^{L4}, R^{X2}, and R^{X2′} is independently unsubstituted C₆ aryl. In embodiments, each of R^{12A}, R^{12B}, R^{13A}, R^{13B}, R^{L4}, R^{X2}, and R^{X2′} is independently unsubstituted 5 to 6 membered heteroaryl.

In embodiments, R^{11A} is independently hydrogen. In embodiments, R^{11A} is independently substituted or unsubstituted alkyl. In embodiments, R^{11A} is independently —CO₂R^{11C}. In embodiments, R^{11A} is independently -L⁴-R¹⁵. In embodiments, R^{11A} is independently substituted alkyl. In embodiments, R^{11A} is independently unsubstituted alkyl. In embodiments, R^{11A} is independently substituted or unsubstituted C₁-C₆ alkyl. In embodiments, R^{11A} is independently substituted C₁-C₆ alkyl. In embodiments, R^{11A} is independently unsubstituted C₁-C₆ alkyl.

In embodiments, R^{11B} is independently hydrogen. In embodiments, R^{11B} is independently halogen. In embodiments, R^{11B} is independently —N₃. In embodiments, R^{11B} is independently —NO₂. In embodiments, R^{11B} is independently —CF₃. In embodiments, R^{11B} is independently —CCl₃. In embodiments, R^{11B} is independently —CBr₃. In embodiments, R^{11B} is independently —Cl₃. In embodiments, R^{11B} is independently —CN. In embodiments, R^{11B} is independently —OR^{11D}. In embodiments, R^{11B} is independently —NR^{11D}R^{11E}. In embodiments, R^{11B} is independently —COOH. In embodiments, R^{11B} is independently —CONH₂. In embodiments, R^{11B} is independently —NO₂. In embodiments, R^{11B} is independently —SH. In embodiments, R^{11B} is independently —SO₂Cl. In embodiments, R^{11B} is independently —SO₃H. In embodiments, R^{11B} is independently —SO₄H. In embodiments, R^{11B} is independently —SO₂NH₂. In embodiments, R^{11B} is independently —NHNH₂. In embodiments, R^{11B} is independently —ONH₂. In embodiments, R^{11B} is independently —OCH₃. In embodiments, R^{11B} is independently —NHCNHNH₂. In embodiments, R^{11B} is independently substituted or unsubstituted alkyl. In embodiments, R^{11B} is independently substituted or unsubstituted heteroalkyl. In embodiments, R^{11B} is independently substituted or unsubstituted cycloalkyl. In embodiments, R^{11B} is independently substituted or unsubstituted heterocycloalkyl. In embodiments, R^{11B} is independently substituted or unsubstituted aryl. In embodiments, R^{11B} is independently substituted or unsubstituted heteroaryl. In embodiments, R^{11B} is independently -L⁴-R¹⁵. In embodiments, R^{11B} is independently substituted alkyl. In embodiments, R^{11B} is independently substituted heteroalkyl. In embodiments, R^{11B} is independently substituted cycloalkyl. In embodiments, R^{11B} is independently substituted heterocycloalkyl. In embodiments, R^{11B} is independently substituted aryl. In embodiments, R^{11B} is independently substituted heteroaryl. In embodiments, R^{11B} is independently unsubstituted alkyl. In embodiments, R^{11B} is independently unsubstituted heteroalkyl. In embodiments, R^{11B} is independently unsubstituted cycloalkyl. In embodiments, R^{11B} is independently unsubstituted heterocycloalkyl. In embodiments, R^{11B} is independently unsubstituted aryl. In embodiments, R^{11B} is independently unsubstituted heteroaryl. In embodiments, R^{11B} is independently substituted or unsubstituted C₁-C₆ alkyl. In embodiments, R^{11B} is independently substituted or unsubstituted 2 to 6 membered heteroalkyl. In embodiments, R^{11B} is independently substituted or unsubstituted C₃-C₈ cycloalkyl. In embodiments, R^{11B} is independently substituted or unsubstituted 3 to 8 membered heterocycloalkyl. In embodiments, R^{11B} is independently substituted or unsubstituted C₆ aryl. In embodiments, R^{11B} is independently substituted or unsubstituted 5 to 6 membered heteroaryl. In embodiments, R^{11B} is independently substituted C₁-C₆ alkyl. In embodiments, R^{11B} is independently substituted 2 to 6 membered heteroalkyl. In embodiments, R^{11B}, is independently substituted C₃-C₈ cycloalkyl. In embodiments, R^{11B} is independently substituted 3 to 8 membered heterocycloalkyl. In embodiments, R^{11B} is independently substituted C₆ aryl. In embodiments, R^{11B} is independently substituted 5 to 6 membered heteroaryl. In embodiments, R^{11B} is independently unsubstituted C₁-C₆ alkyl. In embodiments, R^{11B} is independently unsubstituted 2 to 6 membered heteroalkyl. In embodiments, R^{11B} is independently unsubstituted C₃-C₈ cycloalkyl. In embodiments, R^{11B} is independently unsubstituted 3 to 8 membered heterocycloalkyl. In embodiments, R^{11B} is independently unsubstituted C₆ aryl. In embodiments, R^{11B} is independently unsubstituted 5 to 6 membered heteroaryl.

In embodiments, each of R^{11C}, R^{11D}, and R^{11E} is independently hydrogen. In embodiments, each of R^{11C}, R^{11D}, and R^{11E} is independently substituted or unsubstituted alkyl. In embodiments, each of R^{11C}, R^{11D}, and R^{11E} and R^{11E} is independently substituted or unsubstituted heteroalkyl. In embodiments, each of R^{11C}, R^{11D}, and R^{11E} is independently substituted or unsubstituted cycloalkyl. In embodiments, each of R^{11C}, R^{11D}, and R^{11E} is independently substituted or unsubstituted heterocycloalkyl. In embodiments, each of R^{11C}, R^{11D}, and R^{11E} is independently substituted or unsubstituted aryl. In embodiments, each of R^{11C}, R^{11D}, and R^{11E} is independently substituted or unsubstituted heteroaryl. In embodiments, each of R^{11C}, R^{11D}, and R^{11E} is independently substituted alkyl. In embodiments, each of R^{11C}, R^{11D}, and IC^{11E} is independently substituted heteroalkyl. In embodiments, each of R^{11C}, R^{11D}, and R^{11E} is independently substituted cycloalkyl. In embodiments, each of R^{11C}, R^{11D}, and R^{11E} is independently substituted heterocycloalkyl. In embodiments, each of R^{11C}, R^{11D}, and R^{11E} is independently substituted aryl. In embodiments, each of R^{11C}, R^{11D}, and R^{11E} is independently substituted heteroaryl. In embodiments, each of R^{11C}, R^{11D}, and R^{11E} is independently unsubstituted alkyl. In embodiments, each of R^{11C}, R^{11D}, and R^{E11} is independently unsubstituted heteroalkyl. In embodiments, each of R^{11C}, R^{11D}, and R^{11E} is independently unsubstituted cycloalkyl. In embodiments, each of R^{11C}, R^{11D}, and R^{11E} is independently unsubstituted heterocycloalkyl. In embodiments, each of R^{11C}, R^{11D}, and R^{11E} is independently unsubstituted aryl. In embodiments, each of R^{11C}, R^{11D}, and R^{11E} is independently unsubstituted heteroaryl. In embodiments, each of R^{11C}, R^{11D}, and R^{11E} is independently substituted or unsubstituted C₁-C₆ alkyl. In embodiments, each of R^{11C}, R^{11D}, and R^{11E} is independently substituted or unsubstituted 2 to 6 membered heteroalkyl. In embodiments, each of R^{11C}, R^{11D}, and R^{11E} is independently substituted or unsubstituted C₃-C₈ cycloalkyl. In embodiments, each of R^{11C}, R^{11D}, and R^{11E} is independently substituted or unsubstituted 3 to 8 membered heterocycloalkyl. In embodiments, each of R^{11C}, R^{11D}, and R^{11E} is independently substituted or unsubstituted C₆ aryl. In embodiments, each of R^{11C}, R^{11D}, and R^{11E} is independently substituted or unsubstituted 5 to 6 membered heteroaryl. In embodiments, each of R^{11C}, R^{11D}, and R^{11E} is independently substituted C₁-C₆ alkyl. In embodiments, each of R^{11C}, R^{11D}, and R^{11E} is independently substituted 2 to 6 membered heteroalkyl. In embodiments, each of R^{11C}, R^{11D}, and R^{11E} is independently substituted C₃-C₈ cycloalkyl. In embodiments, each of R^{11C}, R^{11D}, _{and R}^{11E} is independently substituted 3 to 8 membered heterocycloalkyl. In embodiments, each of R^{11C}, R^{11D}, and R^{11E} is independently substituted C₆ aryl. In embodiments, each of R^{11C}, R^{11D}, ₐₙd R^{21E} is independently substituted 5 to 6 membered heteroaryl. In embodiments, each of R^{11C}, R^{11D}, and R^{11E} is independently unsubstituted C₁-C₆ alkyl. In embodiments, each of R^{11C}, R^{11D}, and R^{11E} is independently unsubstituted 2 to 6 membered heteroalkyl. In embodiments, each of R^{11C}, R^{11D}, and R^{11E} is independently unsubstituted C₃-C₈ cycloalkyl. In embodiments, each of R^{11C}, R^{11D}, and R^{11E} is independently unsubstituted 3 to 8 membered heterocycloalkyl. In embodiments, each of R^{11C}, R^{11D}, and R^{11E} is independently unsubstituted C₆ aryl. In embodiments, each of R^{11C}, R^{11D}, and R^{11E} is independently unsubstituted 5 to 6 membered heteroaryl.

In embodiments, X³ is independently O. In embodiments, X³ is independently NR^{X3}R^{X3′}.

In embodiments, R¹⁶ is independently hydrogen. In embodiments, R¹⁶ is independently halogen. In embodiments, R¹⁶ is independently —N₃. In embodiments, R¹⁶ is independently —NO₂. In embodiments, R¹⁶ is independently —CF₃. In embodiments, R¹⁶ is independently —CCl₃. In embodiments, R¹⁶ is independently —CBr₃. In embodiments, R¹⁶ is independently —Cl₃. In embodiments, R¹⁶ is independently —CN. In embodiments, R¹⁶ is independently —OR^{16A.} In embodiments, R¹⁶ is independently —NR^{16A}R^{16B.} In embodiments, R¹⁶ is independently —COOH. In embodiments, R¹⁶ is independently —CONH₂. In embodiments, R¹⁶ is independently —NO₂. In embodiments, R¹⁶ is independently —SH. In embodiments, R¹⁶ is independently —SO₂Cl. In embodiments, R¹⁶ is independently —SO₃H. In embodiments, R¹⁶ is independently —SO₄H. In embodiments, R¹⁶ is independently —SO₂NH₂. In embodiments, R¹⁶ is independently —NHNH₂. In embodiments, R¹⁶ is independently —ONH₂. In embodiments, R¹⁶ is independently —OCH₃. In embodiments, R¹⁶ is independently —NHCNHNH₂. In embodiments, R¹⁶ is independently substituted or unsubstituted alkyl. In embodiments, R¹⁶ is independently substituted or unsubstituted heteroalkyl. In embodiments, R¹⁶ is independently substituted or unsubstituted cycloalkyl. In embodiments, R¹⁶ is independently substituted or unsubstituted heterocycloalkyl. In embodiments, R¹⁶ is independently substituted or unsubstituted aryl. In embodiments, R¹⁶ is independently substituted or unsubstituted heteroaryl. In embodiments, R¹⁶ is independently —OCH═CH₂. In embodiments, R¹⁶ is independently -L⁴-R¹⁵. In embodiments, R¹⁶ is independently substituted alkyl. In embodiments, R¹⁶ is independently substituted heteroalkyl. In embodiments, R¹⁶ is independently substituted cycloalkyl. In embodiments, R¹⁶ is independently substituted heterocycloalkyl. In embodiments, R¹⁶ is independently substituted aryl. In embodiments, R¹⁶ is independently substituted heteroaryl. In embodiments, R¹⁶ is independently unsubstituted alkyl. In embodiments, R¹⁶ is independently unsubstituted heteroalkyl. In embodiments, R¹⁶ is independently unsubstituted cycloalkyl. In embodiments, R¹⁶ is independently unsubstituted heterocycloalkyl. In embodiments, R¹⁶ is independently unsubstituted aryl. In embodiments, R¹⁶ is independently unsubstituted heteroaryl. In embodiments, R¹⁶ is independently substituted or unsubstituted C₁-C₆ alkyl. In embodiments, R¹⁶ is independently substituted or unsubstituted 2 to 6 membered heteroalkyl. In embodiments, R¹⁶ is independently substituted or unsubstituted C₃-C₈ cycloalkyl. In embodiments, R¹⁶ is independently substituted or unsubstituted 3 to 8 membered heterocycloalkyl. In embodiments, R¹⁶ is independently substituted or unsubstituted C₁₆ aryl. In embodiments, R¹⁶ is independently substituted or unsubstituted 5 to 6 membered heteroaryl. In embodiments, R¹⁶ is independently substituted C₁-C₆ alkyl. In embodiments, R¹⁶ is independently substituted 2 to 6 membered heteroalkyl. In embodiments, R¹⁶ is independently substituted C₃-C₈ cycloalkyl. In embodiments, R¹⁶ is independently substituted 3 to 8 membered heterocycloalkyl. In embodiments, R¹⁶ is independently substituted C₆ aryl. In embodiments, R¹⁶ is independently substituted 5 to 6 membered heteroaryl. In embodiments, R¹⁶ is independently unsubstituted C₁-C₆ alkyl. In embodiments, R¹⁶ is independently unsubstituted 2 to 6 membered heteroalkyl. In embodiments, R¹⁶ is independently unsubstituted C₃-C₈ cycloalkyl. In embodiments, R¹⁶ is independently unsubstituted 3 to 8 membered heterocycloalkyl. In embodiments, R¹⁶ is independently unsubstituted C₆ aryl. In embodiments, R¹⁶ is independently unsubstituted 5 to 6 membered heteroaryl.

In embodiments, R¹⁷ is independently hydrogen. In embodiments, R¹⁷ is independently halogen. In embodiments, R¹⁷ is independently —N₃. In embodiments, R¹⁷ is independently —NO₂. In embodiments, R¹⁷ is independently —CF₃. In embodiments, R¹⁷ is independently —CCl₃. In embodiments, R¹⁷ is independently —CBr₃. In embodiments, R¹⁷ is independently —Cl₃. In embodiments, R¹⁷ is independently —CN. In embodiments, R¹⁷ is independently —OR^{17A}. In embodiments, R¹⁷ is independently —NR^{17A}R^{17B.} In embodiments, R¹⁷ is independently —COOH. In embodiments, R¹⁷ is independently —CONH₂. In embodiments, R¹⁷ is independently —NO₂. In embodiments, R¹⁷ is independently —SH. In embodiments, R¹⁷ is independently —SO₂Cl. In embodiments, R¹⁷ is independently —SO₃H. In embodiments, R¹⁷ is independently —SO₄H. In embodiments, R¹⁷ is independently —SO₂NH₂. In embodiments, R¹⁷ is independently —NHNH₂. In embodiments, R¹⁷ is independently —ONH₂. In embodiments, R¹⁷ is independently —OCH₃. In embodiments, R¹⁷ is independently —NHCNHNH₂. In embodiments, R¹⁷ is independently substituted or unsubstituted alkyl. In embodiments, R¹⁷ is independently substituted or unsubstituted heteroalkyl. In embodiments, R¹⁷ is independently substituted or unsubstituted cycloalkyl. In embodiments, R¹⁷ is independently substituted or unsubstituted heterocycloalkyl. In embodiments, R¹⁷ is independently substituted or unsubstituted aryl. In embodiments, R¹⁷ is independently substituted or unsubstituted heteroaryl. In embodiments, R¹⁷ is independently —OCH═CH₂. In embodiments, R¹⁷ is independently -L⁴-R¹⁵. In embodiments, R¹⁷ is independently substituted alkyl. In embodiments, R¹⁷ is independently substituted heteroalkyl. In embodiments, R¹⁷ is independently substituted cycloalkyl. In embodiments, R¹⁷ is independently substituted heterocycloalkyl. In embodiments, R¹⁷ is independently substituted aryl. In embodiments, R¹⁷ is independently substituted heteroaryl. In embodiments, R¹⁷ is independently unsubstituted alkyl. In embodiments, R¹⁷ is independently unsubstituted heteroalkyl. In embodiments, R¹⁷ is independently unsubstituted cycloalkyl. In embodiments, R¹⁷ is independently unsubstituted heterocycloalkyl. In embodiments, R¹⁷ is independently unsubstituted aryl. In embodiments, R¹⁷ is independently unsubstituted heteroaryl. In embodiments, R¹⁷ is independently substituted or unsubstituted C₁-C₆ alkyl. In embodiments, R¹⁷ is independently substituted or unsubstituted 2 to 6 membered heteroalkyl. In embodiments, R¹⁷ is independently substituted or unsubstituted C₃-C₈ cycloalkyl. In embodiments, R¹⁷ is independently substituted or unsubstituted 3 to 8 membered heterocycloalkyl. In embodiments, R¹⁷ is independently substituted or unsubstituted C₆ aryl. In embodiments, R¹⁷ is independently substituted or unsubstituted 5 to 6 membered heteroaryl. In embodiments, R¹⁷ is independently substituted C₁-C₆ alkyl. In embodiments, R¹⁷ is independently substituted 2 to 6 membered heteroalkyl. In embodiments, R¹⁷ is independently substituted C₃-C₈ cycloalkyl. In embodiments, R¹⁷ is independently substituted 3 to 8 membered heterocycloalkyl. In embodiments, R¹⁷ is independently substituted C₆ aryl. In embodiments, R¹⁷ is independently substituted 5 to 6 membered heteroaryl. In embodiments, R¹⁷ is independently unsubstituted C₁-C₆ alkyl. In embodiments, R¹⁷ is independently unsubstituted 2 to 6 membered heteroalkyl. In embodiments, R¹⁷ is independently unsubstituted C₃-C₈ cycloalkyl. In embodiments, R¹⁷ is independently unsubstituted 3 to 8 membered heterocycloalkyl. In embodiments, R¹⁷ is independently unsubstituted C₆ aryl. In embodiments, R¹⁷ is independently unsubstituted 5 to 6 membered heteroaryl.

In embodiments, R¹⁸ is independently hydrogen. In embodiments, R¹⁸ is independently halogen. In embodiments, R¹⁸ is independently —N₃. In embodiments, R¹⁸ is independently —NO₂. In embodiments, R¹⁸ is independently —CF₃. In embodiments, R¹⁸ is independently —CCl₃. In embodiments, R¹⁸ is independently —CBr₃. In embodiments, R¹⁸ is independently —Cl₃. In embodiments, R¹⁸ is independently —CN. In embodiments, R¹⁸ is independently —OR^{18A}. In embodiments, R¹⁸ is independently ^{—NR}^{18A}R^{18B.} In embodiments, R¹⁸ is independently —COOH. In embodiments, R¹⁸ is independently —CONH₂. In embodiments, R¹⁸ is independently —NO₂. In embodiments, R¹⁸ is independently —SH. In embodiments, R¹⁸ is independently —SO₂Cl. In embodiments, R¹⁸ is independently —SO₃H. In embodiments, R¹⁸ is independently —SO₄H. In embodiments, R¹⁸ is independently —SO₂NH₂. In embodiments, R¹⁸ is independently —NHNH₂. In embodiments, R¹⁸ is independently —ONH₂. In embodiments, R¹⁸ is independently —OCH₃. In embodiments, R¹⁸ is independently —NHCNHNH₂. In embodiments, R¹⁸ is independently substituted or unsubstituted alkyl. In embodiments, R¹⁸ is independently substituted or unsubstituted heteroalkyl. In embodiments, R¹⁸ is independently substituted or unsubstituted cycloalkyl. In embodiments, R¹⁸ is independently substituted or unsubstituted heterocycloalkyl. In embodiments, R¹⁸ is independently substituted or unsubstituted aryl. In embodiments, R¹⁸ is independently substituted or unsubstituted heteroaryl. In embodiments, R¹⁸ is independently —OCH═CH₂. In embodiments, R¹⁸ is independently -L⁴-R¹⁵. In embodiments, R¹⁸ is independently substituted alkyl. In embodiments, R¹⁸ is independently substituted heteroalkyl. In embodiments, R¹⁸ is independently substituted cycloalkyl. In embodiments, R¹⁸ is independently substituted heterocycloalkyl. In embodiments, R¹⁸ is independently substituted aryl. In embodiments, R¹⁸ is independently substituted heteroaryl. In embodiments, R¹⁸ is independently unsubstituted alkyl. In embodiments, R¹⁸ is independently unsubstituted heteroalkyl. In embodiments, R¹⁸ is independently unsubstituted cycloalkyl. In embodiments, R¹⁸ is independently unsubstituted heterocycloalkyl. In embodiments, R¹⁸ is independently unsubstituted aryl. In embodiments, R¹⁸ is independently unsubstituted heteroaryl. In embodiments, R¹⁸ is independently substituted or unsubstituted C₁-C₆ alkyl. In embodiments, R¹⁸ is independently substituted or unsubstituted 2 to 6 membered heteroalkyl. In embodiments, R¹⁸ is independently substituted or unsubstituted C₃-C₈ cycloalkyl. In embodiments, R¹⁸ is independently substituted or unsubstituted 3 to 8 membered heterocycloalkyl. In embodiments, R¹⁸ is independently substituted or unsubstituted C₆ aryl. In embodiments, R¹⁸ is independently substituted or unsubstituted 5 to 6 membered heteroaryl. In embodiments, R¹⁸ is independently substituted C₁-C₆ alkyl. In embodiments, R¹⁸ is independently substituted 2 to 6 membered heteroalkyl. In embodiments, R¹⁸ is independently substituted C₃-C₈ cycloalkyl. In embodiments, le is independently substituted 3 to 8 membered heterocycloalkyl. In embodiments, R¹⁸ is independently substituted C₆ aryl. In embodiments, R¹⁸ is independently substituted 5 to 6 membered heteroaryl. In embodiments, R¹⁸ is independently unsubstituted C₁-C₆ alkyl. In embodiments, R¹⁸ is independently unsubstituted 2 to 6 membered heteroalkyl. In embodiments, R¹⁸ is independently unsubstituted C₃-C₈ cycloalkyl. In embodiments, R¹⁸ is independently unsubstituted 3 to 8 membered heterocycloalkyl. In embodiments, R¹⁸ is independently unsubstituted C₆ aryl. In embodiments, R¹⁸ is independently unsubstituted 5 to 6 membered heteroaryl. In embodiments, R¹⁹ is independently hydrogen. In embodiments, R¹⁹ is independently halogen. In embodiments, R¹⁹ is independently —N₃. In embodiments, R¹⁹ is independently —NO₂. In embodiments, R¹⁹ is independently —CF₃. In embodiments, R¹⁹ is independently —CCl₃. In embodiments, R¹⁹ is independently —CBr₃. In embodiments, R¹⁹ is independently —Cl₃. In embodiments, R¹⁹ is independently —CN. In embodiments, R¹⁹ is independently —OR^{19A}. In embodiments, R¹⁹ is independently —NR^{19A}R¹⁹B. In embodiments, R¹⁹ is independently —COOH. In embodiments, R¹⁹ is independently —CONH₂. In embodiments, R¹⁹ is independently —NO₂. In embodiments, R¹⁹ is independently —SH. In embodiments, R¹⁹ is independently —SO₂Cl. In embodiments, R¹⁹ is independently —SO₃H. In embodiments, R¹⁹ is independently —SO₄H. In embodiments, R¹⁹ is independently —SO₂NH₂. In embodiments, R¹⁹ is independently —NHNH₂. In embodiments, R¹⁹ is independently —ONH₂. In embodiments, R¹⁹ is independently —OCH₃. In embodiments, R¹⁹ is independently —NHCNHNH₂. In embodiments, R¹⁹ is independently substituted or unsubstituted alkyl. In embodiments, R¹⁹ is independently substituted or unsubstituted heteroalkyl. In embodiments, R¹⁹ is independently substituted or unsubstituted cycloalkyl. In embodiments, R¹⁹ is independently substituted or unsubstituted heterocycloalkyl. In embodiments, R¹⁹ is independently substituted or unsubstituted aryl. In embodiments, R¹⁹ is independently substituted or unsubstituted heteroaryl. In embodiments, R¹⁹ is independently —OCH═CH₂. In embodiments, R¹⁹ is independently L⁴-R¹⁵. In embodiments, R¹⁹ is independently substituted alkyl. In embodiments, R¹⁹ is independently substituted heteroalkyl. In embodiments, R¹⁹ is independently substituted cycloalkyl. In embodiments, R¹⁹ is independently substituted heterocycloalkyl. In embodiments, R¹⁹ is independently substituted aryl. In embodiments, R¹⁹ is independently substituted heteroaryl. In embodiments, R¹⁹ is independently unsubstituted alkyl. In embodiments, R¹⁹ is independently unsubstituted heteroalkyl. In embodiments, R¹⁹ is independently unsubstituted cycloalkyl. In embodiments, R¹⁹ is independently unsubstituted heterocycloalkyl. In embodiments, R¹⁹ is independently unsubstituted aryl. In embodiments, R¹⁹ is independently unsubstituted heteroaryl. In embodiments, R¹⁹ is independently substituted or unsubstituted C₁-C₆ alkyl.

In embodiments, R¹⁹ is independently substituted or unsubstituted 2 to 6 membered heteroalkyl. In embodiments, R¹⁹ is independently substituted or unsubstituted C₃-C₈ cycloalkyl. In embodiments, R¹⁹ is independently substituted or unsubstituted 3 to 8 membered heterocycloalkyl. In embodiments, R¹⁹ is independently substituted or unsubstituted C₆ aryl. In embodiments, R¹⁹ is independently substituted or unsubstituted 5 to 6 membered heteroaryl. In embodiments, R¹⁹ is independently substituted C₁-C₆ alkyl.

In embodiments, R¹⁹ is independently substituted 2 to 6 membered heteroalkyl. In embodiments, R¹⁹ is independently substituted C₃-C₈ cycloalkyl. In embodiments, R¹⁹ is independently substituted 3 to 8 membered heterocycloalkyl. In embodiments, R¹⁹ is independently substituted C₆ aryl. In embodiments, R¹⁹ is independently substituted 5 to 6 membered heteroaryl. In embodiments, R¹⁹ is independently unsubstituted C₁-C₆ alkyl. In embodiments, R¹⁹ is independently unsubstituted 2 to 6 membered heteroalkyl. In embodiments, R¹⁹ is independently unsubstituted C₃-C₈ cycloalkyl. In embodiments, R¹⁹ is independently unsubstituted 3 to 8 membered heterocycloalkyl. In embodiments, R¹⁹ is independently unsubstituted C₆ aryl. In embodiments, R¹⁹ is independently unsubstituted 5 to 6 membered heteroaryl.

In embodiments, R²⁰ is independently hydrogen. In embodiments, R²⁰ is independently halogen. In embodiments, R²⁰ is independently —N₃. In embodiments, R²⁰ is independently —NO₂. In embodiments, R²⁰ is independently —CF₃. In embodiments, R²⁰ is independently —CCl₃. In embodiments, R²⁰ is independently —CBr₃. In embodiments, R²⁰ is independently —Cl₃. In embodiments, R²⁰ is independently —CN. In embodiments, R²⁰ is independently —OR^{20A.} In embodiments, R²⁰ is independently —NR^{20A}R^{20B.} In embodiments, R²⁰ is independently —COOH. In embodiments, R²⁰ is independently —CONH₂. In embodiments, R²⁰ is independently —NO₂. In embodiments, R²⁰ is independently —SH. In embodiments, R²⁰ is independently —SO₂Cl. In embodiments, R²⁰ is independently —SO₃H. In embodiments, R²⁰ is independently —SO₄H. In embodiments, R²⁰ is independently —SO₂NH₂. In embodiments, R²⁰ is independently —NHNH₂. In embodiments, R²⁰ is independently —ONH₂. In embodiments, R²⁰ is independently —OCH₃. In embodiments, R²⁰ is independently —NHCNHNH₂. In embodiments, R²⁰ is independently substituted or unsubstituted alkyl. In embodiments, R²⁰ is independently substituted or unsubstituted heteroalkyl. In embodiments, R²⁰ is independently substituted or unsubstituted cycloalkyl. In embodiments, R²⁰ is independently substituted or unsubstituted heterocycloalkyl. In embodiments, R²⁰ is independently substituted or unsubstituted aryl. In embodiments, R²⁰ is independently substituted or unsubstituted heteroaryl. In embodiments, R²⁰ is independently —OCH═CH₂. In embodiments, R²⁰ is independently -L⁴-R¹⁵. In embodiments, R²⁰ is independently substituted alkyl. In embodiments, R²⁰ is independently substituted heteroalkyl. In embodiments, R²⁰ is independently substituted cycloalkyl. In embodiments, R²⁰ is independently substituted heterocycloalkyl. In embodiments, R²⁰ is independently substituted aryl. In embodiments, R²⁰ is independently substituted heteroaryl. In embodiments, R²⁰ is independently unsubstituted alkyl. In embodiments, R²⁰ is independently unsubstituted heteroalkyl. In embodiments, R²⁰ is independently unsubstituted cycloalkyl. In embodiments, R²⁰ is independently unsubstituted heterocycloalkyl. In embodiments, R²⁰ is independently unsubstituted aryl. In embodiments, R²⁰ is independently unsubstituted heteroaryl. In embodiments, R²⁰ is independently substituted or unsubstituted C₁-C₆ alkyl. In embodiments, R²⁰ is independently substituted or unsubstituted 2 to 6 membered heteroalkyl. In embodiments, R²⁰ is independently substituted or unsubstituted C₃-C₈ cycloalkyl. In embodiments, R²⁰ is independently substituted or unsubstituted 3 to 8 membered heterocycloalkyl. In embodiments, R²⁰ is independently substituted or unsubstituted C₆ aryl. In embodiments, R²⁰ is independently substituted or unsubstituted 5 to 6 membered heteroaryl. In embodiments, R²⁰ is independently substituted C₁-C₆ alkyl. In embodiments, R²⁰ is independently substituted 2 to 6 membered heteroalkyl. In embodiments, R²⁰ is independently substituted C₃-C₈ cycloalkyl. In embodiments, R²⁰ is independently substituted 3 to 8 membered heterocycloalkyl. In embodiments, R²⁰ is independently substituted C₆ aryl. In embodiments, R²⁰ is independently substituted 5 to 6 membered heteroaryl. In embodiments, R²⁰ is independently unsubstituted C₁-C₆ alkyl. In embodiments, R²⁰ is independently unsubstituted 2 to 6 membered heteroalkyl. In embodiments, R²⁰ is independently unsubstituted C₃-C₈ cycloalkyl. In embodiments, R²⁰ is independently unsubstituted 3 to 8 membered heterocycloalkyl. In embodiments, R²⁰ is independently unsubstituted C₆ aryl. In embodiments, R²⁰ is independently unsubstituted 5 to 6 membered heteroaryl.

In embodiments, R²¹ is independently hydrogen. In embodiments, R²¹ is independently halogen. In embodiments, R²¹ is independently —N₃. In embodiments, R²¹ is independently —NO₂. In embodiments, R²¹ is independently —CF₃. In embodiments, R²¹ is independently —CCl₃. In embodiments, R²¹ is independently —CBr₃. In embodiments, R²¹ is independently —Cl₃. In embodiments, R²¹ is independently —CN. In embodiments, R²¹ is independently —OR^{21A.} In embodiments, R²¹ is independently —NR^{21A}R^{21B.} In embodiments, R²¹ is independently —COOH. In embodiments, R²¹ is independently —CONH₂. In embodiments, R²¹ is independently —NO₂. In embodiments, R²¹ is independently —SH. In embodiments, R²¹ is independently —SO₂Cl. In embodiments, R²¹ is independently —SO₃H. In embodiments, R²¹ is independently —SO₄H. In embodiments, R²¹ is independently —SO₂NH₂. In embodiments, R²¹ is independently —NHNH₂. In embodiments, R²¹ is independently —ONH₂. In embodiments, R²¹ is independently —OCH₃. In embodiments, R²¹ is independently —NHCNHNH₂. In embodiments, R²¹ is independently substituted or unsubstituted alkyl. In embodiments, R²¹ is independently substituted or unsubstituted heteroalkyl. In embodiments, R²¹ is independently substituted or unsubstituted cycloalkyl. In embodiments, R²¹ is independently substituted or unsubstituted heterocycloalkyl. In embodiments, R²¹ is independently substituted or unsubstituted aryl. In embodiments, R²¹ is independently substituted or unsubstituted heteroaryl. In embodiments, R²¹ is independently —OCH═CH₂. In embodiments, R²¹ is independently -L⁴-R¹⁵. In embodiments, R²¹ is independently substituted alkyl. In embodiments, R²¹ is independently substituted heteroalkyl. In embodiments, R²¹ is independently substituted cycloalkyl. In embodiments, R²¹ is independently substituted heterocycloalkyl. In embodiments, R²¹ is independently substituted aryl. In embodiments, R²¹ is independently substituted heteroaryl. In embodiments, R²¹ is independently unsubstituted alkyl. In embodiments, R²¹ is independently unsubstituted heteroalkyl. In embodiments, R²¹ is independently unsubstituted cycloalkyl. In embodiments, R²¹ is independently unsubstituted heterocycloalkyl. In embodiments, R²¹ is independently unsubstituted aryl. In embodiments, R²¹ is independently unsubstituted heteroaryl. In embodiments, R²¹ is independently substituted or unsubstituted C₁-C₆ alkyl. In embodiments, R²¹ is independently substituted or unsubstituted 2 to 6 membered heteroalkyl. In embodiments, R²¹ is independently substituted or unsubstituted C₃-C₈ cycloalkyl. In embodiments, R²¹ is independently substituted or unsubstituted 3 to 8 membered heterocycloalkyl. In embodiments, R²¹ is independently substituted or unsubstituted C₆ aryl. In embodiments, R²¹ is independently substituted or unsubstituted 5 to 6 membered heteroaryl. In embodiments, R²¹ is independently substituted C₁-C₆ alkyl. In embodiments, R²¹ is independently substituted 2 to 6 membered heteroalkyl. In embodiments, R²¹ is independently substituted C₃-C₈ cycloalkyl. In embodiments, R²¹ is independently substituted 3 to 8 membered heterocycloalkyl. In embodiments, R²¹ is independently substituted C₆ aryl. In embodiments, R²¹ is independently substituted 5 to 6 membered heteroaryl. In embodiments, R²¹ is independently unsubstituted C₁-C₆ alkyl. In embodiments, R²¹ is independently unsubstituted 2 to 6 membered heteroalkyl. In embodiments, R²¹ is independently unsubstituted C₃-C₈ cycloalkyl. In embodiments, R²¹ is independently unsubstituted 3 to 8 membered heterocycloalkyl. In embodiments, R²¹ is independently unsubstituted C₆ aryl. In embodiments, R²¹ is independently unsubstituted 5 to 6 membered heteroaryl.

In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently hydrogen. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted or unsubstituted alkyl. In embodiments, each of R^{16A}, R^{16B}, R^{17 A}, R^{17B}, R^{18A}, R^{18B}, R^{19A}, R^{19B}, R^{20A}, R^{20B}, R^{21A}, and R^{21B} is independently substituted or unsubstituted heteroalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21 B} is independently substituted or unsubstituted cycloalkyl. In embodiments, each of R^{16A}, R^{16B}, R^{17 A}, R^{17B}, R^{18A}, R^{18B}, R^{19A}, R^{19B}, R^{20A}, R^{20B}, R^{21A}, and R^{21B} is independently substituted or unsubstituted heterocycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted or unsubstituted aryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted or unsubstituted heteroaryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted alkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted heteroalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted cycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted heterocycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted aryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted heteroaryl. In embodiments, each of R^{16A}, R^{16B}, R^{17A}, R^{17B}, R^{18A}, R^{18B}R^{19A}, R^{19B}, R^{20A}, R^{20B}, R^{21A}, and R^{21B} is independently unsubstituted alkyl. In embodiments, each of R^{16A, R}^{16B}, R^{17A}, R^{17B}, R^{18A}, R^{18B}, R^{19A}, R^{19B}, R^{20A}, R^{20B}, R^{21A}, and R^{21B} is independently unsubstituted heteroalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently unsubstituted cycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently unsubstituted heterocycloalkyl. In embodiments, each of R^{16A}, R^{16B}, R^{17A}, R^{17B}R^{18A}, R^{18B}, R^{19A}, R^{19B}, R^{20A}, R^{20B}, R^{21A}, and R^{21B} is independently unsubstituted aryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently unsubstituted heteroaryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B}, is independently substituted or unsubstituted C₁-C₆ alkyl. In embodiments, each of R^{16A}, R^{16B}, R^{17A}, R^{17B}, R^{18A}, R^{18B}, R^{19A}, R^{19B}, R^{20A}, R^{20B}, R^{21B}, and R^{21B} is independently substituted or unsubstituted 2 to 6 membered heteroalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted or unsubstituted C₃-C₈ cycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted or unsubstituted 3 to 8 membered heterocycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted or unsubstituted C₆ aryl. In embodiments, each of R^{16A}, R^{16B}, R^{17A}, R^{17B}, R¹⁸A, R^{18B}, R^{19A}, R^{19B}, R^{20A}, R^{20B}, R^{21A}, and R^{21B} is independently substituted or unsubstituted 5 to 6 membered heteroaryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted C₁-C₆ alkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted 2 to 6 membered heteroalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted C₃-C₈ cycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted 3 to 8 membered heterocycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A},and R^{21B} is independently substituted C₆ aryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently substituted 5 to 6 membered heteroaryl. In embodiments, each of R^{16A}, R^{16B}, R^{17A}, R^{17B}, R^{18A}, R^{18B}, R^{19A}, R^{19B}, R^{20A}, R^{20B}, R^{21A},and R^{21B} is independently unsubstituted C₁-C₆ alkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently unsubstituted 2 to 6 membered heteroalkyl. In embodiments, each of R^{16A, R}^{16B}, R^{17A}, R^{17B}, R^{18A}, R^{18B}, R^{19A}, R^{19B}, R^{20A}, R^{20B}, R^{21A}, and R^{21B} is independently unsubstituted C₃-C₈ cycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently unsubstituted 3 to 8 membered heterocycloalkyl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently unsubstituted C₆ aryl. In embodiments, each of R^{16A}, R^{16B}, R^{17A}, R^{17B}, R^{18A}, R^{18B}, R^{19A}, R^{19B}, R^{20A}, R^{20B}, R^{21A}, and R^{21B} is independently unsubstituted 5 to 6 membered heteroaryl.

In embodiments, each of R^{X3} and R^{X3′} is independently hydrogen. In embodiments, each of R^{X3} and R^{X3′} is independently substituted or unsubstituted alkyl. In embodiments, each of R^{X3} and R^{X3′} is independently substituted alkyl. In embodiments, each of R^{X3} and R^{X3′} is independently unsubstituted alkyl. In embodiments, each of R^{X3} and R^{X3′} is independently substituted or unsubstituted C₁-C₆ alkyl. In embodiments, each of R^{X3} and R^{X3′} is independently substituted C₁-C₆ alkyl. In embodiments, each of R^{X3} and R^{X3′} is independently unsubstituted C₁-C₆ alkyl.

In embodiments, each of R²² and R²³ is independently hydrogen. In embodiments, each of R²² and R²³ is independently -L⁴-R¹⁵. In embodiments, each of R²² and R²³ is independently substituted or unsubstituted alkyl. In embodiments, each of R²² and R²³ is independently substituted alkyl. In embodiments, each of R²² and R²³ is independently unsubstituted alkyl. In embodiments, each of R²² and R²³ is independently substituted or unsubstituted C₁-C₆ alkyl. In embodiments, each of R²² and R²³ is independently substituted C₁-C₆ alkyl. In embodiments, each of R²² and R²³ is independently unsubstituted C₁-C₆ alkyl.

In embodiments, each of R²⁴, R²⁵, R²⁶, R²⁷, R²⁸, R²⁹, R³⁰, and R³¹ is independently hydrogen. In embodiments, each of R²⁴, R²⁵, R²⁶, R²⁷, R²⁸, R²⁹, R³⁰, and R³¹ is independently halogen. In embodiments, each of R²⁴, R²⁵, R²⁶, R²⁷, R²⁸, R²⁹, R³⁰,and R³¹ is independently —N₃. In embodiments, each of R²⁴, R²⁵, R²⁶, R²⁷, R²⁸, R²⁹, R³⁰, and R³¹ is independently —NO₂. In embodiments, each of R^{24, R}²⁵, R²⁶, R²⁷, R²⁸, R²⁹, R³⁰, and R³¹ is independently —CF₃. In embodiments, each of R²⁴, R²⁵, R²⁶, R²⁷, R²⁸, R²⁹, R³⁰, and R³¹ is independently —CCl₃. In embodiments, each of R²⁴, R²⁵, R²⁶, R²⁷, R²⁸, R²⁹, R³⁰, and R³¹ is independently —CBr₃. In embodiments, each of _{R}²⁴, R²⁵, R²⁶, R²⁷, R²⁸, R²⁹, R³⁰, and R³¹ is independently —Cl₃. In embodiments, each of _{R}²⁴, R²⁵, R²⁶, R²⁷, R²⁸, R²⁹, R³⁰, and R³¹ is independently —CN. In embodiments, each of R²⁴, R²⁵, R²⁶, R²⁷, R²⁸, R²⁹, R³⁰, and R³¹ is independently —OH. In embodiments, each of R²⁴, R²⁵, R²⁶, R²⁷, R²⁸, R²⁹, R³⁰, and R³¹ is independently —NH₂. In embodiments, each of R^{24, R}²⁵, R²⁶, R²⁷, R²⁸, R²⁹, R³⁰, and R³¹ is independently —NMe₂. In embodiments, each of R²⁴, R²⁵, R²⁶, R²⁷, R²⁸, R²⁹, R³⁰, and R³¹ is independently —NEt₂. In embodiments, each of R²⁴, R²⁵, R²⁶, R²⁷, R²⁸, R²⁹, R³⁰, and R³¹ is independently —COOH. In embodiments, each of R²⁴, R²⁵, R²⁶, R²⁷, R²⁸, R²⁹, R³⁰, and R³¹ is independently —CONH₂. In embodiments, each of R²⁴, R²⁵, R²⁶, R²⁷, R²⁸, R²⁹, R³⁰, and R³¹ is independently —NO₂. In embodiments, each of R²⁴, R²⁵, R²⁶, R²⁷, R²⁸, R²⁹, R³⁰, and R³¹ is independently ——SH. In embodiments, each of R²⁴, R²⁵, R²⁶, R²⁷, R²⁸, R²⁹, R³⁰, and R³¹ is independently —SO₂Cl. In embodiments, each of R²⁴, R²⁵, R²⁶, R²⁷, R²⁸, R²⁹, R³⁰, and R³¹ is independently —SO₃H. In embodiments, each of R²⁴, R²⁵, R²⁶, R²⁷, R²⁸, R²⁹, ,R³⁰, and R³¹ is independently —SO₄H. In embodiments, each of R²⁴, R²⁵, R²⁶, R²⁷, R²⁸, R²⁹, R³⁰, and R³¹ is independently —SO₂NH₂. In embodiments, each of R²⁴, R²⁵, R²⁶, R²⁷, R²⁸, R²⁹, R³⁰, and R³¹ is independently —NHNH₂. In embodiments, each of R²⁴, R²⁵, R²⁶, R²⁷, R²⁸, R²⁹, R³⁰, and R³¹ is independently —ONH₂. In embodiments, each of R²⁴, R²⁵, R²⁶, R²⁷, R²⁸, R²⁹, R³⁰, and R³¹ is independently —OCH₃. In embodiments, each of R²⁴, R²⁵, R²⁶, R²⁷, R²⁸, R²⁹, R³⁰, and R³¹, is independently —NHCNHNH₂. In embodiments, each of R²⁴, R²⁵, R²⁶, R²⁷, R²⁸, R²⁹, R³⁰, and R³¹ is independently substituted or unsubstituted alkyl. In embodiments, each of R²⁴, R²⁵, R²⁶, R²⁷, R²⁸, R²⁹, R³⁰, and R³¹ is independently substituted or unsubstituted heteroalkyl. In embodiments, each of R²⁴, R²⁵, R²⁶, R²⁷, R²⁸, R²⁹, R³⁰, and R³¹ is independently substituted or unsubstituted cycloalkyl. In embodiments, R²⁴ is independently substituted or unsubstituted heterocycloalkyl. In embodiments, each of R²⁴, R²⁵, R²⁶, R²⁷, R²⁸, R²⁹, R³⁰, and R³¹ is independently substituted or unsubstituted aryl. In embodiments, each of R²⁴, R²⁵, R²⁶, R²⁷, R²⁸, R²⁹, R³⁰, and R³¹ is independently substituted or unsubstituted heteroaryl. In embodiments, each of R²⁴, R²⁵, R²⁶, R²⁷, R²⁸, R²⁹, R³⁰, and R³¹ is independently -L⁴-R¹⁵. In embodiments, each of R²⁴, R²⁵, R²⁶, R²⁷, R²⁸, R²⁹, R³⁰, and R³¹ is independently substituted alkyl. In embodiments, each of R²⁴, R²⁵, R²⁶, R²⁷, R²⁸, R²⁹, R³⁰, and R³¹ is independently substituted heteroalkyl. In embodiments, each of R²⁴, R²⁵, R²⁶, R²⁷, R²⁸, R²⁹, R³⁰, and R³¹ is independently substituted cycloalkyl. In embodiments, each of R²⁴, R²⁵, R²⁶, R²⁷, R²⁸, R²⁹, R³⁰, and R³¹ is independently substituted heterocycloalkyl. In embodiments, each of R²⁴, R²⁵, R²⁶, R²⁷, R²⁸, R²⁹, R³⁰, and R³¹ independently substituted aryl. In embodiments, each of R²⁴, R²⁵, R²⁶, R²⁷, R²⁸, R²⁹, R³⁰, and R³¹ is independently substituted heteroaryl. In embodiments, each of R²⁴, R²⁵, R²⁶, R²⁷, R²⁸, R²⁹, R³⁰, and R³¹ is independently unsubstituted alkyl. In embodiments, each of R²⁴, R²⁵, R²⁶, R²⁷, R²⁸, R²⁹, R³⁰, and R³¹ is independently unsubstituted heteroalkyl. In embodiments, each of R²⁴, R²⁵, R²⁶, R²⁷, R²⁸, R²⁹, R³⁰, and R³¹ is independently unsubstituted cycloalkyl. In embodiments, each of R²⁴, R²⁵, R²⁶, R²⁷, R²⁸, R²⁹, R³⁰, and R³¹ is independently unsubstituted heterocycloalkyl. In embodiments, R²⁴ is independently unsubstituted aryl. In embodiments, each of R²⁴, R²⁵, R²⁶, R²⁷, R²⁸ , R²⁹, R³⁰, and R³¹ is independently unsubstituted heteroaryl. In embodiments, each of R²⁴, R²⁵, R²⁶, R²⁷ , R²⁸, R²⁹, R³⁰, and R³¹ is independently substituted or unsubstituted C₁-C₆ alkyl. In embodiments, each of R²⁴, R²⁵, R²⁶, R²⁷, R²⁸, R²⁹, R³⁰, and R³¹ is independently substituted or unsubstituted 2 to 6 membered heteroalkyl. In embodiments, each of R²⁴, R²⁵, R²⁶, R²⁷, R²⁸, R²⁹, R³⁰, and R³¹ is independently substituted or unsubstituted C₃-C₈ cycloalkyl. In embodiments, each of R²⁴, R²⁵, R²⁶, R²⁷, R²⁸, R²⁹, R³⁰, and R³¹ is independently substituted or unsubstituted 3 to 8 membered heterocycloalkyl. In embodiments, each of R²⁴, R²⁵, R²⁶, R²⁷, R²⁸, R²⁹, R³⁰, and R³¹ is independently substituted or unsubstituted C₆ aryl. In embodiments, each of R²⁴, R²⁵, R²⁶, R²⁷, R²⁸, R²⁹, R³⁰, and R³¹ is independently substituted or unsubstituted 5 to 6 membered heteroaryl. In embodiments, each of R²⁴, R²⁵, R²⁶, R²⁷, R²⁸, R²⁹, R³⁰ and R³¹ is independently substituted C₁-C₆ alkyl. In embodiments, R²⁴ is independently substituted 2 to 6 membered heteroalkyl. In embodiments, each of R²⁴, R²⁵, R²⁶, R²⁷, R²⁸, R²⁹, R³⁰, and R³¹ is independently substituted C₃-C₈ cycloalkyl. In embodiments, each of R²⁴, R²⁵, R²⁶, R²⁷, R²⁸, R²⁹, R³⁰, and R³¹ is independently substituted 3 to 8 membered heterocycloalkyl. In embodiments, each of R²⁴, R²⁵, R²⁶, R²⁷, R²⁸, R²⁹, R³⁰, and R³¹ is independently substituted C₆ aryl. In embodiments, each of _{R}²⁴, R²⁵, R²⁶, R²⁷, R²⁸, R²⁹, R³⁰, and R³¹ is independently substituted 5 to 6 membered heteroaryl. In embodiments, each of R²⁴, R²⁵, R²⁶, R²⁷, R²⁸, R²⁹, R³⁰, and R³¹ is independently unsubstituted C₁-C₆ alkyl. In embodiments, each of R²⁴, R²⁵, R²⁶, R²⁷, R²⁸, R²⁹, R³⁰, and R³¹ is independently unsubstituted 2 to 6 membered heteroalkyl. In embodiments, each of _{R}²⁴, R²⁵, R²⁶, R²⁷, R²⁸, R²⁹, R³⁰, and R³¹ is independently unsubstituted C₃-C₈ cycloalkyl. In embodiments, each of _{R}²⁴, R²⁵, R²⁶, R²⁷, R²⁸, R²⁹, R³⁰, and R³¹ is independently unsubstituted 3 to 8 membered heterocycloalkyl. In embodiments, each of R²⁴, R²⁵, R²⁶, R²⁷, R²⁸, R²⁹, R³⁰, and R³¹ is independently unsubstituted C₆ aryl. In embodiments, each of _{R}²⁴, R²⁵, R²⁶, R²⁷, R²⁸, R²⁹, R³⁰, and R³¹ is independently unsubstituted 5 to 6 membered heteroaryl.

In embodiments, each of R³² and R³³ is independently hydrogen. In embodiments, each of R³² and R³³ is independently halogen. In embodiments, each of R³² and R³³ is independently —N₃. In embodiments, each of R³² and R³³ is independently —NO₂. In embodiments, each of R³² and R³³ is independently —CF₃. In embodiments, each of R³² and R³³ is independently —CCl₃. In embodiments each of R³² and R³³ is independently —CBr₃. In embodiments, each of R³² and R³³ is independently —Cl₃. In embodiments, each of R³² and R³³ is independently —CN. In embodiments each of R³² and R³³ is independently —OH. In embodiments, each of R³² and R³³ is independently —NH₂. In embodiments each of R³² and R³³ is independently —NMe₂. In embodiments, each of R³² and R³³ is independently —NEt₂. In embodiments, each of R³² and R³³ is independently —COOH. In embodiments each of R³² and R³³ is independently —CONH₂. In embodiments, each of R³² and R³³ is independently —NO₂. In embodiments, each of R³² and R³³ is independently —SH. In embodiments, each of R³² and R³³ is independently —SO₂Cl. In embodiments, each of R³² and R³³ is independently —SO₃H. In embodiments, each of R³² and R³³ is independently —SO₄H. In embodiments, each of R³² and R³³ is independently —SO₂NH₂. In embodiments, each of R³² and R³³ is independently —NHNH₂. In embodiments, each of R³² and R³³ is independently —ONH₂. In embodiments, each of R³² and R³³ is independently —OCH₃. In embodiments, each of R³² and R³³ is independently —NHCNHNH₂. In embodiments, each of R³² and R³³ is independently substituted or unsubstituted alkyl. In embodiments each of R³² and R³³ is independently substituted or unsubstituted heteroalkyl. In embodiments, each of R³² and R³³ is independently substituted or unsubstituted cycloalkyl. In embodiments, each of R³² and R³³ is independently substituted or unsubstituted heterocycloalkyl. In embodiments, each of R³² and R³³ is independently substituted or unsubstituted aryl. In embodiments, each of R³² and R³³ is independently substituted or unsubstituted heteroaryl. In embodiments, each of R³² and R³³ is independently -L⁴-R¹⁵. In embodiments, each of R³² and R³³ is independently substituted alkyl. In embodiments, each of R³² and R³³ is independently substituted heteroalkyl. In embodiments, each of R³² and R³³ is independently substituted cycloalkyl. In embodiments, each of R³² and R³³ is independently substituted heterocycloalkyl. In embodiments, each of R³² and R³³ independently substituted aryl. In embodiments, each of R³² and R³³ is independently substituted heteroaryl. In embodiments, each of R³² and R³³ is independently unsubstituted alkyl. In embodiments, each of R³² and R³³ is independently unsubstituted heteroalkyl. In embodiments, each of R³² and R³³ is independently unsubstituted cycloalkyl. In embodiments, each of R³² and R³³ is independently unsubstituted heterocycloalkyl. In embodiments each of R³² and R³³ is independently unsubstituted aryl. In embodiments, each of R³² and R³³ is independently unsubstituted heteroaryl. In embodiments, each of R³² and R³³ is independently substituted or unsubstituted C₁-C₆ alkyl. In embodiments, each of R³² and R³³ is independently substituted or unsubstituted 2 to 6 membered heteroalkyl. In embodiments, each of R³² and R³³ is independently substituted or unsubstituted C₃-C₈ cycloalkyl. In embodiments, each of R³² and R³³ is independently substituted or unsubstituted 3 to 8 membered heterocycloalkyl. In embodiments, each of R³² and R³³ is independently substituted or unsubstituted C₆ aryl. In embodiments, each of R³² and R³³ is independently substituted or unsubstituted 5 to 6 membered heteroaryl. In embodiments, each of R³² and R³³ is independently substituted C₁-C₆ alkyl. In embodiments, each of R³² and R³³ is independently substituted 2 to 6 membered heteroalkyl. In embodiments, each of R³² and R³³ is independently substituted C₃-C₈ cycloalkyl. In embodiments, each of R³² and R³³ is independently substituted 3 to 8 membered heterocycloalkyl. In embodiments, each of R³² and R³³ is independently substituted C₆ aryl. In embodiments, each of R³² and R³³ is independently substituted 5 to 6 membered heteroaryl. In embodiments, each of R³² and R³³ is independently unsubstituted C₁-C₆ alkyl. In embodiments, each of R³² and R³³ is independently unsubstituted 2 to 6 membered heteroalkyl. In embodiments, each of R³² and R³³ is independently unsubstituted C₃-C₈ cycloalkyl. In embodiments, each of R³² and R³³ is independently unsubstituted 3 to 8 membered heterocycloalkyl. In embodiments, each of R³² and R³³ is independently unsubstituted C₆ aryl. In embodiments, each of R³² and R³³ is independently unsubstituted 5 to 6 membered heteroaryl.

In embodiments, R³⁴ is independently hydrogen. In embodiments, R³⁴ is independently halogen. In embodiments, R³⁴ is independently —N₃. In embodiments, R³⁴ is independently —NO₂. In embodiments, R³⁴ is independently —CF₃. In embodiments, R³⁴ is independently —CCl₃. In embodiments, R³⁴ is independently —CBr₃. In embodiments, R³⁴ is independently —Cl₃. In embodiments, R³⁴ is independently CN. In embodiments, R³⁴ is independently —OR^{34A}. In embodiments, R³⁴ is independently —NR^{34A}R^{34B.} In embodiments, R³⁴ is independently —COOH. In embodiments, R³⁴ is independently —CONH₂. In embodiments, R³⁴ is independently —NO₂. In embodiments, R³⁴ is independently —SH. In embodiments, R³⁴ is independently —SO₂Cl. In embodiments, R³⁴ is independently —SO₃H. In embodiments, R³⁴ is independently —SO₄H. In embodiments, R³⁴ is independently —SO₂NH₂.

In embodiments, R³⁴ is independently —NHNH₂. In embodiments, R³⁴ is independently —ONH₂. In embodiments, R³⁴ is independently —OCH₃. In embodiments, R³⁴ is independently —NHCNHNH₂. In embodiments, R³⁴ is independently substituted or unsubstituted alkyl. In embodiments, R³⁴ is independently substituted or unsubstituted heteroalkyl. In embodiments, R³⁴ is independently substituted or unsubstituted cycloalkyl. In embodiments, R³⁴ is independently substituted or unsubstituted heterocycloalkyl. In embodiments, R³⁴ is independently substituted or unsubstituted aryl. In embodiments, R³⁴ is independently substituted or unsubstituted heteroaryl. In embodiments, R³⁴ is independently substituted alkyl. In embodiments, R³⁴ is independently substituted heteroalkyl. In embodiments, R³⁴ is independently substituted cycloalkyl. In embodiments, R³⁴ is independently substituted heterocycloalkyl. In embodiments, R³⁴ independently substituted aryl. In embodiments, R³⁴ is independently substituted heteroaryl. In embodiments, R³⁴ is independently unsubstituted alkyl. In embodiments, R³⁴ is independently unsubstituted heteroalkyl. In embodiments, R³⁴ is independently unsubstituted cycloalkyl. In embodiments, R³⁴ is independently unsubstituted heterocycloalkyl. In embodiments, R³⁴ is independently unsubstituted aryl. In embodiments, R³⁴ is independently unsubstituted heteroaryl. In embodiments, R³⁴ is independently substituted or unsubstituted C₁-C₆ alkyl. In embodiments, R³⁴ is independently substituted or unsubstituted 2 to 6 membered heteroalkyl. In embodiments, R³⁴ is independently substituted or unsubstituted C₃-C₈ cycloalkyl. In embodiments, R³⁴ is independently substituted or unsubstituted 3 to 8 membered heterocycloalkyl. In embodiments, R³⁴ is independently substituted or unsubstituted C₆ aryl. In embodiments, R³⁴ is independently substituted or unsubstituted 5 to 6 membered heteroaryl. In embodiments, R³⁴ is independently substituted C₁-C₆ alkyl. In embodiments, R³⁴ is independently substituted 2 to 6 membered heteroalkyl. In embodiments, R³⁴ is independently substituted C₃-C₈ cycloalkyl. In embodiments, R³⁴ is independently substituted 3 to 8 membered heterocycloalkyl. In embodiments, R³⁴ is independently substituted C₆ aryl. In embodiments, R³⁴ is independently substituted 5 to 6 membered heteroaryl. In embodiments, R³⁴ is independently unsubstituted C₁-C₆ alkyl. In embodiments, R³⁴ is independently unsubstituted 2 to 6 membered heteroalkyl. In embodiments, R³⁴ is independently unsubstituted C₃-C₈ cycloalkyl. In embodiments, R³⁴ is independently unsubstituted 3 to 8 membered heterocycloalkyl. In embodiments, R³⁴ is independently unsubstituted C₆ aryl. In embodiments, R³⁴ is independently unsubstituted 5 to 6 membered heteroaryl.

In embodiments, L⁵ is independently a bond. In embodiments, L⁵ is independently —NR¹⁻⁵-. In embodiments, L⁵ is independently O. In embodiments, L⁵ is independently S. In embodiments, L⁵ is independently substituted or unsubstituted alkylene. In embodiments, L⁵ is independently substituted or unsubstituted alkenylene. In embodiments, L⁵ is independently substituted or unsubstituted alkynylene. In embodiments, L⁵ is independently substituted or unsubstituted heteroalkylene. In embodiments, L⁵ is independently substituted or unsubstituted cycloalkylene. In embodiments, L⁵ is independently substituted or unsubstituted heterocycloalkylene. In embodiments, L⁵ is independently substituted or unsubstituted arylene. In embodiments, L⁵ is independently substituted or unsubstituted heteroarylene. In embodiments, L⁵ is independently substituted alkylene. In embodiments, L⁵ is independently substituted alkenylene. In embodiments, L⁵ is independently substituted alkynylene. In embodiments, L⁵ is independently substituted heteroalkylene. In embodiments, L⁵ is independently substituted cycloalkylene. In embodiments, L⁵ is independently substituted heterocycloalkylene. In embodiments, L⁵ is independently substituted arylene. In embodiments, L⁵ is independently substituted heteroarylene. In embodiments, L⁵ is independently unsubstituted alkylene. In embodiments, L⁵ is independently unsubstituted alkenylene. In embodiments, L⁵ is independently unsubstituted alkynylene. In embodiments, L⁵ is independently unsubstituted heteroalkylene. In embodiments, L⁵ is independently unsubstituted cycloalkylene. In embodiments, L⁵ is independently unsubstituted heterocycloalkylene. In embodiments, L⁵ is independently unsubstituted arylene. In embodiments, L⁵ is independently unsubstituted heteroarylene. In embodiments, L⁵ is independently substituted or unsubstituted C₁-C₆ alkylene. In embodiments, L⁵ is independently substituted or unsubstituted C₂-C₆ alkenylene. In embodiments, L⁵ is independently substituted or unsubstituted C₂-C₆ alkynylene. In embodiments, L⁵ is independently substituted or unsubstituted 2 to 6 membered heteroalkylene. In embodiments, L⁵ is independently substituted or unsubstituted C₃-C₈ cycloalkylene. In embodiments, L⁵ is independently substituted or unsubstituted 3 to 8 membered heterocycloalkylene. In embodiments, L⁵ is independently substituted or unsubstituted C₆ arylene. In embodiments, L⁵ is independently substituted or unsubstituted 5 to 6 membered heteroarylene. In embodiments, L⁵ is independently substituted C₁-C₆ alkylene. In embodiments, L⁵ is independently substituted C₂-C₆ alkenylene. In embodiments, L⁵ is independently substituted C₂-C₆ alkynylene. In embodiments, L⁵ is independently substituted 2 to 6 membered heteroalkylene. In embodiments, L⁵ is independently substituted C₃-C₈ cycloalkylene. In embodiments, L⁵ is independently substituted 3 to 8 membered heterocycloalkylene. In embodiments, L⁵ is independently substituted C₆ arylene. In embodiments, L⁵ is independently substituted 5 to 6 membered heteroarylene. In embodiments, L⁵ is independently unsubstituted C₁-C₆ alkylene. In embodiments, L⁵ is independently unsubstituted C₂-C₆ alkenylene. In embodiments, L⁵ is independently unsubstituted C₂-C₆ alkynylene. In embodiments, L⁵ is independently unsubstituted 2 to 6 membered heteroalkylene. In embodiments, L⁵ is independently unsubstituted C₃-C₈ cycloalkylene. In embodiments, L⁵ is independently unsubstituted 3 to 8 membered heterocycloalkylene. In embodiments, L⁵ is independently unsubstituted C₆ arylene. In embodiments, L⁵ is independently unsubstituted 5 to 6 membered heteroarylene.

In embodiments, each of R^{34A}, R^{34B}, and R^{L5} is independently hydrogen. In embodiments, each of R^{34A}, R^{34B}, and R^{L5} is independently substituted or unsubstituted alkyl. In embodiments, each of R^{34A}, R^{34B}, and R^{L5} is independently substituted or unsubstituted heteroalkyl. In embodiments, each of R^{34A}, R^{34B}, and R^{L5} is independently substituted or unsubstituted cycloalkyl. In embodiments, each of R^{34A}, R^{34B}, and R^{L5} is independently substituted or unsubstituted heterocycloalkyl. In embodiments, each of R^{34A}, R^{34B}, and R^{L5} is independently substituted or unsubstituted aryl. In embodiments, each of R^{34A}, R^{34B}, and R^{L5} is independently substituted or unsubstituted heteroaryl. In embodiments, each of R^{34A}, R^{34B}, and R^{L5} is independently substituted alkyl. In embodiments, each of R^{34A}, R^{34B}, and R^{L5} is independently substituted heteroalkyl. In embodiments, each of R^{34A}, R^{34B}, and R^{L5} is independently substituted cycloalkyl. In embodiments, each of R^{34A}, R^{34B}, and R^{L5} is independently substituted heterocycloalkyl. In embodiments, each of R^{34A}, R^{34B}, and R^{L5} independently substituted aryl. In embodiments, each of R^{34A}, R^{34B}, and R^{L5} is independently substituted heteroaryl. In embodiments, each of R^{34A}, R^{34B}, and R^{L5} is independently unsubstituted alkyl. In embodiments, each of R^{34A}, R^{34B}, and R^{L5} is independently unsubstituted heteroalkyl. In embodiments each of R^{34A}, _{R}^{34B}, and R^{L5}, is independently unsubstituted cycloalkyl. In embodiments, each of R^{34A}, R^{34B}, and R^{L5} is independently unsubstituted heterocycloalkyl. In embodiments, each of R^{34A}, R^{34B}, and R^{L5} is independently unsubstituted aryl. In embodiments, each of R^{34A}, R^{34B}, and R^{L5} is independently unsubstituted heteroaryl. In embodiments, each of R^{34A}, R^{34B}, and R^{L5} is independently substituted or unsubstituted C₁-C₆ alkyl. In embodiments, each of R^{34A}, R^{34B}, and R^{L5} is independently substituted or unsubstituted 2 to 6 membered heteroalkyl. In embodiments, each of R^{34A}, R^{34B}, and R^{L5} is independently substituted or unsubstituted C₃-C₈ cycloalkyl. In embodiments, each of R^{34A}, R^{34B}, and R^{L5} is independently substituted or unsubstituted 3 to 8 membered heterocycloalkyl. In embodiments, each of R^{34A}, R^{34B}, and R^{L5} is independently substituted or unsubstituted C₆ aryl. In embodiments, each of R^{34A}, R^{34B}, and R^{L5} is independently substituted or unsubstituted 5 to 6 membered heteroaryl. In embodiments, each of R^{34A}, R^{34B}, and R^{L5} is independently substituted C₁-C₆ alkyl. In embodiments, each of R^{34A}, R^{34B}, and R^{L5} is independently substituted 2 to 6 membered heteroalkyl. In embodiments, each of R^{34A}, R^{34B}, and R^{L5} is independently substituted C₃-C₈ cycloalkyl. In embodiments, each of R^{34A}, R^{34B}, and R^{L5} is independently substituted 3 to 8 membered heterocycloalkyl. In embodiments, each of R^{34A}, R^{34B}, and R^{L5} is independently substituted C₆ aryl. In embodiments, each of R^{34A}, R^{34B}, and R^{L5} is independently substituted 5 to 6 membered heteroaryl. In embodiments, each of R^{34A}, R^{34B}, and R^{L5} is independently unsubstituted C₁-C₆ alkyl. In embodiments, each of R^{34A}, R^{34B}, and R^{L5} is independently unsubstituted 2 to 6 membered heteroalkyl. In embodiments, each of R^{34A}, R^{34B}, and R^{L5} is independently unsubstituted C₃-C₈ cycloalkyl. In embodiments, each of R^{34A}, R^{34B}, and R^{L5} is independently unsubstituted 3 to 8 membered heterocycloalkyl. In embodiments, each of R^{34A}, R^{34B}, and R^{L5} is independently unsubstituted C₆ aryl. In embodiments, each of R^{34A}, R^{34B}, and R^{L5} is independently unsubstituted 5 to 6 membered heteroaryl.

In embodiments, L¹ is independently a bond, —NR^{A}—, O, S, R³⁶-substituted or unsubstituted alkylene, R³⁶-substituted or unsubstituted alkenylene, R³⁶-substituted or unsubstituted alkynylene, R³⁶-substituted or unsubstituted heteroalkylene, R³⁶-substituted or unsubstituted cycloalkylene, R³⁶-substituted or unsubstituted heterocycloalkylene, R³⁶-substituted or unsubstituted arylene, or R³⁶-substituted or unsubstituted heteroarylene.

In embodiments, L^{R1} is independently a bond, —NR^{B}—, O, S, R³⁷-substituted or unsubstituted alkylene, R³⁷-substituted or unsubstituted alkenylene, R³⁷-substituted or unsubstituted alkynylene, R³⁷-substituted or unsubstituted heteroalkylene, R³⁷-substituted or unsubstituted cycloalkylene, R³⁷-substituted or unsubstituted heterocycloalkylene, R³⁷-substituted or unsubstituted arylene, or R³⁷-substituted or unsubstituted heteroarylene.

In embodiments, R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R³⁸-substituted or unsubstituted alkyl, R³⁸-substituted or unsubstituted heteroalkyl, R³⁸-substituted or unsubstituted cycloalkyl, R³⁸-substituted or unsubstituted heterocycloalkyl, R³⁸-substituted or unsubstituted aryl, or R³⁸-substituted or unsubstituted heteroaryl.

In embodiments, R^{A} is independently hydrogen, R³⁹-substituted or unsubstituted alkyl, R³⁹-substituted or unsubstituted heteroalkyl, R³⁹-substituted or unsubstituted cycloalkyl, R³⁹-substituted or unsubstituted heterocycloalkyl, R³⁹-substituted or unsubstituted aryl, or R³⁹-substituted or unsubstituted heteroaryl.

In embodiments, R^{B} is independently hydrogen, e-substituted or unsubstituted alkyl, e-substituted or unsubstituted heteroalkyl, e-substituted or unsubstituted cycloalkyl, e-substituted or unsubstituted heterocycloalkyl, e-substituted or unsubstituted aryl, or e-substituted or unsubstituted heteroaryl.

In embodiments, each of R^{1A}, R^{1B}, R^{1B′}, R^{1C}, and R^{1C′}, is independently hydrogen or R⁴¹-substituted or unsubstituted alkyl.

In embodiments, each of R^{1F}, R^{1G′} , R^{1H}, R^{1I}, R^{1J} and R^{1K} is independently hydrogen, halogen, R⁴²-substituted or unsubstituted alkyl, R⁴²-substituted or unsubstituted aryl, R⁴²-substituted or unsubstituted heteroaryl, or -L^{R1}-X¹.

In embodiments, each of R¹ and R⁴ is independently hydrogen, R⁴³-substituted or unsubstituted alkyl, R⁴³-substituted or unsubstituted heteroalkyl, or -L²-R⁹.

In embodiments, R⁵ is independently hydrogen, halogen, —N₃, —NO₂, —CF₃, —CCl₃, —CBr₃, —Cl₃, —CN, —OR^{5A}, —NR^{5A}R^{5B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁴⁴-substituted or unsubstituted alkyl, R⁴⁴-substituted or unsubstituted heteroalkyl, R⁴⁴-substituted or unsubstituted cycloalkyl, R⁴⁴-substituted or unsubstituted heterocycloalkyl, R⁴⁴-substituted or unsubstituted aryl, R⁴⁴-substituted or unsubstituted heteroaryl, or -L²—R⁹.

In embodiments, R⁶ is independently hydrogen, halogen, —N₃, —NO₂, —CF₃, —CCl₃, —CBr₃, —Cl₃, —CN, —OR^{6A}, —NR^{6A}R^{6B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁴⁵-substituted or unsubstituted alkyl, R⁴⁵-substituted or unsubstituted heteroalkyl, R⁴⁵-substituted or unsubstituted cycloalkyl, R⁴⁵-substituted or unsubstituted heterocycloalkyl, R⁴⁵-substituted or unsubstituted aryl, R⁴⁵-substituted or unsubstituted heteroaryl, or -L²-R⁹.

In embodiments, R⁷ is independently hydrogen, halogen, —N₃, —NO₂, —CF₃, —CCl₃, —CBr₃, —Cl₃, —CN, OR^{7A}, —NR^{7A}R^{7B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁴⁶-substituted or unsubstituted alkyl, R⁴⁶-substituted or unsubstituted heteroalkyl, R⁴⁶-substituted or unsubstituted cycloalkyl, R⁴⁶-substituted or unsubstituted heterocycloalkyl, R⁴⁶-substituted or unsubstituted aryl, R⁴⁶-substituted or unsubstituted heteroaryl, or -L²-R⁹.

In embodiments, R⁸ is independently hydrogen, halogen, —N₃, —NO₂, —CF₃, —CCl₃, —CBr₃, —Cl₃, —CN, —OR^{8A}, —NR^{8A}R^{8B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, R⁴⁷-substituted or unsubstituted heteroaryl, or -L²-R⁹.

In embodiments, L² is independently a bond, —NR^{L2}—, R⁴⁸-substituted or unsubstituted alkylene, R⁴⁸-substituted or unsubstituted heteroalkylene, R⁴⁸-substituted or unsubstituted cycloalkylene, R⁴⁸-substituted or unsubstituted heterocycloalkylene, R⁴⁸-substituted or unsubstituted arylene, or R⁴⁸-substituted or unsubstituted heteroarylene.

In embodiments, R¹⁰ is independently hydrogen, —OR^{10A}, —NR^{10A}R^{10B}, or R⁴⁹— substituted or unsubstituted alkyl.

In embodiments, each R^{5A}, R^{5B}, R^{6A}, R^{6B}, R^{7A}, R^{7B, R}^{8A}, R^{8B}, R^{10A}, and R^{10B} is independently hydrogen, R⁵⁰-substituted or unsubstituted alkyl, R⁵⁰-substituted or unsubstituted heteroalkyl, R⁵⁰-substituted or unsubstituted cycloalkyl, R⁵⁰-substituted or unsubstituted heterocycloalkyl, R⁵⁰-substituted or unsubstituted aryl, or R⁵⁰-substituted or unsubstituted heteroaryl.

In embodiments, each R^{XA}, R^{XB}, and R^{XC} is independently hydrogen, R⁵¹-substituted or unsubstituted alkyl, R⁵¹-substituted or unsubstituted heteroalkyl, R⁵¹-substituted or unsubstituted cycloalkyl, R⁵¹-substituted or unsubstituted heterocycloalkyl, R⁵¹-substituted or unsubstituted aryl, R⁵¹-substituted or unsubstituted heteroaryl, or -L²-R⁹.

In embodiments, R^{L2} is independently hydrogen, R⁵²-substituted or unsubstituted alkyl, R⁵²-substituted or unsubstituted heteroalkyl, R⁵²-substituted or unsubstituted cycloalkyl, R⁵²-substituted or unsubstituted heterocycloalkyl, R⁵²-substituted or unsubstituted aryl, or R⁵²-substituted or unsubstituted heteroaryl.

In embodiments, R¹¹ is hydrogen, R⁵³-substituted or unsubsituted alkyl, R⁵³-substituted or unsubstituted heteroalkyl, or -L³-R¹⁴.

In embodiments, R¹² is independently hydrogen, halogen, —N₃, —NO₂, —CF₃, —CCl₃, —CBr₃, —Cl₃, —CN, —OR^{12A}, —NR^{12A}R^{12B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁵⁴-substituted or unsubstituted alkyl, R⁵⁴-substituted or unsubstituted heteroalkyl, R⁵⁴-substituted or unsubstituted cycloalkyl, R⁵⁴-substituted or unsubstituted heterocycloalkyl, R⁵⁴-substituted or unsubstituted aryl, R⁵⁴-substituted or unsubstituted heteroaryl, or -L⁴-R¹⁵.

In embodiments, R¹³ is independently hydrogen, halogen, —N₃, —NO₂, —CF₃, —CCl₃, —CBr₃, —Cl₃, —CN, —OR^{13A}, —NR^{13A}R^{13B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁵⁵-substituted or unsubstituted alkyl, R⁵⁵-substituted or unsubstituted heteroalkyl, R⁵⁵-substituted or unsubstituted cycloalkyl, R⁵⁵-substituted or unsubstituted heterocycloalkyl, R⁵⁵-substituted or unsubstituted aryl, R⁵⁵-substituted or unsubstituted heteroaryl, or -L⁴-R¹⁵.

In embodiments, L³ is a bond, R⁵⁶-substituted or unsubstituted cycloalkylene, R⁵⁶-substituted or unsubstituted heterocycloalkylene, R⁵⁶-substituted or unsubstituted arylene, or substituted or unsubstituted R⁵⁶-heteroarylene.

In embodiments, L⁴ is independently a bond, —NR^{L4}-, R⁵⁷-substituted or unsubstituted alkylene, R⁵⁷-substituted or unsubstituted heteroalkylene, R⁵⁷-substituted or unsubstituted cycloalkylene, R⁵⁷-substituted or unsubstituted heterocycloalkylene, R⁵⁷-substituted or unsubstituted arylene, or R⁵⁷-substituted or unsubstituted heteroarylene.

In embodiments, each R^{12A}, R^{12B}, R^{13A}, R^{13B}, R^{L4}, X²,andR^{X2′} is independently hydrogen, R⁵⁸-substituted or unsubstituted alkyl, R⁵⁸-substituted or unsubstituted heteroalkyl, R⁵⁸-substituted or unsubstituted cycloalkyl, R⁵⁸-substituted or unsubstituted heterocycloalkyl, R⁵⁸-substituted or unsubstituted aryl, or substituted or unsubstituted heteroaryl.

In embodiments, R^{11A} is independently hydrogen, R⁵⁹-substituted or unsubstituted alkyl, —CO₂R^{11C}, or -L⁴-R¹⁵.

In embodiments, R^{11B} is independently hydrogen, halogen, —N₃, —NO₂, —CF₃, —CCl₃, —CBr₃, —Cl₃, —CN, —OR^{11D}, —NR^{11D}R^{11E}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁶⁰-substituted or unsubstituted alkyl, R⁶⁰-substituted or unsubstituted heteroalkyl, R⁶⁰-substituted or unsubstituted cycloalkyl, R⁶⁰-substituted or unsubstituted heterocycloalkyl, R⁶⁰-substituted or unsubstituted aryl, R⁶⁰-substituted or unsubstituted heteroaryl, or -L⁴-R¹⁵.

In embodiments, each R^{11C}, R^{11D}, and R^{11E} is independently hydrogen, R⁶¹-substituted or unsubstituted alkyl, R⁶¹-substituted or unsubstituted heteroalkyl, R⁶¹-substituted or unsubstituted cycloalkyl, R⁶¹-substituted or unsubstituted heterocycloalkyl, R⁶¹-substituted or unsubstituted aryl, or R⁶¹-substituted or unsubstituted heteroaryl.

In embodiments, R¹⁶ is independently hydrogen, halogen, —N₃, —NO₂, —CF₃, —CCl₃, —CBr₃, —Cl₃, —CN, —OR¹⁶A, —NR^{16A}R^{16B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁶²-substituted or unsubstituted alkyl, R⁶²-substituted or unsubstituted heteroalkyl, R⁶²-substituted or unsubstituted cycloalkyl, R⁶²-substituted or unsubstituted heterocycloalkyl, R⁶²-substituted or unsubstituted aryl, R⁶²-substituted or unsubstituted heteroaryl, —OCH═CH₂, or -L⁴-R¹⁵.

In embodiments, R¹⁷ is independently hydrogen, halogen, —N₃, —NO₂, —CF₃, —CCl₃, —CBr₃, —Cl₃, —CN, —OR^{17A}, —NR^{17A}R^{17B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁶³-substituted or unsubstituted alkyl, R⁶³-substituted or unsubstituted heteroalkyl, R⁶³-substituted or unsubstituted cycloalkyl, R⁶³-substituted or unsubstituted heterocycloalkyl, R⁶³-substituted or unsubstituted aryl, R⁶³-substituted or unsubstituted heteroaryl, —OCH═CH₂, or -L⁴-R¹⁵.

In embodiments, R¹⁸ is independently hydrogen, halogen, —N₃, —NO₂, —CF₃, —CCl₃, —CBr₃, —Cl₃, —CN, —OR^{18A}, —NR^{18A}R^{18B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁶⁴-substituted or unsubstituted alkyl, R⁶⁴-substituted or unsubstituted heteroalkyl, R⁶⁴-substituted or unsubstituted cycloalkyl, R⁶⁴-substituted or unsubstituted heterocycloalkyl, R⁶⁴-substituted or unsubstituted aryl, R⁶⁴-substituted or unsubstituted heteroaryl, —OCH═CH₂, or -L⁴-R¹⁵.

In embodiments, R¹⁹ is independently hydrogen, halogen, —N₃, —NO₂, —CF₃, —CCl₃, —CBr₃, —Cl₃, —CN, —OR^{19A}, —NR^{19A}R^{19B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁶⁵-substituted or unsubstituted alkyl, R⁶⁵-substituted or unsubstituted heteroalkyl, R⁶⁵-substituted or unsubstituted cycloalkyl, R⁶⁵-substituted or unsubstituted heterocycloalkyl, R⁶⁵-substituted or unsubstituted aryl, R⁶⁵-substituted or unsubstituted heteroaryl, —OCH═CH₂, or -L⁴-R¹⁵.

In embodiments, R²⁰ is independently hydrogen, halogen, —N₃, —NO₂, —CF₃, —CCl₃, —CBr₃, —Cl₃, —CN, —OR^{20A}, —NR^{20A} R^{20B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁶⁶-substituted or unsubstituted alkyl, R⁶⁶-substituted or unsubstituted heteroalkyl, R⁶⁶-substituted or unsubstituted cycloalkyl, R⁶⁶-substituted or unsubstituted heterocycloalkyl, R⁶⁶-substituted or unsubstituted aryl, R⁶⁶-substituted or unsubstituted heteroaryl, or L⁴-R¹⁵.

In embodiments, R²¹ is independently hydrogen, halogen, —N₃, —NO₂, —CF₃, —CCl₃, —CBr₃, —Cl₃, —CN, —OR^{21A}, —NR^{21A}R^{21B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁶⁷-substituted or unsubstituted alkyl, R⁶⁷-substituted or unsubstituted heteroalkyl, R⁶⁷-substituted or unsubstituted R⁶⁷-cycloalkyl, substituted or unsubstituted heterocycloalkyl, R⁶⁷-substituted or unsubstituted aryl, R⁶⁷-substituted or unsubstituted heteroaryl, or -L_{L}4_{-R}¹⁵.

In embodiments, each R^{16A}, R^{16B}, R^{17A}, R^{17B}, R^{18A}, R^{18B}, R^{19A}, R^{19B}, R^{20A}, R^{20B}, R^{21A}, and R^{21B} is independently hydrogen, R⁶⁸-substituted or unsubstituted alkyl, R⁶⁸-substituted or unsubstituted heteroalkyl, R⁶⁸-substituted or unsubstituted cycloalkyl, R⁶⁸-substituted or unsubstituted heterocycloalkyl, R⁶⁸-substituted or unsubstituted aryl, or R⁶⁸-substituted or unsubstituted heteroaryl.

In embodiments, each R^{X3} and R^{X3′} is independently hydrogen or R⁶⁹-substituted or unsubstituted alkyl.

In embodiments, L⁴ is independently a bond, —NR^{L4}—, R⁷⁰-substituted or unsubstituted alkylene, R⁷⁰-substituted or unsubstituted heteroalkylene, R⁷⁰-substituted or unsubstituted cycloalkylene, R⁷⁰-substituted or unsubstituted heterocycloalkylene, R⁷⁰-substituted or unsubstituted arylene, or R⁷⁰-substituted or unsubstituted heteroarylene.

In embodiments, R²¹ is independently hydrogen, OR^{21A, NR}^{21A}R^{21B}, R⁷¹-substituted or unsubstituted alkyl, or -L⁴-R¹⁵.

In embodiments, R²² and R²³ are independently hydrogen, R⁷²-substituted or unsubstituted alkyl, or -L⁴-R¹⁵.

In embodiments, each R²⁴, R²⁵, R²⁶, R²⁷, R²⁸, R²⁹, R³⁰, and R³¹ is independently hydrogen, halogen, —N₃, —NO₂, —CF₃, —CCl₃, —CBr₃, —Cl₃, —CN, —OH, —NH₂, —NMe₂, —NEt₂, —COOH, —CONH₂, —NO₂, ——SH, —SO₂Cl, —SO₃H, —SO₄H, —SO₂NH₂, —NHNH₂, —ONH₂, —OCH₃, —NHCNHNH₂, R⁷³-substituted or unsubstituted alkyl, R⁷³-substituted or unsubstituted heteroalkyl, R⁷³-substituted or unsubstituted cycloalkyl, R⁷³-substituted or unsubstituted heterocycloalkyl, R⁷³-substituted or unsubstituted aryl, R⁷³-substituted or unsubstituted heteroaryl, or -L⁴-R¹⁵.

In embodiments, each R³² and R³³ is independently hydrogen, halogen, —N₃, —NO₂, —CF₃, —CCl₃, —CBr₃, —Cl₃, —CN, —OH, —NH₂, —NMe₂, —NEt₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁷⁴-substituted or unsubstituted alkyl, R⁷⁴-substituted or unsubstituted heteroalkyl, R⁷⁴-substituted or unsubstituted cycloalkyl, R⁷⁴-substituted or unsubstituted heterocycloalkyl, R⁷⁴-substituted or unsubstituted aryl, R⁷⁴-substituted or unsubstituted heteroaryl, or -L⁴-R¹⁵.

In embodiments, R³⁴ is independently hydrogen, halogen, —N₃, —NO₂, —CF₃, —CCl₃, —CBr₃, —Cl₃, —CN, —NR^{34A}R^{34B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, R⁷⁵-substituted or unsubstituted alkyl, R⁷⁵-substituted or unsubstituted heteroalkyl, R⁷⁵-substituted or unsubstituted cycloalkyl, R⁷⁵-substituted or unsubstituted heterocycloalkyl, R⁷⁵-substituted or unsubstituted aryl, or R⁷⁵-substituted or unsubstituted heteroaryl.

In embodiments, L⁵ is independently a bond, —NR¹⁻⁵—, O, S, R⁷⁶-substituted or unsubstituted alkylene, R⁷⁶-substituted or unsubstituted alkenylene, R⁷⁶-substituted or unsubstituted alkynylene, R⁷⁶-substituted or unsubstituted heteroalkylene, R⁷⁶-substituted or unsubstituted cycloalkylene, R⁷⁶-substituted or unsubstituted heterocycloalkylene, R⁷⁶-substituted or unsubstituted arylene, or R⁷⁶-substituted or unsubstituted heteroarylene.

In embodiments, each R^{34A}, R^{34B}, and R^{L5} is independently hydrogen, R⁷⁷-substituted or unsubstituted alkyl, R⁷⁷-substituted or unsubstituted heteroalkyl, R⁷⁷-substituted or unsubstituted cycloalkyl, R⁷⁷-substituted or unsubstituted heterocycloalkyl, R⁷⁷-substituted or unsubstituted aryl, or R⁷⁷-substituted or unsubstituted heteroaryl.

In embodiments, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵²R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³R⁷⁴, R⁷⁵, R⁷⁶, and R⁷⁷ are independently hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

In some embodiments, a compound as described herein may include multiple instances of variables described herein. In such embodiments, each variable may optionally be different and be appropriately labeled to distinguish each group for greater clarity. In some embodiments, the compound is a compound described herein (e.g., in an aspect, embodiment, example, claim, table, scheme, drawing, or figure).

### IV. EXAMPLES

### Example 1

### General Methods, Synthesis, and General Procedures for Examples 2-6

1.1. General Methods

All reagents were purchased from Sigma-Aldrich and used without further purification unless otherwise noted. The TLC plates used for purification were purchased from Agela Technologies (silica 200×200 mm, PH=5, MF=254, glass back). Other chromatographic purifications were conducted using 40-63 μm silica gel. The microwave reaction was done using a CEM microwave reactor. All mixtures of solvents are given in v/v ratio. ¹H and ¹³C NMR spectroscopy were performed on a Varian NMR at 400 (¹H) or 100 (¹³C) MHz or a Jeol NMR at 500 (¹³H) or 125 (¹³C) MHz. All ¹³C NMR spectra were proton decoupled. Fluorescence measurements were performed on a HORIBA FLUOROMAX®-P spectrophotometer equipped with a single cuvette reader. Oligonucleotide concentrations were measured on a ThermoScientific NANODROP™ 2000c UV-Vis Spectrophotometer with absorption wavelength of 260 nm.

1.1.1 Cell culture conditions. The SKBR3, MCF-7, and HeLa cells were cultured in McCoy's 5A high glucose media (CORNING® CELLGRO®, Manassas, Va.) supplemented with 2mM L-Glutamine (Carl Roth, Karlsruhe, Germany), 10% FBS (Thermo Fisher Scientific Inc., Waltham, Mass., USA) 100 U/mL penicillin and 100 μg/mL streptomycin (PAA Laboratories, Pasching, Austria). Cultures were incubated at 37° C. with 5% CO₂ and 95% humidity.

1.1.2 Cellular imaging of tetrazine-azanorbornadiene probes. For imaging experiments 7×10⁴ cells were seeded in an 8-well LAB-TEK™ chamber slide one day prior to the experiment. To transfect the cells we incubated 8 ul of LIPOFECTAMINE™ 2000 in 200 ul OPTI-MEM™ containing 800 nM of Tetrazine and Azanorbornadiene probes (1:1). We incubated the probes for 10 mins at RT to allow the LIPOFECTAMINE™-oligo complex to form then we did a 4× dilution using OPTI-MEM™. The cell media was aspirated and replaced with 200 ul of the newly made OPTI-MEM™ solution and incubated for two hours at 37° C. with 5% CO₂ and 95% humidity. The cells were washed three times using PBS with 10% FBS. Images were acquired on an Olympus FV1000 Confocal inverted microscope (Olympus, Tokyo, Japan) with a 63×, 1.40 NA oil immersion objective. Environmental conditions were maintained at 37° C, and 5% CO². The BODIPY® fluorescent moiety was excited with a 488 nm, 100 mw OPSL laser, green.

1.1.3 Turn on of tetrazine-azanorbornadiene probes in lysate. Cultured cells were grown on a 100 mm plate until 60-90% confluency was reached. They were detached using TRYPLE™ then washed once with cell media. Cells were then counted with the countess®, followed by centrifugation at 0.7 RCF. The appropriate volume of RIPA buffer was used to resuspended the cells to obtain 1.5×10⁶ cells/ml. RNASEOUT™ was added at 400 U/ml (final concentration) to slow RNA degradation. Cells were kept on ice for 15 mins with intermittent vortexing using glass disruption beads. The debris was pelleted in a centrifuge and the supernatant was removed. The oligo probes were added to the supernatant at a final concentration of 100 nM. The solution was incubated at 37° C. for two hours before measuring the fluorescence on a HORIBA FLUOROMAX®-P spectrophotometer

1.2. Tag Synthesis

1.2.1 Synthesis of ABN and ABN-NHS

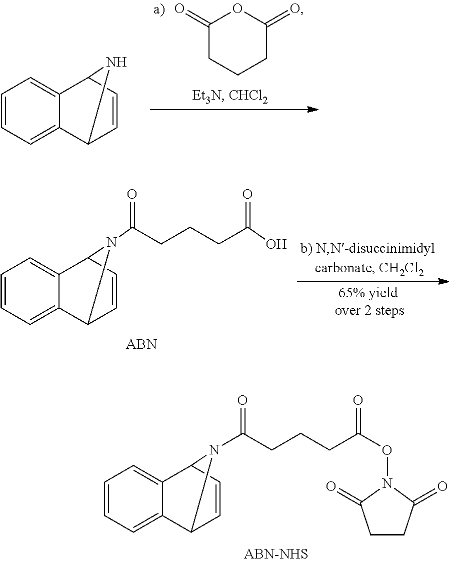 [SMILES: C1=CC2NC1c1ccccc12 ; O=C1CCCC(=O)O1 ; O=C(O)CCCC(=O)N1C2C=CC1c1ccccc12 ; O=C(CCCC(=O)N1C2C=CC1c1ccccc12)ON1C(=O)CCC1=O]

To a stirred solution of 7-azabenzonorbornadiene, (28.6 mg, 0.2 mmol) in CH₂Cl₂ TEA (0.03 mL, 0.22 mmol) was added followed by glutaric anhydride (23 mg, 0.2 mmol). The solution was stirred at room temperature for 1 h to produce ABN, after which N,N′-disuccinimidyl carbonate (51.0 mg, 0.2 mmol) was added. After stirring at room temperature for 30 minutes, the reaction solution was concentrated in vacuo and the residue was purified by preparative TLC (Hex : EtOAc=1:1) to afford 43.5 mg of ABN-NHS as a colorless solid (63% yield in 2 steps).

NMR and HRMs data For ABN: ¹H NMR (500 MHz, CDCl₃) δ 10.25 (br, 1H), 7.61-7.50 (m, 2H), 7.34 (dd, J=5.6, 2.5 Hz, 1H), 7.28-7.23 (m, 3H), 6.21 (s, 1H), 5.91 (s, 1H), 2.68-2.53 (m, 4H), 2.20-2.11 (m, 2H). ¹³C NMR (126 MHz, CDCl₃) δ 177.86, 168.06, 148.09, 147.88, 144.28, 142.46, 125.43, 125.19, 121.46, 120.49, 65.58, 63.30, 33.01, 32.70, 19.71. HRMS [M+Na]⁺ m/z calcd. for [C₁₅H₁₅NNaO₃]⁺280.0950, found 280.0948.

NMR and HRMs data for ABN-NHS: ¹H NMR (500 MHz, CDCl₃) δ 7.31-7.28 (m, 1H), 7.27 (s, 1H), 7.06 (dd, J=5.5, 2.5 Hz, 1H), 6.99 (dd, J=5.6, 2.4 Hz, 1H), 6.97 (dd, J=5.1, 3.0 Hz, 2H), 5.90 (s, 1H), 5.66 (s, 1H), 2.86 (d, J=5.6 Hz, 4H), 2.69-2.56 (m, 2H), 2.41 (dt, J=15.1, 7.5 Hz, 1H), 2.37-2.28 (m, 1H), 2.00 (dd, J=14.6, 7.1 Hz, 2H). ¹³C NMR (126 MHz, CDCl₃) δ 169.25, 168.54, 167.86, 148.32, 148.12, 144.37, 142.64, 125.42, 125.20, 121.48, 120.58, 65.66, 63.46, 32.10, 30.31, 25.75, 19.93. HRMS [M+H]⁺ m/z calcd. for [C₁₉H₁₈N₂O₅Na]⁺377.1108, found 377.1109.

1.2.2 Synthesis of Tetrazine BH-Tz

To a 10-mL microwave reaction tube equipped with a stir bar, Pd₂(dba)₃ (0.92mg, 0.001 mmol) and ligands 1,2,3,4,5-pentaphenyl-1′-(di-tert-butylphosphino) ferrocene (2.1 mg 0.003 mmol), tetrazine 1a (Wu, H., Yang,J., Seckute,J., and Devaraj,N. K. Angew. Chem. Int. Ed., 51, 7476-7479.) (2.2 mg, 0.01 mmol), BD-0 (Ziessel, R., Ulrich, G., Haefele, A. and Harriman, A. J. Am. Chem. Soc. 2013, 135, 11330-11344) (4.63 mg, 0.01 mmol), and N,N-dicyclohexylmethylamine (0.015 mL, 0.03 mmol) were dissolved in anhydrous DMF (1.5 mL). The reaction was protected with N₂ gas and then heated by microwave irradiation (50° C., 40 min). The reaction solution was cooled to room temperature and EtOAc (20 mL) was added before washing with water (20 mL×3). The organic layer was dried over Na₂SO₄ and concentrated in vacuo. The residue was purified preparative TLC (Hexanes: DCM=1:2) to afford pure TzH (3.6 mg, yield 74%) as a dark pink solid.

¹H NMR (500 MHz, CDCl₃) δ 8.36 (d, J=16.3 Hz, 1H), 7.82 (d, J=8.1 Hz, 2H), 7.55 (d, J=16.3 Hz, 1H), 7.39 (d, J=8.2 Hz, 2H), 6.00 (s, 2H), 3.08 (s, 3H), 2.57 (s, 6H), 1.44 (s, 6H). ¹³C NMR (126 MHz, CDCl₃) δ 166.69, 164.71, 155.97, 143.07, 140.73, 139.95, 137.03, 135.85, 132.85, 132.63, 131.30, 129.02, 128.84, 127.42, 127.08, 121.84, 121.54, 29.85, 21.42, 14.78.

1.2.3 Synthesis of Pyridazine compound PzH

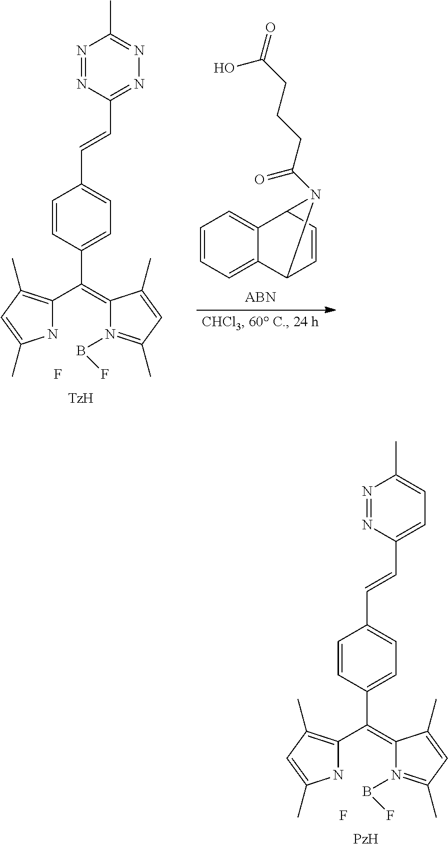

To a tube equipped with a stirring bar, TzH (2.22 mg, 0.005 mmol), and ABN (1.3 mg, 0.005 mmol), was dissolved in 0.5 mL of CHCl₃, the reaction was heating in oil bath at 60° C. for 24 h, when TLC indicated that the reaction had completed. The reaction was concentrated in vacuo and the residue was purified by preparative TLC (CH₂Cl₂: MeOH=50: 1), to give PzH (2.12 mg, yield 95%) as a light pink solid.

¹H NMR (500 MHz, CDCl₃-D) δ 7.73 (d, J=8.1 Hz, 2H), 7.67 (d, J=16.4 Hz, 1H), 7.56 (d, J=8.7 Hz, 1H), 7.44 (d, J=16.5 Hz, 1H), 7.36-7.29 (m, 3H), 5.99 (s, 2H), 2.75 (s, 3H), 2.56 (s, 6H), 1.44 (s, 6H). ¹³C NMR (126 MHz, CDCl₃) δ 158.68, 155.91, 155.75, 143.16, 141.20, 136.92, 135.52, 133.20, 131.41, 128.72, 128.01, 127.14, 126.59, 124.14, 121.43, 29.86, 22.36, 14.78, 14.75. HRMS [M+H]⁺ m/z calcd. for [C₂₆H₂₆BF₂N₄]⁺ 442.2245, found 442.2244.

1.2.4 Synthesis scheme of Tz-NHS

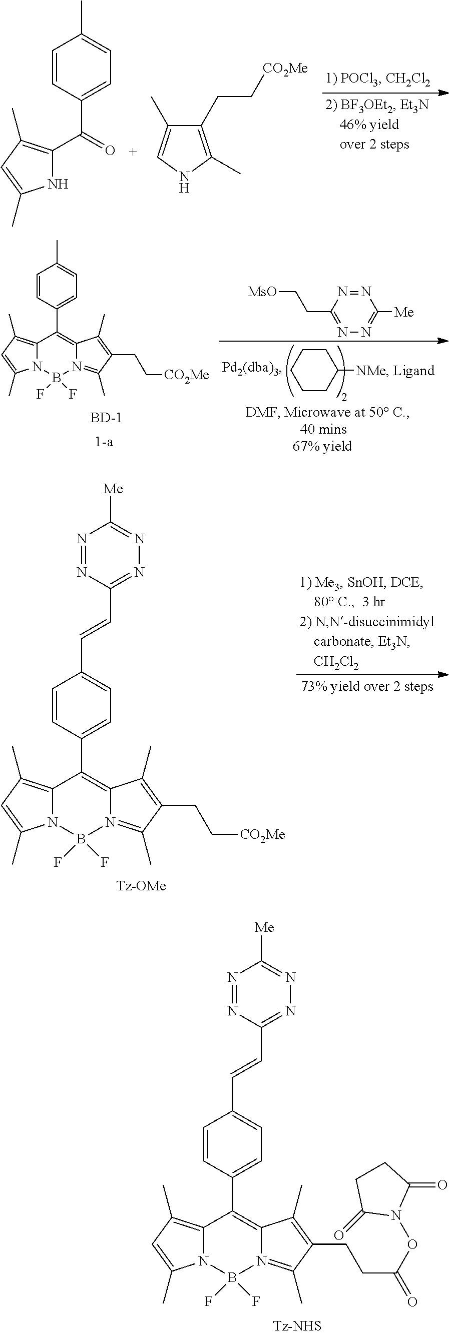

1.2.4.1 Synthesis of BD-1

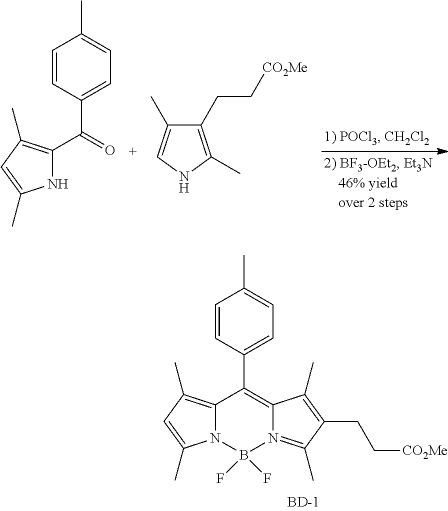

2-(4-Iodobenzoyl)-3,5-dimethylpyrrole (160 mg, 0.5 mmol) (Wallace, D. M.; Leung, S. H.; Senge, M. O.; Smith, K. M, J. Org. Chem. 1993, 58, 7245) was dissolved in CH₂Cl₂ (15.0 mL) at 0° C. 1H-Pyrrole-3-propanoic acid was added followed by 2,4-dimethyl-, methyl ester (90 mg, 0.5 mmol). Phosphoryl chloride (0.05 mL, 0 5 mmol) was then added dropwise and the reaction was stirred overnight at room temperature. The reaction was then cooled to 0 ° C. and Et₃N (0.69 mL, 5 mmol) was added dropwise followed by BF₃.OEt₂ (0.62 mL, 5 mmol). The reaction was then stirred at room temperature for 10 hours. The resultant dark solution was dissolved in CH₂Cl₂ (100 mL), then washed with water and saturated sodium chloride. The organic layer was dried over Na₂SO₄, filtered, and concentrated in vacuo. The residue was purified by silica column chromatography (Hexanes:EtOAc=15:1) to afford 24.8 mg product BD-1 as a dark pink solid (46% yield over 2 steps).

¹H NMR (500 MHz, CDCl₃) δ 7.83 (d, J=8.1 Hz, 2H), 7.02 (d, J=8.1 Hz, 2H), 5.96 (s, 1H), 3.64 (s, 3H), 2.67-2.59 (m, 2H), 2.53 (s, 3H), 2.52 (s, 3H), 2.38-2.31 (m, 2H), 1.38 (s, 3H), 1.34 (s, 3H). ¹³C NMR (126 MHz, CDCl₃) δ 173.08, 155.45, 155.09, 142.67, 139.70, 139.56, 138.45, 134.81, 131.05, 130.77, 130.06, 129.69, 121.35, 94.86, 51.83, 34.22, 19.35, 14.79, 14.68, 12.78, 12.27. HRMS [M+Na]⁺m/z calcd. for [C₂₃H₂₄BF₂IN₂O₂Na]⁺558.0872, found 558.0873.

1.2.4.2 Synthesis of the Tz-OMe

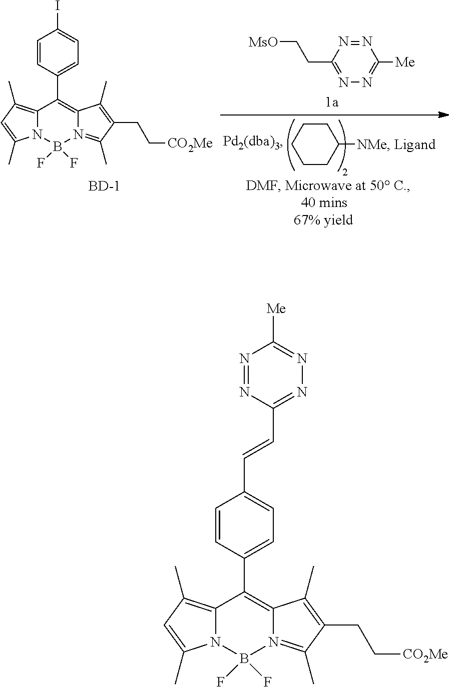

To a 10-mL microwave reaction tube equipped with a stir bar, Pd₂(dba)₃ (0.92 mg 0.001 mmol), ligand 1,2,3,4,5-pentaphenyl-1′-(di-tent-butylphosphino) ferrocene (2.1 mg 0.003 mmol), tetrazine 1a (Wu, H., Yang, J., Seckute, J., and Devaraj,N. K. Angew. Chem. Int. Ed., 51, 7476-7479.) (2.2 mg, 0.01 mmol), BD-1 (5.3 mg, 0.01 mmol), and N,N-dicyclohexylmethylamine (0.015 mL, 0.03 mmol) were dissolved in anhydrous DMF (1.5 mL). The reaction was protected with N₂ gas and then heated by microwave irradiation (50° C., 40 min). The reaction solution was cooled to room temperature and EtOAc (20 mL) was added before washing with water (20 mL×3). The organic layer was dried over Na₂SO₄, filtered, and concentrated in vacuo. The residue was purified by preparative TLC (Hexanes: DCM=1:2) to afford pure Tz-OMe (3.55 mg, yield 67%) as a dark pink solid.

¹H NMR (500 MHz, CDCl₃) δ 8.36 (d, J=16.3 Hz, 1H), 7.82 (d, J=8.1 Hz, 2H), 7.56 (d, J=16.3 Hz, 1H), 7.41-7.35 (m, 2H), 5.98 (s, 1H), 3.66 (s, 3H), 3.08 (s, 3H), 2.65 (dd, J=8.8, 7.0 Hz, 2H), 2.56 (d, J=2.9 Hz, 6H), 2.39-2.33 (m, 2H), 1.42 (s, 3H), 1.37 (s, 3H). ¹³C NMR (126 MHz, CDCl₃) δ 173.15, 166.69, 164.70, 155.49, 155.09, 142.74, 140.30, 139.94, 139.62, 137.23, 135.85, 132.60, 131.17, 130.87, 129.07, 128.85, 121.85, 121.36, 51.88, 34.29, 29.85, 21.42, 19.43, 14.78, 12.85, 12.26. HRMS [M+Na]⁺m/z calcd. for [C₂₈H₂₉BF₂N₆O₂Na]⁻ 552.2342, found 552.2341.

1.2.4.3 Synthesis of the Tz-NHS

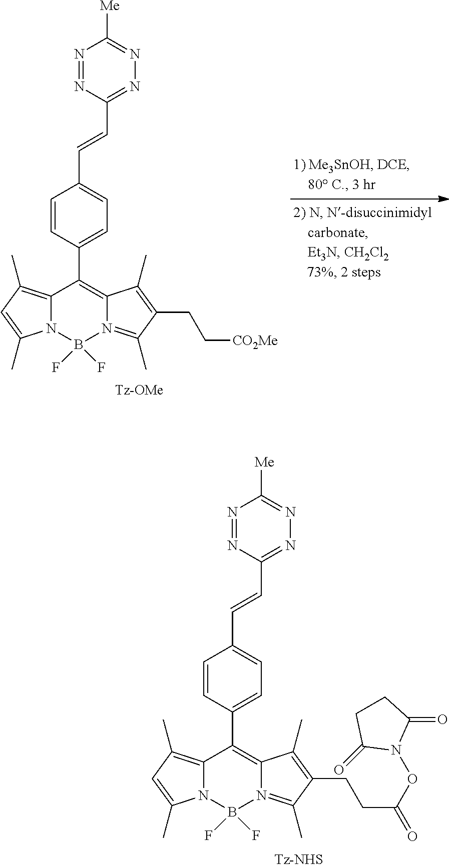

In a 10-mL reaction tube equipped with a stir bar, Tz-OMe (5.3 mg, 0.01 mmol) was dissolved in 1,2-dichloroethane which Me₃SnOH (10 mg, 0.05 mmol) was added. The solution was heated to 80° C. and stirred for 3 hours. The reaction solution was evaporated and purified by flash column chromatography (CH₂Cl₂: MeOH=10:1) to afford the carboxylic acid intermediate. The intermediate was dissolved in CH₂Cl₂ followed by addition of Et₃N (0.01 mL, 0.07 mmol) and N,N′-disuccinimidyl carbonate (13.0 mg, 0.05 mmol). This solution was stirred at room temperature for 1 hour. The reaction solution was evaporated and purified by preparative TLC plate (CH₂Cl₂:MeOH=50:1) to afford Tz-NHS (4.6 mg, yield 73% in 2 steps).

¹H NMR (500 MHz, CDCl₃) δ 8.37 (d, J=16.3 Hz, 1H), 7.83 (d, J=8.1 Hz, 2H), 7.56 (d, J=16.4 Hz, 1H), 7.39 (d, J=8.1 Hz, 2H), 6.00 (s, 1H), 3.08 (s, 3H), 2.85 (s, 4H), 2.79-2.72 (m, 2H), 2.68-2.62 (m, 2H), 2.57 (d, J=5.6 Hz, 6H), 1.43 (s, 3H), 1.39 (s, 3H). ¹³C NMR (126 MHz, CDCl₃) δ 169.14, 168.73, 167.77, 166.68, 164.71, 156.16, 154.38, 143.24, 140.58, 139.93, 139.52, 137.11, 135.91, 131.41, 130.74, 129.07, 128.89, 128.20, 121.89, 121.64, 57.54, 31.28, 25.71, 25.59, 21.41, 19.12, 14.83, 12.87, 12.32.

1.3. General Procedure for Oligonucleotide Modification

Amine-modified oligonucleotide sequences at selected terminus were dissolved in a 0.1 mM sodium bicarbonate buffer (pH=8.45). A 3 mM solution of either ABN-NHS or Tz-NHS in DMSO (30 eq) was added in one portion. The reaction was left at room temperature for 3 hours and reaction progress was monitored by HPLC. The main product formed without significant side reactions.

LC-MS conditions: An Agilent 1260 HPLC system coupled with an Agilent 6230 time of flight mass spectrometer (TOFMS) was employed for LC-TOFMS analysis. The instrument was operated under negative ion mode use Jetstream electrospray ionization (ESI) as the ion source. A Phenomenex CLARITY® Oligo-MS column (2.1 mm×150 mm, 2.6 um) was used for separation by using TEA/HFIP/H₂O (0.4:30/1000 v/v) as mobile phase A and MeOH as mobile phase B.

It is important to carefully purify the oligonucleotide after modification and to lyophilize the sample to remove solvent immediately after HPLC. The purity of all compounds was verified by LCMS prior to activation experiments.

1.3.1 Synthesis and characterization of d27′-Tz

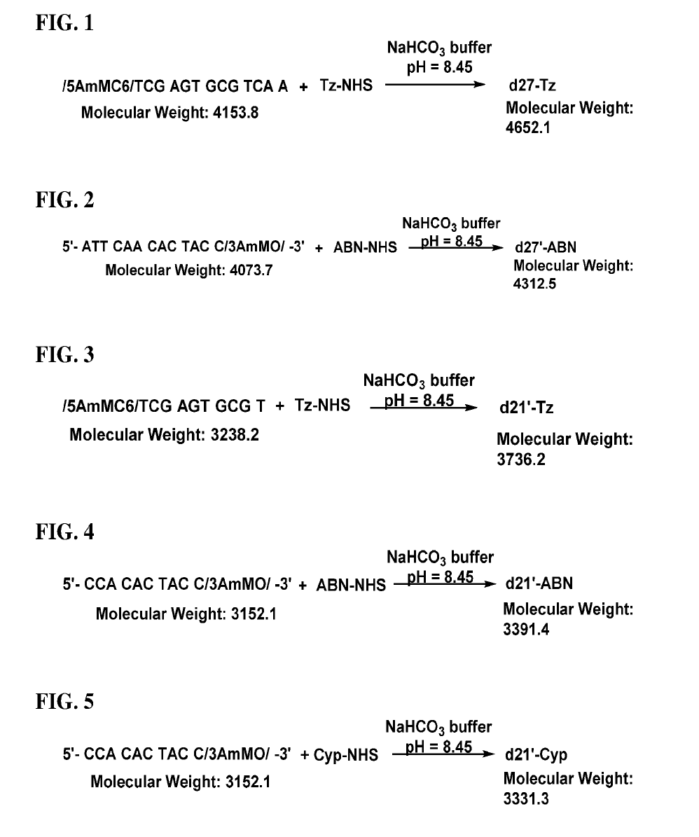

According to the schematic of FIG. 1, HPLC traces (260 nm) and ESI-TOF MS were used to demonstrate absorption peaks for detected oligonucleotide species.

1.3.2 Synthesis and characterization of d27′-ABN

According to the schematic of FIG. 2, HPLC traces (260 nm) and ESI-TOF MS were used demonstrate absorption peaks for detected oligonucleotide species.

1.3.3 Synthesis and characterization of d21′-Tz

According to the schematic of FIG. 3, HPLC traces (260 nm) and ESI-TOF MS were used demonstrate absorption peaks for detected oligonucleotide species.

1.3.4 Synthesis and characterization of d21′-ABN

According to the schematic of FIG. 4, HPLC traces (260 nm) and ESI-TOF MS were used demonstrate absorption peaks for detected oligonucleotide species.

1.3.5 Synthesis and characterization of d21′-Cyp

According to the schematic of FIG. 5, HPLC traces (260 nm) and ESI-TOF MS were used demonstrate absorption peaks for detected oligonucleotide species.

1.3.6 Synthesis and characterization of mir21′-Tz

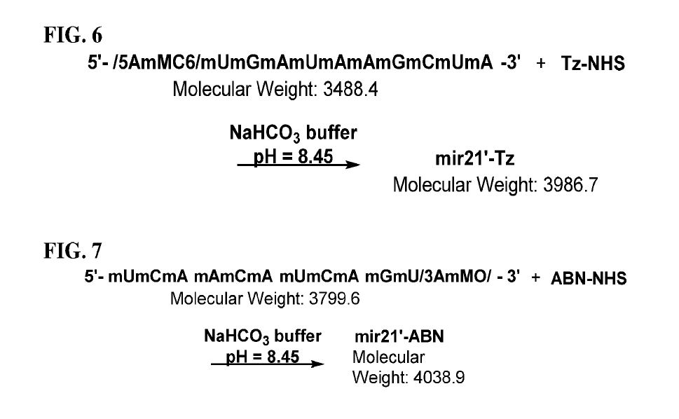

According to the schematic of FIG. 6, HPLC traces (260 nm) and ESI-TOF MS were used demonstrate absorption peaks for detected oligonucleotide species.

1.3.7 Synthesis and characterization of mir21′-ABN

According to the schematic of FIG. 7, HPLC traces (260 nm) and ESI-TOF MS were used demonstrate absorption peaks for detected oligonucleotide species.

1.4. Kinetic Measurements

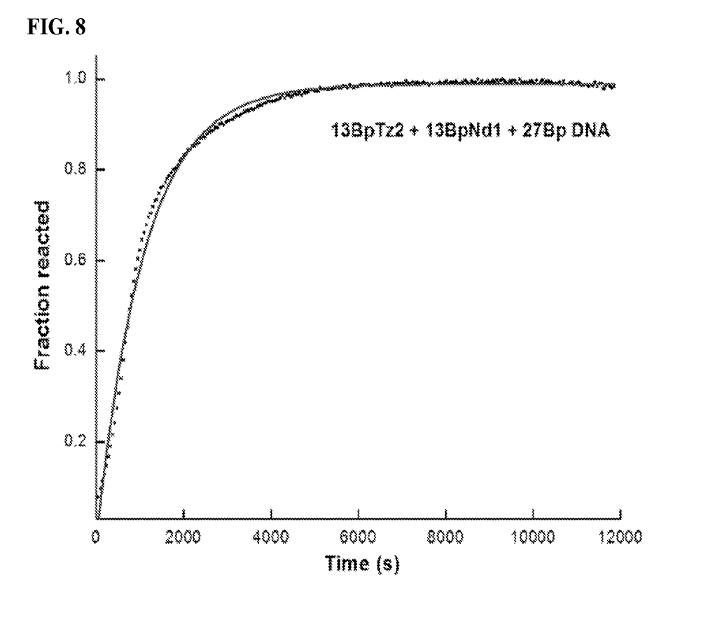

A TECAN Genios Pro/384-well multifunction microplate reader was used in the kinetic measurements, and the increase of the fluorescence intensity was measured over time. A solution of d27′-Tz and d27′-ABN at 1 μM was reacted with 1 μM of d27. Measurement commenced immediately upon the addition of d27. Solution conditions were maintained at 100 mM Tris-HCl buffer at pH 7.4 with 200 mM MgCl₂, and all the measurements were done at room temperature. The increase in fluorescence intensity over time is shown in FIG. 1.8 (black dots). The fluorescence data was fitted with a one-phase exponential association curve (FIG. 8) and the observed first order rate constant was determined to be 0.00091±0.00002 s⁻¹ with a t_{1/2}=12.7 mins.

1.5. Fluorescence Turn-on Measurements

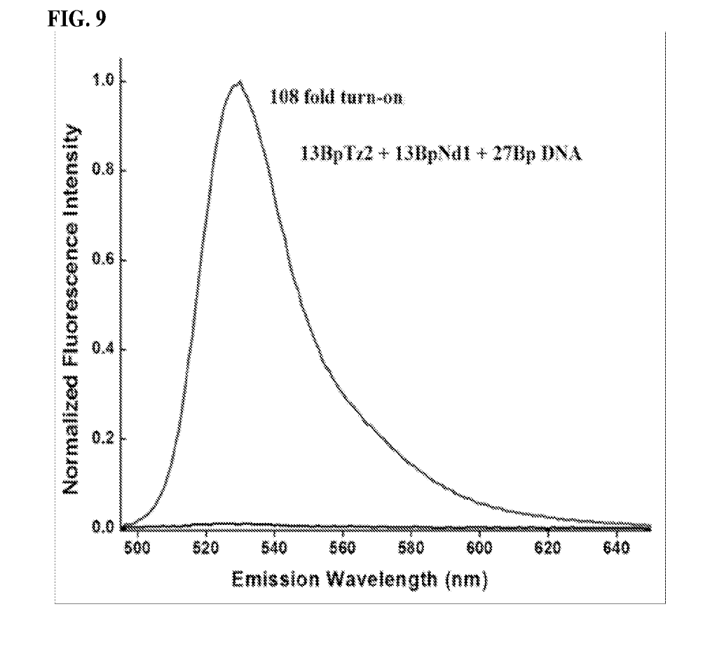

Fluorescence emission spectra of template driven reaction. Fluorescence scans were done before (black line in FIG. S2) and 1.5 hours after (red line in FIG. 9) the adding the DNA template. Reaction conditions: 1 μM template 1, 1 μM 13BpTz2, and 1 μM 13BpNd1 in 100 mM Tris-HCl, 200 mM MgCl₂ buffer (pH=7.4) 37° C. The excitation wavelength used was 480 nm. Activation ratios were calculated from the peak emission intensity of the reaction product and the corresponding baseline intensity, and all the intensity data was background subtracted.

1.6. DNA-Templated Tetrazine Transfer Reaction with Turnover

General reaction conditions: In a 500-μl microcentrifuge tube, d21′-Tz and d′21-ABN were added to Tris-buffer (pH=7.4) followed by a MgCl₂ solution, and the additive (if any). Then the appropriate amount of template d21 was added. The final volume was 200 μl and the final concentrations of the individual components were: [Tris]=100 mM, [MgCl_{2]}=200 mM, [d21′-Tz]=100 nM, and [d21′-ABN]=200 nM. Then, the centrifuge tubes vortexed for 30s then incubated at 37° C. for 7 hours. Then fluorescence measurements were done by using spectrophotometer equipped with a single cuvette reader (excitation at 480/3 nm slit). The fluorescence emission signals were reported as average of values from three times. Integration of the area under the emission intensity curves was used to validate the activation ratios. The turnover number was obtained by dividing the number of moles of the fluorescent product by the number of moles of the template following a reaction period of 7 h.

1.6.1. Comparison of fluorescence intensity between DrD reaction and ligation reaction.

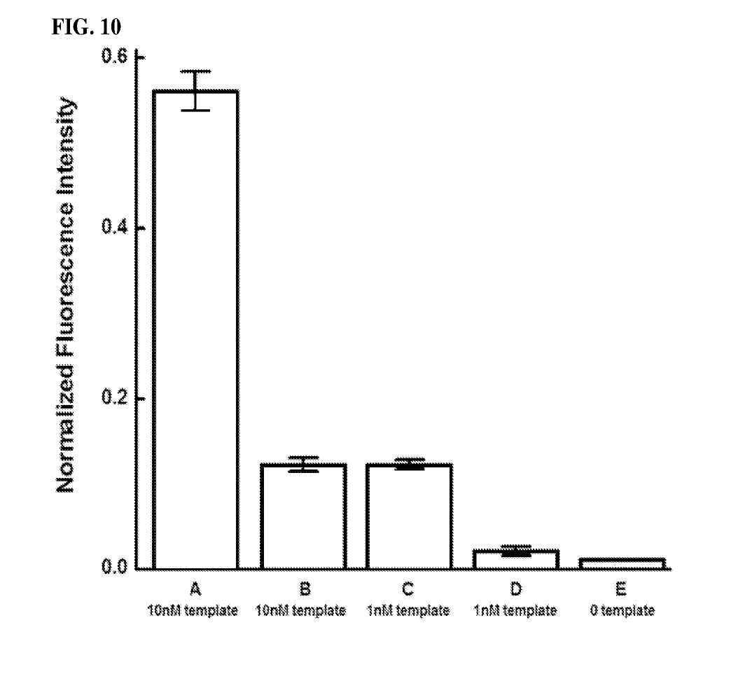

Histogram of FIG. 10 depicts comparison of normalized fluorescence intensity between DrD reaction (d21′-Tz +d21′-ABN), and ligation reaction (d21′-Tz+d21′-Cyp) at different template concentration.

1.6.2. Comparison of fluorescence intensity between reaction with perfect match template and mismatched template.

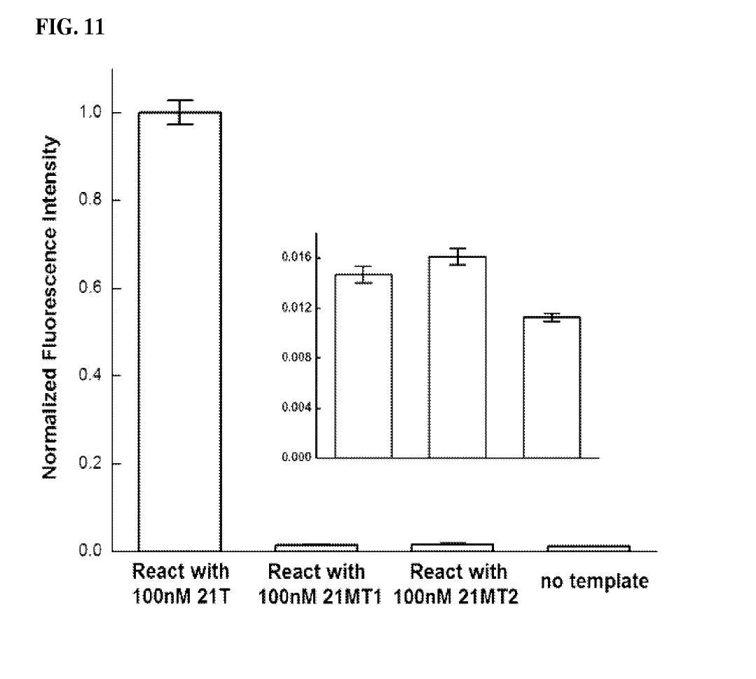

Histogram of FIG. 11 depicts the normalized fluorescence emission signal of d21′-Tz and d21′-ABN incubated with perfect match template (d21), mismatch templates (d21a and d21b), and no template after 37° C. for 1.5 hour.

1.6.3. Tetrazine Transfer Reaction Challenge Conditions

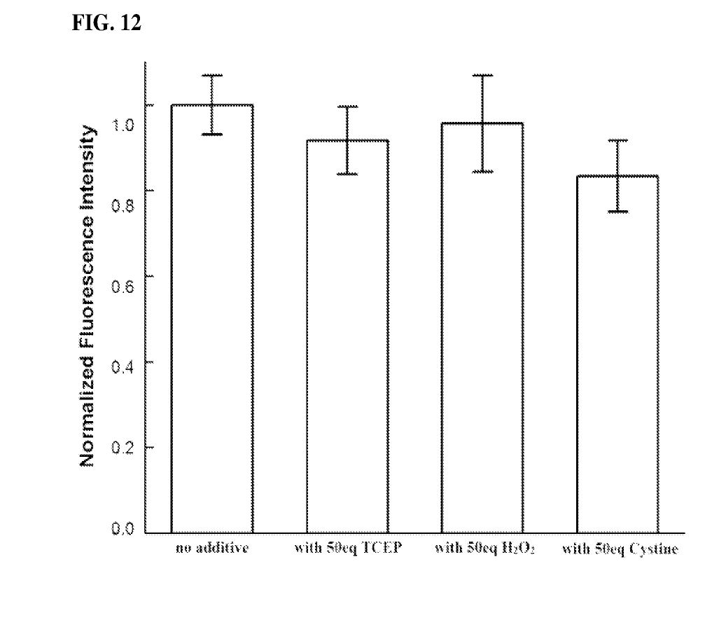

Histogram of FIG. 12 depicts the normalized fluorescence emission signal of d21′-Tz and d21′-ABN with 0.01 eq template (d21) in different additive reaction solution after 7 hours incubation.

1.7. RNA-Templated Tetrazine Transfer Reaction

General reaction condition: In a 500 ul microcentrifuge tubes, mir21′-Tz and mir21′-ABN were added into Tris-buffer (pH=7.4), follow by NaCl solution, and appropriate amount of template mir21. The final volume was 200 uL, the final concentration was Tris 100 mM, NaCl 100 mM, 10BpMeTz2 100 nM, 11BpMeNd2 200 nM. The microcentrifuge tubes were then vortexed for 30s then incubated at 37° C. for 7 hours. Fluorescence measurements were taken using spectrophotometer equipped with a single cuvette reader (excitation at 480/3 nm). The fluorescence emission signals were reported as average of three replicates. The turnover number was obtained by dividing the number of moles of the fluorescent product by the number of moles of the template following a reaction period of 7 h.

1.7.1. Oligonucleotide probe stability in DMEM.

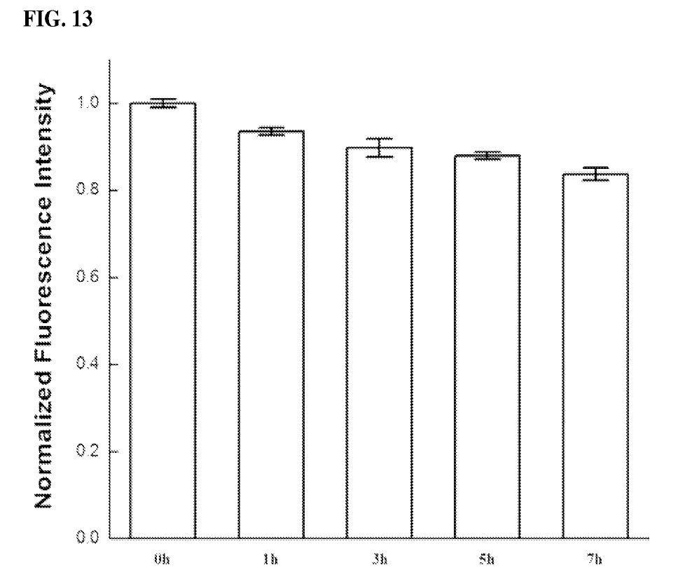

Histogram of FIG. 13 depicts Oligonucleotide probe stability in DMEM.

1.7.2. Detecting Mir21 in different cell-line lysate.

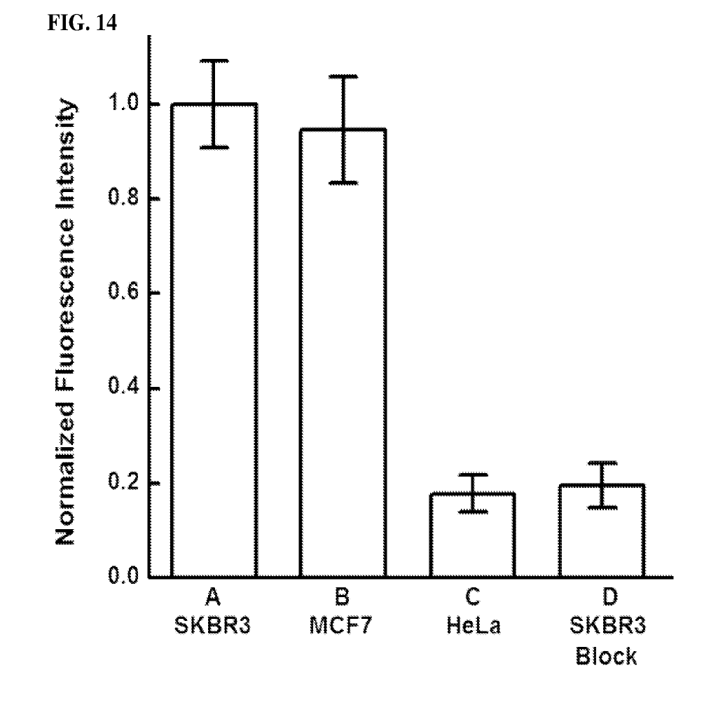

Histogram of FIG. 14 depict Normalized fluorescence intensity of oligonucleotide probes mir21′-Tz and mir21′-ABN upon reaction with different cell lysates. Column D: 10 eq of unmodified oligonucleotide probe added as a competitive inhibitor.

1.7.3. Detecting discrimination between matched and single mismatch RNA targets.

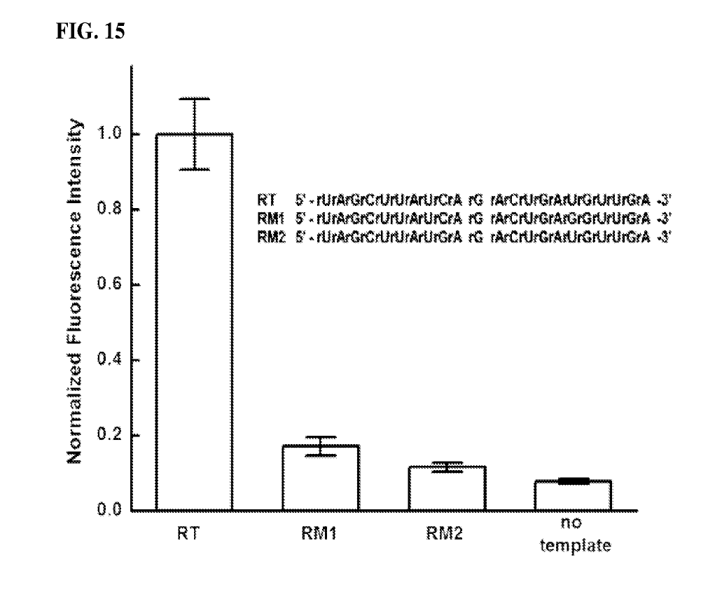

Histogram of FIG. 15 depicts Normalized fluorescence intensity of oligonucleotide probe 10BpMeTz2, 11BpMeNd2 reacted with 0.01 eq of match or mismatch template after 7 hours incubation using SEQ ID NOS:27-29.

1.8. Flight Mass Spectrometer (TOFMS) Characterization of Templated Reaction Products.

1.8.1. ESI-TOFMS characterization the reaction products of d27′-Tz with d27′-ABN

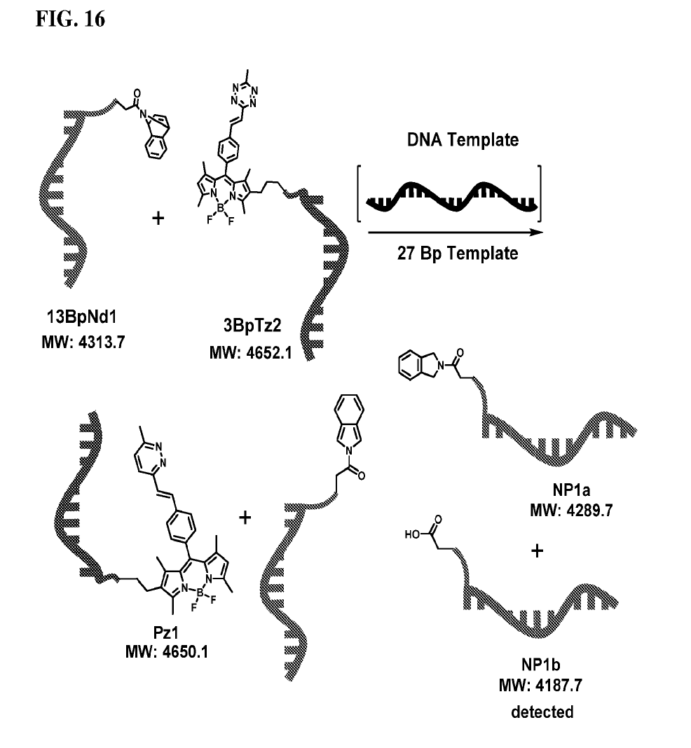

A reaction scheme is provided in FIG. 16 for ESI-TOFMS characterization of d27′-Tz with d27′-ABN. ESI-TOFMS spectra of Pz1, deconvoluted mass, and zoom in of deconvoluted mass were obtained. ESI-TOFMS spectra of NP 1a, deconvoluted mass, and zoom in of deconvoluted mass were also obtained. ESI-TOFMS spectra of NP 1b, deconvoluted mass, and zoom in of deconvoluted mass were also obtained.

1.8.2. ESI-TOFMS characterization the reaction products of d21′-Tz with d21′-ABN

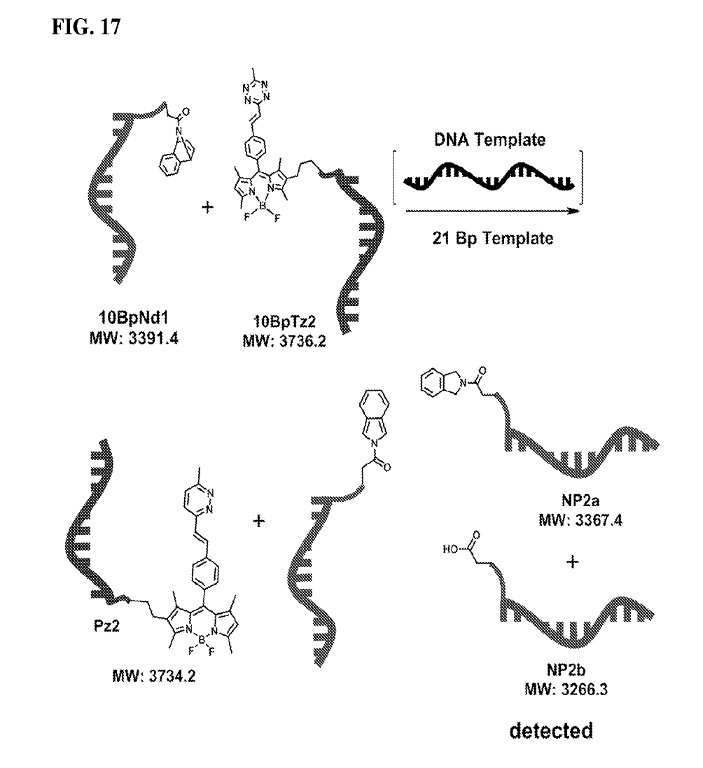

FIG. 17 provides a characterization scheme for the reaction products of d21′-Tz with d21′-ABN. ESI-TOFMS spectra of Pz2, zoom in of spectra, deconvoluted mass, and zoom in of deconvoluted mass were obtained. ESI-TOFMS spectra of Pz2, zoom in of spectra, deconvoluted mass, and zoom in of deconvoluted mass were obtained. ESI-TOFMS spectra of Np2a, zoom in of spectra, deconvoluted mass, and zoom in of deconvoluted mass were obtained. ESI-TOFMS spectra of Np2b, deconvoluted mass, and zoom in of deconvoluted mass were obtained.

1.8.3. ESI-TOFMS characterization the reaction products of mir21′-Tz with mir21′-ABN

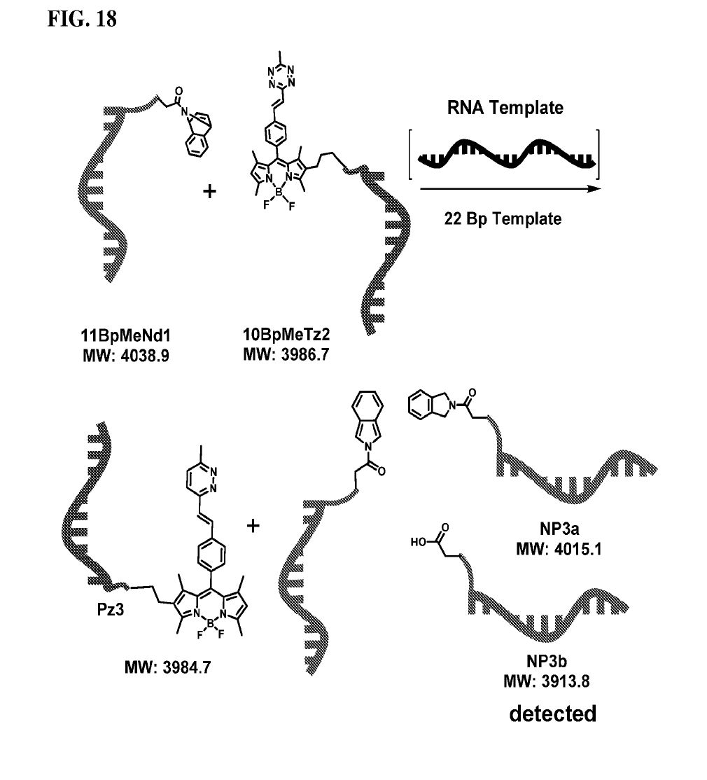

FIG. 18 provides a characterization scheme for the reaction products of mir21′-Tz with mir21′-ABN. ESI-TOFMS spectra of Pz3 and deconvoluted mass spectrum were obtained. ESI-TOFMS spectra of NP3a and deconvoluted mass spectrum were obtained. ESI-TOFMS spectra of NP3b and deconvoluted mass spectrum were obtained.

NMR data have also been obtained for certain compounds as set forth herein.

Template sequences for Example 1 are set forth in Table 1.1 following. Probe sequences are set forth in Table 1.2 following.

TABLE 1.1
Template Sequence

| | | SEQ | |
|---|---|---|---|
| | | ID | |
| Name | Type | NO: | 5′-Sequence |
| d27 | DNA | 1 | 5′-TTG ACG CCA TCG AAG GTA GTG |
| | | | TTG AAT |
| d21 | DNA | 2 | 5′-ACG CCA TCG AAG GTA GTG TTG |
| Template | | | |
| d21-A | DNA | 3 | 5′-ACG CCA TAG AAG GTA GTATT G |
| d21-B | DNA | 4 | 5′-AAG CCA TAG AAG GTA GTG TTG |
| mir21 | RNA | 5 | 5′-UAG CUU AUC AGA CUG AUG UUG A |
| Template | | | |
| mir21-A | RNA | 6 | 5′-UAG CUU AUC AGA CUG AGG UUG A |
| mir21-B | RNA | 7 | 5′-UAG CUU AUG AGA CUG AUG UUG A |

TABLE 1.2
Probe sequences

| | | SEQ ID | | |
|---|---|---|---|---|
| Name | Type | NO: | 5′-Sequence | Modification |
| d27′-B- | DNA | 8 | /5AmMC6/ TCG ATG GCG TCA A | 5′-B-Tz-NHS |
| Tz | | | | |
| d27′- | DNA | 9 | ATT CAA CAC TAC C /3AmMO/ | 3′-ABN-NHS |
| ABN | | | | |
| d21′ -B- | DNA | 10 | /5AmMC6/ TCG ATG GCG T/ | 5′-B-Tz-NHS |
| Tz | | | | |
| d21′- | DNA | 11 | CAA CAC TAC C /3AmMO/ | 3′-ABN-NHS |
| ABN | | | | |
| d21′-Cyp | DNA | 12 | CAA CAC TAC C /3AmMO/ | 3′-Cyp-NHS |
| mir21′- | 2′-O- | 13 | /5AmMC6/ AGA UAA GCU A | 5′-B-Tz-NHS |
| B-Tz | Methyl | | | |
| RNA | | | | |
| mir21′- | 2′-O- | 14 | UCA ACA UCA GU /3AmMO/ | 3′-ABN-NHS |
| ABN | Methyl | | | |
| RNA | | | | |

### Example 2

### Bioorthogonal Tetrazine Transfer Reactions for In-situ Detection of microRNA

Tetrazine ligations are an emerging class of bioorthogonal chemistry that has found increasing use in a wide variety of applications ranging from live-cell imaging, detection of proteins, in vivo imaging, and probing of glycosylation patterns.^{[1]} Tetrazine-based cycloadditions benefit from tunable kinetics, chemoselectivity, and the opportunity for fluorogenic reactions.^{[2]} An exciting application for tetrazine chemistry is the detection of DNA/RNA templates by driving fluorogenic reaction between tetrazine-quenched fluorescent moieties and dienophiles located on matched antisense probes. However, a fundamental limitation with tetrazine ligation is the irreversible coupling, which results in products with higher stability. High product stability prevents signal amplification by turnover, which would be necessary for detecting low abundant nucleic acids of interest, for instance endogenous microRNAs (miRNAs). Here we disclose a tetrazine transfer reaction that proceeds via cycloaddition to form a ligation product that subsequently spontaneously fragments by a retro Diels-Alder reaction. The reaction elicits strong fluorogenic responses from highly quenched tetrazine probes, and we utilize it to detect nucleic acids such as DNA at femtomolar concentrations. Furthermore, we demonstrate that this approach is suitable for highly sensitive and specific detection of miRNA templates. This enables live cell and lysate detection of endogenous miRNA. Remarkably, the technique is also able to differentiate between miRNA templates bearing a single mismatch, with signal to background ratios in excess of 20-fold. We imagine tetrazine transfer reactions could find wide application for amplified detection of clinically relevant nucleic acid templates, with possible application in live cell imaging and point-of-care diagnostics.^{[3]}

Recent synthetic advances have improved the utility of tetrazines as biological probes, including improved catalytic synthesis of tetrazines and the development of second-generation fluorogenic tetrazine-quenched fluorescent moieties.^{[4]} One potential application for such probes would be to develop a sensitive method of detecting DNA and RNA sequences. We recently demonstrated that DNA/RNA templates could greatly accelerate tetrazine cycloadditions, albeit using first-generation moderately-fluorogenic probes.^{[5]} The use of second-generation, highly fluorogenic alkenyl-tetrazine probes, which we recently reported,^{[4c]} could improve the ability to detect nucleic acid templates. However, a major barrier is that typical tetrazine ligation chemistry is poorly suited for facilitating high turnover in nucleic acid-templated reactions. The ligation product between the two oligonucleotide probes has a higher affinity for the template than its reactive precursors.^{[5]} The tightly-bound product limits further signal amplification produced by reactant turnover on a single template.^{[6]} Several groups have successfully explored DNA- or RNA-templated turnover of non-ligating fluorogenic reactions, such as acyl-transfer, reductions, or alternative nucleophilic/electrophilic chemistry.^{[7]} However, developing methods that show high sensitivity, specificity, stability, and single mismatch sensitivity against miRNA templates has been challenging, particularly without the use of enzymatic reactions or external stimuli.^{[7b,8]} The favorable properties of tetrazine reactions would seem to be well suited for miRNA detection. However highly-fluorogenic tetrazine reactions that proceed by transfer of functionality, as opposed to irreversible ligation, have not been previously explored.

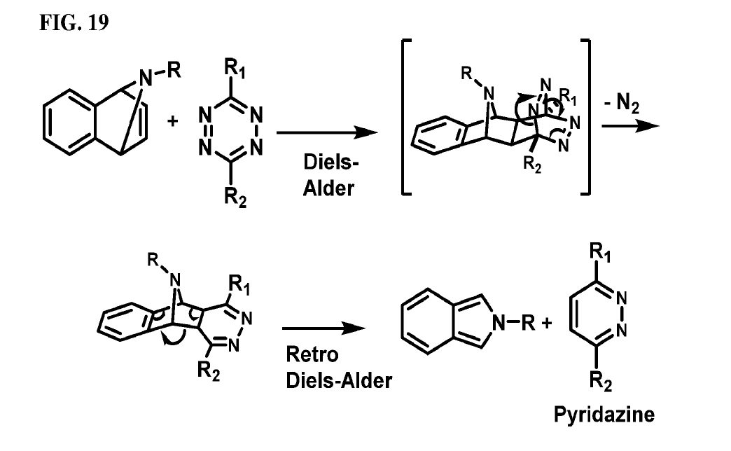

To address this fundamental problem, we utilized 7-azabenzonorbornadiene derivatives as novel strained dienophiles which can undergo irreversible inverse Diels-Alder reactions with tetrazines to release dinitrogen and form a dihydropyridazine coupling adducts.^{[9]} In contrast to typical strained dienophiles such as trans-cyclooctenes, norbornenes, or cyclopropenes, the product dihydropyridazine does not remain a stable conjugate but instead spontaneously undergoes a retro-Diels-Alder reaction to aromatize and fragment (FIG. 19).^{[9-10]} Along with loss of dinitrogen, the net result of this process is effectively a functional group transfer between the dienophile and the tetrazine. We hypothesized that the lack of a ligation product from this tetrazine-azabenzonorbornadiene transfer reaction would make it ideal for enabling oligonucleotide template-driven turnover, and we tested the suitability of using this reaction in probes for detecting DNA and RNA templates.

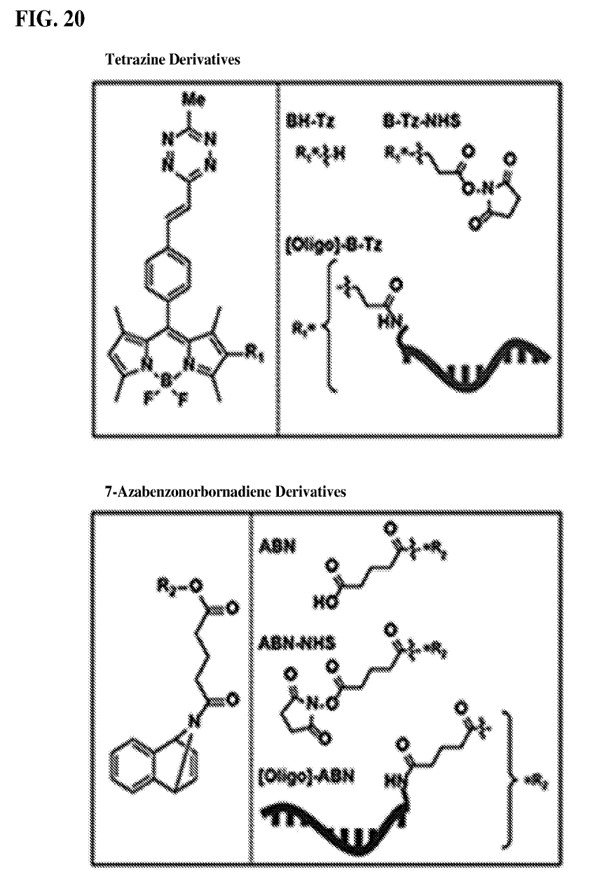

We first assessed the reactivity between a simple 7-azabenzonorbornadiene derivative ABN and the previously-described highly-fluorogenic tetrazine- BODIPY® compound TzH in chloroform. The reaction reached completion after 24 hours and produced the highly fluorescent pyridazine product with a 95% yield. This validated the notion that a tetrazine transfer reaction could indeed elicit fluorogenic response from quenched tetrazine probes. We next designed compounds Tz-NHS and ABN-NHS for oligonucleotide modification (FIG. 20) through straightforward NHS coupling chemistry. A series of 5′ tetrazine-oligonucleotide probes and 3′ 7-azabenzonorbornadiene-oligonucleotide probes were made by reaction of Tz-NHS or ABN-NHS with 5′ or 3′ amino-modified oligos (Table 2.1, sequence definitions Table 2.2) and were characterized by ESI-TOFMS. These antisense probes were designed such that the 5′ tetrazine and 3′ dienophile would be brought into close proximity when hybridized to a complementary template oligonucleotide strand (FIG. 21).

TABLE 2.1
Probe sequences for Example 2.

| | | SEQ ID | | |
|---|---|---|---|---|
| Name | Type | NO: | 5′-Sequence | Modification |
| d27′-Tz | DNA | 15 | /5AmMC6/ TCG ATG GCG TCA A | 5′-Tz-NHS |
| d27′- | DNA | 16 | ATT CAA CAC TAC C | 3′-ABN- |
| ABN | | | /3AmMO/ | NHS |
| d21′-Tz | DNA | 17 | /5AmMC6/ TCG ATG GCG T/ | 5′-Tz-NHS |
| d21′- | DNA | 18 | CAA CAC TAC C | 3′-ABN- |
| ABN | | | /3AmMO/ | NHS |
| d21′-Cyp | DNA | 19 | CAA CAC TAC C | 3′-Cyp-NHS |
| | | | /3AmMO/ | |
| mir21′- | 2′-O- | 20 | /5AmMC6/ AGA UAA GCU A | 5′-Tz-NHS |
| Tz | Methyl | | | |
| | RNA | | | |
| mir21′- | 2′-O- | 21 | UCA ACA UCA GU | 3′-ABN- |
| ABN | Methyl | | /3AmMO/ | NHS |
| | RNA | | | |

TABLE 2.2
Sequences of templates
Templates

| | | SEQ ID | |
|---|---|---|---|
| Name | Type | NO: | 5′-Sequence |
| d27 | DNA | 22 | 5′-TTG ACG CCA TCG AAG GTA GTG |
| | | | TTG AAT |
| d21 | DNA | 23 | 5′-ACG CCA TCG AAG GTA GTG TTG |
| mir21 | RNA | 24 | 5′-UAG CUU AUC AGA CUG AUG |
| | | | UUG A |
| mir21-A | RNA | 25 | 5′-UAG CUU AUC AGA CUG AGG |
| | | | UUG A |
| mir21-B | RNA | 26 | 5′-UAG CUU AUG AGA CUG AUG |
| | | | UUG A |

FIG. 22 provides an exemplary signal amplification cycle.

To study the kinetics of the DNA-templated tetrazine transfer reaction with azabenzonorbornadiene we synthesized probes d27′-Tz and d27′-ABN, and the corresponding DNA template d27. When 1 μM of each probe was allowed to react in hybridization buffer over 1.5 hours, insignificant reaction was observed by fluorescence or HPLC. In contrast, when 1 μM of each probe was allowed to react in the presence of 1 μM of d27, a reaction was immediately detectable by fluorescence measurements and HPLC. The fluorescence increased with an observed first order rate constant of 9.1±0.2×10⁻⁴ s⁻¹ and a reaction half-life of 12.7 mins The sample was found to have a fluorescence turn-on of 108-fold after reaction (details in SI). This represents over an order of magnitude improvement compared to previously described fluorogenic tetrazine oligonucleotide probes,^{[5]} and is comparable to some of the best visible spectrum turn-on probes that have been utilized for oligonucleotide-templated chemistry.^{[7c, 7d, 7f]}

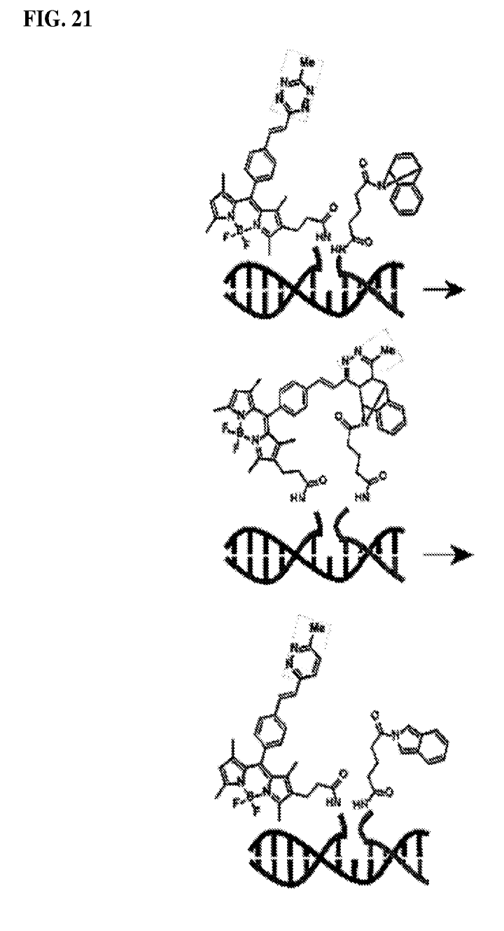

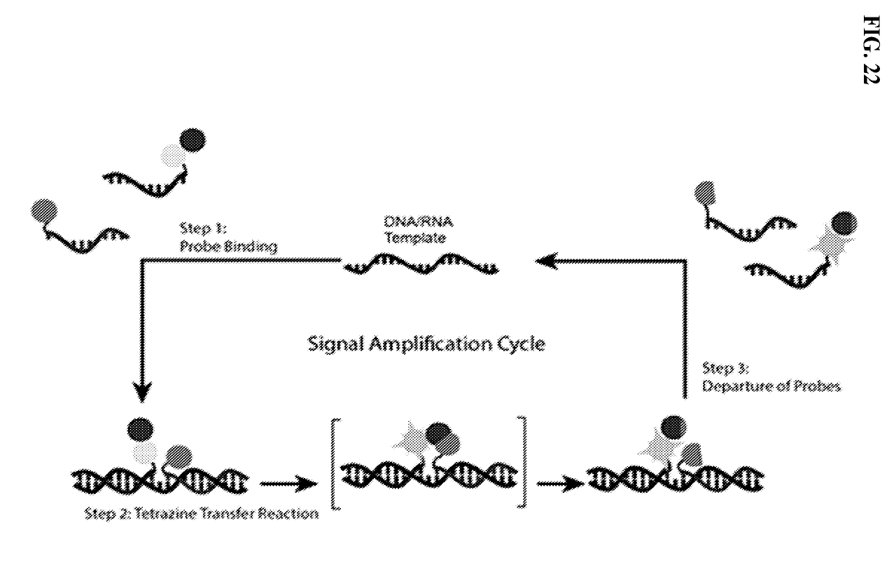

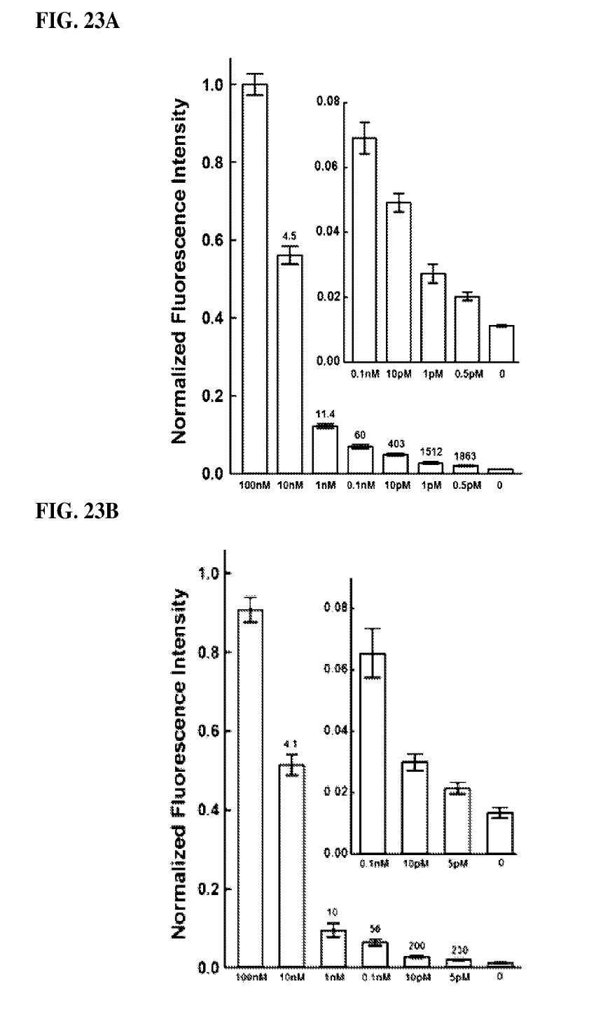

Given that the oligonucleotide template greatly accelerated the reaction kinetics between d27′-Tz and d27′-ABN and elicited a high fluorescence turn-on, we turned our attention to studying template driven reaction turnover. As shown in FIGS. 23A and 23B, the proposed turnover reaction is a dynamic strand exchange process, which would likely benefit from occurring near the probes' melting temperatures (Tₘ). Since we envisioned eventual application to physiologically relevant conditions, we chose to design probes with Tₘ close to 37° C. With this parameter in mind we synthesized shorter probes, d21′-Tz and d21′-ABN, for our template-driven turnover study. In hybridization buffer, we measured the Tₘ of these probes to be 40° C. and 42° C., respectively.

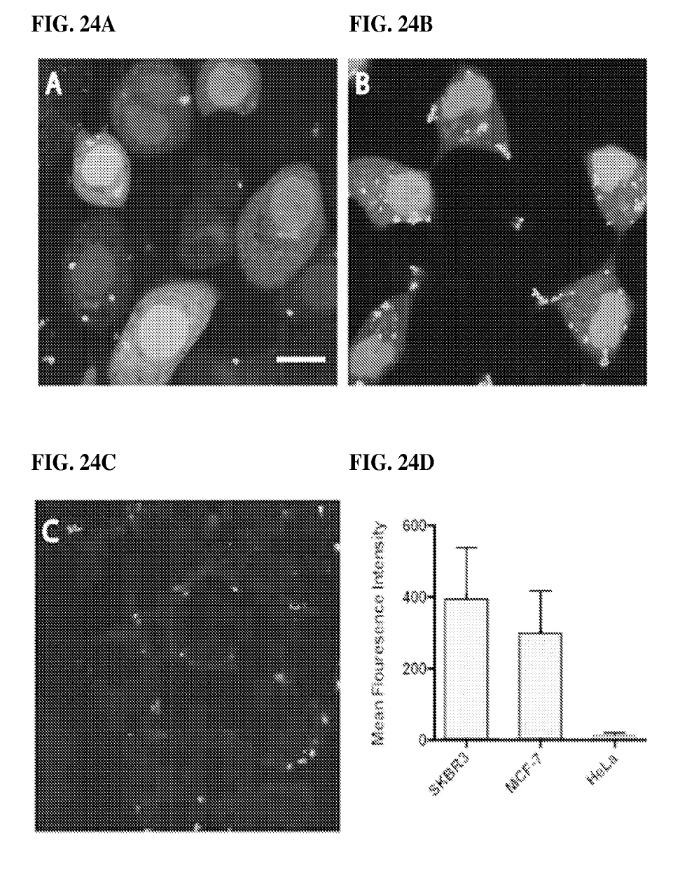

To test template-driven turnover, we incubated d21′-Tz and d21′-ABN, with decreasing amounts of DNA template. Fluorescence increase above background was measured and compared to the signal elicited from a stoichiometric template concentration to determine the extent of turnover. As shown in FIG. 24A, after 7 h we observed significant fluorescence signal amplification using DNA template ranging from nanomolar to femtomolar concentration. Sub-stochiometric template concentrations were capable of allowing multiple reactions per template and the number of reaction turnovers substantially increased as the template concentration decreased. This is similar to previous observations and is likely due to the increased ratio of probe to template.^{[7e]} Template could be discriminated from background down to a remarkable 500 fM. At this concentration of template, around 1% yield of product was formed, representing of turnover of ˜1863 reactions for each template. Mismatch discrimination assays that were performed also demonstrated the sequence-specific nature of this effect. To study the benefit of utilizing a transfer reaction, we compared the tetrazine transfer reaction of d21′-Tz and d21′-ABN to the tetrazine ligation between d21′-Tz and d21′-Cyp, an oligo probe modified with a 3′-cyclopropene derivative. The DNA templated reaction with the cyclopropene probe was previously found to proceed at a similar rate, but the cyclopropene forms a ligation adduct with tetrazine instead of undergoing a transfer reaction.^{[5]} As expected, the reaction with the cyclopropene probe at substoichiometric template concentrations allowed fewer reactions per template and resulted in a much smaller increase in fluorescence intensity compared to the reaction with the azabenzonorbornadiene probe. This is further evidence that, while tetrazine ligation inhibits turnover, tetrazine transfer reactions facilitate strand exchange leading to signal amplification.^{[6b, 7c]}

There have been numerous exciting chemistries applied to fluorogenic DNA-templated chemistry, but tetrazine ligation probes have already been established as robust and bioorthogonal. To test whether our tetrazine transfer reaction probes were similarly robust, we incubated d21′-Tz and d21′-ABN with excess oxidants, reductants, and thiol nucleophiles for 7 hours. To our delight, the tetrazine-azabenzonorbornadiene turnover reaction elicited nearly full fluorogenic response in the presence of 1% template when compared to control reactions.

Having demonstrated the potential for tetrazine transfer reactions to detect low concentrations of an oligonucleotide template in the chemical conditions found in physiological media, we next turned our interest to developing nuclease-resistant oligonucleotide probes for endogenous microRNA (miRNA) detection.^{[11]} MicroRNAs are a class of single-stranded non-coding regulatory RNAs that can regulate a wide variety of biological processes through the degradation of mRNA targets. The targets of miRNAs are often involved in critical cellular processes such as proliferation, growth, apoptosis, and differentiation. Altered expression of miRNAs has been linked to large number of human diseases and disorders.^{[12]} Thus, there is tremendous interest in methods to reliably detect, quantitate, and profile miRNA levels.^{[11, 13]} In particular, methods are needed that can rapidly profile miRNA levels in a small sample with high sensitivity and specificity, and which can be used for in situ miRNA quantification.^{[13a]} Methods that meet these criteria would have a high likelihood of translation into clinical settings.^{[3a]}

As a proof of concept, we explored the use of tetrazine transfer reactions to detect microRNA-21 (mir-21), a 22 base pair miRNA that was one of the first oncogenic miRNAs, known as oncomirs, to be identified.^{[14]} Mir-21 expression is associated with a variety of human cancers. Of note, it has been shown to be highly expressed in human breast cancer cells.^{[13b, 15]} Due to the importance of mir-21 in human oncology, it has been commonly targeted by alternative detection techniques, which also provides a method for benchmarking our approach.

To develop probes to detect endogenous miRNA, we first synthesized new tetrazine- and azabenzonorbornadiene-modified oligonucleotides using 2′-O-methyloligoribonucleotides (2′-O-methyl RNA). 2′-O-methyl RNA is comparable to RNA but has faster kinetics of hybridization to complementary targets and displays better discrimination between matched and mismatched RNA targets.^{[16]} Perhaps most significantly for in-situ detection of miRNA, 2′-O-methyl RNA is highly resistant to degradation by nucleases which would otherwise degrade RNA probes before they could hybridize with their target.^{[16b]} We synthesized 2′-O-methyl RNA probes mir21′-B-Tz and mir21′-ABN that contain complementary sequences to mir-21. Initially, we explored turnover reaction using a synthetic mir-21 template. In buffer mir21′-B-Tz and mir21′-ABN showed excellent signal amplification in the presence of mir-21 at multiple template concentrations after 7 h (FIG. 2.1B), with a detection limit of approximately 5 pM.

A significant challenge in detecting miRNA in biological samples is the need to be able to discriminate between similar miRNA sequences.^{[13a]} It has been shown that miRNAs within a family can differ by a single base.^{[11-12, 17]} Thus, probes designed to detect a specific miRNA need to possess the ability to distinguish between templates bearing a single mismatch. We tested the ability of mir21′-Tz and mir21′-ABN to discriminate between mir-21 and two alternative templates that each bore a single mismatch. Template mir21A contained a mismatch separated from the site of tetrazine reaction by a single nucleotide while template mir21B had a mismatch that was 4 nucleobases away from the site of reaction. Remarkably, at 1% equivalent template concentration, our probes showed high discrimination against both mismatch templates mir21A and mir21B, with a perfect match/mismatch (PM/MM) ratio of X and Y respectively. The PM/MM ratio was higher with mir21A compared to mir21B, in line with previous work that has demonstrated that templated chemical reactions are more sensitive to mismatches near the site of chemical reaction. The sensitivity to a single base mismatch is particularly impressive considering past results with miRNA templated chemistry, which have only demonstrated discrimination between miRNA templates differing by two or more mismatches.^{[8b,18]}

As an oncomir, mir-21 has been shown to be upregulated in multiple cancers.^{[19]} In particular, several studies have demonstrated that a variety of breast cancers and breast cancer derived cell lines show upregulation of mir-21 expression.^{[13b, 15b]}) Furthermore, mir-21 expression shows pronounced increases well cell lines or cancers develop resistance to chemotherapeutics.^{[15a]} Thus, a rapid method to profile mir-21 in cell samples could allow mir-21 expression to be a valuable diagnostic marker. With this in mind, we investigated the use of probes mir21′-Tz and mir21′-ABN for detection of mir21 in live breast cancer cells. We chose to utilize the breast cancer derived cell lines SKBR3 and MCF-7 due to previous work demonstrating their high mir-21 expression^{[13b, 15]}. As a comparison, we also imaged our probes in HeLa cells, a cervical cancer cell line that has been shown to have lower mir-21 expression compared to breast cancer lines such as MCF-7.

For imaging studies, the cell lines were transfected with 200 nM of mir21′-Tz and mir21′-ABN probes using LIPOFECTAMINE™ 2000 (Invitrogen, Calif., USA) as a transfection agent. After a two-hour incubation period the cells where imaged using confocal microscopy. Both the MCF-7 and SKBR³ breast cancer cells displayed strong fluorescent staining, indicating the presence of mir-21. In comparison, much less staining was observed in the HeLa cell line. Cells were also discerned by bright-field microscopy and nuclei were counterstained using Hoechst 33342. To eliminate variability that might be caused by differential probe uptake between cell lines, mir21′-Tz and mir21′-ABN were also tested in cell-derived lysates After 1.5 h incubation at 37° C., lysates derived from breast cancer cell lines SKBR3 and MCF7 exhibited a ˜5-fold increase in mir-21 signal compared to lysate derived from HeLa cells. To verify the specificity of the fluorogenic signal to mir-21 template, mir21′-Tz and mir21′-ABN were incubated along with a 10-fold excess of unmodified oligonucleotide probes with identical sequences as a competitive inhibitor. Competition significantly reduced the signal by approximately 5-fold, demonstrating that that the resulting fluorescent signal is due to mir-21 templated induced ligation. The ability to detect mir-21 in cell derived lysates is a distinct advantage of this technique compared to alternatives such as Northern blot and qPCR, which require careful extraction of RNA before detection.

In summary, we have developed a fluorogenic tetrazine transfer reaction using 7-azabenzonorbornadiene derivatives and have utilized the reaction to detect oligonucleotides with high sensitivity and sequence specificity. Critical to achieving signal amplification is template driven turnover of antisense probes, which is enabled due to spontaneous diazine release after the initial tetrazine ligation takes place. The use of a highly quenched alkenyl-fluorogenic tetrazine enables >100-fold increase in fluorescence in response to the tetrazine transfer reaction. By using RNA to template the tetrazine transfer reaction on appropriate antisense probes, we were able to detect mir-21 down to the low picomolar level, and with high sensitivity to the presence of single base mismatches in the miRNA template. The probes were capable of detecting endogenous mir-21 both in live cells and cell lysates. We believe oligonucleotide template directed fluorogenic tetrazine transfer reactions will useful for numerous applications that require detecting specific nucleic acids in either live cells or biological samples. Specifically, these probes are likely to be useful for profiling endogenous miRNA levels in living cells, circulating exosomes, and tissues.^{[13a]}

### REFERENCES (EXAMPLE 2)

[1] aM. L. Blackman, M. Royzen, J. M. Fox, Journal of the American Chemical Society 2008, 130, 13518-13519; bN. K. Devaraj, G. M. Thurber, E. J. Keliher, B. Marinelli, R. Weissleder, Proceedings of the National Academy of Sciences of the United States of America 2012, 109, 4762-4767; cN. K. Devaraj, R. Weissleder, S. A. Hilderbrand, Bioconjugate chemistry 2008, 19, 2297-2299; dR. S. Erdmann, H Takakura, A. D. Thompson, F. Rivera-Molina, E. S. Allgeyer, J. Bewersdorf, D. Toomre, A. Schepartz, Angewandte Chemie 2014, 53, 10242-10246; eD. N. Kamber, L. A. Nazarova, Y. Liang, S. A. Lopez, D. M. Patterson, H. W. Shih, K. N. Houk, J. A. Prescher, Journal of the American Chemical Society 2013, 135, 13680-13683; fB. Nichols, Z. Qin, J. Yang, D. R. Vera, N. K. Devaraj, Chem Commun (Camb) 2014, 50, 5215-5217; gI. Nikic, T. Plass, 0. Schraidt, J. Szymanski, J. A. Briggs, C.

Schultz, E. A. Lemke, Angewandte Chemie 2014, 53, 2245-2249; hU. Rieder, N. W. Luedtke, Angewandte Chemie 2014, 53, 9168-9172; iA. K. Spate, H. Busskamp, A. Niederwieser, V. F. Schart, A. Marx, V. Wittmann, Bioconjugate chemistry 2014, 25, 147-154; jM. T. Taylor, M. L. Blackman, O. Dmitrenko, J. M. Fox, Journal of the American Chemical Society 2011, 133, 9646-9649; kC. M. Cole, J. Yang, J. Seckute, N. K. Devaraj, Chembiochem : a European journal of chemical biology 2013, 14, 205-208.;

[2] aN. K. Devaraj, S. Hilderbrand, R. Upadhyay, R. Mazitschek, R. Weissleder, Angewandte Chemie 2010, 49, 2869-2872; bN. K. Devaraj, R. Upadhyay, J. B. Haun, S. A. Hilderbrand, R. Weissleder, Angewandte Chemie 2009, 48, 7013-7016; cM. R. Karver, R. Weissleder, S. A. Hilderbrand, Bioconjugate chemistry 2011, 22, 2263-2270; dM. R. Karver, R. Weissleder, S. A. Hilderbrand,

Angewandte Chemie 2012, 51, 920-922; eZ. Li, H. Cai, M. Hassink, M. L. Blackman, R. C. Brown, P. S. Conti, J. M. Fox, Chem Commun (Camb) 2010, 46, 8043-8045.;

[3] aR. Garzon, G. Marcucci, Current opinion in oncology 2012, 24, 655-659; bB. A. Armitage, Current opinion in chemical biology 2011, 15, 806-812.;

[4] aJ. C. Carlson, L. G. Meimetis, S. A. Hilderbrand, R. Weissleder, Angewandte Chemie 2013, 52, 6917-6920; bL. G. Meimetis, J. C. Carlson, R. J. Giedt, R. H. Kohler, R. Weissleder, Angewandte Chemie 2014, 53, 7531-7534; cH. Wu, J. Yang, J. Seckute, N. K. Devaraj, Angewandte Chemie 2014, 53, 5805-5809; dJ. Yang, M. R. Karver, W. Li, S. Sahu, N. K. Devaraj, Angewandte Chemie 2012, 51, 5222-5225.;

[5] J. Seckute, J. Yang, N. K. Devaraj, Nucleic acids research 2013, 41, e148.;

[6] aK. Gorska, N. Winssinger, Angewandte Chemie 2013, 52, 6820-6843; bJ. Michaelis, A. Roloff, O. Seitz, Organic & biomolecular chemistry 2014, 12, 2821-2833.;

[7] aJ. Cai, X. Li, X. Yue, J. S. Taylor, Journal of the American Chemical Society 2004, 126, 16324-16325; bK. Gorska, N. Winssinger, Methods Mol Biol 2014, 1050, 179-192; cT. N. Grossmann, O. Seitz, Journal of the American Chemical Society 2006, 128, 15596-15597; dZ. Pianowski, K. Gorska, L. Oswald, C. A. Merten, N. Winssinger, Journal of the American Chemical Society 2009, 131, 6492-6497; eA. Shibata, T. Uzawa, Y. Nakashima, M. Ito, Y. Nakano, S. Shuto, Y. Ito, H. Abe, Journal of the American Chemical Society 2013, 135, 14172-14178; fR. M. Franzini, E. T. Kool, Journal of the American Chemical Society 2009, 131, 16021-16023; gR. M. Franzini, E. T. Kool, Bioconjugate chemistry 2011, 22, 1869-1877; hR. M. Franzini, E. T. Kool, Chemistry 2011, 17, 2168-2175.;

[8] aE. M. Harcourt, E. T. Kool, Nucleic acids research 2012, 40, e65; bK. K. Sadhu, N. Winssinger, Chemistry 2013, 19, 8182-8189.;

[9] aJ. Plumet, R. Rincon, Synlett 2008, 2008, 911-913; bG. M. Priestley, R. N. Warrener, Tetrahedron Letters 1972, 42, 4295-4298; cG. Y. Yang, M. Hanack, Y. W. Lee, Y. Chen, M. K. Lee, D. Dini, Chemistry 2003, 9, 2758-2762; dG. W. Gribble, C. S. Lehoullier, M. P. Sibi, R. W. Allen, J. Org. Chem 1985, 50, 1611-1616; eT. Sasaki, T. Manabe, S. Nishida, J. Org. Chem 1980, 45, 476-479.;

[10] aJ. E. Clements, J. R. Price, S. M. Neville, C. J. Kepert, Angewandte Chemie 2014, 53, 10164-10168; bE. Dalkilic, M. Güney, A. Daştan, N. Saracoglu, O. D. Lucchi, F. Fabris, Tetrahedron Letters 2009, 50, 1989-1991; cF. Thebault, A. J. Blake, C. Wilson, N. R. Champness, M. Schroder, New J Chem 2006, 30, 1498-1508.;

[11] C. C. Pritchard, H. H. Cheng, M. Tewari, Nature reviews. Genetics 2012, 13, 358-369.;

[12] aN. Bushati, S. M. Cohen, Annual review of cell and developmental biology 2007, 23, 175-205; bR. W. Carthew, E. J. Sontheimer, Cell 2009, 136, 642-655.;

[13] aH. Dong, J. Lei, L. Ding, Y. Wen, H. Ju, X. Zhang, Chemical reviews 2013, 113, 6207-6233; bM. V. Iorio, M. Ferracin, C. G. Liu, A. Veronese, R. Spizzo, S. Sabbioni, E. Magri, M. Pedriali, M. Fabbri, M. Campiglio, S. Menard, J. P. Palazzo, A. Rosenberg, P. Musiani, S. Volinia, I. Nenci, G. A. Calin, P. Querzoli, M. Negrini, C. M. Croce, Cancer research 2005, 65, 7065-7070.;

[14] M. Lagos-Quintana, R. Rauhut, W. Lendeckel, T. Tuschl, Science 2001, 294, 853-858.;

[15] aC. Gong, Y. Yao, Y. Wang, B. Liu, W. Wu, J. Chen, F. Su, H. Yao, E. Song, The Journal of biological chemistry 2011, 286, 19127-19137; bM. Shi, N. Guo, Cancer treatment reviews 2009, 35, 328-334.;

[16] aM. Majlessi, N. C. Nelson, M. M. Becker, Nucleic acids research 1998, 26, 2224-2229; bE. A. Lesnik, C. J. Guinosso, A. M. Kawasaki, H. Sasmor, M. Zounes, L. L. Cummins, D. J. Ecker, P.

D. Cook, S. M. Freier, Biochemistry 1993, 32, 7832-7838; cB. S. Sproat, A. I. Lamond, B. Beijer, P. Neuner, U. Ryder, Nucleic acids research 1989, 17, 3373-3386.;

[17] C. Chen, D. A. Ridzon, A. J. Broomer, Z. Zhou, D. H. Lee, J. T. Nguyen, M. Barbisin, N. L. Xu, V. R. Mahuvakar, M. R. Andersen, K. Q. Lao, K. J. Livak, K. J. Guegler, Nucleic acids research 2005, 33, e179.;

[18] K. Gorska, I. Keklikoglou, U. Tschulena, N. Winssinger, Chem Sci 2011, 2, 1969-1975.;

[19] aV. Jazbutyte, T. Thum, Curr Drug Targets 2010, 11, 926-935; bP. P. Medina, M. Nolde, F. J. Slack, Nature 2010, 467, 86-U119; cA. E. Frampton, T. M. H. Gall, L. Castellano, J. Stebbing, L. R. Jiao, J. Krell, Expert Rev Mol Diagn 2013, 13, 31-34; dJ. C. Akers, V. Ramakrishnan, R. Kim, J. Skog, I. Nakano, S. Pingle, J. Kalinina, W. Hua, S. Kesari, Y. Mao, X. O. Breakefield, F. H. Hochberg, E. G. Van Meir, B. S. Carter, C. C. Chen, PloS one 2013, 8, e78115; eN. M. Teplyuk, B. Mollenhauer, G. Gabriely, A. Giese, E. Kim, M. Smolsky, R. Y. Kim, M. G. Saria, S. Pastorino, S. Kesari, A. M. Krichevsky, Neuro-Oncology 2012, 14, 689-700.

### Example 3

### Exemplary No Wash Detection of Nanomolar Concentration of Protein

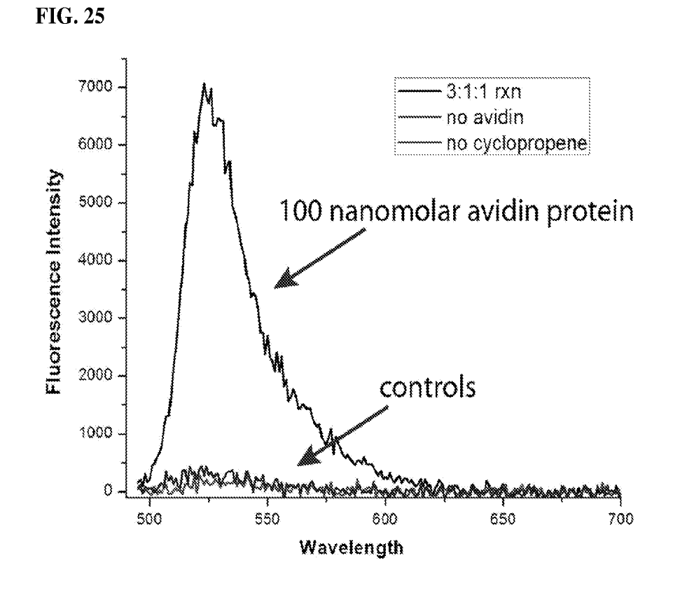

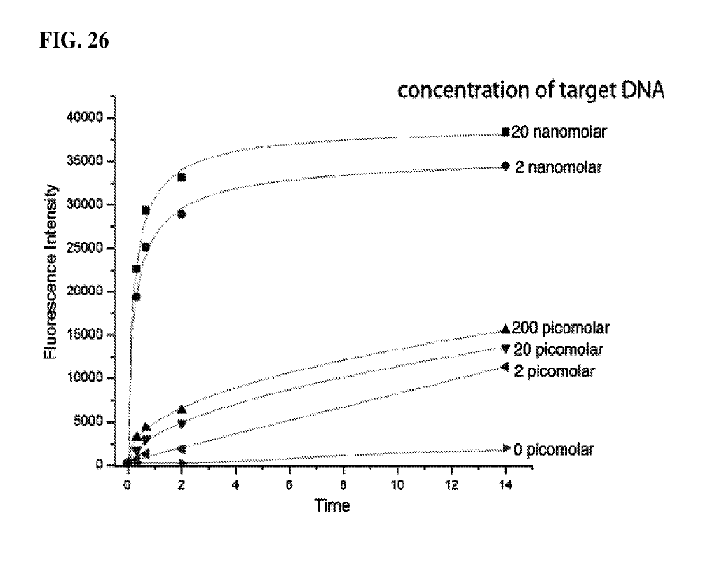

As depicted in FIG. 25, methods disclosed herein are useful for detection of avidin at the nanomolar level. As depicted in FIG. 26, methods disclosed herein are useful for detection of target DNA at the picomolar level.

### Example 4

### Synthesis of Dienophile.

Dienophiles can be synthesized by methods known in the art, and method disclosed herein. Representative synthetic schemes follow as Schemes 4.1 to 4.4.

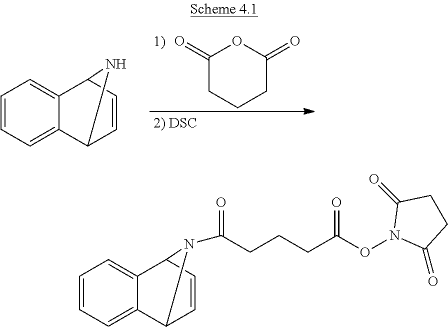 [SMILES: C1=CC2NC1c1ccccc12 ; O=C1CCCC(=O)O1 ; O=C(CCCC(=O)N1C2C=CC1c1ccccc12)ON1C(=O)CCC1=O]

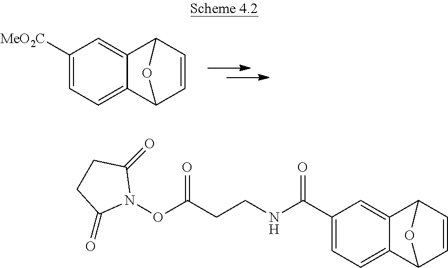

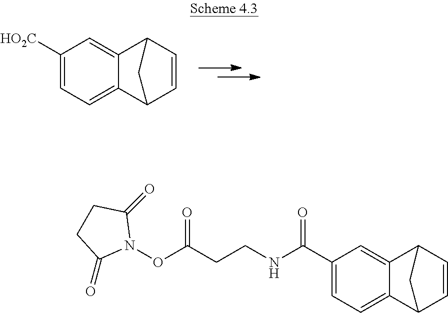 [SMILES: O=C(O)c1ccc2c(c1)C1C=CC2C1 ; O=C(CCNC(=O)c1ccc2c(c1)C1C=CC2C1)ON1C(=O)CCC1=O]

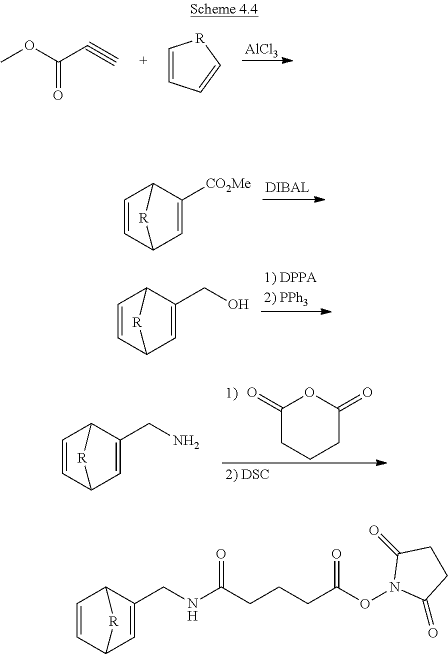

For Scheme 4.4, R is substituted or unsubstituted alkyl, O, or NBoc.

### Example 5

### Exemplar Synthesis of Resorufin Dienophile.

A synthetic scheme for synthesis of resorufin dienophile is set forth in Scheme 5.1 following.

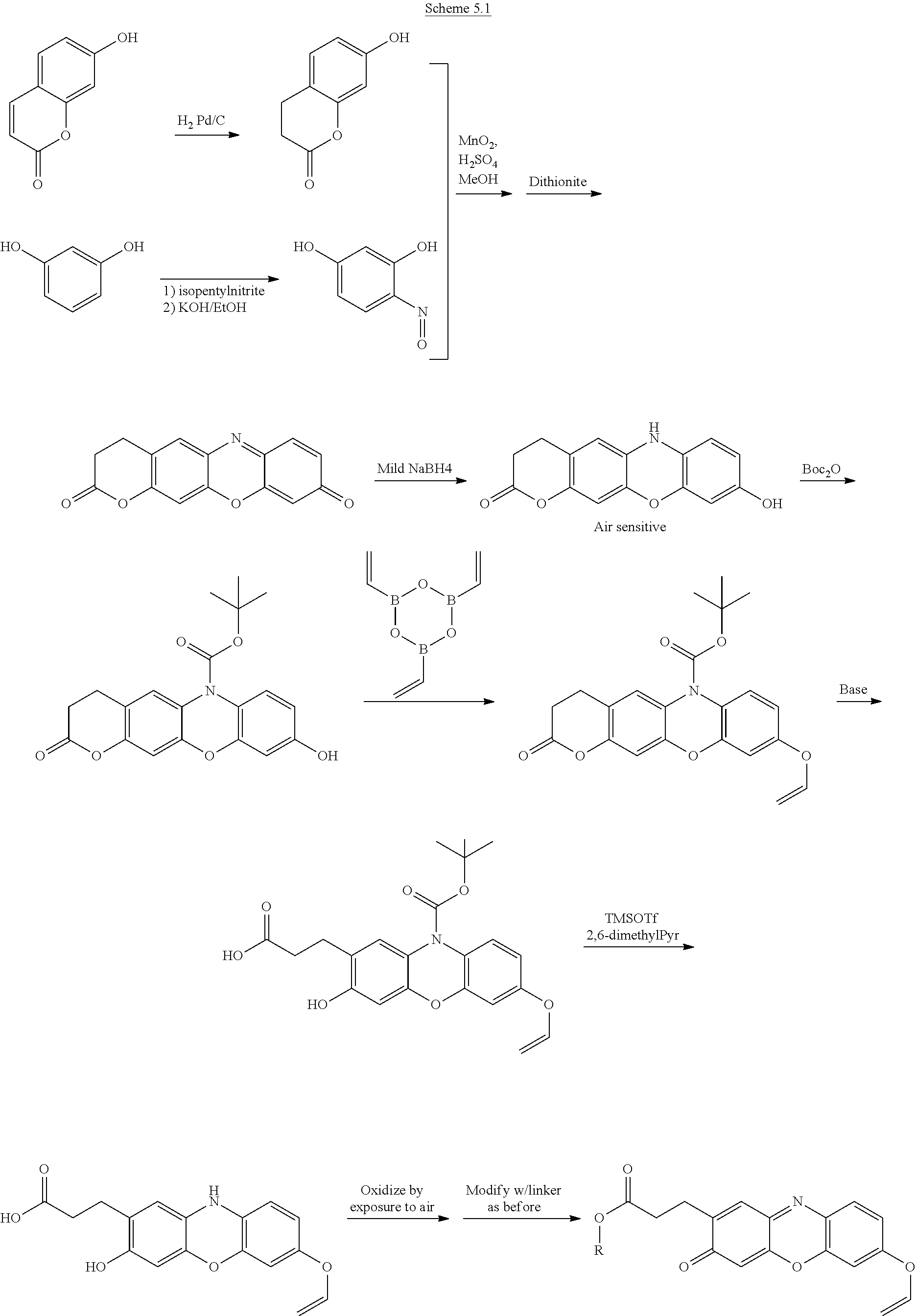

### Example 6

### Synthetic Schemes for Vinyl-Ethyl Quenched Dienophile/Fluorescent Moieties.

Scheme 6.1 following depicts possible synthetic routes to vinyl-ether quenched dienophile/fluorescent moieties. Specific targets include a coumarin carboxylic acid (2-oxo-6-(vinyloxy)-2H-chromene-3-carboxylic acid), a fluorescein carboxylic acid (5-((3-methyl-4-(3-oxo-6-(vinyloxy)-3H-xanthen-9-yl)phenyl)amino)-5-oxopentanoic acid) and a cyanine carboxylic acid (2-((E)-5-((E)-2-(1-(carboxymethyl)-3 ,3 -dimethyl-3H-1λ⁴-indol -2-yl)vinyl)-2-(vinyloxy) styryl)-1,3 ,3-trimethyl-3H-indol-1-ium)

### Example 7

### General Protocols for Bioorthogonal Tetrazine-Mediated Near-Infrared Fluorogenic Probe for mRNA Visualization (Example 8)

All reagents were purchased from Sigma-Aldrich and used without further purification unless otherwise noted, all oligonucleotides were purchased from Integrated DNA Technologies. The TLC plates used for purification were purchased from Agela Technologies (silica 200×200 mm, PH=5, MF =254, glass back). Other chromatographic purifications were conducted using 40-63 μm silica gel. All mixtures of solvents are given in v/v ratio. ¹H and ¹³C NMR spectroscopy were performed on a Jeol NMR at 500 (¹H) or 125 (¹³C) MHz. All ¹³C NMR spectra were proton decoupled. Fluorescence measurements were performed on a HORIBA FluoroMax-P spectrophotometer equipped with a single cuvette reader. Oligonucleotide concentrations were measured on a ThermoScientific NanoDrop 2000c UV—Vis Spectrophotometer with absorption wavelength of 260 nm.

### Example 8

### Bioorthogonal Tetrazine-Mediated Near-infrared Fluorogenic Probe for mRNA Visualization

Profiting from the recent tetrazine synthetic methodology development, ^{1.2} a battery of tetrazine fluorogenic probes have been uncovered.²⁻⁵ Several skeleton types of fluorescent moieties can be quenched by adjacent tetrazine, and after the tetrazine bioorthogonal reaction, more than hundreds fold fluorescence increase can be observed. These highly fluorogenic probes can be used for diverse applications such as no-wash live-cell imaging,^{4.5} endogenous oncogenic miRNA detection,⁶ and systemic fluorescence imaging.⁷ Tetrazines have an inherent absorbance around 510-550 nm. Thus, far-red and near infrared fluorescent moieties with relatively longer absorption wavelength may be challenging to quench by tetrazine participant energy transfer pathway. The design of tetrazine near infrared fluorogenic probe through a different quench mechanism can therefore afford new and useful methods for, e.g., detection of biomolecules.

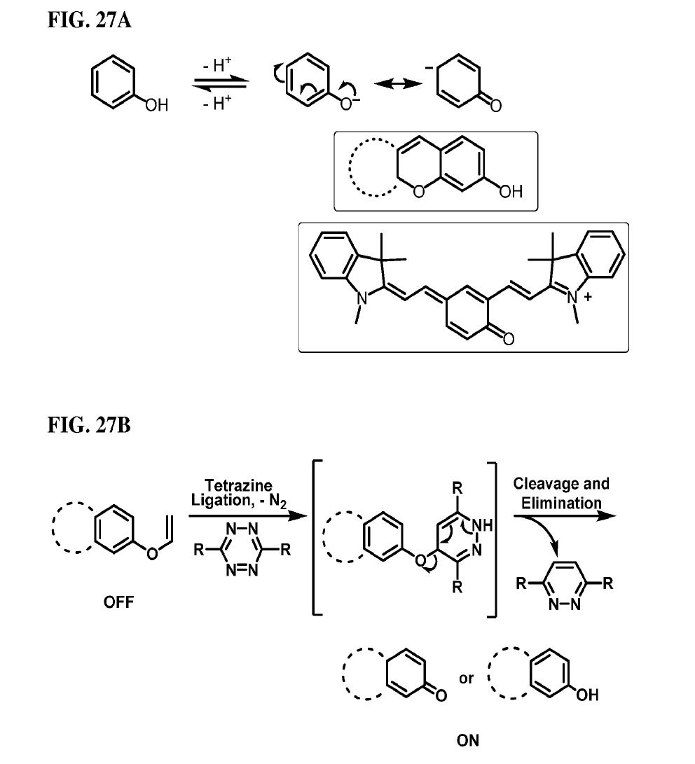

In addition to TBET and FRET, fluorescence can also be quenched by Internal Charge Transfer (ICT) process.⁸ Functional groups used as a trigger can mask the fluorescent moieties either by interrupting the pull-push conjugated π-electron system^{9,10} or holding the fluorescent moieties in a nonionizable form.¹¹⁻¹³ Recently, several bioorthogonal reactions with a click-release feature have been reported, which facilitate the application on bioluminescence imaging,¹¹ endogenous oncogenic miRNA detection,⁶ antibody-drug-conjugate release:¹⁴ protein decaging.¹⁵ To the best of our knowledge, there is no NIR fluorogenic probe that is uncovered by using bioorthogonal click-release reaction. A panel of fluorogenic probes triggered by tetrazine bioorthogonal chemistry has been designed. A phenol functional group is common existing in various fluorescent moieties such as coumarin and fluorescein. More interestingly the phenoxide anion is equally to cyclohexadienone anion (FIG. 27A), which can constitute the novel quinone cyanine dye.^{]} Therefore, we envisaged developing a tetrazine bioorthogonal click-release reaction: the reaction group can act as a cage to mask the phenol group and quench the fluorescence, after bioorthogonal reaction the cage can be released resulting free phenol to recover different fluorogenic structures.

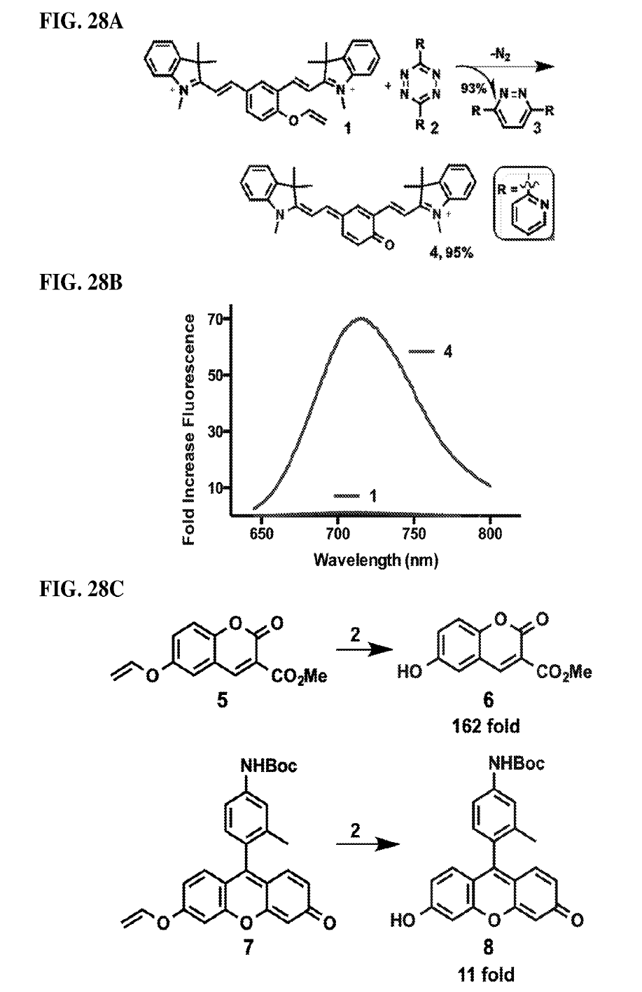

To achieve an unmasked phenol group in tetrazine reactions, we utilized phenyl vinyl ether as novel dienophiles that can undergo tetrazine-mediated transfer (TMT) reactions (FIG. 27B). Vinyl ethers react with tetrazines through a cascaded ligation-elimination process, resulting in a pyridazine and a free hydroxyl compound in almost quantitative yield.^{16,17} To verify our fluorogenic probe design, a model vinyl ether cyanine compound VE-1 was prepared. Starting from commercial available compound 4-hydroxyisophthalaldehyde, VE-1 can be obtained in 3 steps as a fully quenched light yellow solid. The reaction between VE-1 and dipyridyl tetrazine Tz-0 proceeded with high-efficiency, resulting two products Cy-1, Pz-0 in 95% and 93% yield (FIG. 28A). After Cy-1 was dissolved into PBS buffer, the solution turned cyan immediately, and 70-fold of fluorescence increase was observed compare to caged precursor VE-1 (FIG. 28B). We also investigated the absorption spectrums of VE-1 and Cy-1. At 620 nm, where is around the excitation wavelength, Cy-1 has an obvious absorption peak. However after caged by vinyl ether, the peak blue shifted to 550 nm on VE-1 spectrum, and the absorption at 620 nm is nearly nothing. The change of absorption spectrum indicated our vinyl ether ICT strategy for fluorescence quenching is highly effective.¹⁸

The quench ability of vinyl ether to other fluorescent moieties was also shown. Coumarin and fluorescein vinyl ether derivatives 2 and 5 can be straightforwardly synthesized by a few steps separately. After caged by the vinyl ether, the peak at 420 nm was disappeared on the absorption spectrum of 2, which resulting up to 162 fold turn-on after fully reacted with tetrazine. In contrast, the fluorescein vinyl ether only exhibited 11 fold turn-on after decaging, which is consistent with the absorption spectrum: the vinylation only weakened the peak intensity at 480 nm (. However, for green zone we can use our old strategy to achieve highly fluorogenic probes.²

After establishing the fluorogenic property of tetrazine-mediated vinyl ether probes, we investigated the reaction kinetics of this reaction. As we expected, the reaction is slow for direct labeling in cell at micromolar concentration.¹⁹ However, this can be overcome by using nucleic acid templated reactions, which can dramatically increase the effective concentration of two reaction partners by hybridization promoted spatial proximity. ^{C} More importantly, the nucleic acid templated reaction is of highly specifity and sensitivity. The reaction can take place only with full matched antisense template at nanomolar concentration, and templated reactions have been widely used for nucleic acids in situ imaging and detection in recent years.

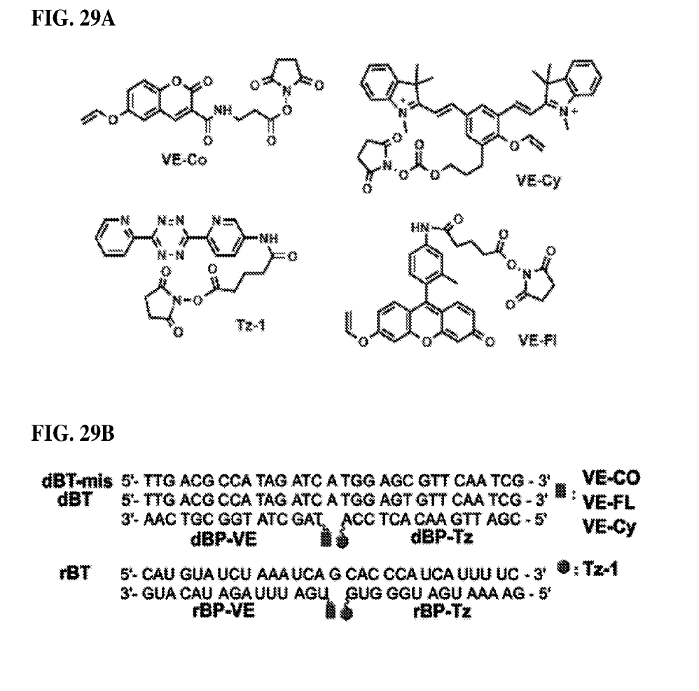

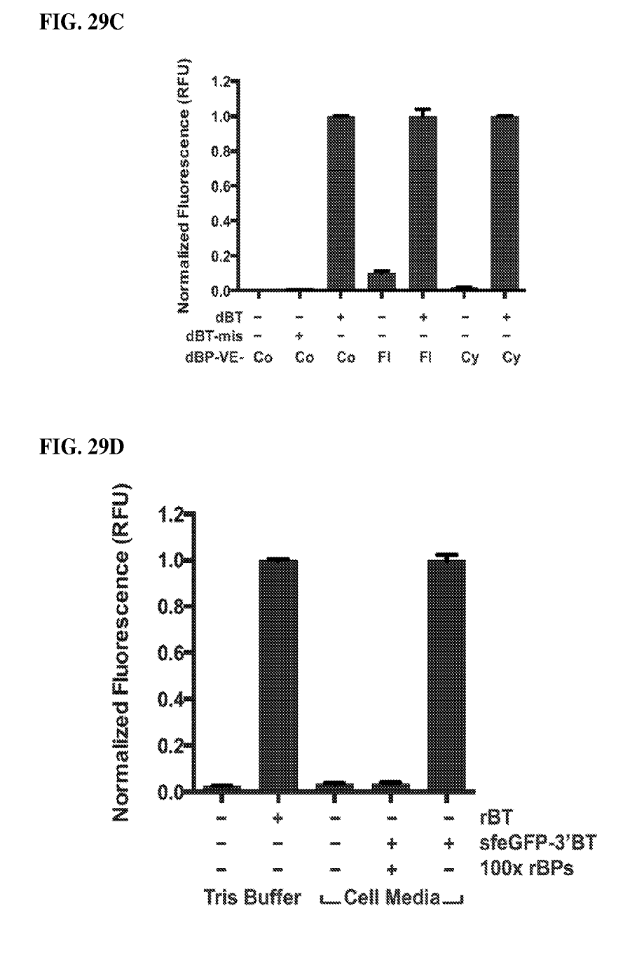

We designed a series of N-hydroxysuccinimide (NHS) esters compounds for oligonucleotide modification with NHS-coupling chemistry. Vinyl ether NHS compounds VE-2, VE-3, and VE-4 were obtained by post-functionalization from previous model compounds 2 and 5 or slightly change the synthetic scheme. By using classical NHS-coupling protocol, a panel of nucleic vinyl ether probes (d-VE and r-VE) and tetrazine probes (d-Tz and r-Tz) have been produced, which were characterized by ESI-TOF-MS. We first investigated the kinetics of templated vinyl ether TMT reaction. In the presence of DNA template d31, the reaction between coumarin vinyl ether probe d31-VE2 and tetrazine probe d31-Tzl immediately took place with fluorimetry readout. The first-order rate constant was measured as (2.1 ±0.03)×10⁴ s⁻¹ with a reaction half-life of 54.9 mins. Thus, the reaction kinetics is suitable for live-cell imaging After the templated TMT reaction was completed, more than 100 fold of fluorescence increase have been detected compared to the reaction without a d31 template (FIG. 29C column 3 and 1). We also verified the specifity of TMT templated reaction. Single mismatch template d31-mis (FIG. 29B) was picked for the discrimination experiment (FIG. 29C column 2) to compare to the experiment without a template (FIG. 29C column 1) after 8 hours incubation. Beside a coumarin DNA vinyl ether probe, we also tested the turn-on ability of other fluorogenic oligo probes. As we expect, after 8 hours incubation, the VE-3 and VE-4 probes also exhibited similar turn-on ratio as the model reactions (FIG. 29C column 4-7).

We next turned our interest to applying our vinyl ether oligo on mRNA visualization. mRNAs. We focused on using NIR vinyl ether probe for mRNA imaging which attracted numerous interest for the in vivo tissue imaging Instead of regular RNA backbone, we used 2′—O—methylation and phosphorothiolation modified RNA as probe backbond. With these modification, RNA probes have better in vivo stability and cell permeability. Moreover, modification will increase the binding ability and specifity to the complementary targets and against to the mismatch targets. All these features are in favour of live-cell mRNA imaging. We first studied the vinyl ether TMT reaction on synthetic RNA templated r31. The reaction went smoothly, an excellent NIR signal has been observed after 8 hours (FIG. 29D, column 1 and 2). Then we tested the binding and reaction property on more complicated RNA structures, which bearing our target sequence also have m-cherry GFP express domain. We carried out our experiment in cell-media. To our delight, we observed remarkable signal increase (column 4 and 7 in FIG. 29D) compared with the control experiment which don't contain m-cherry or GFP RNA as template (column 3 and 5 in FIG. 29D). In order to confirm the fluorescence signal is come from the templated hybridization, we added 50 equivalent unmodified 2—OMe—RNA probes as binding inhibitors. After 8 hours incubation, there is no significant signal increase has been detected compare to the control experiment (column 6 in FIG. 29D).

8.1 Model Reaction Kinetic Measurement

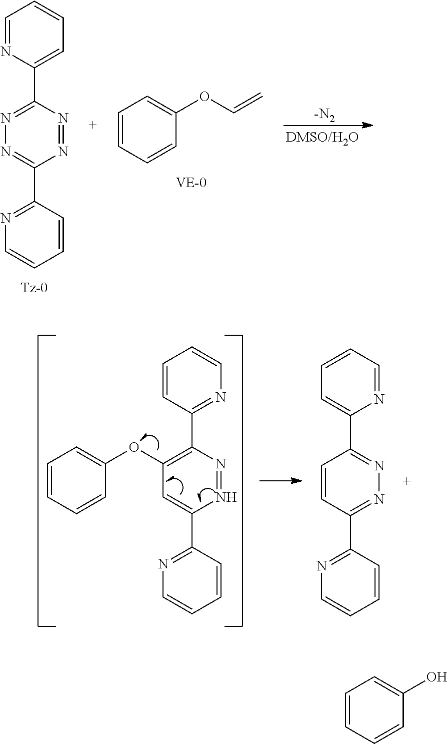

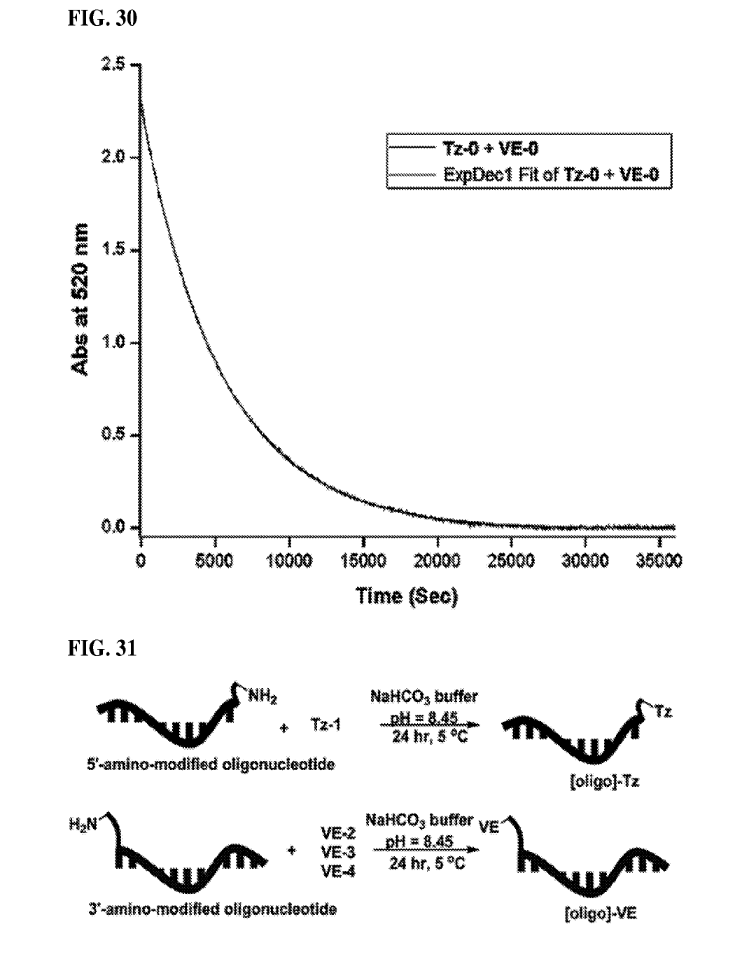

A Kinetic experiment was conducted under pseudo-first order conditions. Phenyl vinyl ether (VE-0, 250 mM) was reacted with 3,6-di-(2-pyridyl)-s-tetrazine (Tz-0, 5 mM) in a 1 mL solution of DMSO/H₂O=9:1. The decrease of the absorption at 520 nm was measured every 30 seconds (FIG. 30, black dots). The reaction was carried out at 37° C. (±0.5° C.) in a 1-mL UV cuvette. The data was fitted with a one-phase exponential decay curve (FIG. 30, red line) an observed first-order rate constant (k_{obs}), which was used to calculate the second order rate constant. The kinetics constants observed k_{obs} was 1.824 ±.003×10⁻⁴ (t_{1/2=}3799 s). The second order rate constant was calculated to be 7.296 (±0.012)×10⁻⁴M⁻¹_{S}⁻¹

8.2 Tag Synthesis

8.2.1 Synthesis of VE-1

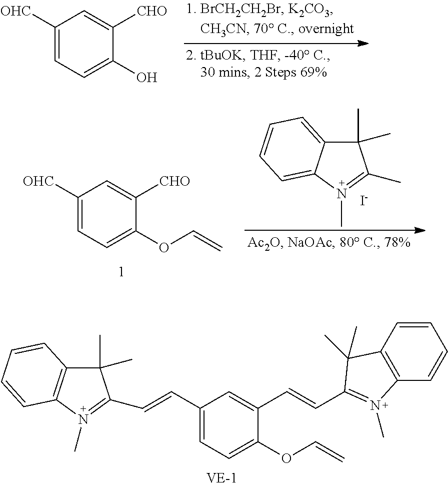

Synthesis of Compound 1:

4-Hydroxyisophthalaldehyde (150 mg, 1 mmol) was dissolved in anhydrous acetonitrile (5.0 mL). 1,2-Dibromoethane (1.9 g, 10 mmol) was added followed by K₂CO₃ (276 mg, 2 mmol) at room temperature. Then the reaction was stirred at 70° C. for overnight. After the reaction accomplished, the reaction solution was filtered, and the solvent was removed in vacuo. The residue was dissolved in anhydrous THF (10 mL) at −40° C. tBuOK (0.5 mmol) was slowly added in 10 mins. Then the reaction was stirred at −40° C. for 30 mins. After the reaction accomplished, the reaction was quenched by water, then the reaction solution was extracted by EtOAc (30 mL×3). The organic layer was dried over Na₂SO₄, filtered, and concentrated in vacuo. The residue was purified by silica column (Hexanes: EtOAc=25:1) to afford pure 1 (138 mg, yield 78%, 2 steps) as a white solid.

‘H NMR: (500 MHz, methylene chloride-d2) δ10.46 (d, J=0.9 Hz, 1H), 9.96 (s, 1H), 8.37-8.28 (m, 1H), 8.13-8.07 (m, 1H), 7.25 (d, J=8.6 Hz, 1H), 6.85-6.76 (m, 1H), 5.10-5.05 (m, 1H), 4.82-4.78 (m, 1H).

¹³C NMR: (126 MHz, methylene chloride-d2) δ190.52, 188.42, 163.28, 146.39, 135.99, 132.02, 131.64, 126.07, 116.81, 100.43.

HRMS: [M+H]⁺ m/z calcd. for [C₁₀H₉O₃]⁺ 177.0546, found 177.0545

Synthesis of Compound VE-1:

A mixture of compound 1 (88 mg, 0.5 mmol), NaOAc (99 mg, 1.2 mmol), and 1,2,3,3,-tetramethyl-3H-indolium iodide (345 mg, 1.15 mmol) was dissolved in 2 mL Ac₂O. The reaction mixture stirred for 30 min at 80° C. under an Ar atmosphere and monitored by HPLC-MS. After reaction completed, the solvent was removed under vacuo. The resulting crude product was dissolved in MeOH, and purified by preparative RP-HPLC to give compound VE-1 (190 mg, 78%) as a yellow solid.

‘H NMR: (500 MHz, methylene chloride-d2) δ 9.09 (s, 1H), 8.63 (d, J=14.6 Hz, 1H), 8.40-8.29 (m, 1H), 8.16-8.00 (m, 2H), 7.84 (d, J=10.3 Hz, 1H), 7.65 (d, J=10.3 Hz, 8H), 7.29 (t, J=8.4 Hz, 1H), 6.87 (dd, J=13.5, 5.9 Hz, 1H), 5.16 (dd, J=13.4, 2.1 Hz, 1H), 4.90 (dd, J=5.7, 2.1 Hz, 1H), 4.26 (d, J=20.1 Hz, 6H), 1.85 (s, 12H).

¹³C NMR: (126 MHz, cd₂cl₂) δ 182.89, 182.78, 160.00, 153.15, 147.08, 145.70, 143.32, 143.30, 141.31, 141.29, 137.44, 131.52, 130.56, 130.30, 129.74, 129.65, 129.58, 124.68, 122.79, 122.77, 116.09, 114.79, 114.68, 114.54, 112.71, 100.41, 52.87, 52.84, 34.86, 34.74, 26.51, 26.51, 26.08, 26.08.

HRMS: [M]²⁺ m/z calcd. for [C₃₄H₃₆N₂O]²⁺ 244.1408, found 244.1411

8.2.2 Synthesis of Pyridazine compound Pz-1 and cyanine compound Cy-1

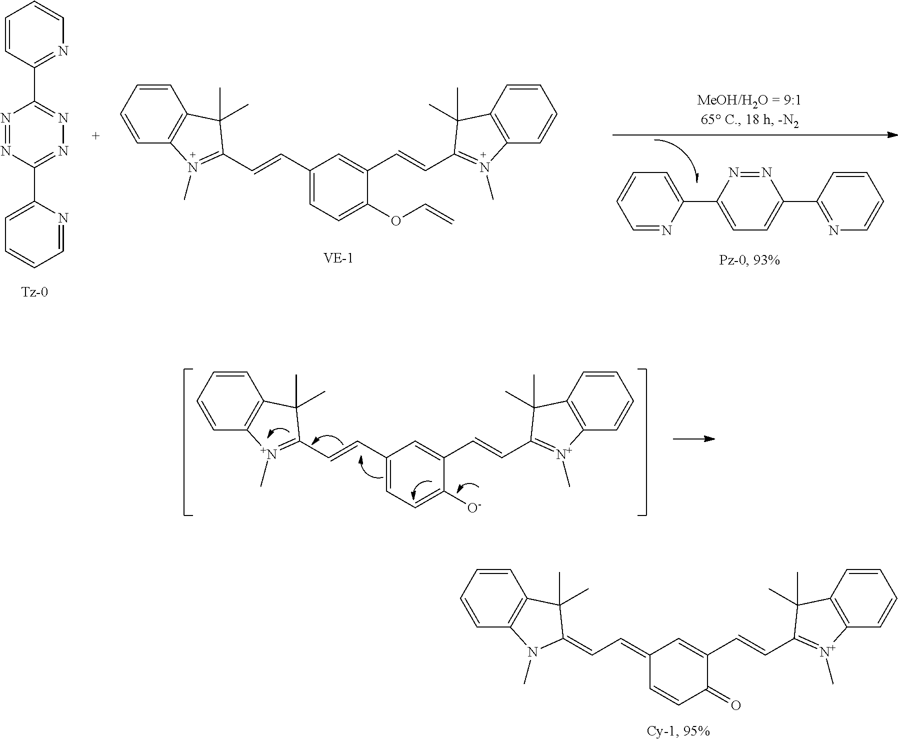

To a 5 mL reaction tube equipped with a stirring bar, 3,6-di-(2-pyridyl)-s-tetrazine Tz-0 (4.7 mg, 0.02 mmol), and VE-1 (9.8 mg, 0.02 mmol), was dissolved in 0.5 mL solution of MeOH/H₂O=9:1, the reaction was heating in oil bath at 65° C. for 18 h, when TLC indicated that the reaction had completed. The reaction was concentrated in vacuo and then residue was purified by preparative TLC (CH₂Cl₂: MeOH=50 : 1), to give Pz-0 (4.4 mg, yield 93%) as a white solid, and Cy-1 (8.5 mg. yield 95%)

‘H NMR of Pz-0: (500 MHz, methylene chloride-d2) δ8.76-8.70 (m, 4H), 8.67 (d, J=0.7 Hz, 2H), 7.95-7.89 (m, 2H), 7.45-7.39 (m, 2H).

‘H NMR of Cy-1: (500 MHz, methylene chloride-d2) δ8.82 (s, 1H), 8.71 (d, J=16.3 Hz, 1H), 8.29 (d, J=15.9 Hz, 1H), 8.08 (d, J=16.0 Hz, 1H), 7.82 (d, J=8.6 Hz, 1H), 7.63-7.49 (m, 9H), 7.46 (d, J=15.9 Hz, 1H), 4.14 (s, 3H), 4.09 (s, 3H), 1.85 (s, 6H), 1.82 (s, 6H).

8.2.3 Synthesis of VE-2

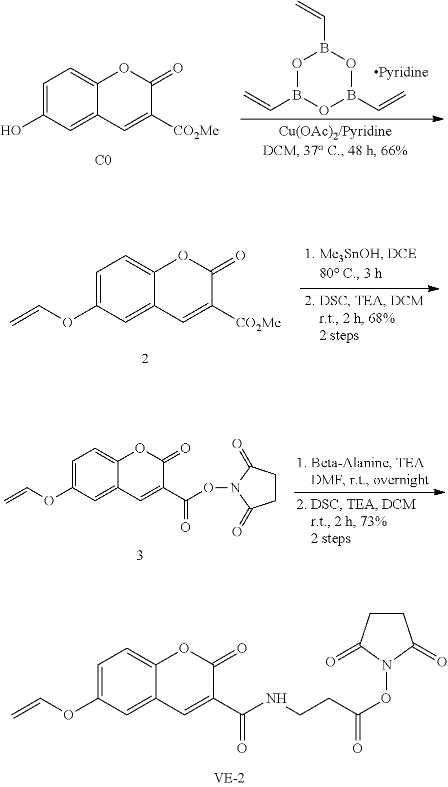

Synthesis of Compound 2:

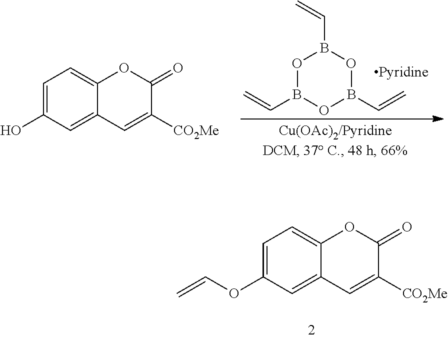 [SMILES: COC(=O)c1cc2cc(O)ccc2oc1=O ; C=CB1OB(C=C)OB(C=C)O1 ; C=COc1ccc2oc(=O)c(C(=O)OC)cc2c1]

In a 25 mL round bottom flask, Cu(OAc)₂ (36 mg, 0 2 mmol) was stirred at room temperature in dry CH₂Cl₂ (5 mL) for 10 min. 2,4,6-Trivinylcyclotriboroxane-pyridine complex (30 mg, 0 2 mmol), Methyl 6-hydroxycoumarin-3-carboxylate C0⁽¹⁾(44 mg, 0.2 mmol), and pyridine (160 mg, 2 mmol) were added and the reaction stirred at room temperature for 48 h. The reaction solution was concentrated in vacuo. The residue was purified by silica column to afford pure 2 as a gray solid 33 mg, 66%.

¹H NMR: (500 MHz, CHLOROFORM-D) δ 8.55 (s, 1H), 7.59-7.54 (m, 1H), 6.98 (dd, J=8.6, 2.4 Hz, 1H), 6.93 (d, J=2.3 Hz, 1H), 6.67 (dd, J=13.5, 6.0 Hz, 1H), 5.02 (dd, J=13.5, 2.0 Hz, 1H), 4.72 (dd, J=5.9, 2.0 Hz, 1H), 3.94 (s, 3H).

¹³C NMR: (126 MHz, CHLOROFORM-D) δ 164.05, 162.06, 157.23, 156.92, 149.26, 145.69, 131.23, 115.14, 114.52, 113.27, 103.54, 99.78, 53.00.

HRMS: [M+Na]⁺ m/z calcd. for [C₁₃ H₁₀ O₅Na]⁻ 269.0420, found 269.0422

Synthesis of Compound 3:

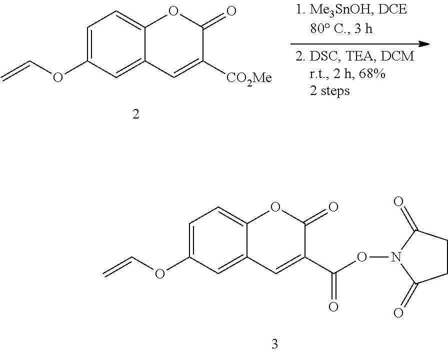 [SMILES: C=COc1ccc2oc(=O)c(C(=O)OC)cc2c1 ; C=COc1ccc2oc(=O)c(C(=O)ON3C(=O)CCC3=O)cc2c1]

In a 10 mL reaction tube equipped with a stir bar, Compound 2 (10.6 mg, 0.04 mmol) was dissolved in 1,2-dichloroethane which Me₃SnOH (20 mg, 0.12 mmol) was added. The solution was heated to 80° C. and stirred for 3 hours. The reaction solution was evaporated and purified by flash column chromatography to afford the carboxylic acid intermediate. The intermediate was dissolved in anhydrous CH₂Cl₂ followed by addition of Et3N (6 uL, 0.044 mmol) and N,N’-disuccinimidyl carbonate (11 mg, 0.044 mmol). This solution was stirred at room temperature for 1 hour. The reaction solution was evaporated and purified by preparative TLC plate to afford Compound 3 (9 mg, yield 68% in 2 steps).

¹H NMR: (500 MHz, CHLOROFORM-D) 6 8.89 (s, 1H), 7.71 (d, J=8.7 Hz, 1H), 7.12 (dd, J=8.7, 2.3 Hz, 1H), 7.05 (d, J=2.3 Hz, 1H), 6.71 (dd, J=13.5, 5.9 Hz, 1H), 5.09 (dd, J=13.5, 2.1 Hz, 1H), 4.81 (dd, J=5.9, 2.1 Hz, 1H), 3.20 (d, J=2.1 Hz, 4H).

¹³C NMR: (126 MHz, CHLOROFORM-D) δ 169.01, 169.01, 163.22, 158.06, 157.83, 155.44, 151.70, 145.16, 131.91, 114.90, 112.58, 109.93, 103.39, 100.51, 25.66, 25.66.

HRMS: [M+Na]⁺m/z calcd. for [C₃₆11₃₃NO₇Na]⁺352.0428, found 352.0425.

Synthesis of VE-2

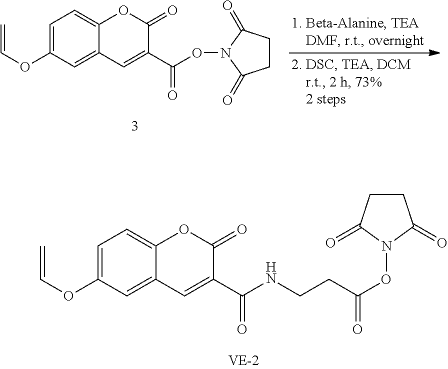 [SMILES: C=COc1ccc2oc(=O)c(C(=O)ON3C(=O)CCC3=O)cc2c1 ; C=COc1ccc2oc(=O)c(C(=O)NCCC(=O)ON3C(=O)CCC3=O)cc2c1]

In a 10-mL reaction tube equipped with a stir bar, Compound 3 (9.9 mg, 0.03 mmol) was dissolved in anhydrous DMF which β-Alanine (3 mg, 0.036 mmol) and TEA (14 uL, 0.11 mmol) was added sequentially. The solution was stirred at room temperature for overnight. Then reaction solution was evaporated and purified by flash column chromatography to afford the carboxylic acid intermediate. The intermediate was dissolved in anhydrous CH₂Cl₂ followed by addition of Et₃N (4.4 uL, 0.032 mmol) and N,N′-disuccinimidyl carbonate (8.2 mg, 0.032 mmol). This solution was stirred at room temperature for 2 hour. The reaction solution was evaporated and purified by preparative TLC plate to afford VE-2 (8.7 mg, yield 73% in 2 steps).

¹H NMR: (500 MHz, CHLOROFORM-D) δ 9.12 (s, 1H), 8.87-8.81 (m, 1H), 7.64 (d, J=8.7 Hz, 1H), 7.02 (dd, J=8.6, 2.3 Hz, 1H), 6.97 (d, J=2.5 Hz, 1H), 6.68 (dd, J=13.5, 6.0 Hz, 1H), 5.02 (dd, J=13.5, 2.0 Hz, 1H), 4.72 (dd, J=5.9, 2.0 Hz, 1H), 3.84 (q, J=6.3 Hz, 2H), 3.00 (t, J=6.4 Hz, 2H), 2.84 (s, 4H).

¹³C NMR: (126 MHz, CHLOROFORM-D) δ 169.05, 167.31, 162.40, 161.75, 161.44, 156.34, 148.39, 145.74, 131.47, 115.67, 114.96, 114.91, 113.96, 103.47, 99.75, 35.26, 31.36, 25.71, 25.71.

HRMS: [M+Na]⁺ m/z calcd. for [C₁₉H₁₆N₂O₈Na]⁺ 423.0799, found 423.0797.

8.2.4 Synthesis of VE-3

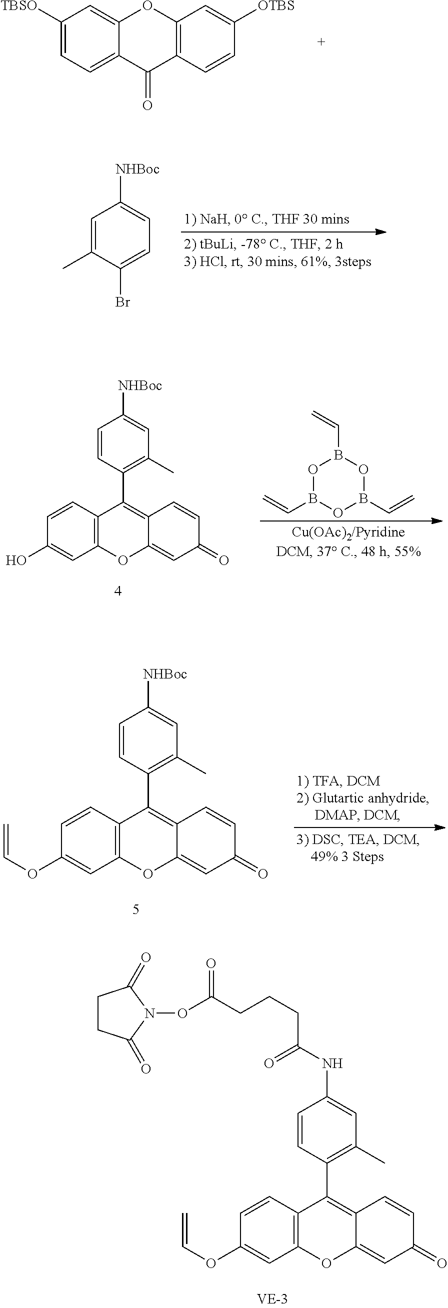 [SMILES: CC(C)(C)[Si](C)(C)Oc1ccc2c(=O)c3ccc(O[Si](C)(C)C(C)(C)C)cc3oc2c1 ; Cc1cc(NC(=O)OC(C)(C)C)ccc1Br ; Cc1cc(NC(=O)OC(C)(C)C)ccc1-c1c2ccc(=O)cc-2oc2cc(O)ccc12 ; C=CB1OB(C=C)OB(C=C)O1 ; C=COc1ccc2c(-c3ccc(NC(=O)OC(C)(C)C)cc3C)c3ccc(=O)cc-3oc2c1 ; C=COc1ccc2c(-c3ccc(NC(=O)CCCC(=O)ON4C(=O)CCC4=O)cc3C)c3ccc(=O)cc-3oc2c1]

Synthesis of Compound 4:

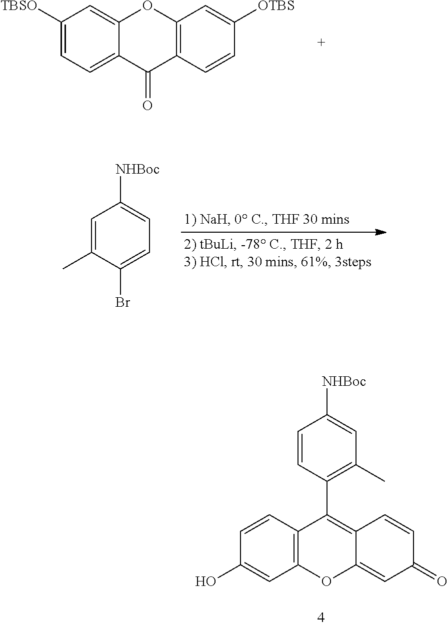 [SMILES: CC(C)(C)[Si](C)(C)Oc1ccc2c(=O)c3ccc(O[Si](C)(C)C(C)(C)C)cc3oc2c1 ; Cc1cc(NC(=O)OC(C)(C)C)ccc1Br ; Cc1cc(NC(=O)OC(C)(C)C)ccc1-c1c2ccc(=O)cc-2oc2cc(O)ccc12]

The synthesis of Compound 4 is similar as previous report (²) (4-Bromo-3-methylphenyl) carbamic acid tert-butyl ester (140 mg, 0.5 mmol) was dissolved in 20 mL anhydrous THF and cooled to 0° C. Then, sodium hydride (14 mg, 0.6 mmol) was added. After stirring for 15 min, the solution was cooled to −78 ° C. Then, tert-butyllithium (1.7 M, 0.59 mL, 1.00 mmol) was added dropwise to the flask. After stirring for 30 min, a solution of 3,6-bis(tert-butyldimethylsilyloxy)-9H-xanthen-9-one (160 mg, 0.35 mmol) in 3 mL anhydrous THF was slowly added into the flask. After stirring for 2 h at −78° C., the reaction was allowed to warm to room temperature over 15 min. The reaction was quenched with the careful addition of 2 M HCl. After stirring for another 15 min, the solvent was removed in vacuo and the remaining residue was purified by silica flash column, yielding Compound 4 (90 mg, 61%) as a yellow solid.

¹H NMR: (500 MHz, METHANOL-D3) δ 7.53 (s, 1H), 7.50 (dd, J=8.3, 2.0 Hz, 1H), 7.13 (m, 7.5 Hz, 3H), 6.77-6.69 (m, 4H), 2.02 (s, 3H), 1.56 (s, 9H).

¹³C NMR: (126 MHz, METHANOL-D3) δ 159.73, 159.28, 156.12, 155.11, 155.11, 142.32, 138.00, 132.47, 132.47, 130.78, 130.78, 129.01, 127.35, 121.17, 117.11, 116.67, 114.81, 104.35, 104.35, 103.19, 81.17, 28.68, 28.68, 28.68, 19.97.

HRMS: [M+H]⁺m/z calcd. for [C₂₅H₂₄NO₅]⁺418.1649, found 418.1648.

Synthesis of Compound 5:

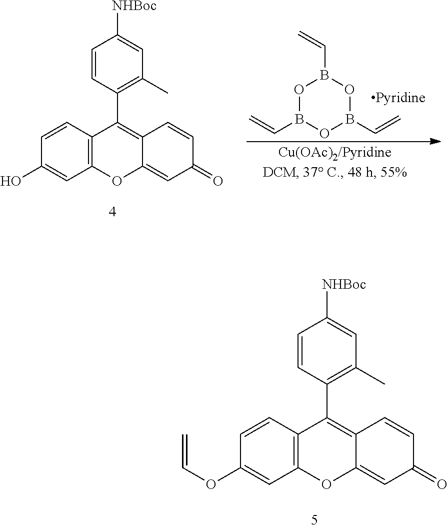 [SMILES: Cc1cc(NC(=O)OC(C)(C)C)ccc1-c1c2ccc(=O)cc-2oc2cc(O)ccc12 ; C=CB1OB(C=C)OB(C=C)O1 ; C=COc1ccc2c(-c3ccc(NC(=O)OC(C)(C)C)cc3C)c3ccc(=O)cc-3oc2c1]

In a 25 mL round bottom flask, Cu(OAc)₂ (18 mg, 0 1 mmol) was stirred at room temperature in dry CH₂Cl₂ (5 mL) for 10 min. 2,4,6-trivinylcyclotriboroxane-pyridine complex (16 mg, 0.07 mmol), Compound 4 (42 mg, 0 1 mmol), and pyridine (80 mg, 1 mmol) were added and the reaction stirred at room temperature for 48 h. The reaction solution was concentrated in vacuo. The residue was purified by silica column to afford pure 5 as a gray solid 25 mg, 55%.

¹H NMR: (500 MHz, CHLOROFORM-D) δ 7.51 (s, 1H), 7.33 (dd, J=8.2, 2.1 Hz, 1H), 7.07 (dd, J=10.7, 7.3 Hz, 2H), 7.05 (s, 1H), 7.00-6.97 (m, 1H), 6.84 (dd, J=8.8, 2.4 Hz, 1H), 6.70 (dd, J=13.6, 6.0 Hz, 1H), 6.66 (s, 1H), 6.57 (dd, J=9.7, 1.9 Hz, 1H), 6.45-6.43 (m, 1H), 5.01 (dd, J=13.5, 2.0 Hz, 1H), 4.70 (dd, J=6.0, 2.0 Hz, 1H), 2.05 (s, 3H), 1.56 (s, 9H).

¹³C NMR: (126 MHz, CHLOROFORM-D) δ 186.06, 161.05, 158.90, 154.24, 152.81, 148.88, 145.96, 139.73, 137.51, 130.83, 130.51, 130.03, 129.93, 126.79, 120.21, 119.50, 116.25, 116.16, 114.12, 106.10, 103.59, 99.32, 81.26, 28.46, 28.46, 28.46, 20.07.

HRMS: [M+H]⁺ m/z calcd. for [C₂₇H₂₆NO₅]⁺ 444.1805, found 444.1806.

Synthesis of VE-3:

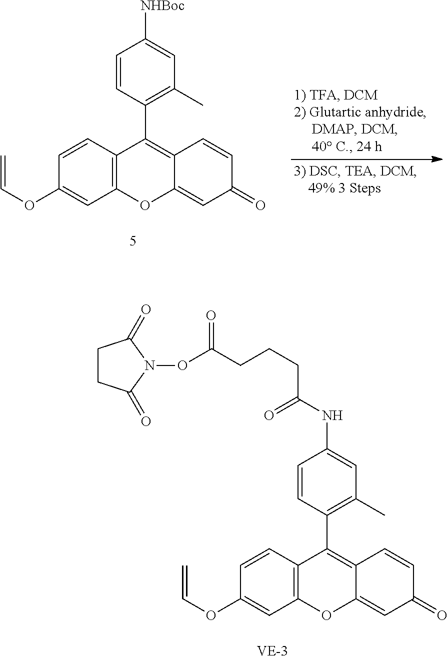 [SMILES: C=COc1ccc2c(-c3ccc(NC(=O)OC(C)(C)C)cc3C)c3ccc(=O)cc-3oc2c1 ; C=COc1ccc2c(-c3ccc(NC(=O)CCCC(=O)ON4C(=O)CCC4=O)cc3C)c3ccc(=O)cc-3oc2c1]

In a 10 mL round bottom flask, Compound 5 (14.5 mg, 0.033 mmol) was stirred at 0° C. in 15% TFA anhydrous CH₂Cl₂ solution (3 mL) for 20 min. After reaction was accomplished, reaction solution was removed in vacuo. The residue was directly used for next step without purification. The residue was dissolved in 5 mL anhydrous DCM, glutaric anhydride (7.6 mg, 0.066 mmol) and DMAP (8.2 mg, 0.066 mmol) was added into reaction solution sequentially. Then the reaction solution was kept at 40° C. for 24 hours. After the reaction was finish, reaction solution was evaporated and purified by flash column chromatography to afford the carboxylic acid intermediate. The intermediate was dissolved in anhydrous CH₂Cl₂ followed by addition of Et₃N (5.0 uL, 0.036 mmol) and N,N′-disuccinimidyl carbonate (10 mg, 0.036 mmol). This solution was stirred at room temperature for 3 hour. The reaction solution was evaporated and purified by preparative TLC plate to afford VE-3 (8.4 mg, yield 49% in 3 steps).

¹H NMR: (500 MHz, CD₃OD) δ 7.69 (s, 1H), 7.63 (d, J=8.2 Hz, 1H), 7.28 (d, J=2.4 Hz, 1H), 7.23-7.11 (m, 3H), 7.03 (dd, J=9.0, 2.4 Hz, 1H), 6.95 (dd, J=13.4, 6.0 Hz, 1H), 6.59 (dd, J=9.7, 2.0 Hz, 1H), 6.45 (d, J=2.0 Hz, 1H), 5.00-4.95 (m, 1H), 4.72-4.68 (m, 1H), 4.60 (s, 1H), 2.82 (d, J=1.7 Hz, 4H), 2.76 (t, J=7.2 Hz, 2H), 2.55 (t, J=7.3 Hz, 2H), 2.14-2.06 (m, 2H), 2.02 (s, 3H).

¹³C NMR: (126 MHz, CD₃OD) δ 187.65, 173.40, 171.90, 171.90, 169.96, 163.46, 161.41, 156.11, 154.19, 147.30, 141.51, 138.24, 132.98, 131.69, 130.89, 130.06, 128.66, 122.80, 119.82, 118.76, 117.28, 115.96, 105.79, 104.25, 99.36, 36.21, 30.86, 26.51, 26.51, 21.57, 19.96.

HRMS: [M+H]⁺ m/z calcd. for [C₃₁ H₂₇ N₂O₈]⁺ 555.1762, found 555.1761.

8.2.5 Synthesis of VE-4

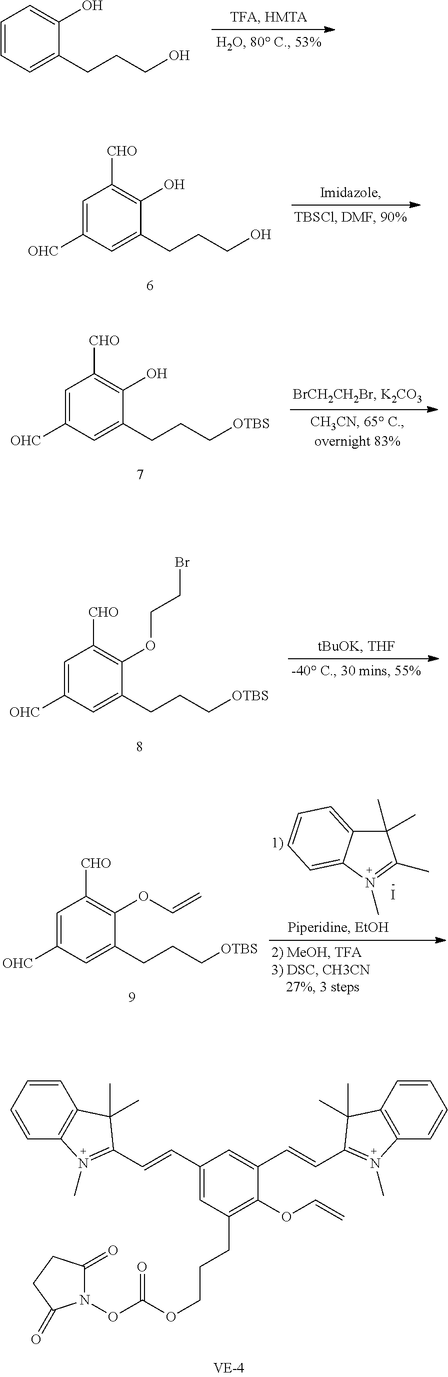

Synthesis of Compound 6

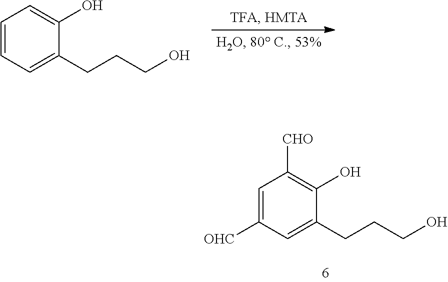 [SMILES: OCCCc1ccccc1O ; O=Cc1cc(C=O)c(O)c(CCCO)c1]

A mixture of 2-(3-Hydroxypropyl)-phenol (910 mg, 6 mmol) and Hexamethylenetetramine (3.5 g, 24.8 mmol) were dissolved in TFA 7 ml. The reaction was refluxed overnight, and cooled to room temperature. 40 ml of water were added and the reaction was heated to 80° C. for 2 hours. After cooling to room temperature the product was extracted by EtOAc (50 mL×5). The organic layer was dried over Na₂SO₄, filtered, and concentrated in vacuo. The residue was purified by silica column (Hexanes: EtOAc =5:1) to afford pure Compound 6 (678 mg, yield 53%) as a colorless liquid.

¹H NMR: (500 MHz, CHLOROFORM-D) δ 11.92 (s, 1H), 9.99 (s, 1H), 9.92 (s, 1H), 8.00 (d, J =2.1 Hz, 1H), 7.97 (d, J=2.0 Hz, 1H), 3.68 (t, J=6.2 Hz, 2H), 2.88-2.81 (m, 2H), 1.97-1.88 (m, 2H).

¹³C NMR: (126 MHz, CHLOROFORM-D) δ 196.37, 189.58, 164.56, 136.75, 134.83, 132.10, 128.97, 119.88, 61.79, 31.96, 25.21.

HRMS: [M−H]⁻ m/z calcd. for [C₁₁H₁₁O₄]⁻ 207.0663, found 207.0664

Synthesis of Compound 7

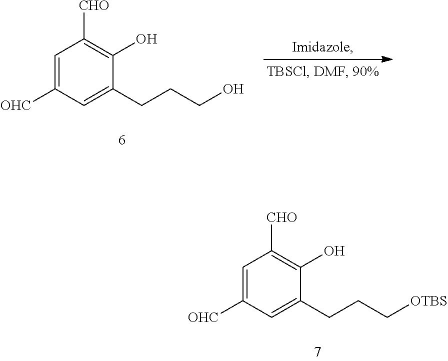 [SMILES: O=Cc1cc(C=O)c(O)c(CCCO)c1 ; CC(C)(C)[Si](C)(C)OCCCc1cc(C=O)cc(C=O)c1O]

Compound 6 (208 mg, 1 mmol) was dissolved in anhydrous DMF (15.0 mL) at 0° C. Imidazole (75 mg, 1.1 mmol) was added followed by TBSCl (165 mg, 1.1 mmol) at 0° C. After addition, the reaction was stirred at room temperature for 30 mins. After the reaction accomplished, 50 ml of EtOAc was added into the reaction solution, and washed by water (50 mL×3). The organic layer was dried over Na₂SO₄, filtered, and concentrated in vacuo. The residue was purified by silica column (Hexanes: EtOAc =30:1) to afford pure 7 (298 mg, yield 90%) as a colorless liquid.

‘H NMR: (500 MHz, CHLOROFORM-D) δ 11.82 (s, 1H), 9.98 (s, 1H), 9.90 (s, 1H), 7.98 (d, J=2.1 Hz, 1H), 7.94 (d, J=1.9 Hz, 1H), 3.65 (t, J=6.2 Hz, 2H), 2.82-2.77 (m, 2H), 1.89-1.82 (m, 2H), 0.90 (s, 9H), 0.04 (s, 6H).

¹³C NMR: (126 MHz, CHLOROFORM-D) δ 196.49, 189.82, 164.87, 136.81, 134.83, 132.65, 128.91, 120.00, 62.40, 31.92, 26.06, 26.06, 26.06, 25.74, 18.43, −5.19, −5.19.

HRMS: [M+H]⁺m/z calcd. for [C₁₇ H₂₇O₄Si]⁺ 345.1493, found 345.1499.

Synthesis of Compound 8

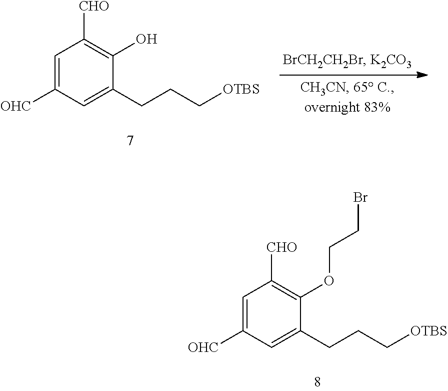 [SMILES: CC(C)(C)[Si](C)(C)OCCCc1cc(C=O)cc(C=O)c1O ; CC(C)(C)[Si](C)(C)OCCCc1cc(C=O)cc(C=O)c1OCCBr]

Compound 7 (161 mg, 0.5 mmol) was dissolved in anhydrous acetonitrile (10.0 mL). 1,2-Dibromoethane (0.94 g, 5 mmol) was added followed by K₂CO₃ (138 mg, 1 mmol) at room temperature. Then the reaction was stirred at 65° C. for Overnight. After the reaction accomplished, the reaction solution was filtered, and concentrated in vacuo. The residue was purified by silica column (Hexanes: EtOAc=30:1) to afford pure Compound 8 (178 mg, yield 83%) as a light yellow oil.

¹H NMR: (500 MHz, CHLOROFORM-D) δ 10.42 (s, 1H), 10.00 (s, 1H), 8.20 (d, J=2.2 Hz, 1H), 8.04 (d, J=2.2 Hz, 1H), 4.40-4.32 (m, 2H), 3.76-3.71 (m, 2H), 3.69 (t, J=6.0 Hz, 2H), 2.90-2.83 (m, 2H), 1.93-1.85 (m, 2H), 0.91 (s, 9H), 0.06 (s, 6H).

¹³C NMR: (126 MHz, CHLOROFORM-D) δ 190.51, 189.21, 163.62, 138.35, 135.88, 132.99, 130.97, 129.81, 75.95, 62.34, 33.13, 29.49, 26.39, 26.11, 26.11, 26.11, 18.50, −5.15, −5.15.

HRMS: [M+H]⁺ m/z calcd. for [C₁₉ H₃₀ BrO₄Si]⁺429.1091, found 429.1092.

Synthesis of Compound 9

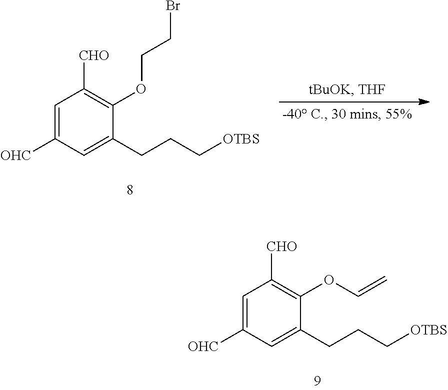 [SMILES: CC(C)(C)[Si](C)(C)OCCCc1cc(C=O)cc(C=O)c1OCCBr ; C=COc1c(C=O)cc(C=O)cc1CCCO[Si](C)(C)C(C)(C)C]

Compound 8 (108 mg, 0.25 mmol) was dissolved in anhydrous THF (5 mL) at −40° C. t-BuOK (0.5 mmol) was slowly added in 10 mins. Then the reaction was stirred at −40° C. for 30 mins. After the reaction accomplished, the reaction was quenched by water, then the reaction solution was extracted by EtOAc (30 mL×3). The organic layer was dried over Na₂SO₄, filtered, and concentrated in vacuo. The residue was purified by silica column (Hexanes: EtOAc=50:1) to afford pure Compound 9 (47 mg, yield 55%) as a light yellow oil.

¹H NMR: (500 MHz, methylene chloride-d2D) δ 10.25 (s, 1H), 10.02 (s, 1H), 8.23 (s, 1H), 8.08 (s, 1H), 6.89-6.75 (m, 1H), 4.38 (dd, J=6.3, 2.8 Hz, 1H), 4.16 (dd, J=14.1, 2.8 Hz, 1H), 3.66 (dd, J=8.2, 3.8 Hz, 2H), 2.84-2.73 (m, 2H), 1.86 (dd, J=7.5, 6.0 Hz, 2H), 0.91 (s, 9H), 0.05 (s, 6H).

¹³C NMR: (126 MHz, methylene chloride-d2D) 6 191.03, 189.07, 160.12, 152.37, 138.72, 136.32, 134.33, 130.19, 129.36, 92.85, 62.73, 33.28, 26.51, 26.24, 26.24, 26.24, 18.67, −5.09, −5.09. HRMS: [M+H]⁺ m/z calcd. for [C₁₉ H_{29 O}₄Si]⁻ 349.1830, found 349.1830.

Synthesis of VE-4

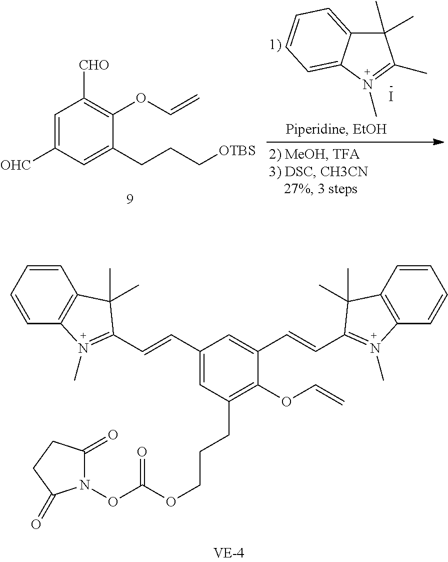

A mixture of compound 9 (7 mg, 0.02 mmol), NaOAc (4 mg, 0.048 mmol), and 1,2,3,3,-tetramethyl-3H-indolium iodide (14.5 mg, 0.048 mmol) was dissolved in 1 mL Ac₂O. The reaction mixture stirred for 30 min at 80° C. under an Ar atmosphere and monitored by HPLC-MS. After reaction completed, the solvent was removed under vacuo. The resulting crude product was dissolved in 2 mL 5% TFA methanol solution. The reaction mixture stirred for lh at room temperature and monitored by HPLC-MS. After reaction completed, the solvent was removed under vacuo. The crude intermediate was dissolved in 1 mL anhydrous CH₃CN followed by addition of N,N′-disuccinimidyl carbonate (10 mg, 0.036 mmol). This solution was stirred at room temperature for 6 hour. Then the reaction solvent was removed under vacuo, the residue was dissolved in MeOH, purified by preparative RP-HPLC to give compound VE-4 (4 mg, 27% for 3 steps) as a yellow solid.

¹ H NMR: (500 MHz, methylene chloride-d2) δ 8.88 (s, 1H), 8.35 (m, 2H), 8.00 (m, 2H), 7.80 (s, 1H), 7.63 (m, 9H), 6.90 (dd, J=14.0, 6.3 Hz, 1H), 4.48 (dd, J=6.1, 2.7 Hz, 1H), 4.39 (t, J=5.8 Hz, 2H), 4.32-4.21 (m, 7H), 2.88 (t, J=7.4 Hz, 2H), 2.83 (s, 4H), 2.17 (d, J=6.8 Hz, 2H), 1.86 (s, 6H), 1.82 (s, 6H).

¹³C NMR: (126 MHz, dichloroethane) δ 181.23, 181.07, 167.31, 167.31, 158.62, 154.96, 151.14, 149.88, 149.33, 149.11, 145.78, 141.78, 139.65, 139.59, 135.83, 135.67, 134.91, 130.77, 129.29, 128.89, 128.32, 128.09, 127.46, 121.31, 121.12, 121.09, 113.20, 112.28, 91.53, 68.78, 51.46, 51.35, 33.55, 33.26, 33.14, 26.79, 24.78, 24.66, 24.58, 24.30, 24.28, 23.95.

HRMS: [M]²⁺ m/z calcd. for [C₄₂ H₄₅N₃O₆]²⁺ 343.6649, found 343.6647.

### Example 8.3

### General Procedure for Oligonucleotide Probe Modification

FIG. 31 provides an exemplary general schematic for oligonucleotide probe modification.

Amine-modified oligonucleotide sequences at selected terminus (1 eq) were dissolved in a 0.1 mM sodium bicarbonate buffer (pH=8.45). A 3 mM solution of the appropriate label (typically Vinylether-NHS (VE-2, VE-3 and VE-4), Tetrazine-NHS (Tz -1)) in DMSO (30 eq) was added in one portion. The reaction was left at 5° C. for 24 hours and reaction progress was monitored by HPLC. The main product formed without significant side reactions.

It was important to carefully purify the oligonucleotide after modification and to lyophilize the sample to remove solvent immediately after HPLC. The purity of all compounds was verified by LC-MS prior to activation experiments.

LC-MS conditions: An Agilent 1260 HPLC system coupled with an Agilent 6230 time of flight mass spectrometer (TOFMS) was employed for LC-TOFMS analysis. The instrument was operated under negative ion mode use Jetstream electrospray ionization (ESI) as the ion source. A Phenomenex Clarity Oligo-MS column (2.1 mm×150 mm, 2.6 um) was used for separation by using TEA: HFIP: H₂O (0.4:30:1000 v/v) as mobile phase A and MeOH as mobile phase B. LC gradient are as followed: Compounds were eluted with a 1 minute with 5% MeOH followed by a 15 minutes gradient to 50% MeOH.

8.3.1 Probe and Template Sequences

Tables 3.1 and 3.2 provide exemplary probe and template series used in the present examples.

TABLE 3.1
Table of probe sequences
Probe Sequences

| | | SEQ ID | | |
|---|---|---|---|---|
| Name | Type | NO: | 5′-Sequence | Modification |
| d31-Tz | DNA | 30 | /5AmMC6/ GAT CTA TGG CGT CAA | Tz-1 |
| d31-VE | DNA | 31 | CGA TTG AAC ACT CCA /3AmMO/ | VE-2, VE-3, |
| | | | | VE-4 |
| r31-Tz | 2′-O- | 32 | /5AmMC6/U*GA*UU*UA*GA*UA*CA | Tz-1 |
| | Methyl | | *UG | |
| r31-VE | RNA | 33 | G*AA*AA*UG*AU*GG*GU*G | VE-4 |
| | with | | /3AmMO/ | |
| | Phosphor | | | |
| | othioate | | | |
| | Bond | | | |

TABLE 3.2
Table of template sequences
Template Sequences

| | | SEQ | |
|---|---|---|---|
| | | ID | |
| Name | Type | NO: | 5′-Sequence |
| d31 | DNA | 34 | 5′-TTG ACG CCA TAG ATC A TGG AGT |
| | | | GTT CAA TCG |
| d31-mis | DNA | 35 | 5′-TTG ACG CCA TAG ATC A TGG AGC |
| | | | GTT CAA TCG |
| mRNA- | RNA | 36 | 5′-CAU GUA UCU AAA UCA G CAC CCA |
| target | | | UCA UUU UC |

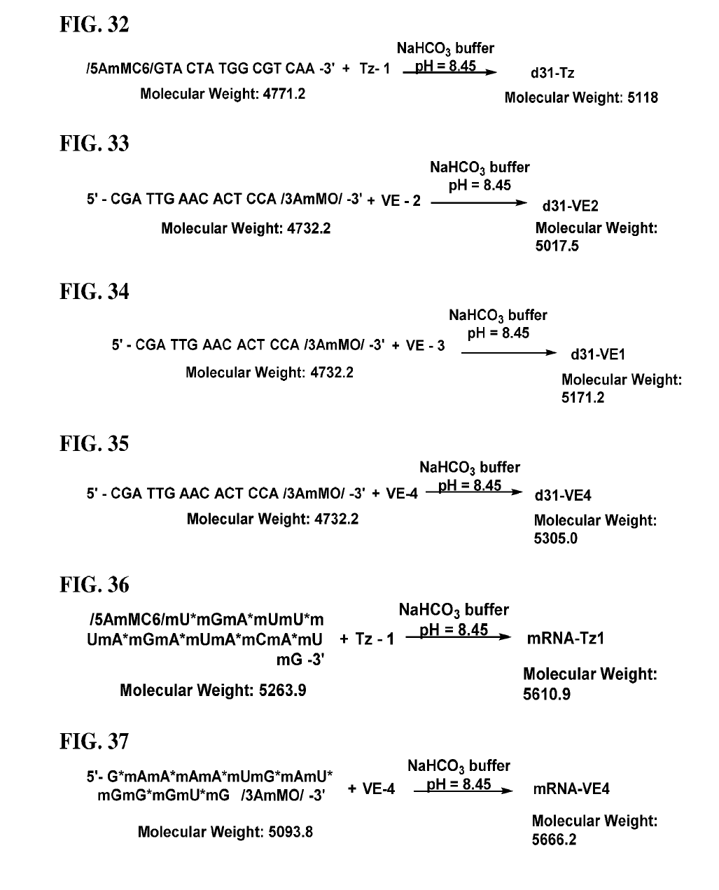

FIG. 32 provides an exemplary scheme for characterization of d31-Tz, which was characterized by HPLC and ESI-TOF-MS.

FIG. 33 provides an exemplary scheme for characterization of d31-VE2, which was characterized by HPLC and ESI-TOF-MS.

FIG. 34 provides an exemplary scheme for characterization of d31-VE3, which was characterized by HPLC and ESI-TOF-MS.

FIG. 35 provides an exemplary scheme for characterization of d31-VE4, which was characterized by HPLC and ESI-TOF-MS.

FIG. 36 provides an exemplary scheme for characterization of mRNA-Tz1, which was characterized by HPLC and ESI-TOF-MS.

FIG. 37 provides an exemplary scheme for characterization of R³¹-VE4, which was characterized by HPLC and ESI-TOF-MS.

### Example 8.4.

### Templated Reaction Kinetic Measurement

A TECAN Genios Pro/384-well multifunction microplate reader was used in the kinetic measurements, and the increase of the fluorescence intensity (excitation at 420 nm) was measured over time. A solution of d31’-Tz and d31′-VE1 at 1 μM was reacted with 1μM of d31 in a 100 mM Tris-HCl (pH=7.4) buffer containing 200 mM MgCl₂, consistent with previous studies. Measurement commenced immediately upon the addition of d27. All the measurements were done at 37° C.

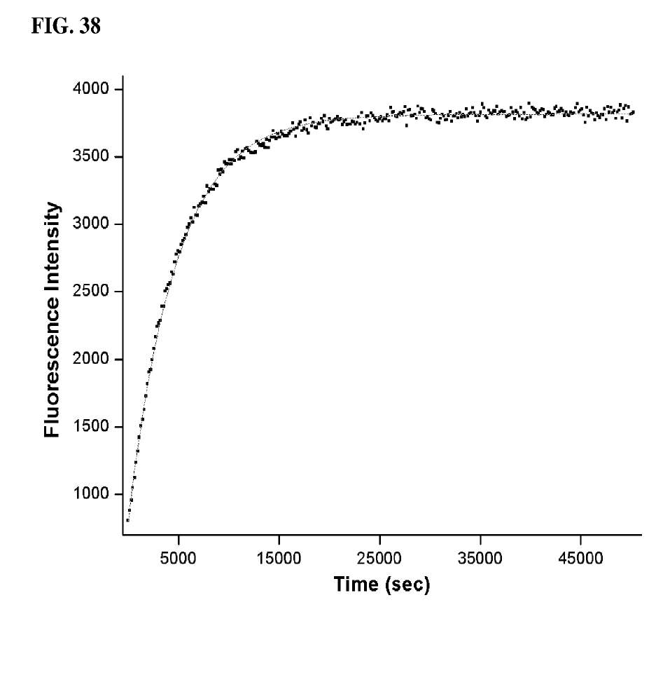

The increase in fluorescence intensity over time is shown in FIG. 38 (black dots). The fluorescence data was fitted with a one-phase exponential association curve (FIG. 38, red line) and the observed first order rate constant was determined to be 0.00021±0.000003 s⁻¹ with a t_{1/2} =54.9 mins.

### Example 8.5

### Fluorescence Turn-On Measurement

Fluorescence emission spectra of template driven reaction. Fluorescence scans were done 1.5 hours after without (blue line in FIG. 2D) and with (red line in FIG. 2D) the adding the DNA template. Reaction conditions: 1μM template d27, 1μM d27′-Tz, and 1μM d27′-ABN in 100 mM Tris-HCl, 200 mM MgCl₂ buffer (pH=7.4) at 25° C. The excitation wavelength used was 480/2 nm.

Activation ratios were calculated from the peak emission intensity of the reaction product and the corresponding baseline intensity, and all the intensity data was background subtracted by just buffer.

### REFERENCES

Mertens M D, Hinz S, Müller C E, Gütschow M. Bioorg. Med. Chem. 2014, 22, 1916-1928.

Teeuwen R. L. M., van Berkel, S S, van Dulmen T H H, Schoffelen S, Meeuwissen S A, Zuilhof H, de Wolf F A, van Hest J C M, Chem. Comm. 2009, 27, 4022-4024.

### REFERENCES (Example 8)

(1) Yang, J.; Karver, M. R.; Li, W.; Sahu, S.; Devaraj, N. K. Angewandte Chemie International Edition in English 2012, 51, 5222.

(2) Wu, H.; Yang, J.; Seckute, J.; Devaraj, N. K. Angewandte Chemie 2014, 53, 5805.

(3) Devaraj, N. K.; Hilderbrand, S.; Upadhyay, R.; Mazitschek, R.; Weissleder, R. Angewandte Chemie International Edition in English 2010, 49, 2869.

(4) Carlson, J. C.; Meimetis, L. G.; Hilderbrand, S. A.; Weissleder, R. Angew Chem Int Ed 2013, 52, 6917.

(5) Meimetis, L. G.; Carlson, J. C. T.; Giedt, R. J.; Kohler, R. H.; Weissleder, R. Angewandte Chemie International Edition 2014, 53, 7531.

(6) Wu, H.; Cisneros, B. T.; Cole, C. M.; Devaraj, N. K. Journal of the American Chemical Society 2014, 136, 17942.

(7) Agarwal, P.; Beahm, B. J.; Shieh, P.; Bertozzi, C. R. Angewandte Chemie International Edition 2015, 54, 11504.

(8) de Silva, A. P.; Gunaratne, H. Q. N.; Gunnlaugsson, T.; Huxley, A. J. M.; McCoy, C. P.; Rademacher, J. T.; Rice, T. E. Chemical Reviews 1997, 97, 1515.

(9) Karton-Lifshin, N.; Segal, E.; Omer, L.; Portnoy, M.; Satchi-Fainaro, R.; Shabat, D. Journal of the American Chemical Society 2011, 133, 10960.

(10) Vaughan, J. C.; Dempsey, G. T.; Sun, E.; Zhuang, X. Journal of the American Chemical Society 2013, 135, 1197.

(11) Cohen, A. S.; Dubikovskaya, E. A.; Rush, J. S.; Bertozzi, C. R. Journal of the American Chemical Society 2010, 132, 8563.

(12) Mitchison, T. J.; Sawin, K. E.; Theriot, J. A.; Gee, K.; Mallavarapu, A. In Methods in Enzymology; Academic Press: 1998; Vol. Volume 291, p 63.

(13) Wysocki, L. M.; Grimm, J. B.; Tkachuk, A. N.; Brown, T. A.; Betzig, E.; Lavis, L. D. Angewandte Chemie International Edition 2011, 50, 11206.

(14) Versteegen, R. M.; Rossin, R.; ten Hoeve, W.; Janssen, H. M.; Robillard, M. S. Angewandte Chemie International Edition 2013, 52, 14112.

(15) Li, J.; Jia, S.; Chen, P. R. Nat Chem Biol 2014, 10, 1003.

(16) Hamasaki, A.; Ducray, R.; Boger, D. L. The Journal of Organic Chemistry 2006, 71, 185.

(17) Boger, D. L.; Sakya, S. M. The Journal of Organic Chemistry 1988, 53, 1415.

(18) Shieh, P.; Bertozzi, C. R. Organic & Biomolecular Chemistry 2014, 12, 9307.

(19) Selvaraj, R.; Fox, J. M. Current Opinion in Chemical Biology 2013, 17, 753.

1. A method of detecting binding of a first affinity ligand and a second affinity ligand, the method comprising
(i) contacting a tetrazine-containing compound with a dienophile-containing compound, said tetrazine-containing compound comprising a first affinity ligand covalently attached to a tetrazine moiety and said dienophile-containing comprising a second affinity ligand covalently attached to a dienophile moiety;
(ii) allowing said first affinity ligand to bind to said second affinity ligand or allowing said first affinity ligand and said second affinity ligand to bind to a third affinity ligand;
(iii) allowing said tetrazine moiety to react with said dienophile moiety to form a pyridazine moiety within a detectable compound, said detectable compound comprising a fluorescent moiety rendered detectable upon formation of said pyridazine moiety.

2.-3. (canceled)

4. The method of claim 1, wherein said dienophile-containing compound comprises a fluorophore and said dienophile moiety is a vinyl ether functional group.

5. The method of claim 4, wherein said fluorophore is a xanthene, a coumarin, or a cyanine group, wherein said xanthene group is a fluorescein or a rhodamine group.

6. (canceled)

7. The method of claim 4, wherein said dienophile-containing compound has a structure according to formula (III), 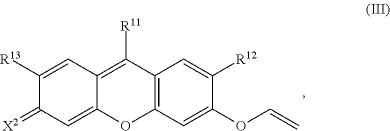
wherein
X² is O or NR^{X2}R^{X2′};
R¹¹ is hydrogen, substituted or unsubsituted alkyl, substituted or unsubstituted heteroalkyl, or -L³-R¹⁴;
R¹² is independently hydrogen, halogen, —N₃, —NO₂, —CF₃, —CCl₃, —CBr₃, —Cl₃, —CN, —OR^{12A}, —NR^{12A}R^{12B}, —COOH, —CONH₂, —NO₂, —SH, —SO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴—R¹⁵;
R¹³ is independently hydrogen, halogen, —N₃, —NO₂, —CF₃, —CCl₃, —CBr₃, —Cl₃, —CN, —OR^{13A}, —NR^{13A}R^{13B}, —COOH, —CONH₂, —NO₂,—SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵;
R¹⁴ is hydrogen or -L⁴-R¹⁵;
L³ is a bond or substituted or unsubstituted arylene;
L⁴ is independently a bond, —NR^{L4}, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁵ is independently said second affinity ligand;
each R^{12A}, R^{12B}R^{13A}, R^{13B}, R^{L4}, R^{X2}, and R^{X2′} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
wherein one of R¹², R¹³, and R¹⁴ is -L⁴-R¹⁵.

8. The method of claim 7, wherein:
R¹¹ is hydrogen, substituted or unsubsituted alkyl, or substituted or unsubstituted heteroalkyl ^{R}¹³ is -L⁴-R¹⁵ ; and ^{R}¹² and R¹³ are independently hydrogen, F, Br, I, or SO₃H.

9. -10. (canceled)

11. The method of claim 7, wherein the compound of formula (III) has the following structure,

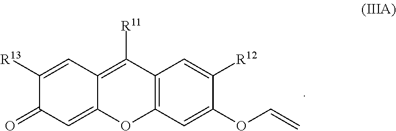

12. The method of claim 11, wherein the compound of formula (IIIA) has the following structure: 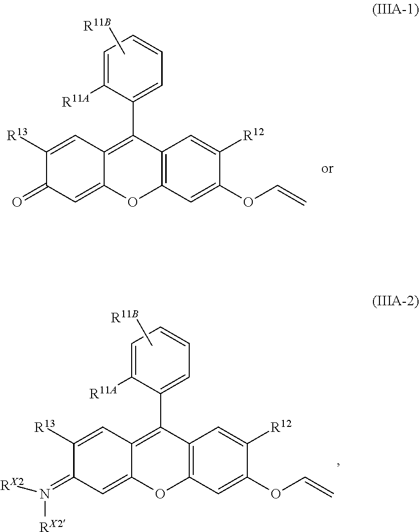
wherein:
R^{11A} is independently hydrogen, substituted or unsubstituted alkyl, —CO₂R^{11C}, or -L⁴-R¹⁵;
R^{11B} is independently hydrogen, halogen, —N₃, —NO₂, —CF₃, —CCl₃, —CBr₃, —Cl₃, —CN, —OR^{11D}, —NR^{11D}R^{11E}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵; and
each R^{11C}, R^{11D}, and R^{11E} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
each R^{X2} and R^{X2′} is independently hydrogen or unsubstituted alkyl.

13.-14. (canceled)

15. The method of claim 4, wherein said dienophile-containing compound has a structure according to formula (IV), 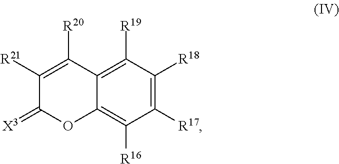
wherein
X³ is O or NR^{X3}R^{X3}′;
R¹⁶ is independently hydrogen, halogen, —N₃, —NO₂, —CF₃, —CCl₃, —CBr₃, —Cl₃, —CN, —OR^{16A}, —NR^{16A}R^{16B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵;
R¹⁷ is independently hydrogen, halogen, —N₃, —NO₂, —CF₃, —CCl₃, —CBr₃, —Cl₃, —CN, —OR^{17A}R^{17B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵;
R¹⁸ is independently hydrogen, halogen, —N₃, —NO₂, —CF₃, —CCl₃, —CBr₃, —Cl₃, —CN, —OR^{18A}, —NR^{18A}R^{18B}, —COOH —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵;
R¹⁹ is independently hydrogen, halogen, —N₃, —NO₂, —CF₃, —CCl₃, —CBr₃, —Cl₃, —CN, —OR^{19A}, —NR^{19A}R^{19B}, —COOH —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —OCH═CH₂, or -L⁴-R¹⁵;
R²⁰ is independently hydrogen, halogen, —N₃, —NO₂, —CF₃, —CCl₃, —CBr₃, —Cl₃, —CN, —OR_{20A}, —NR^{20A}R^{20B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵;
R²¹ is independently hydrogen, halogen, —N₃, —NO₂, —CF₃, —CCl₃, —CBr₃, —Cl₃, —CN, —OR^{21A}, —NR^{21A}R^{21B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴-R¹⁵;
each R^{16A}, R^{16B}, R^{17 A}, R^{17B}, R^{18A}, R^{18B}, R^{19A}, R^{19B}, R^{20A}, R^{20B}, each R^{21A} , and R^{21B} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{X3} and R^{X3}′ is independently hydrogen or substituted or unsubstituted alkyl;
L⁴ is independently a bond, —NR^{L4}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁵ is independently said second affinity ligand;
wherein one of R¹⁶, R¹⁷, R¹⁸, and R¹⁹ is —OCH═CH_{2;} and
wherein one of R¹⁶, R¹⁷, R¹⁸, R¹⁹, R²⁰ and R²¹ is -L‘-R¹⁵.

16. The method of claim 4, wherein said dienophile-containing compound has a structure according to formula (V), 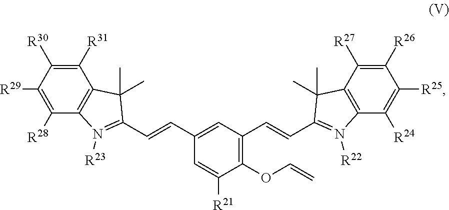
wherein
R²¹ is independently hydrogen, OR^{21A}, NR^{21A}R^{21B}, substituted or unsubstituted alkyl, or -L⁴-R¹⁵;
R²³ and R²⁴ are independently hydrogen, substituted or unsubstituted alkyl, or -L⁴-R¹⁵, and
each R²⁴, R²⁵, R²⁶, R²⁷, R²⁸, R²⁹, R³⁰, and R³¹ is independently hydrogen, halogen, —N₃, —NO₂, —CF₃, —CCl₃, —CBr₃, —Cl₃, —CN, —OH, —NH₂, —NMe₂, —NEt₂, —COOH, —CONH₁₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴—R¹⁵;
each R^{21A} and R^{21B} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is independently a bond, —NR^{L4}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁵ is independently said second affinity ligand; and
wherein one of R²¹, R²², R²³, R²⁴, R²⁵, R²⁶, R²⁷, R²⁸, R²⁹, R³⁰, and R³¹ is -L⁴R¹⁵.

17. The method of claim 16, wherein R²² and R²³ are independently substituted or unsubstituted alkyl.

18. -20. (canceled)

21. The method of claim 4, wherein said dienophile-containing compound has a structure according to formula (VIa) or (VI-b): 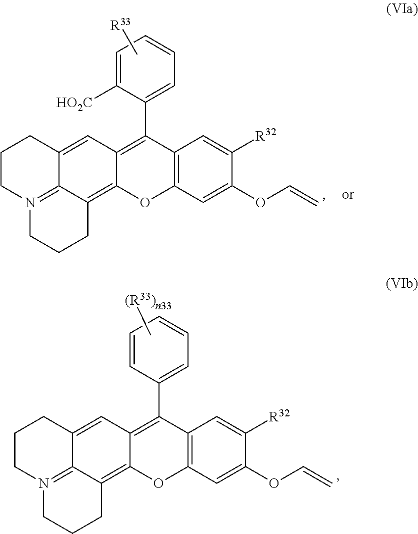
wherein each R³² and R³³ is independently hydrogen, halogen, —N₃, —NO₂, —CF₃, —CCl₃, —CBr₃, —Cl₃, —CN, —OH, —NH₂, —NMe₂, —NEt₂, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L⁴—R¹⁵;
L⁴ is independently a bond, —NW^{L4}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁵ is independently said second affinity ligand;
n33 is 0, 1, or 2, and
wherein one of R³² and R³³ is -L⁴-R¹⁵.

22. The method of claim 4, wherein said tetrazine-containing compound has a structure according to formula (VII): 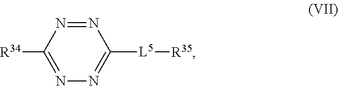
wherein:
R³⁴ is independently hydrogen, halogen, —N₃, —NO₂, —CF₃, —CCl₃, —CBr₃, —Cl₃, —CN, —OR^{34A}, —NR^{34A}, R^{34B}, —COOH, —CONH₂, —NO₂, —SH, —SO₂Cl, —SO₃H, —SO₄H, —SO₂NH₂, NHNH₂, ONH₂, —OCH₃, NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁵ is independently a bond, —NR^{L5}-, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³⁵ is independently said first affinity ligand; and
each R^{34A}, R^{34B}, and R^{L5} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

23. The method of claim 1, wherein said tetrazine-containing compound has a structure according to formula (I): 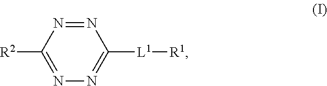
wherein:
L¹ is independently a bond, —NR^{A}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently a binding-detecting group comprising a fluorescent
R² is independently hydrogen, halogen, —N₃, —NO₂, —CF₃, —CCl₃, —CBr₃, —Cl₃, —CN, —OH, —NH₂, —COOH, —CONH₂, —NO₂, —SH, —SO₂Cl, —SO₃H, —SO₄H, —SO₂NH₂, —NHNH₂, ONH₂, —OCH₃, NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
each R^{A} and R^{B} is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

24. The method of claim 23, wherein:
R² is independently hydrogen or substituted or unsubstituted alkyl:
L¹ is independently substituted or unsubstituted alkenylene, and
said fluorescent moiety is a xanthene, cyanine, or a boron-dipyrromethene (BODIPY) group.

25.-27. (canceled)

28. The method of claim 23, wherein the compound of formula (I) has the following structure, 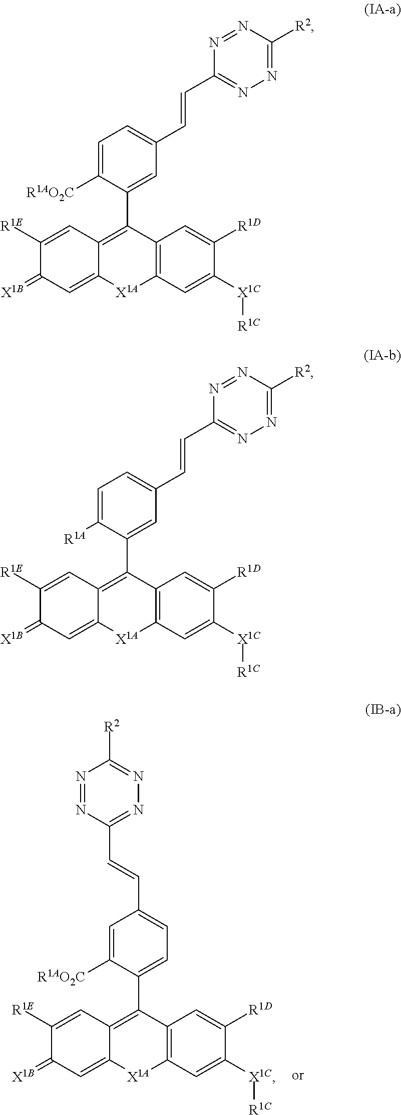 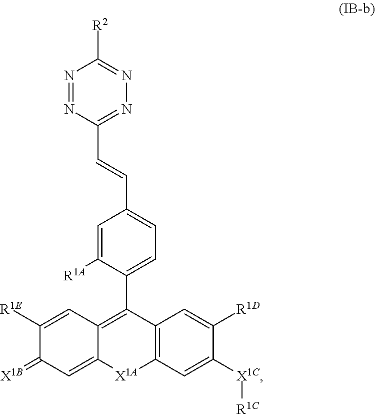
wherein
X^{1A} is independently O, SiMe₂, GeMe₂, SnMe₂, or TeO;
X^{1B} is independently O or NR^{1B}R^{1B′;}
X^{1C} is independently O or NR^{1C′};
R^{1A} is independently hydrogen or substituted or unsubstituted alkyl;
each of R¹B, R^{1B′}, R^{1C}, and R^{1C′} is independently hydrogen or substituted or unsubstituted alkyl;
each of R^{1D} and R^{1B} is independently hydrogen, halogen, —SO₃H, or -L^{R1}-X¹;
L^{R1} is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
X¹ is aid first affinity ligand; and
wherein one of R^{1D} and R^{1E} is -L^{R1}-X¹.

29. The method of claim 28, wherein the compound of formula (I) has the following structure:

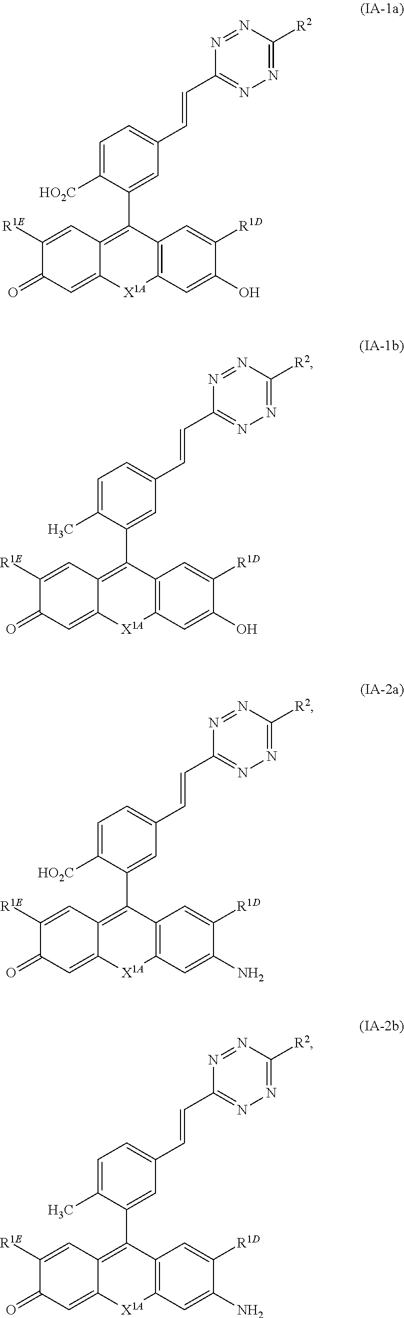

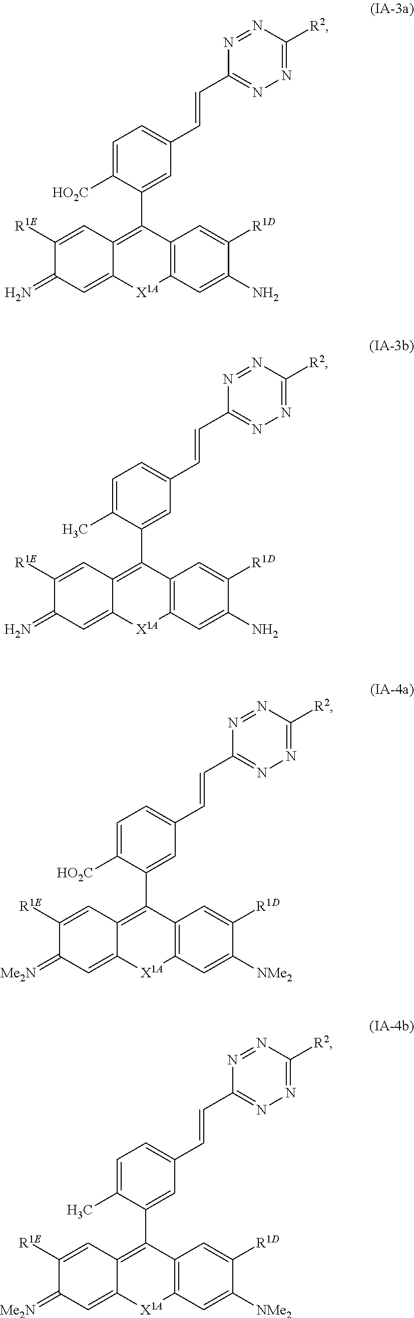

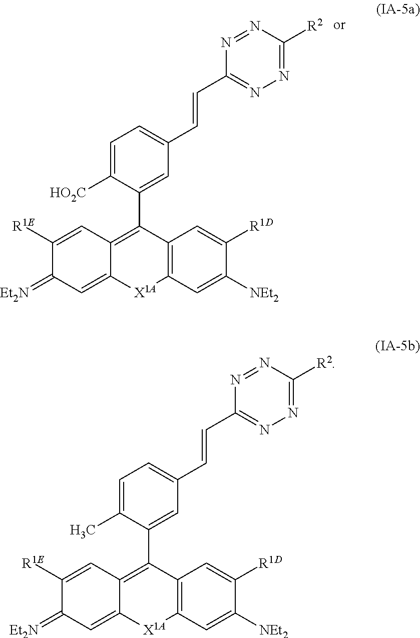

30.-35. (canceled)

36. The method of claim 23, wherein the compound of formula (I) has the following structure: 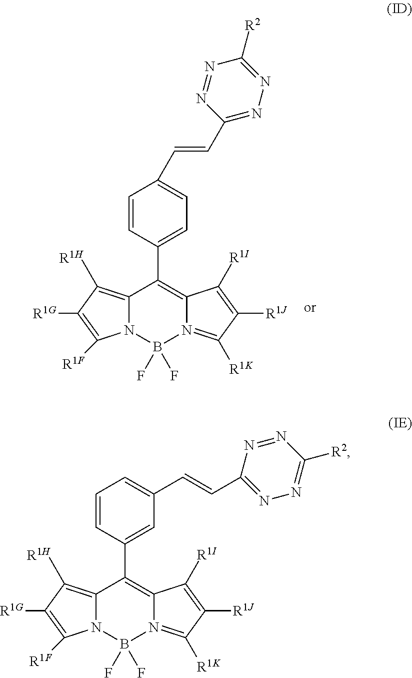
wherein
each R^{1F}, R^{IG}, R^{1H}, R^{1I}, R^{1J}, and R^{1K} is independently hydrogen, halogen, substituted or unsubstituted alkyl, substituted or unsubstituted aryl, substituted or unsubstituted heteroaryl, or -L^{R1}-X¹, and
wherein one of R^{1F}, R^{1G}, R^{1H}, R^{1I}, R^{1J}, and R^{1K} is -L^{R1}-X¹; or 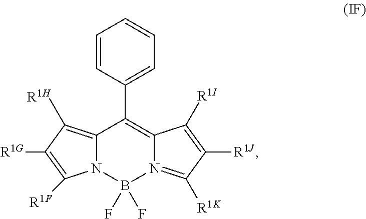
wherein each R^{1F}, R^{IG}, and R^{IH} is independently hydrogen, substituted or unsubstituted alkyl, substituted or unsubstituted aryl, substituted or unsubstituted heteroaryl, -L^{R1}-X¹, 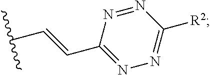
or
each R″, R^{1I}, and R′ is independently hydrogen, substituted or unsubstituted alkyl, substituted or unsubstituted aryl, substituted or unsubstituted heteroaryl, or -L^{R1}-X¹,
L^{R1} is independently a bond, —NR^{B}—, O, S, substituted or unsubstituted alkylene, substituted or unsubstituted alkenylene, substituted or unsubstituted alkynylene, substituted or unsub stituted heteroalkyl ene, substituted or unsub stituted cycl oalkylene, substituted or unsubstituted heterocycloalkylene, substituted or unsubstituted aryl ene, or substituted or unsubstituted heteroarylene;
X¹ is aid first affinity ligand; and
wherein one of R^{1F}, R^{1G}, R^{1H}, R^{1I}, R^{1J}, and R^{1K} is -L^{R1}—X¹; and 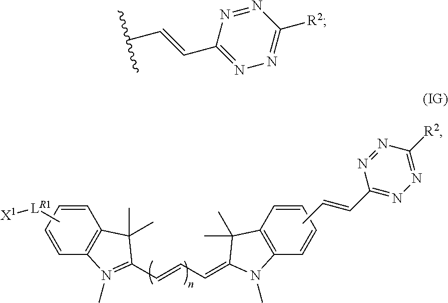
wherein
L^{R1} is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;
X¹ is aid first affinity ligand; and
n is 1, 2, or 3;
or 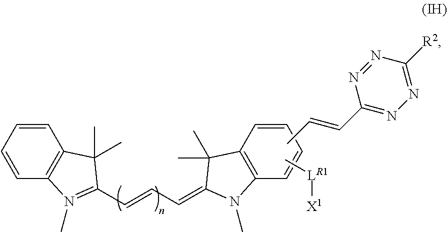
wherein
L^{R1} is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene;
X¹ is aid first affinity ligand; and
n is 1, 2, or 3;
or 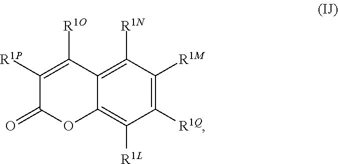
wherein
each R^{1L} R^{1M}, R^{1N},R^{1O} and R^{1P} is independently hydrogen, -L^{R1}-X¹, or 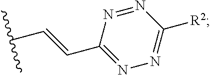
R^{1Q} is independently OR^{1Q′} or NR^{1Q′}R^{1Q″}, and
each R^{1Q′} and R^{1Q″} is independently hydrogen or substituted or unsubstituted alkyl;
L^{R1} is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
X¹ is aid first affinity ligand;
wherein one of R^{1L}, R^{M}, R^{1N}, R^{1O}, and R^{1P} is -L^{R1}-X¹; and
wherein one of _{R}^{1L}, R^{1M}, R^{1N}, R^{1O}, and R^{1P} is

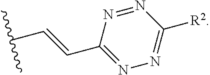

37.-40. (canceled)

41. The method of claim 23, wherein said tetrazine-containing compound has a structure according to formula (I): 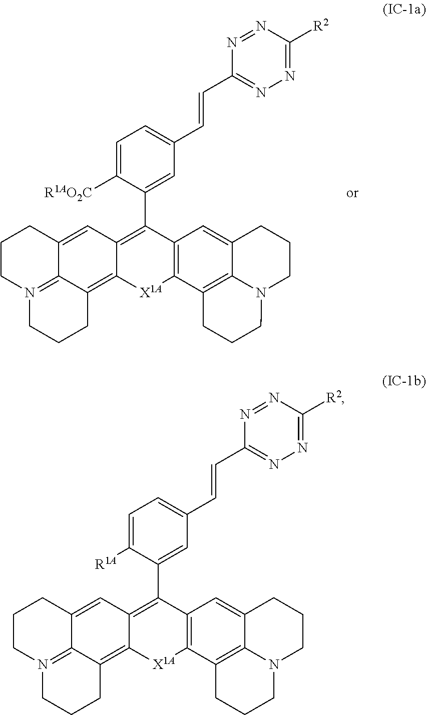
wherein
X^{1A} is independently O, SiMe₂, GeMe₂, SnMe₂, or TeO;
R² is -L^{R1}-X¹;
R^{1A} is independently hydrogen or substituted or insubstituted alkyl;
L^{R1} is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
X¹ is aid first affinity ligand.

42. (canceled)

43. The method of claim 1, wherein said dienophile-containing compound has the following structure, 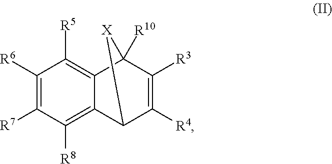
wherein
X is independently O, S, NR^{XA}, or CR^{XB}R^{XC};
each of R³ and R⁴ is independently hydrogen, substituted or unsubstituted alkyl, substituted or unsubstituted heteroalkyl, or -L²-R⁹;
R⁵ is independently hydrogen, halogen, —N₃, —NO₂, —CF₃, —CCl₃, —CBr₃, —Cl₃, —CN, —OR^{5A}, —NR^{5A}R^{5B}, —COOH, —CONH₂, —NO₂, —SH, —SO₂Cl,—SO₃H, —SO₄H, —SO₂NH₂, —NHNH₂, ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²-R⁹;
R⁶ is independently hydrogen, halogen, —N₃, —NO₂, —CF₃, —CCl₃, —CBr₃, —Cl₃, —CN, —OR^{6A}, —NR^{6A}R^{6B},—COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²—R⁹;
R⁷ is independently hydrogen, halogen, —N₃, —NO₂, —CF₃, —CCl₃, —CBr₃, —Cl₃, —CN, —OR^{7A}, —NR^{7A}R^{7B}, —COOH, —CONH₂, —NO₂, —SH, —SO₂Cl, —SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²-R⁹;
R⁸ is independently hydrogen, halogen, —N₃, —NO₂, —CF₃, —CCl₃, —CBr₃, —Cl₃, —CN, —OR^{8A}, —NR^{8A}R^{8B}, —COOH, —CONH₂, —NO₂, —SH, —SO₂Cl,—SO₃H, —SO₄H, —SO₂NH₂, —NHNH₂, —ONH₂, —OCH₃, —NHCNHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²-R⁹;
L² is independently a bond, —NR^{L2}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁹ is independently said second affinity ligand;
R¹⁰ is independently hydrogen —OR^{10A}, —NR^{10A}R^{10B} or substituted or unsubstituted alkyl;
each R^{5A}, R^{5B}, R^{6A}, R^{6B}, R^{7A}, R^{7B}, R^{8A}, R^{8B}, R^{10A}, and R^{10B} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{XA}, R^{XB}, and R^{XC} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or -L²-R⁹;
R^{L2} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
wherein one of R³, R⁴, R⁵, R⁶, R⁷, , R⁸, R^{XA}, R^{XB}, and R^{XC} is -L²-R⁹.

44. The method of claim 43, wherein:
R³, R⁴, and R¹⁰ are hydrogen, and
one of R⁵, R⁶, R⁷, and R⁸ is -L²-R⁹, and the others are hydrogen.

45.-48.(canceled)

49. The method of any one of claim 1, wherein said dienophile-containing compound comprises a first affinity ligand and a second affinity ligand, wherein said first and second affinity ligand that is is a biomolecule or nanomaterial, and wherein said biomolecule is microRNA, telomeres, genomic loci, non-coding RNA, mRNA, a disease associated antibody, a protein of diagnostic utility, or a glycan.

50-118. (canceled)


SEQUENCE LISTING (36 sequences)

SEQ ID NO: 1 - DNA, 27 bp, Artificial Sequence
  Synthetic polynucleotide
ttgacgccatcgaaggtagtgttgaat

SEQ ID NO: 2 - DNA, 21 bp, Artificial Sequence
  Synthetic polynucleotide
acgccatcgaaggtagtgttg

SEQ ID NO: 3 - DNA, 21 bp, Artificial Sequence
  Synthetic polynucleotide
acgccatagaaggtagtattg

SEQ ID NO: 4 - DNA, 21 bp, Artificial Sequence
  Synthetic polynucleotide
aagccatagaaggtagtgttg

SEQ ID NO: 5 - RNA, 22 nt, Artificial Sequence
  Synthetic polynucleotide
uagcuuaucagacugauguuga

SEQ ID NO: 6 - RNA, 22 nt, Artificial Sequence
  Synthetic polynucleotide
uagcuuaucagacugagguuga

SEQ ID NO: 7 - RNA, 22 nt, Artificial Sequence
  Synthetic polynucleotide
uagcuuaugagacugauguuga

SEQ ID NO: 8 - DNA, 13 bp, Artificial Sequence
  Synthetic polynucleotide
  misc_feature (1)..(1): Residue modified with AmMC6
tcgatggcgtcaa

SEQ ID NO: 9 - DNA, 13 bp, Artificial Sequence
  Synthetic polynucleotide
  misc_feature (13)..(13): Residue modified with AmMO
attcaacactacc

SEQ ID NO: 10 - DNA, 10 bp, Artificial Sequence
  Synthetic polynucleotide
  misc_feature (1)..(1): Residue modified with AmMC6
tcgatggcgt

SEQ ID NO: 11 - DNA, 10 bp, Artificial Sequence
  Synthetic polynucleotide
  misc_feature (10)..(10): Residue modified with AmMO
caacactacc

SEQ ID NO: 12 - DNA, 10 bp, Artificial Sequence
  Synthetic polynucleotide
  misc_feature (10)..(10): Residue modified with AmMO
caacactacc

SEQ ID NO: 13 - RNA, 10 nt, Artificial Sequence
  Synthetic polynucleotide
  misc_feature (1)..(10): Residue modified with 2′-0-methyl at ribose
  misc_feature (1)..(1): Residue modified with AmMC6
agauaagcua

SEQ ID NO: 14 - RNA, 11 nt, Artificial Sequence
  Synthetic polynucleotide
  misc_feature (1)..(11): Residue modified with 2′-O-methyl at ribose
  misc_feature (11)..(11): Residue modified with AmMO
ucaacaucagu

SEQ ID NO: 15 - DNA, 13 bp, Artificial Sequence
  Synthetic polynucleotide
  misc_feature (1)..(1): Residue modified with AmMC6
tcgatggcgtcaa

SEQ ID NO: 16 - DNA, 13 bp, Artificial Sequence
  Synthetic polynucleotide
  misc_feature (13)..(13): Residue modified with AmMO
attcaacactacc

SEQ ID NO: 17 - DNA, 10 bp, Artificial Sequence
  Synthetic polynucleotide
  misc_feature (1)..(1): Residue modified with AmMC6
tcgatggcgt

SEQ ID NO: 18 - DNA, 10 bp, Artificial Sequence
  Synthetic polynucleotide
  misc_feature (10)..(10): Residue modified with AmMO
caacactacc

SEQ ID NO: 19 - DNA, 10 bp, Artificial Sequence
  Synthetic polynucleotide
  misc_feature (10)..(10): Residue modified with AmMO
caacactacc

SEQ ID NO: 20 - RNA, 10 nt, Artificial Sequence
  Synthetic polynucleotide
  misc_feature (1)..(10): Residue modified with 2′-O-methyl at ribose
  misc_feature (1)..(1): Residue modified with AmMC6
agauaagcua

SEQ ID NO: 21 - RNA, 11 nt, Artificial Sequence
  Synthetic polynucleotide
  misc_feature (1)..(11): Residue modified with 2′-O-methyl at ribose
  misc_feature (11)..(11): Residue modified with AmMO
ucaacaucagu

SEQ ID NO: 22 - DNA, 27 bp, Artificial Sequence
  Synthetic polynucleotide
ttgacgccatcgaaggtagtgttgaat

SEQ ID NO: 23 - DNA, 21 bp, Artificial Sequence
  Synthetic polynucleotide
acgccatcgaaggtagtgttg

SEQ ID NO: 24 - RNA, 22 nt, Artificial Sequence
  Synthetic polynucleotide
uagcuuaucagacugauguuga

SEQ ID NO: 25 - RNA, 22 nt, Artificial Sequence
  Synthetic polynucleotide
uagcuuaucagacugagguuga

SEQ ID NO: 26 - RNA, 22 nt, Artificial Sequence
  Synthetic polynucleotide
uagcuuaugagacugauguuga

SEQ ID NO: 27 - RNA, 22 nt, Artificial Sequence
  Synthetic polynucleotide
uagcuuaucagacugauguuga

SEQ ID NO: 28 - RNA, 22 nt, Artificial Sequence
  Synthetic polynucleotide
uagcuuaucagacugagguuga

SEQ ID NO: 29 - RNA, 22 nt, Artificial Sequence
  Synthetic polynucleotide
uagcuuaugagacugauguuga

SEQ ID NO: 30 - DNA, 15 bp, Artificial Sequence
  Synthetic polynucleotide
  misc_feature (1)..(1): Residue modified with AmMC6
gatctatggcgtcaa

SEQ ID NO: 31 - DNA, 15 bp, Artificial Sequence
  Synthetic polynucleotide
  misc_feature (15)..(15): Residue moidified with AmMO
cgattgaacactcca

SEQ ID NO: 32 - RNA, 15 nt, Artificial Sequence
  Synthetic polynucleotide
  misc_feature (1)..(15): Residue modiified with 2′-O-methyl at ribose
  misc_feature (1)..(1): Residue modiified with phosphorothioate
  misc_feature (1)..(1): Residue modiified with AmMC6
  misc_feature (3)..(3): Residue modiified with phosphorothioate
  misc_feature (5)..(5): Residue modiified with phosphorothioate
  misc_feature (7)..(7): Residue modiified with phosphorothioate
  misc_feature (9)..(9): Residue modiified with phosphorothioate
  misc_feature (11)..(11): Residue modiified with phosphorothioate
  misc_feature (13)..(13): Residue modiified with phosphorothioate
ugauuuagauacaug

SEQ ID NO: 33 - RNA, 14 nt, Artificial Sequence
  Synthetic polynucleotide
  misc_feature (1)..(14): Residue modified with 2′-O-methyl at ribose
  misc_feature (1)..(1): Residue modified with phosphorothioate
  misc_feature (3)..(3): Residue modified with phosphorothioate
  misc_feature (5)..(5): Residue modified with phosphorothioate
  misc_feature (7)..(7): Residue modified with phosphorothioate
  misc_feature (9)..(9): Residue modified with phosphorothioate
  misc_feature (11)..(11): Residue modified with phosphorothioate
  misc_feature (13)..(13): Residue modified with phosphorothioate
  misc_feature (14)..(14): Residue modified with AmMO
gaaaaugaugggug

SEQ ID NO: 34 - DNA, 31 bp, Artificial Sequence
  Synthetic polynucleotide
ttgacgccatagatcatggagtgttcaatcg

SEQ ID NO: 35 - DNA, 31 bp, Artificial Sequence
  Synthetic polynucleotide
ttgacgccatagatcatggagcgttcaatcg

SEQ ID NO: 36 - RNA, 30 nt, Artificial Sequence
  Synthetic polynucleotide
cauguaucuaaaucagcacccaucauuuuc